SEQUENCE LISTING (50 sequences)

SEQ ID NO: 1 - protein, 992 aa, synthetic construct
  REGION 1..992: note = Synthetic Polypeptide
MTVPSPLADPSIVPDPYPVYADLAQRRPVHWVERLNAWAVLTYADCAAGLKDPRLTADRGTEVLAAKFPGQPLPPDNIFHRWTKNVVMYTDPPLHDALRRSVRAGFTRAAHQHYDQVLQKVAHDLVASIPAGATEIDAVPALAAELPVRSAVHAFGVPEEDLGFLIPRVNTIMTYHSGPKDQPVTQEIILEKLTDLHTYASELLQGMRGKVLPDTVIARLAAAQDGLTETTPEQTVHQLALVFIALFAPTTPGSLSSGTLAFARNPRQVERFLADQACVDNTANEVLRYNASNQFTWRVAAKDVEMGGVRIEAGQTLALFLGSANRDANMFERPNDFDLDRPNSARHLSFGQGVHACLAAQLISLQLKWFYVALLNRFPGIRTAGEPIWNENLEFRSLRSLPLSLRELQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 2 - DNA, 2985 bp, synthetic construct
  misc_feature 1..2985: note = Synthetic Polynucleotide
  source 1..2985: mol_type = other DNA
atgggcatgaccgtgccgagcccgctggctgatccgagcatcgtgccagatccgtacccggtttacgcggatctggcgcaacgtcgcccggtgcactgggttgagcgtctgaacgcctgggcagtgctgacctatgcagattgcgcggcgggtctgaaggacccgcgtttgaccgcggatagaggtaccgaggtgctggcagcgaagtttccgggtcagccactgccgccggataacatctttcaccgttggaccaagaatgtggttatgtacaccgatccgccgctgcatgatgctttgcgtcgcagcgtgcgtgcaggtttcacccgcgcggctcaccaacattatgatcaggtcctgcaaaaagtagcccacgatctggttgcaagcatcccggcgggtgcaaccgagattgatgctgttccagcactggcggcggagctgccggtgcgtagcgcggtgcatgcattcggtgttccggaggaagatttgggttttctgatcccgcgtgtgaacacgattatgacttaccactctggtccgaaggatcagccggttacccaagagatcattctggagaaactgaccgatctgcacacgtatgcgtcggagctgttgcagggtatgcgtggtaaggtcctgccggataccgtaattgcaagactggctgcggcgcaagatggtctgaccgaaaccaccccggaacagacggtccaccaactggcgttggtgttcattgctctgtttgcaccaaccaccccgggtagcctgagcagcggtaccctggcatttgcaagaaacccgcgtcaggtggagcgttttctggccgatcaagcgtgcgttgataacaccgcgaatgaagtgctgcgttacaacgcgtctaatcagttcacctggcgcgtcgccgcgaaggatgtggagatgggtggtgttcgtattgaagctggtcaaaccctggcgctgttcctgggtagcgctaaccgtgatgcaaatatgttcgagcgcccgaacgattttgatctggatcgtccgaacagcgcaagacacctgagcttcggtcagggtgttcatgcgtgtctggctgcacagttgatcagcctgcaactgaaatggttctatgtggcgctgttgaaccgttttccgggtattcgcaccgcgggtgaaccgatttggaatgagaacctggagtttcgtagcctgcgtagcctgccgctgagcctgcgtgagctccagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctgggtaa

SEQ ID NO: 3 - DNA, 1092 bp, synthetic construct
  misc_feature 1..1092: note = Synthetic Polynucleotide
  source 1..1092: mol_type = other DNA
ttggaagaaaaagaaatactctggaacgaagcgaaagcgtttattgccgcatgctatcaggaattgggaaaggaggaggaagtgaaagaccgtctcgcggacattaaaagtgaaattgacctgaccggaagctatgtacatacgaaggaagagctggagcacggagcgaaaatggcttggagaaacagcaaccgctgcatcggcagattgttctggaattcgctgaatgttatcgacagacgagacgtccggacgaaggaggaagtgcgtgatgccctctttcaccatattgaaaccgccaccaataacgggaaaatcagaccgaccattacgattttccctccggaagagaagggtgaaaagcaagtcgagatctggaatcatcagctgatccggtacgctggatatgagtcagacggagaaagaatcggcgacccggcttcctgttccctgacagcagcctgcgaagagctcggctggcgcggagagcgaacggattttgacctgctgccgctcatttttcgcatgaaaggggacgagcagcctgtctggtatgagctgccgcgttcacttgtgattgaggttccaatcacacatccggacatcgaggcgttttctgatttggagctgaagtggtacggcgtgcctattatttctgatatgaagcttgaggtcgggggcattcattataatgccgcgccatttaacggctggtatatgggcacggagatcggagcgagaaacctcgcagatgaaaagcggtacgacaagctcaaaaaagtagcgtccgtgatcggcatcgccgctgattacaatacggatttatggaaggatcaagcgctagttgaattgaataaagctgtgctgcactcgtataaaaagcagggtgtcagcatcgttgaccatcatacagcggcaagccagtttaaacggtttgaagaacaggaggaagaagcgggcagaaagctgacgggggactggacgtggctgattccgccaatttcacccgctgccactcatatcttccaccgctcctatgataactcaatcgttaagccgaactatttttatcaagataagccttatgagtaa

SEQ ID NO: 4 - DNA, 1092 bp, synthetic construct
  misc_feature 1..1092: note = Synthetic Polynucleotide
  source 1..1092: mol_type = other DNA
atggaagaaaaagaaatcctgtggaacgaagccaaagcattcatcgcagcgtgctaccaagaactgggcaaagaagaagaagtcaaagatcgcctggcggacattaaaagtgaaatcgatctgaccggttcctatgttcatacgaaagaagaactggaacacggcgcaaaaatggcttggcgtaacagcaatcgctgcattggtcgtctgttttggaactctctgaatgtgatcgatcgtcgcgacgttcgcacgaaagaagaagtccgtgatgcgctgtttcatcacattgaaaccgccacgaacaatggtaaaatccgtccgaccattacgatcttcccgccggaagaaaaaggcgaaaaacaggttgaaatttggaaccatcaactgatccgctatgcaggctacgaaagcgacggcgaacgtattggtgatccggctagctgctctctgaccgcggcctgtgaagaactgggctggcgtggtgaacgcacggattttgacctgctgccgctgattttccgcatgaaaggtgatgaacagccggtgtggtatgaactgccgcgttctctggtgattgaagttccgatcacccatccggacatcgaagcctttagtgatctggaactgaaatggtacggcgtcccgattatctccgatatgaaactggaagtgggcggtattcactataacgcagctccgttcaatggctggtacatgggcaccgaaatcggcgcgcgcaatctggccgacgaaaaacgttacgataaactgaaaaaagtcgcatcagtgattggtatcgcggccgattacaacacggacctgtggaaagatcaggcactggtggaactgaataaagctgttctgcactcatacaaaaaacaaggcgtttcgattgtggatcatcacaccgcagcttcacagtttaaacgcttcgaagaacaggaagaagaagcgggtcgtaaactgaccggcgattggacgtggctgattccgccgatctcgccggcagcaacccatatcttccaccgctcgtatgacaatagcatcgtgaaaccgaattacttctaccaggacaaaccgtatgaatag

SEQ ID NO: 5 - protein, 363 aa, synthetic construct
  REGION 1..363: note = Synthetic Polypeptide
MEEKEILWNEAKAFIAACYQELGKEEEVKDRLADIKSEIDLTGSYVHTKEELEHGAKMAWRNSNRCIGRLFWNSLNVIDRRDVRTKEEVRDALFHHIETATNNGKIRPTITIFPPEEKGEKQVEIWNHQLIRYAGYESDGERIGDPASCSLTAACEELGWRGERTDFDLLPLIFRMKGDEQPVWYELPRSLVIEVPITHPDIEAFSDLELKWYGVPIISDMKLEVGGIHYNAAPFNGWYMGTEIGARNLADEKRYDKLKKVASVIGIAADYNTDLWKDQALVELNKAVLHSYKKQGVSIVDHHTAASQFKRFEEQEEEAGRKLTGDWTWLIPPISPAATHIFHRSYDNSIVKPNYFYQDKPYE

SEQ ID NO: 6 - DNA, 786 bp, synthetic construct
  misc_feature 1..786: note = Synthetic Polynucleotide
  source 1..786: mol_type = other DNA
atgtataaagatctggaaggtaaagtggtggtgattaccggcagcagcaccggtctgggcaaagcaatggcgattcgttttgcgaccgaaaaagcgaaagtggtggttaactatcgcagcaaagaagaagaagcgaacagcgttctggaagaaattaaaaaagtgggtggcgaagcgattgcggtgaaaggtgatgtgaccgtggaaagcgatgtgattaacctggtgcagagcagcattaaagaatttggcaaactggatgtgatgattaacaatgcgggtatggaaaatccggtgagcagccatgaaatgagcctgagcgattggaacaaagtgattgataccaacctgaccggtgcgtttctgggcagccgtgaagcgattaaatacttcgtggaaaacgatattaaaggcaccgtgattaacatgagcagcgtgcatgaaaaaattccgtggccgctgtttgtgcattatgcagcgagcaaaggcggtatgaaactgatgaccgaaaccctggccctggaatatgcaccgaaaggcattcgtgtgaacaacattggtccgggtgcgattaacaccccgattaacgcggaaaaatttgccgatccggaacagcgtgcggatgtggaaagcatgattccgatgggctatattggcgaaccggaagaaattgcagcggtggcagcgtggctggcaagcagcgaagcgagctatgtgaccggcattaccctgtttgcggatggcggtatgacccagtatccgagctttcaggcaggtcgtggctaa

SEQ ID NO: 7 - protein, 261 aa, synthetic construct
  REGION 1..261: note = Synthetic Polypeptide
MYKDLEGKVVVITGSSTGLGKAMAIRFATEKAKVVVNYRSKEEEANSVLEEIKKVGGEAIAVKGDVTVESDVINLVQSSIKEFGKLDVMINNAGMENPVSSHEMSLSDWNKVIDTNLTGAFLGSREAIKYFVENDIKGTVINMSSVHEKIPWPLFVHYAASKGGMKLMTETLALEYAPKGIRVNNIGPGAINTPINAEKFADPEQRADVESMIPMGYIGEPEEIAAVAAWLASSEASYVTGITLFADGGMTQYPSFQAGRG

SEQ ID NO: 8 - DNA, 8215 bp, synthetic construct
  misc_feature 1..8215: note = Synthetic Polynucleotide
  source 1..8215: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagcccagcccacacgtcttttgcgtatcggcctttttcttctagctgctgcagccataagcgagcgtctgcttcactcacttggtgaacgtcagcatagcttttcataagcgttgcttcaacggcaggtgccatttggcttccgtctccgcaaatatagaagtgcgctccttgatcaagaagttcaatcaatttcttgccgtcttgttccattacgtgctgaacgtatgttttcggctgatttggcatgcgagaaaaagcggtatgaagcgtaatgatgccttcgctttgggcgttttcaagctcttcttgatacagatagtcttcatgaggtgaacggcagccgaagtataaatgtgcttctccaagtgactgtccttgttcttttagctgtttgcgcgcctgcacaaagcctctaaacggcgcgacgcctgttcccggtccgaccatgataagcggcgtttcagggtcttttggcagcgtaaattctgactgcggtgtggaaataaagcacgtaatcgtatctccttcttgcagctcggcaagatagttcgacgcaattcctttatattctccatatccgctccacgcttctcctgagacaacgctgaccgtgatgcttgcttgtttttcatcgacacgaggtgatgaagaaatcgagtaatagcgcgggcgtatgcttggcagaagggcgataaattcgctgaatttcatttcacacgccgggtatttttcaagcagttcaagcattgttaaacgttttgccagcacttgttctttgtaggcttgcttttcaagcaaggcttcaagctctactttatgcggcgggcagaccgttttagcagccattgcgcgaagctgcgtgcgcgtaacaggatcttgaagctccacgtattgcagaagctcttctacggatactgttttagcgagtggcaaatgagctaatttttcttcttctgcttccagacggatttgctgtgatgcatctaggccgaaccttgctgttacacggtttactattccttcatagttgcgaggaataacacctaaatgatctccttcttgataagaagcttcttttggaagttcaatttcaagatgtcgcgtgcttcgtgcactgcctggctgttgaagttctttgcttgctacgacgttcgttgaaaacgcaccgtgcattttcgcaagcggcatatccgcggcgctgtcgacaaattgaagtgaaagagtagatttattatcttcactgttttcaatgtcgaggttaaagtaggctgctacgtcactccacatatgttcacgccattcttcatatgtgccttcaaagtcgtcgcttgcatctgcttcaccgcggtcagcgatgttttctgcccctttagcggcaagcgtttcatcgataaaagcaggcactttttgatacgtagtagcccagtttttatcgccgcatccaaatacggagtagcgaacgccttttacttcatcagcagacgcttggtctaaccagtcgacaaattgctttgcgttatcaggcggatgaccgttataagacgccgttacaattaatacagctccttcgcgcggaagatttccggcgtgtgaatcaagcgttgcgacctgcggtgcaaatcctttgctcattgcaatatctgctaaatcacgcgccgttccttcagctgttcccatatttgaaccgtatagcacaagcagcggcgtattatgagcgttttctgcctttttgcgtacttttttagcagactggagctcacgcaggctcagcggcaggctacgcaggctacgaaactccaggttctcattccaaatcggttcacccgcggtgcgaatacccggaaaacggttcaacagcgccacatagaaccatttcagttgcaggctgatcaactgtgcagccagacacgcatgaacaccctgaccgaagctcaggtgtcttgcgctgttcggacgatccagatcaaaatcgttcgggcgctcgaacatatttgcatcacggttagcgctacccaggaacagcgccagggtttgaccagcttcaatacgaacaccacccatctccacatccttcgcggcgacgcgccaggtgaactgattagacgcgttgtaacgcagcacttcattcgcggtgttatcaacgcacgcttgatcggccagaaaacgctccacctgacgcgggtttcttgcaaatgccagggtaccgctgctcaggctacccggggtggttggtgcaaacagagcaatgaacaccaacgccagttggtggaccgtctgttccggggtggtttcggtcagaccatcttgcgccgcagccagtcttgcaattacggtatccggcaggaccttaccacgcataccctgcaacagctccgacgcatacgtgtgcagatcggtcagtttctccagaatgatctcttgggtaaccggctgatccttcggaccagagtggtaagtcataatcgtgttcacacgcgggatcagaaaacccaaatcttcctccggaacaccgaatgcatgcaccgcgctacgcaccggcagctccgccgccagtgctggaacagcatcaatctcggttgcacccgccgggatgcttgcaaccagatcgtgggctactttttgcaggacctgatcataatgttggtgagccgcgcgggtgaaacctgcacgcacgctgcgacgcaaagcatcatgcagcggcggatcggtgtacataaccacattcttggtccaacggtgaaagatgttatccggcggcagtggctgacccggaaacttcgctgccagcacctcggtacctctatccgcggtcaaacgcgggtccttcagacccgccgcgcaatctgcataggtcagcactgcccaggcgttcagacgctcaacccagtgcaccgggcgacgttgcgccagatccgcgtaaaccgggtacggatctggcacgatgctcggatcagccagcgggctcggcacggtcatcgccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 9 - DNA, 6135 bp, synthetic construct
  misc_feature 1..6135: note = Synthetic Polynucleotide
  source 1..6135: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttttagccacgacctgcctgaaagctcggatactgggtcataccgccatccgcaaacagggtaatgccggtcacatagctcgcttcgctgcttgccagccacgctgccaccgctgcaatttcttccggttcgccaatatagcccatcggaatcatgctttccacatccgcacgctgttccggatcggcaaatttttccgcgttaatcggggtgttaatcgcacccggaccaatgttgttcacacgaatgcctttcggtgcatattccagggccagggtttcggtcatcagtttcataccgcctttgctcgctgcataatgcacaaacagcggccacggaattttttcatgcacgctgctcatgttaatcacggtgcctttaatatcgttttccacgaagtatttaatcgcttcacggctgcccagaaacgcaccggtcaggttggtatcaatcactttgttccaatcgctcaggctcatttcatggctgctcaccggattttccatacccgcattgttaatcatcacatccagtttgccaaattctttaatgctgctctgcaccaggttaatcacatcgctttccacggtcacatcacctttcaccgcaatcgcttcgccacccacttttttaatttcttccagaacgctgttcgcttcttcttctttgctgcgatagttaaccaccactttcgctttttcggtcgcaaaacgaatcgccattgctttgcccagaccggtgctgctgccggtaatcaccaccactttaccttccagatctttatacatggatcccgacccatttgctgtccaccagtcatgctagccatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 10 - DNA, 9371 bp, synthetic construct
  misc_feature 1..9371: note = Synthetic Polynucleotide
  source 1..9371: mol_type = other DNA
ggggaattgtgagcggataacaattcccctctagaaataattttgtttaactttaagaaggagatataccatgggcatgaccgtgccgagcccgctggctgatccgagcatcgtgccagatccgtacccggtttacgcggatctggcgcaacgtcgcccggtgcactgggttgagcgtctgaacgcctgggcagtgctgacctatgcagattgcgcggcgggtctgaaggacccgcgtttgaccgcggatagaggtaccgaggtgctggcagcgaagtttccgggtcagccactgccgccggataacatctttcaccgttggaccaagaatgtggttatgtacaccgatccgccgctgcatgatgctttgcgtcgcagcgtgcgtgcaggtttcacccgcgcggctcaccaacattatgatcaggtcctgcaaaaagtagcccacgatctggttgcaagcatcccggcgggtgcaaccgagattgatgctgttccagcactggcggcggagctgccggtgcgtagcgcggtgcatgcattcggtgttccggaggaagatttgggttttctgatcccgcgtgtgaacacgattatgacttaccactctggtccgaaggatcagccggttacccaagagatcattctggagaaactgaccgatctgcacacgtatgcgtcggagctgttgcagggtatgcgtggtaaggtcctgccggataccgtaattgcaagactggctgcggcgcaagatggtctgaccgaaaccaccccggaacagacggtccaccaactggcgttggtgttcattgctctgtttgcaccaaccaccccgggtagcctgagcagcggtaccctggcatttgcaagaaacccgcgtcaggtggagcgttttctggccgatcaagcgtgcgttgataacaccgcgaatgaagtgctgcgttacaacgcgtctaatcagttcacctggcgcgtcgccgcgaaggatgtggagatgggtggtgttcgtattgaagctggtcaaaccctggcgctgttcctgggtagcgctaaccgtgatgcaaatatgttcgagcgcccgaacgattttgatctggatcgtccgaacagcgcaagacacctgagcttcggtcagggtgttcatgcgtgtctggctgcacagttgatcagcctgcaactgaaatggttctatgtggcgctgttgaaccgttttccgggtattcgcaccgcgggtgaaccgatttggaatgagaacctggagtttcgtagcctgcgtagcctgccgctgagcctgcgtgagctccagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctgggtaaaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggaagaaaaagaaatcctgtggaacgaagccaaagcattcatcgcagcgtgctaccaagaactgggcaaagaagaagaagtcaaagatcgcctggcggacattaaaagtgaaatcgatctgaccggttcctatgttcatacgaaagaagaactggaacacggcgcaaaaatggcttggcgtaacagcaatcgctgcattggtcgtctgttttggaactctctgaatgtgatcgatcgtcgcgacgttcgcacgaaagaagaagtccgtgatgcgctgtttcatcacattgaaaccgccacgaacaatggtaaaatccgtccgaccattacgatcttcccgccggaagaaaaaggcgaaaaacaggttgaaatttggaaccatcaactgatccgctatgcaggctacgaaagcgacggcgaacgtattggtgatccggctagctgctctctgaccgcggcctgtgaagaactgggctggcgtggtgaacgcacggattttgacctgctgccgctgattttccgcatgaaaggtgatgaacagccggtgtggtatgaactgccgcgttctctggtgattgaagttccgatcacccatccggacatcgaagcctttagtgatctggaactgaaatggtacggcgtcccgattatctccgatatgaaactggaagtgggcggtattcactataacgcagctccgttcaatggctggtacatgggcaccgaaatcggcgcgcgcaatctggccgacgaaaaacgttacgataaactgaaaaaagtcgcatcagtgattggtatcgcggccgattacaacacggacctgtggaaagatcaggcactggtggaactgaataaagctgttctgcactcatacaaaaaacaaggcgtttcgattgtggatcatcacaccgcagcttcacagtttaaacgcttcgaagaacaggaagaagaagcgggtcgtaaactgaccggcgattggacgtggctgattccgccgatctcgccggcagcaacccatatcttccaccgctcgtatgacaatagcatcgtgaaaccgaattacttctaccaggacaaaccgtatgaatagctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 11 - DNA, 7214 bp, synthetic construct
  misc_feature 1..7214: note = Synthetic Polynucleotide
  source 1..7214: mol_type = other DNA
ggggaattgtgagcggataacaattcccctctagaaataattttgtttaactttaagaaggagatataccatgggcagcagccatcaccatcatcaccacagccaggatccgatgtataaagatctggaaggtaaagtggtggtgattaccggcagcagcaccggtctgggcaaagcaatggcgattcgttttgcgaccgaaaaagcgaaagtggtggttaactatcgcagcaaagaagaagaagcgaacagcgttctggaagaaattaaaaaagtgggtggcgaagcgattgcggtgaaaggtgatgtgaccgtggaaagcgatgtgattaacctggtgcagagcagcattaaagaatttggcaaactggatgtgatgattaacaatgcgggtatggaaaatccggtgagcagccatgaaatgagcctgagcgattggaacaaagtgattgataccaacctgaccggtgcgtttctgggcagccgtgaagcgattaaatacttcgtggaaaacgatattaaaggcaccgtgattaacatgagcagcgtgcatgaaaaaattccgtggccgctgtttgtgcattatgcagcgagcaaaggcggtatgaaactgatgaccgaaaccctggccctggaatatgcaccgaaaggcattcgtgtgaacaacattggtccgggtgcgattaacaccccgattaacgcggaaaaatttgccgatccggaacagcgtgcggatgtggaaagcatgattccgatgggctatattggcgaaccggaagaaattgcagcggtggcagcgtggctggcaagcagcgaagcgagctatgtgaccggcattaccctgtttgcggatggcggtatgacccagtatccgagctttcaggcaggtcgtggctaaaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggaagaaaaagaaatcctgtggaacgaagccaaagcattcatcgcagcgtgctaccaagaactgggcaaagaagaagaagtcaaagatcgcctggcggacattaaaagtgaaatcgatctgaccggttcctatgttcatacgaaagaagaactggaacacggcgcaaaaatggcttggcgtaacagcaatcgctgcattggtcgtctgttttggaactctctgaatgtgatcgatcgtcgcgacgttcgcacgaaagaagaagtccgtgatgcgctgtttcatcacattgaaaccgccacgaacaatggtaaaatccgtccgaccattacgatcttcccgccggaagaaaaaggcgaaaaacaggttgaaatttggaaccatcaactgatccgctatgcaggctacgaaagcgacggcgaacgtattggtgatccggctagctgctctctgaccgcggcctgtgaagaactgggctggcgtggtgaacgcacggattttgacctgctgccgctgattttccgcatgaaaggtgatgaacagccggtgtggtatgaactgccgcgttctctggtgattgaagttccgatcacccatccggacatcgaagcctttagtgatctggaactgaaatggtacggcgtcccgattatctccgatatgaaactggaagtgggcggtattcactataacgcagctccgttcaatggctggtacatgggcaccgaaatcggcgcgcgcaatctggccgacgaaaaacgttacgataaactgaaaaaagtcgcatcagtgattggtatcgcggccgattacaacacggacctgtggaaagatcaggcactggtggaactgaataaagctgttctgcactcatacaaaaaacaaggcgtttcgattgtggatcatcacaccgcagcttcacagtttaaacgcttcgaagaacaggaagaagaagcgggtcgtaaactgaccggcgattggacgtggctgattccgccgatctcgccggcagcaacccatatcttccaccgctcgtatgacaatagcatcgtgaaaccgaattacttctaccaggacaaaccgtatgaatagctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 12 - DNA, 7959 bp, synthetic construct
  misc_feature 1..7959: note = Synthetic Polynucleotide
  source 1..7959: mol_type = other DNA
ggggaattgtgagcggataacaattcccctgtagaaataattttgtttaactttaataaggagatataccatgggcatgaccgtgccgagcccgctggctgatccgagcatcgtgccagatccgtacccggtttacgcggatctggcgcaacgtcgcccggtgcactgggttgagcgtctgaacgcctgggcagtgctgacctatgcagattgcgcggcgggtctgaaggacccgcgtttgaccgcggatagaggtaccgaggtgctggcagcgaagtttccgggtcagccactgccgccggataacatctttcaccgttggaccaagaatgtggttatgtacaccgatccgccgctgcatgatgctttgcgtcgcagcgtgcgtgcaggtttcacccgcgcggctcaccaacattatgatcaggtcctgcaaaaagtagcccacgatctggttgcaagcatcccggcgggtgcaaccgagattgatgctgttccagcactggcggcggagctgccggtgcgtagcgcggtgcatgcattcggtgttccggaggaagatttgggttttctgatcccgcgtgtgaacacgattatgacttaccactctggtccgaaggatcagccggttacccaagagatcattctggagaaactgaccgatctgcacacgtatgcgtcggagctgttgcagggtatgcgtggtaaggtcctgccggataccgtaattgcaagactggctgcggcgcaagatggtctgaccgaaaccaccccggaacagacggtccaccaactggcgttggtgttcattgctctgtttgcaccaaccaccccgggtagcctgagcagcggtaccctggcatttgcaagaaacccgcgtcaggtggagcgttttctggccgatcaagcgtgcgttgataacaccgcgaatgaagtgctgcgttacaacgcgtctaatcagttcacctggcgcgtcgccgcgaaggatgtggagatgggtggtgttcgtattgaagctggtcaaaccctggcgctgttcctgggtagcgctaaccgtgatgcaaatatgttcgagcgcccgaacgattttgatctggatcgtccgaacagcgcaagacacctgagcttcggtcagggtgttcatgcgtgtctggctgcacagttgatcagcctgcaactgaaatggttctatgtggcgctgttgaaccgttttccgggtattcgcaccgcgggtgaaccgatttggaatgagaacctggagtttcgtagcctgcgtagcctgccgctgagcctgcgtgagctccagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctgggtaaaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggaagaaaaagaaatcctgtggaacgaagccaaagcattcatcgcagcgtgctaccaagaactgggcaaagaagaagaagtcaaagatcgcctggcggacattaaaagtgaaatcgatctgaccggttcctatgttcatacgaaagaagaactggaacacggcgcaaaaatggcttggcgtaacagcaatcgctgcattggtcgtctgttttggaactctctgaatgtgatcgatcgtcgcgacgttcgcacgaaagaagaagtccgtgatgcgctgtttcatcacattgaaaccgccacgaacaatggtaaaatccgtccgaccattacgatcttcccgccggaagaaaaaggcgaaaaacaggttgaaatttggaaccatcaactgatccgctatgcaggctacgaaagcgacggcgaacgtattggtgatccggctagctgctctctgaccgcggcctgtgaagaactgggctggcgtggtgaacgcacggattttgacctgctgccgctgattttccgcatgaaaggtgatgaacagccggtgtggtatgaactgccgcgttctctggtgattgaagttccgatcacccatccggacatcgaagcctttagtgatctggaactgaaatggtacggcgtcccgattatctccgatatgaaactggaagtgggcggtattcactataacgcagctccgttcaatggctggtacatgggcaccgaaatcggcgcgcgcaatctggccgacgaaaaacgttacgataaactgaaaaaagtcgcatcagtgattggtatcgcggccgattacaacacggacctgtggaaagatcaggcactggtggaactgaataaagctgttctgcactcatacaaaaaacaaggcgtttcgattgtggatcatcacaccgcagcttcacagtttaaacgcttcgaagaacaggaagaagaagcgggtcgtaaactgaccggcgattggacgtggctgattccgccgatctcgccggcagcaacccatatcttccaccgctcgtatgacaatagcatcgtgaaaccgaattacttctaccaggacaaaccgtatgaatagctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 13 - DNA, 5802 bp, synthetic construct
  misc_feature 1..5802: note = Synthetic Polynucleotide
  source 1..5802: mol_type = other DNA
ggggaattgtgagcggataacaattcccctgtagaaataattttgtttaactttaataaggagatataccatgggcagcagccatcaccatcatcaccacagccaggatccgatgtataaagatctggaaggtaaagtggtggtgattaccggcagcagcaccggtctgggcaaagcaatggcgattcgttttgcgaccgaaaaagcgaaagtggtggttaactatcgcagcaaagaagaagaagcgaacagcgttctggaagaaattaaaaaagtgggtggcgaagcgattgcggtgaaaggtgatgtgaccgtggaaagcgatgtgattaacctggtgcagagcagcattaaagaatttggcaaactggatgtgatgattaacaatgcgggtatggaaaatccggtgagcagccatgaaatgagcctgagcgattggaacaaagtgattgataccaacctgaccggtgcgtttctgggcagccgtgaagcgattaaatacttcgtggaaaacgatattaaaggcaccgtgattaacatgagcagcgtgcatgaaaaaattccgtggccgctgtttgtgcattatgcagcgagcaaaggcggtatgaaactgatgaccgaaaccctggccctggaatatgcaccgaaaggcattcgtgtgaacaacattggtccgggtgcgattaacaccccgattaacgcggaaaaatttgccgatccggaacagcgtgcggatgtggaaagcatgattccgatgggctatattggcgaaccggaagaaattgcagcggtggcagcgtggctggcaagcagcgaagcgagctatgtgaccggcattaccctgtttgcggatggcggtatgacccagtatccgagctttcaggcaggtcgtggctaaaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggaagaaaaagaaatcctgtggaacgaagccaaagcattcatcgcagcgtgctaccaagaactgggcaaagaagaagaagtcaaagatcgcctggcggacattaaaagtgaaatcgatctgaccggttcctatgttcatacgaaagaagaactggaacacggcgcaaaaatggcttggcgtaacagcaatcgctgcattggtcgtctgttttggaactctctgaatgtgatcgatcgtcgcgacgttcgcacgaaagaagaagtccgtgatgcgctgtttcatcacattgaaaccgccacgaacaatggtaaaatccgtccgaccattacgatcttcccgccggaagaaaaaggcgaaaaacaggttgaaatttggaaccatcaactgatccgctatgcaggctacgaaagcgacggcgaacgtattggtgatccggctagctgctctctgaccgcggcctgtgaagaactgggctggcgtggtgaacgcacggattttgacctgctgccgctgattttccgcatgaaaggtgatgaacagccggtgtggtatgaactgccgcgttctctggtgattgaagttccgatcacccatccggacatcgaagcctttagtgatctggaactgaaatggtacggcgtcccgattatctccgatatgaaactggaagtgggcggtattcactataacgcagctccgttcaatggctggtacatgggcaccgaaatcggcgcgcgcaatctggccgacgaaaaacgttacgataaactgaaaaaagtcgcatcagtgattggtatcgcggccgattacaacacggacctgtggaaagatcaggcactggtggaactgaataaagctgttctgcactcatacaaaaaacaaggcgtttcgattgtggatcatcacaccgcagcttcacagtttaaacgcttcgaagaacaggaagaagaagcgggtcgtaaactgaccggcgattggacgtggctgattccgccgatctcgccggcagcaacccatatcttccaccgctcgtatgacaatagcatcgtgaaaccgaattacttctaccaggacaaaccgtatgaatagctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 14 - protein, 406 aa, synthetic construct
  REGION 1..406: note = Synthetic Polypeptide
MTVPSPLADPSIVPDPYPVYADLAQRRPVHWVERLNAWAVLTYADCAAGLKDPRLTADRGTEVLAAKFPGQPLPPDNIFHRWTKNVVMYTDPPLHDALRRSVRAGFTRAAHQHYDQVLQKVAHDLVASIPAGATEIDAVPALAAELPVRSAVHAFGVPEEDLGFLIPRVNTIMTYHSGPKDQPVTQEIILEKLTDLHTYASELLQGMRGKVLPDTVIARLAAAQDGLTETTPEQTVHQLALVFIALFAPTTPGSLSSGTLAFARNPRQVERFLADQACVDNTANEVLRYNASNQFTWRVAAKDVEMGGVRIEAGQTLALFLGSANRDANMFERPNDFDLDRPNSARHLSFGQGVHACLAAQLISLQLKWFYVALLNRFPGIRTAGEPIWNENLEFRSLRSLPLSLR

SEQ ID NO: 15 - protein, 572 aa, synthetic construct
  REGION 1..572: note = Synthetic Polypeptide
AHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 16 - protein, 25 aa, synthetic construct
  REGION 1..25: note = Synthetic Polypeptide
KIPLGGIPSPSTEQSAKKVRKKAEN

SEQ ID NO: 17 - DNA, 26 bp, synthetic construct
  misc_feature 1..26: note = Synthetic Polynucleotide
  source 1..26: mol_type = other DNA
ataccatggtgaccgtcccctcgccg

SEQ ID NO: 18 - DNA, 30 bp, synthetic construct
  misc_feature 1..30: note = Synthetic Polynucleotide
  source 1..30: mol_type = other DNA
atcaagcttcccagcccacacgtcttttgc

SEQ ID NO: 19 - DNA, 39 bp, synthetic construct
  misc_feature 1..39: note = Synthetic Polynucleotide
  source 1..39: mol_type = other DNA
caggatccgatgtataaagatctggaaggtaaagtggtg

SEQ ID NO: 20 - DNA, 30 bp, synthetic construct
  misc_feature 1..30: note = Synthetic Polynucleotide
  source 1..30: mol_type = other DNA
caaagcttttagccacgacctgcctgaaag

SEQ ID NO: 21 - DNA, 27 bp, synthetic construct
  misc_feature 1..27: note = Synthetic Polynucleotide
  source 1..27: mol_type = other DNA
actcatatgatggaagaaaaagaaatc

SEQ ID NO: 22 - DNA, 27 bp, synthetic construct
  misc_feature 1..27: note = Synthetic Polynucleotide
  source 1..27: mol_type = other DNA
actaagcttctattcatacggtttgtc

SEQ ID NO: 23 - DNA, 26 bp, synthetic construct
  misc_feature 1..26: note = Synthetic Polynucleotide
  source 1..26: mol_type = other DNA
ctacatatggtgactttcgaagtcgc

SEQ ID NO: 24 - DNA, 28 bp, synthetic construct
  misc_feature 1..28: note = Synthetic Polynucleotide
  source 1..28: mol_type = other DNA
ctcaagcttctgatgagggtaaaagttg

SEQ ID NO: 25 - DNA, 30 bp, synthetic construct
  misc_feature 1..30: note = Synthetic Polynucleotide
  source 1..30: mol_type = other DNA
actcatatggtgactttcgaagtcgccctg

SEQ ID NO: 26 - DNA, 31 bp, synthetic construct
  misc_feature 1..31: note = Synthetic Polynucleotide
  source 1..31: mol_type = other DNA
actaagcttctgatgagggtaaaagttgggg

SEQ ID NO: 27 - DNA, 70 bp, synthetic construct
  misc_feature 1..70: note = Synthetic Polynucleotide
  source 1..70: mol_type = other DNA
acatccttatagccactctgtagtattaattaaacttctttaagttttgcattccggggatccgtcgacc

SEQ ID NO: 28 - DNA, 70 bp, synthetic construct
  misc_feature 1..70: note = Synthetic Polynucleotide
  source 1..70: mol_type = other DNA
aatattcacagggatcactgtaattaaaataaatgaaggattatgtaatgtgtaggctggagctgcttcg

SEQ ID NO: 29 - DNA, 70 bp, synthetic construct
  misc_feature 1..70: note = Synthetic Polynucleotide
  source 1..70: mol_type = other DNA
tacaaccgggggaggcattttgcttcccccgctaacaatggcgacatattgtgtaggctggagctgcttc

SEQ ID NO: 30 - DNA, 67 bp, synthetic construct
  misc_feature 1..67: note = Synthetic Polynucleotide
  source 1..67: mol_type = other DNA
attcggtgcacgatgcctgatgcgccacgtcttatcaggcctacaaaacatatgaatatcctcctta

SEQ ID NO: 31 - DNA, 70 bp, synthetic construct
  misc_feature 1..70: note = Synthetic Polynucleotide
  source 1..70: mol_type = other DNA
ggcctcccaaatcggggggccttttttattgataacaaaaaggcaacactgtgtaggctggagctgcttc

SEQ ID NO: 32 - DNA, 69 bp, synthetic construct
  misc_feature 1..69: note = Synthetic Polynucleotide
  source 1..69: mol_type = other DNA
gccagtaataatccagtgccggatgattcacatcatccggcaccttttcacatatgaatatcctcctta

SEQ ID NO: 33 - DNA, 70 bp, synthetic construct
  misc_feature 1..70: note = Synthetic Polynucleotide
  source 1..70: mol_type = other DNA
tcaggatctgaacgggcagctgacggctcgcgtggcttaagaggtttattgtgtaggctggagctgcttc

SEQ ID NO: 34 - DNA, 69 bp, synthetic construct
  misc_feature 1..69: note = Synthetic Polynucleotide
  source 1..69: mol_type = other DNA
caacctgatgaaaaggtgccggatgatgtgaatcatccggcactggattacatatgaatatcctcctta

SEQ ID NO: 35 - DNA, 26 bp, synthetic construct
  misc_feature 1..26: note = Synthetic Polynucleotide
  source 1..26: mol_type = other DNA
ataccatggtgaccgtcccctcgccg

SEQ ID NO: 36 - DNA, 30 bp, synthetic construct; identical to SEQ ID NO: 18
  misc_feature 1..30: note = Synthetic Polynucleotide
  source 1..30: mol_type = other DNA

SEQ ID NO: 37 - DNA, 39 bp, synthetic construct; identical to SEQ ID NO: 19
  misc_feature 1..39: note = Synthetic Polynucleotide
  source 1..39: mol_type = other DNA

SEQ ID NO: 38 - DNA, 30 bp, synthetic construct; identical to SEQ ID NO: 20
  misc_feature 1..30: note = Synthetic Polynucleotide
  source 1..30: mol_type = other DNA

SEQ ID NO: 39 - DNA, 27 bp, synthetic construct
  misc_feature 1..27: note = Synthetic Polynucleotide
  source 1..27: mol_type = other DNA
actcatatgatggaagaaaaagaaatc

SEQ ID NO: 40 - DNA, 27 bp, synthetic construct
  misc_feature 1..27: note = Synthetic Polynucleotide
  source 1..27: mol_type = other DNA
actaagcttctattcatacggtttgtc

SEQ ID NO: 41 - DNA, 32 bp, synthetic construct
  misc_feature 1..32: note = Synthetic Polynucleotide
  source 1..32: mol_type = other DNA
actggatccgatggaaaactttaaacatctcc

SEQ ID NO: 42 - DNA, 33 bp, synthetic construct
  misc_feature 1..33: note = Synthetic Polynucleotide
  source 1..33: mol_type = other DNA
actgaattcgaaacttctttaagttttgcggtg

SEQ ID NO: 43 - DNA, 70 bp, synthetic construct; identical to SEQ ID NO: 29
  misc_feature 1..70: note = Synthetic Polynucleotide
  source 1..70: mol_type = other DNA

SEQ ID NO: 44 - DNA, 67 bp, synthetic construct; identical to SEQ ID NO: 30
  misc_feature 1..67: note = Synthetic Polynucleotide
  source 1..67: mol_type = other DNA

SEQ ID NO: 45 - DNA, 70 bp, synthetic construct; identical to SEQ ID NO: 31
  misc_feature 1..70: note = Synthetic Polynucleotide
  source 1..70: mol_type = other DNA

SEQ ID NO: 46 - DNA, 69 bp, synthetic construct; identical to SEQ ID NO: 32
  misc_feature 1..69: note = Synthetic Polynucleotide
  source 1..69: mol_type = other DNA

SEQ ID NO: 47 - DNA, 70 bp, synthetic construct; identical to SEQ ID NO: 33
  misc_feature 1..70: note = Synthetic Polynucleotide
  source 1..70: mol_type = other DNA

SEQ ID NO: 48 - DNA, 69 bp, synthetic construct; identical to SEQ ID NO: 34
  misc_feature 1..69: note = Synthetic Polynucleotide
  source 1..69: mol_type = other DNA

SEQ ID NO: 49 - DNA, 59 bp, synthetic construct
  misc_feature 1..59: note = Synthetic Polynucleotide
  source 1..59: mol_type = other DNA
ggatcactgtaattaaaataaatgaaggattatgtaatggtgtaggctggagctgcttc

SEQ ID NO: 50 - DNA, 58 bp, synthetic construct
  misc_feature 1..58: note = Synthetic Polynucleotide
  source 1..58: mol_type = other DNA
gtggctaacatccttatagccactctgtagtattaattacatatgaatatcctcctta

(19) United States
(12) Patent Application Publication
(10) Pub. No.: US 2025/0263760 A1
(43) Pub. Date: Aug. 21, 2025

(54) MODIFIED BACTERIA FOR PRODUCTION OF NITROAROMATICS

(71) Applicant: University of Florida Research Foundation, Incorporated, Gainesville, FL (US)

(72) Inventors: Yousong Ding, Gainesville, FL (US); Ran Zuo, Gainesville, FL (US)

(73) Assignee: University of Florida Research Foundation, Incorporated, Gainesville, FL (US)

(21) Appl. No.: 19/198,441

(22) Filed: May 5, 2025

Related U.S. Application Data
(63) Continuation of application No. 18/529,216, filed on Dec. 5, 2023, now Pat. No. 12,319,946.
Application No. 18/529,216 is a division of application No. 18/152,329, filed on Jan. 10, 2023, now Pat. No. 11,879,144.
Application No. 18/152,329 is a division of application No. 16/982,087, filed on Sep. 18, 2020, now Pat. No. 11,591,627.
(60) Provisional application No. 62/818,024, filed on Mar. 13, 2019.
(60) Provisional application No. 62/645,873, filed on Mar. 21, 2018.

(51) Int. Cl. C12P 13/22; C12N 9/02; C12N 9/04; C12N 15/62

(52) U.S. Cl. CPC C12P 13/227; C12N 9/0006; C12N 9/0042; C12N 9/0075; C12N 15/62; C12Y 101/9901; C12Y 106/02004; C12Y 114/13039

(57) ABSTRACT

The disclosure relates, in some aspects, to compositions and methods useful for production of nitrated aromatic molecules. The disclosure is based, in pan, on whole cell systems expressing artificial fusion proteins comprising cytochrome P450 enzymes linked to reductase enzymes. In some aspects, the disclosure relates to methods of producing nitrated aromatic molecules in whole cell systems having artificial fusion proteins comprising cytochrome P450 enzymes linked to reductase enzymes.

### RELATED APPLICATIONS

This Application claims the benefit under 35 U.S.C. § 119(e) of the filing date of U.S. Provisional Application Ser. No. 62/645,873, filed Mar. 21, 2018, entitled “WHOLE CELL PROCESSES TO PRODUCE NITROAROMATICS”, AND 62/818,024, filed Mar. 13, 2019, entitled “DIRECT AROMATIC NITRATION SYSTEM FOR SYNTHESIS OF NITROTRYPTOPHANS IN ESCHERICHIA COLI”, the entire contents of each of which are incorporated by reference herein.

### GOVERNMENT SUPPORT

This invention was made with government support under FA9550-16-1-0186 awarded by the United States Air Force. The government has certain rights in the invention.

### BACKGROUND

The nitro (—NO₂) group acts as an essential unit in a number of pharmaceuticals, exemplified by anticancer drug nilutamine, anti-Parkinson agent tolcapone, and anti-infective agents chloramphenicol and the recently approved delamanid and nifurtimox-eflornithine combination. Drug candidates bearing the —NO₂ group also commonly appear in drug pipelines for treating a variety of existing and emerging diseases. Additionally, the nitro group in particular is a versatile synthetic handle present in numerous building blocks in the synthesis of complex drug molecules. The fundamental importance of the nitro group in pharmaceutical industry has driven the development of chemical nitration methods. Classical electrophilic nitration methods with nitric acid as the nitrating reagent dominate current industrial processes. The limitations of the electrophilic method, however, is that it is generally non-selective, poorly tolerates other functional groups, potentially raises safety concerns, and generates large quantities of acidic waste.

### SUMMARY

The disclosure relates, in some aspects, to compositions and methods useful for production of nitrated aromatic molecules. The disclosure is based, in part, on whole cell systems expressing artificial fusion proteins comprising cytochrome P450 enzymes linked to reductase enzymes. In some aspects, the disclosure relates to methods of producing nitrated aromatic molecules in whole cell systems having artificial fusion proteins comprising cytochrome P450 enzymes linked to reductase enzymes. In some aspects, the disclosure relates to methods of producing nitrated tryptophan molecules in whole cell systems having artificial fusion proteins comprising cytochrome P450 enzymes linked to reductase enzymes.

One significant advantage of whole cell nitration systems described by the disclosure compared to in vitro nitration reactions is the in situ production of NO from L-Arg. Typically, expensive NO donors are a major barrier for industrial application of nitration biocatalysts (e.g., TxtE fusion proteins, for example TB14). With the help of functional helper genes, such as Bacillus subtilis nitric oxide synthase (BsNOS) in whole cell nitration systems described herein, recombinant bacterial cells produce NO from L-Arg, which is synthesized by the E. coli cell from cheap carbon and nitrogen sources, and hence greatly lower the cost of biocatalytic nitration processes.

Accordingly, in some aspects, the disclosure relates to a recombinant bacterial cell comprising one or more isolated nucleic acids engineered to express: a fusion protein comprising a TxtE enzyme linked to a catalytic domain of a CYP102A1 (P450BM3) reductase enzyme via an amino acid linker sequence that can be varied in terms of identities and length, e.g., between 14 and 27 amino acids in length; and a nitric oxide synthase (NOS) enzyme.

In some embodiments, a recombinant bacterial cell is a Gram-negative bacterial cell. In sone embodiments, a recombinant bacterial cell is an E. coli bacterial cell.

In some embodiments, a fusion protein is a TB14 fusion protein having the sequence set forth in SEQ ID NO: 1. In some embodiments, a fusion protein is a TB14 fusion protein encoded by the sequence set forth in SEQ ID NO: 2.

In some embodiments, a NOS enzyme is a bacterial NOS enzyme. In some embodiments, a NOS enzyme is a Bacillus subtilis NOS enzyme. In some embodiments, a Bacillus subtilis NOS enzyme is encoded by the sequence set forth in SEQ ID NO: 3. In some embodiments, a Bacillus subtilis NOS enzyme comprises the amino acid sequence set forth in SEQ ID NO: 5.

In some embodiments, a recombinant bacterial cell further comprises an isolated nucleic acid engineered to express an enzyme that is able to regenerate reducing agent, e.g., NADH and/or NADPH. In some embodiments, this enzyme is a glucose I-dehydrogenase (GDH) enzyme. In some embodiments, a GDH enzyme is a bacterial GDH enzyme. In some embodiments, a bacterial GDH enzyme is a Bacillus megaterium GDH enzyme. In some embodiments, a Bacillus megaterium GDH enzyme comprises the sequence set forth in SEQ ID NO: 6. In some embodiments, a Bacillus megaterium GDH enzyme is encoded by the sequence set forth in SEQ ID NO: 7.

In some aspects, one or more isolated nucleic acids are located (e.g., situated) on a plasmid, for example a bacterial plasmid. In some embodiments, a bacterial cell comprises one or more plasmids comprising the one or more isolated nucleic acids. In some embodiments, an isolated nucleic acid engineered to express the NOS enzyme and an isolated nucleic acid engineered to express the GDH enzyme are located on the same plasmid. In some embodiments, an isolated nucleic acid engineered to express the fusion protein is located on a plasmid that does not contain an isolated nucleic acid engineered to express the NOS enzyme and/or an isolated nucleic acid engineered to express the GDH enzyme.

In some embodiments, one or more isolated nucleic acids (e.g., one or more isolated nucleic acids encoding a fusion protein, a NOS enzyme, and/or a GDH enzyme) are integrated into a chromosome of a bacterial cell.

In some embodiments, one or more isolated nucleic acid is operably linked to a promoter sequence. In some embodiments, an isolated nucleic acid engineered to express a fusion protein is operably linked to a first promoter, an isolated nucleic acid engineered to express a NOS enzyme is operably linked to a second promoter, and an isolated nucleic acid engineered to express a GDH enzyme is operably linked to a third promoter. In some embodiments, a first promoter, a second promoter, and/or a third promoter is a T7 promoter. In some embodiments, a promoter is an inducible promoter.

In some embodiments, a bacterial cell is genetically modified to lack expression of one or more of the following genes: traA (tryptophanase), trpR (tryptophan repressor), tyrA (T protein), and pheA (P protein), in some embodiments, a bacterial cell comprises the genotype ΔtrpRΔtryrAΔpheA (e.g., is a triple deletion mutant for trpR, tyrA, and pheA).

In some aspects, the disclosure relates to an isolated nucleic acid comprising the sequence set forth in any one of SEQ ID NOs: 8-13.

In some aspects, the disclosure relates to a composition comprising one or more of a recombinant bacterial cell as described by the disclosure, and a bacterial culture media. In some embodiments, a composition comprises a plurality of recombinant bacterial cells as described herein.

In some embodiments, a bacterial culture media is selected from the group consisting of M9, Lysogeny Broth (LB), SOC media, and Terrific Broth (TB).

In some embodiments, a composition further comprises one or more antibiotic agents. In some embodiments, one or more antibiotic agent is ampicillin or kanamycin.

In some embodiments, a composition further comprises a tryptophan or tryptophan analogue. In some embodiments, a composition further comprises one or more of the following: L-tryptophan (L-Trp), L-arginine (L-Arg), or an analogue of L-tryptophan. In some embodiments, an analogue of L-tryptophan is selected from the group consisting of α-Me-Trp, 4-F-Trp, 4-Me-Trp, 5-MeO-Trp, 5-Me-Trp, 5-F-Trp, 6-F-Trp, and 7-Me-Trp.

In some embodiments, the tryptophan or tryptophan analogue is a compound of Formula Ia:

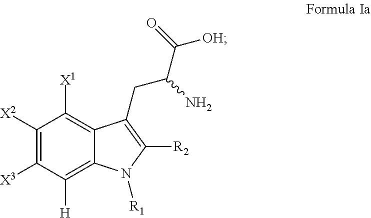

wherein:
each X¹ is independently halogen, substituted or unsubstituted Ca alkyl, substituted or unsubstituted C₂₋₆ alkenyl, substituted or unsubstituted C₂₋₆ alkynyl, substituted or unsubstituted, monocyclic, 3- to 6-membered carbocyclyl, substituted or unsubstituted, monocyclic, 3- to 6-membered heterocyclyl, substituted or unsubstituted phenyl, substituted or unsubstituted, monocyclic, 5- to 6-membered heteroaryl, —OR^{Ala}, —N(R^{Ala})₂, or —SR^{Ala};
   wherein each R^{Ala} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{Ala} are joined to form a substituted or unsubstituted, heterocyclic ring, or substituted or unsubstituted, heteroaryl ring;
each of X² and X³ is, independently, hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted, monocyclic, 3- to 6-membered carbocyclyl, substituted or unsubstituted, monocyclic, 3- to 6-membered heterocyclyl, substituted or unsubstituted phenyl, substituted or unsubstituted, monocyclic, 5- to 6-membered heteroaryl, —OR^{Ala}, —N(R^{Ala)}₂, or —SR^{Ala};
wherein each R^{Ala} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{Ala} are joined to form a substituted or unsubstituted, heterocyclic ring, or substituted or unsubstituted, heteroaryl ring;
R₁ is H or optionally substituted alkyl; and
R₂ is H, optionally substituted alkyl, optionally substituted alkenyl, optionally substituted alkynyl, optionally substituted aryl, or optionally substituted heteraryl. In another aspect, R₁ is H or alkyl. In another aspect, R₁ is H. In another aspect. R₁ is alkyl. In another aspect, R₁ is H methyl. In another aspect, R₂ is H. In another aspect, R₁ and R₂ are each H. In another aspect, R₁ is alkyl and R₂ is H. In another aspect, R₁ is methyl and R₂ is H.

In some embodiments, the tryptophan or tryptophan analogue is a compound of Formula IVa:

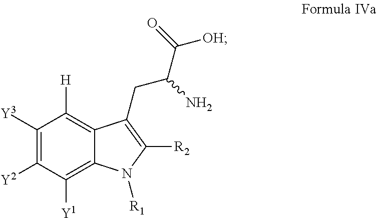

wherein:
each of Y¹, Y², and Y³ is, independently, hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted, monocyclic, 3- to 6-membered carbocyclyl, substituted or unsubstituted, monocyclic, 3- to 6-membered heterocyclyl, substituted or unsubstituted phenyl, substituted or unsubstituted, monocyclic, 5- to 6-membered heteroaryl, —OR^{Ala}, —N(R^{Ala})₂, or SR^{Ala}; and
wherein each R^{Ala} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{Ala} are joined to form a substituted or unsubstituted, heterocyclic ring, or substituted or unsubstituted, heteroaryl ring;
R₁ is H or optionally substituted alkyl; and
R₂ is H, optionally substituted alkyl, optionally substituted alkenyl, optionally substituted alkynyl, optionally substituted aryl, or optionally substituted heteroaryl. In another aspect, R₁ is H or alkyl, in another aspect. R₁ is H. In another aspect, R₁ is alkyl. In another aspect, R₁ is H or methyl. In another aspect, R₂ is H. In another aspect, R₁ and R₂ are each H. In another aspect, R₁ is alkyl and R₂ is H. In another aspect. R₁ is methyl and R₂ is H.

In some embodiments, a composition further comprises one or more of a nitrated tryptophan or a nitrated tryptophan analogue. In some embodiments, a composition further comprises one or more of the following: 4-NO₂-L-Trp, nitrated 4-NO₂-α-Me-Trp, 4-F-7-NO₂-Trp, 4-Me-7-NO₂-Trp, 5-MeO-4-NO₂-Trp, 5-Me-4-NO₂-Trp, nitrated 5-F-4-NO₂-Trp, 6-F-4-NO₂-Trp, or 7-Me-4-NO₂-Trp.

In some embodiments, the nitrated tryptophan or nitrated tryptophan analogue is a compound of Formula I, or a pharmaceutically acceptable salt, prodrug, hydrate, or solvate thereof:

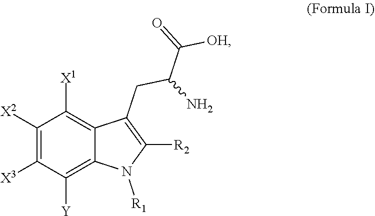

wherein:
X¹ is halogen, substituted or unsubstituted C₁₋₆ alkyl, substituted or unsubstituted C₂₋₆ alkenyl, substituted or unsubstituted C₂₋₆ alkynyl, substituted or unsubstituted, monocyclic, 3- to 6-membered carbocyclyl, substituted or unsubstituted, monocyclic, 3- to 6-membered heterocyclyl, substituted or unsubstituted phenyl, substituted or unsubstituted, monocyclic, 5- to 6-membered heteroaryl, —OR^{Ala}, —N(R^{Ala})₂, or —SR^{Ala},
   wherein each R^{Ala} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{Ala} are joined to form a substituted or unsubstituted, heterocyclic ring, or substituted or unsubstituted, heteroaryl ring;
each of X² and X³ is, independently, hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted, monocyclic, 3- to 6-membered carbocyclyl, substituted or unsubstituted, monocyclic, 3- to 6-membered heterocyclyl, substituted or unsubstituted phenyl, substituted or unsubstituted, monocyclic, 5- to 6-membered heteroaryl, —OR^{Ala}, —N(R^{Ala})₂, or —SR^{Ala},
   wherein each R^{Ala} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{Ala} are joined to form a substituted or unsubstituted, heterocyclic ring, or substituted or unsubstituted, heteroaryl ring;
R₁ is H or optionally substituted alkyl;
R₂ is H, optionally substituted alkyl, optionally substituted alkenyl, optionally substituted alkynyl, optionally substituted aryl, or optionally substituted heteroaryl; and
Y is NO₂. In another aspect, R₁ is H or alkyl. In another aspect, R₁ is H. In another aspect, R₁ is alkyl. In another aspect, R₁ is H or methyl. In another aspect, R₂ is H. In another aspect, R₁ and R₂ are each H. In another aspect, R₁ is alkyl and R₂ is H. In another aspect. R₁ is methyl and R₂ is H.

In some aspects, the compound disclosure elates to a compound of Formula I, wherein at least one of X¹, X², or X³ is a “weakly deactivating group”, a “weakly activating group”, a “moderately activating group”, or a “strongly activating group”, as known in the art and as defined herein. In other aspects, at least one of X¹, X², or X³ is H, halogen (e.g., F, Cl, Br, I), substituted or unsubstituted C₁₋₆ alkyl (e.g., methyl, CH₃), substituted or unsubstituted C₂₋₆ alkenyl, substituted or unsubstituted C₂₋₆ alkynyl, substituted or unsubstituted, monocyclic, 3- to 6-membered carbocyclyl, substituted or unsubstituted, monocyclic, 3- to 6-membered heterocyclyl, substituted or unsubstituted phenyl, substituted or unsubstituted, monocyclic, 5- to 6-membered heteroaryl, —OR^{Ala}, —N(R^{Ala})₂, or —SR^{Ala}.

In another aspect, X¹ is halogen (e.g., F, Cl, Br, I), substituted or unsubstituted C₁₋₆ alkyl (e.g., methyl, CH₃), substituted or unsubstituted C₂₋₆ alkenyl, substituted or unsubstituted C₂₋₆ alkynyl, substituted or unsubstituted, monocyclic, 3- to 6-membered carbocyclyl, substituted or unsubstituted, monocyclic, 3- to 6-membered heterocyclyl, substituted or unsubstituted phenyl, substituted or unsubstituted, monocyclic, 5- to 6-membered heteroaryl, —OR^{Ala}, —N(R^{Ala})₂, or —SR^{Ala}; and X² and X³ are each independently H, halogen (e.g., F, Cl, Br, I), substituted or unsubstituted C₁₋₆ alkyl (e.g., methyl, CH₃), substituted or unsubstituted C₂₋₆ alkenyl, substituted or unsubstituted C₂₋₆ alkynyl, substituted or unsubstituted, monocyclic, 3- to 6-membered carbocyclyl, substituted or unsubstituted, monocyclic, 3- to 6-membered heterocyclyl, substituted or unsubstituted phenyl, substituted or unsubstituted, monocyclic, 5- to 6-membered heteroaryl, —OR^{Ala}, —N(R^{Ala})₂, or —SR^{Ala}. In another aspect, X¹ is halogen or C₁₋₆ alkyl (e.g., methyl, CH₃). In another aspect, X¹ is halogen. In another aspect, X¹ is C₁₋₆ alkyl (e.g., methyl, CH₃). In another aspect. X¹ is halogen or C₁₋₆ alkyl (e.g., methyl, CH₃) and at least one of X² and X³ is hydrogen. In another aspect, X¹ is halogen and each of X² and X³ is hydrogen. In another aspect, X¹ if fluorine and each of X² and X³ is hydrogen. In another aspect. X¹ is C₁₋₆ alkyl and each of X² and X³ is hydrogen. In another aspect, X¹ is methyl and each of X² and X³ is hydrogen.

In some embodiments, the compound of Formula I is a compound of Formula II:

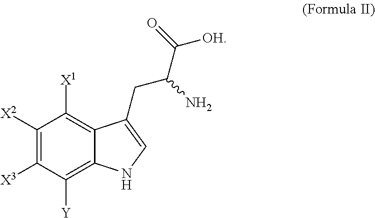

In certain embodiments, the compound of Formula I is a compound of Formula III:

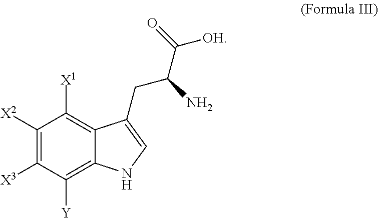

In some embodiments, the nitrated tryptophan or nitrated tryptophan analogue is a compound of Formula IV, or a pharmaceutically acceptable salt, prodrug, hydrate, or solvate thereof:

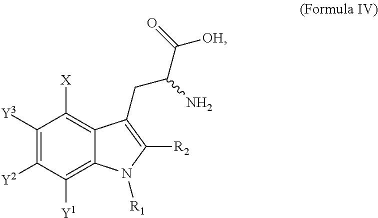

wherein:
each of Y¹, Y², and Y³ is, independently, hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted, monocyclic, 3- to 6-membered carbocyclyl, substituted or unsubstituted, monocyclic, 3- to 6-membered heterocyclyl, substituted or unsubstituted phenyl, substituted or unsubstituted, monocyclic, 5- to 6-membered heteroaryl, —OR^{Ala}, —N(R^{Ala})₂, or —SR^{Ala},
   wherein each R^{Ala} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{Ala} are joined to form a substituted or unsubstituted, heterocyclic ring, or substituted or unsubstituted, heteroaryl ring;
R₁ is H or optionally substituted alkyl;
R₂ is H, optionally substituted alkyl, optionally substituted alkenyl, optionally substituted alkynyl, optionally substituted aryl, or optionally substituted heteroaryl; and
X is NO₂. In another aspect, R₁ is H or alkyl. In another aspect, R₁ is H. In another aspect, R₁ is alkyl. In another aspect, R₁ is H or methyl. In another aspect, R₂ is H. In another aspect. R₁ and R₂ are each H. In another aspect, R₁ is alkyl and R₂ is H. In another aspect, R₁ is methyl and R₂ is H.

In some aspects, the compound disclosure relates to a compound of Formula IV, wherein at least one of Y¹, Y², or Y³ is a “weakly deactivating group”, a “weakly activating group”, a “moderately activating group”, or a “strongly activating group”, as known in the art and as defined herein. In other aspects, at least one of Y¹, Y², or Y³ is H, halogen (e.g. F, Cl, Br, I), substituted or unsubstituted C₁₋₆ alkyl (e.g. methyl, CH₃), substituted or unsubstituted C₂₋₆alkenyl, substituted or unsubstituted C₂₋₆ alkynyl, substituted or unsubstituted, monocyclic, 3- to 6-membered carbocyclyl, substituted or unsubstituted, monocyclic, 3- to 6-membered heterocyclyl, substituted or unsubstituted phenyl, substituted or unsubstituted, monocyclic, 5- to 6-membered heteroaryl, —OR^{Ala}, —N(R^{Ala})₂, or —SR^{Ala}.

In embodiments, Y¹, Y², or Y³ is halogen and the halogen is fluorine. In embodiments, Y¹, Y², or Y³ is unsubstituted C₁-C₆ alkyl. In embodiments, the unsubstituted C₁-C₆ alkyl is methyl (—CH₃). In embodiments, two of Y¹, Y² and Y³ are hydrogen. In embodiments, Y² and Y³ are hydrogen. In embodiments, Y¹ and Y³ are hydrogen. In embodiments, Y¹ and Y² are hydrogen.

In some aspects, the disclosure relates to a compound of Formula IV, wherein at least one of Y¹, Y² or Y³ is halogen or C₁₋₆ alkyl (e.g. methyl, CH₃). In another aspect, Y³ is halogen (e.g. F, Cl, Br, I), substituted or unsubstituted C₁₋₆ alkyl (e.g. methyl, CH₃), substituted or unsubstituted C₂₋₆ alkenyl, substituted or unsubstituted C₂₋₆ alkynyl, substituted or unsubstituted, monocyclic, 3- to 6-membered carbocyclyl, substituted or unsubstituted, monocyclic, 3- to 6-membered heterocyclyl, substituted or unsubstituted phenyl, substituted or unsubstituted, it monocyclic, 5- to 6-membered heteroaryl, —OR^{Ala}, —N(R^{Ala})₂, or —SR^{Ala}; and Y¹ and Y² are each independently H, halogen (e.g. F, Cl, Br, I), substituted or unsubstituted C₁₋₆alkyl (e.g. methyl, CH₃), substituted or unsubstituted C₂₋₆ alkenyl, substituted or unsubstituted C₂₋₆ alkynyl, substituted or unsubstituted, monocyclic, 3- to 6-membered carbocyclyl, substituted or unsubstituted, monocyclic, 3- to 6-membered heterocyclyl, substituted or unsubstituted phenyl, substituted or unsubstituted, monocyclic, 5- to 6-membered heteroaryl, —OR^{Ala}, —N(R^{Ala})₂, or —SR^{Ala}). In another aspect, Y³ is halogen or C₁₋₆ alkyl (e.g. methyl, CH₃). In another aspect, Y³ is halogen or C₁₋₆alkyl (e.g. methyl, CH₃) and at least one of Y¹ and Y² is hydrogen. In another aspect, Y³ is halogen or C₁₋₆ alkyl (e.g. methyl, CH₃) and Y¹ and Y² are each hydrogen. In another aspect, Y³ is halogen. In another aspect, Y³ is halogen and at least one of Y₁ and Y² is hydrogen. In another aspect, Y³ is halogen and Y¹ and Y² are each hydrogen. In certain embodiments, Y³ is fluorine and at least one of Y¹ and Y² is hydrogen. In another aspect, Y³ is fluorine and Y¹ and Y² are each hydrogen. In another aspect. Y³ is C₁₋₆ alkyl. In another aspect, Y³ is C₁₋₆ alkyl and at least one of Y¹ and Y² is hydrogen. In another aspect, Y³ is C₁₋₆ alkyl and Y¹ and Y² are each hydrogen. In certain embodiments, Y³ is methyl and at least one of Y¹ and Y² is hydrogen. In another aspect, Y³ is methyl and Y¹ and Y² are each hydrogen.

In certain embodiments, the compound of Formula IV is a compound of Formula V:

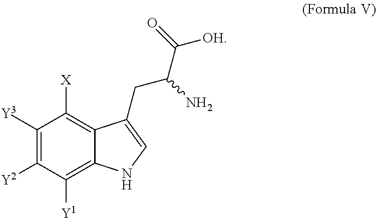

In certain embodiments, the compound of Formula IV is a compound of Formula VI:

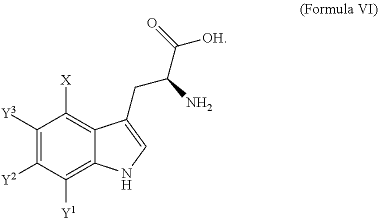

In another aspect, the invention is directed to a compound of Formulae I-VI, wherein the compound is:
(S)-2-amino-3-(4-methyl-5-nitro-1H-indol-3-yl)propanoic acid;
(S)-2-amino-3-(5-methyl-4-nitro-1H-indol-3-yl)propanoic acid;
(S)-2-amino-3-(6-methyl-4-nitro-1H-indol-3-yl)propanoic acid;
(S)-2-amino-3-(7-methyl-4-nitro-1H-indol-3-yl)propanoic acid;
(S)-2-amino-3-(4-methyl-7-nitro-1H-indol-3-yl)propanoic acid;
(S)-2-amino-3-(5-fluoro-4-nitro-1H-indol-3-yl)propanoic acid;
(S)-2-amino-3-(6-fluoro-4-nitro-1H-indol-3-yl)propanoic acid;
(S)-2-amino-3-(7-fluoro-4-nitro-1H-indol-3-yl)propanoic acid;
(S)-2-amino-3-(4-fluoro-7-nitro-1H-indol-3-yl)propanoic acid;
(S)-2-amino-3-(5-chloro-4-nitro-1H-indol-3-yl)propanoic acid;
(S)-2-amino-3-(6-chloro-4-nitro-1H-indol-3-yl)propanoic acid;
(S)-2-amino-3-(7-chloro-4-nitro-1H-indol-3-yl)propanoic acid;
(S)-2-amino-3-(4-chloro-7-nitro-1H-indol-3-yl)propanoic acid;
(S)-2-amino-3-(5-bromo-4-nitro-1H-indol-3-yl)propanoic acid;
(S)-2-amino-3-(6-bromo-4-nitro-1H-indol-3-yl)propanoic acid;
(S)-2-amino-3-(7-bromo-4-nitro-1H-indol-3-yl)propanoic acid;
(S)-2-amino-3-(4-bromo-7-nitro-1H-indol-3-yl)propanoic acid;
(S)-2-amino-3-(5-methoxy-4-nitro-1H-indol-3-yl)propanoic acid;
(S)-2-amino-3-(6-methoxy-4-nitro-1H-indol-3-yl)propanoic acid;
(S)-2-amino-3-(7-methoxy-4-nitro-1H-indol-3-yl)propanoic acid;
(S)-2-amino-3-(4-methoxy-7-nitro-1H-indol-3-yl)propanoic acid;
(S)-2-amino-3-(5-amino-4-nitro-1H-indol-3-yl)propanoic acid;
(S)-2-amino-3-(6-amino-4-nitro-1H-indol-3-yl)propanoic acid;
(S)-2-amino-3-(7-amino-4-nitro-1H-indol-3-yl)propanoic acid;
(S)-2-amino-3-(4-amino-7-nitro-1H-indol-3-yl)propanoic acid;
(S)-2-amino-3-(5-hydroxy-4-nitro-1H-indol-3-yl)propanoic acid;
(S)-2-amino-3-(6-hydroxy-4-nitro-1H-indol-3-yl)propanoic acid;
(S)-2-amino-3-(7-hydroxy-4-nitro-1H-indol-3-yl)propanoic acid;
(S)-2-amino-3-(4-hydroxy-7-nitro-1H-indol-3-yl)propanoic acid;
(S)-2-amino-3-(4-nitro-5-phenyl-1H-indol-3-yl)propanoic acid;
(S)-2-amino-3-(4-nitro-6-phenyl-1H-indol-3-yl)propanoic acid;
(S)-2-amino-3-(4-nitro-7-phenyl-1H-indol-3-yl)propanoic acid;
(S)-2-amino-3-(7-nitro-4-phenyl-1H-indol-3-yl)propanoic acid;
(S)-2-amino-3-(5-cyclopropyl-4-nitro-1H-indol-3-yl)propanoic acid;
(S)-2-amino-3-(6-cyclopropyl-4-nitro-1H-indol-3-yl)propanoic acid;
(S)-2-amino-3-(7-cyclopropyl-4-nitro-1H-indol-3-yl)propanoic acid;
(S)-2-amino-3-(4-cyclopropyl-7-nitro-1H-indol-3-yl)propanoic acid;
(S)-2-amino-3-(4-nitro-5-vinyl-1H-indol-3-yl)propanoic acid;
(S)-2-amino-3-(4-nitro-6-vinyl-1H-indol-3-yl)propanoic acid;
(S)-2-amino-3-(4-nitro-7-vinyl-1H-indol-3-yl)propanoic acid;
(S)-2-amino-3-(7-nitro-4-vinyl-1H-indol-3-yl)propanoic acid;
(S)-2-amino-3-(5-ethynyl-4-nitro-1H-indol-3-yl)propanoic acid;
(S)-2-amino-3-(6-ethynyl-4-nitro-1H-indol-3-yl)propanoic acid;
(S)-2-amino-3-(7-ethynyl-4-nitro-1H-indol-3-yl)propanoic acid;
(S)-2-amino-3-(4-ethynyl-7-nitro-1H-indol-3-yl)propanoic acid;
(S)-2-amino-3-(5-morpholino-4-nitro-1H-indol-3-yl)propanoic acid;
(S)-2-amino-3-(6-morpholino-4-nitro-1H-indol-3-yl)propanoic acid;
(S)-2-amino-3-(7-morpholino-4-nitro-1H-indol-3-yl)propanoic acid;
(S)-2-amino-3-(4-morpholino-7-nitro-1H-indol-3-yl)propanoic acid;
(S)-2-amino-3-(5-(methylthio)-4-nitro-1H-indol-3-yl)propanoic acid;
(S)-2-amino-3-(6-(methylthio)-4-nitro-1H-indol-3-yl)propanoic acid;
(S)-2-amino-3-(7-(methylthio)-4-nitro-1H-indol-3-yl)propanoic acid;
(S)-2-amino-3-(4-(methylthio)-7-nitro-1H-indol-3-yl)propanoic acid;
(S)-2-amino-3-(4-nitro-5-(pyridin-4-yl)-1H-indol-3-yl)propanoic acid;
(S)-2-amino-3-(4-nitro-6-(pyridin-4-yl)-1H-indol-3-yl)propanoic acid;
(S)-2-amino-3-(4-nitro-7-(pyridin-4-yl)-1H-indol-3-yl)propanoic acid;
(S)-2-amino-3-(7-nitro-4-(pyridin-4-yl)-1H-indol-3-yl)propanoic acid;
2-amino-3-(4-methyl-5-nitro-1H-indol-3-yl)propanoic acid;
2-amino-3-(5-methyl-4-nitro-1H-indol-3-yl)propanoic acid;
2-amino-3-(6-methyl-4-nitro-1H-indol-3-yl)propanoic acid;
2-amino-3-(7-methyl-4-nitro-1H-indol-3-yl)propanoic acid;
2-amino-3-(4-methyl-7-nitro-1H-indol-3-yl)propanoic acid;
2-amino-3-(6-fluoro-4-nitro-1H-indol-3-yl)propanoic acid;
2-amino-3-(7-fluoro-4-nitro-1H-indol-3-yl)propanoic acid;
2-amino-3-(4-fluoro-7-nitro-1H-indol-3-yl)propanoic acid;
2-amino-3-(5-chloro-4-nitro-1H-indol-3-yl)propanoic acid;
2-amino-3-(6-chloro-4-nitro-1H-indol-3-yl)propanoic acid;
2-amino-3-(7-chloro-4-nitro-1H-indol-3-yl)propanoic acid;
2-amino-3-(4-chloro-7-nitro-1H-indol-3-yl)propanoic acid;
2-amino-3-(5-bromo-4-nitro-1H-indol-3-yl)propanoic acid;
2-amino-3-(6-bromo-4-nitro-1H-indol-3-yl)propanoic acid;
2-amino-3-(7-bromo-7-nitro-1H-indol-3-yl)propanoic acid;
2-amino-3-(4-bromo-7-nitro-1H-indol-3-yl)propanoic acid;
2-amino-3-(5-methoxy-4-nitro-1H-indol-3-yl)propanoic acid;
2-amino-3-(6-methoxy-4-nitro-1H-indol-3-yl)propanoic acid;
2-amino-3-(7-methoxy-4-nitro-1H-indol-3-yl)propanoic acid;
2-amino-3-(4-methoxy-7-nitro-1H-indol-3-yl)propanoic acid;
2-amino-3-(5-amino-4-nitro-1H-indol-3-yl)propanoic acid;
2-amino-3-(6-amino-4-nitro-1H-indol-3-yl)propanoic acid;
2-amino-3-(7-amino-4-nitro-1H-indol-3-yl)propanoic acid;
2-amino-3-(4-amino-7-nitro-1H-indol-3-yl)propanoic acid;
2-amino-3-(5-hydroxy-4-nitro-1H-indol-3-yl)propanoic acid;
2-amino-3-(6-hydroxy-4-nitro-1H-indol-3-yl)propanoic acid;
2-amino-3-(7-hydroxy-4-nitro-1H-indol-3-yl)propanoic acid;
2-amino-3-(4-hydroxy-7-nitro-1H-indol-3-yl)propanoic acid;
2-amino-3-(4-nitro-5-phenyl-1H-indol-3-yl)propanoic acid;
2-amino-3-(4-nitro-6-phenyl-1H-indol-3-yl)propanoic acid;
2-amino-3-(4-nitro-7-phenyl-1H-indol-3-yl)propanoic acid;
2-amino-3-(7-nitro-4-phenyl-1H-indol-3-yl)propanoic acid;
2-amino-3-(5-cyclopropyl-4-nitro-1H-indol-3-yl)propanoic acid;
2-amino-3-(6-cyclopropyl-4-nitro-1H-indol-3-yl)propanoic acid;
2-amino-3-(7-cyclopropyl-4-nitro-1H-indol-3-yl)propanoic acid;
2-amino-3-(4-cyclopropyl-7-nitro-1H-indol-3-yl)propanoic acid;
2-amino-3-(4-nitro-5-vinyl-1H-indol-3-yl)propanoic acid;
2-amino-3-(4-nitro-6-vinyl-1H-indol-3-yl)propanoic acid;
2-amino-3-(4-nitro-7-vinyl-1H-indol-3-yl)propanoic acid;
2-amino-3-(7-nitro-4-vinyl-1H-indol-3-yl)propanoic acid;
2-amino-3-(5-ethynyl-4-nitro-1H-indol-3-yl)propanoic acid;
2-amino-3-(6-ethynyl-4-nitro-1H-indol-3-yl)propanoic acid;
2-amino-3-(7-ethynyl-7-nitro-1H-indol-3-yl)propanoic acid;
2-amino-3-(4-ethynyl-7-nitro-1H-indol-3-yl)propanoic acid;
2-amino-3-(5-morpholino-4-nitro-1H-indol-3-yl)propanoic acid;
2-amino-3-(6-morpholino-4-nitro-1H-indol-3-yl)propanoic acid;
2-amino-3-(7-morpholino-4-nitro-1H-indol-3-yl)propanoic acid;
2-amino-3-(4-morpholino-7-nitro-1H-indol-3-yl)propanoic acid;
2-amino-3-(5-(methylthio)-4-nitro-1H-indol-3-yl)propanoic acid;
2-amino-3-(6-(methylthio)-4-nitro-1H-indol-3-yl)propanoic acid;
2-amino-3-(7-(methylthio)-4-nitro-1H-indol-3-yl)propanoic acid;
2-amino-3-(4-(methylthio-7-nitro-1H-indol-3-yl)propanoic acid;
2-amino-3-(4-nitro-5-(pyridin-4-yl)-1H-indol-3-yl)propanoic acid;
2-amino-3-(4-nitro-6-(pyridin-4-yl)-1H-indol-3-yl)propanoic acid;
2-amino-3-(7-nitro-7-(pyridin-4-yl)-1H-indol-3-yl)propanoic acid;
2-amino-3-(7-nitro-4-(pyridin-4-yl)-1H-indol-3-yl)propanoic acid;
2-amino-3-(1,5-dimethyl-4-nitro-1H-indol-3-yl)propanoic acid;
2-amino-3-(1,6-dimethyl-4-nitro-1H-indol-3-yl)propanoic acid;
2-amino-3-(1,7-dimethyl-4-nitro-1H-indol-3-yl)propanoic acid;
2-amino-3-(1,4-dimethyl-7-nitro-1H-indol-3-yl)propanoic acid;
2-amino-3-(6-fluoro-1-methyl-4-nitro-1H-indol-3-yl)propanoic acid;
2-amino-3-(7-fluoro-1-methyl-4-nitro-1H-indol-3-yl)propanoic acid;
2-amino-3-(4-fluoro-1-methyl-7-nitro-1H-indol-3-yl)propanoic acid;
2-amino-3-(5-chloro-1-methyl-4-nitro-1H-indol-3-yl)propanoic acid;
2-amino-3-(6-chloro-1-methyl-4-nitro-1H-indol-3-yl)propanoic acid;
2-amino-3-(7-chloro-1-methyl-4-nitro-1H-indol-3-yl)propanoic acid;
2-amino-3-(4-chloro-1-methyl-7-nitro-1H-indol-3-yl)propanoic acid;
2-amino-3-(5-bromo-1-methyl-4-nitro-1H-indol-3-yl)propanoic acid;
2-amino-3-(6-bromo-1-methyl-4-nitro-1H-indol-3-yl)propanoic acid;
2-amino-3-(7-bromo-1-methyl-4-nitro-1H-indol-3-yl)propanoic acid;
2-amino-3-(4-bromo-1-methyl-7-nitro-1H-indol-3-yl)propanoic acid;
2-amino-3-(5-methoxy-1-methyl-4-nitro-1H-indol-3-yl)propanoic acid;
2-amino-3-(6-methoxy-1-methyl-4-nitro-1H-indol-3-yl)propanoic acid;
2-amino-3-(7-methoxy-1-methyl-4-nitro-1H-indol-3-yl)propanoic acid;
2-amino-3-(4-methoxy-1-methyl-7-nitro-1H-indol-3-yl)propanoic acid;
2-amino-3-(5-amino-1-methyl-4-nitro-1H-indol-3-yl)propanoic acid;
2-amino-3-(6-amino-1-methyl-4-nitro-1H-indol-3-yl)propanoic acid;
2-amino-3-(7-amino-1-methyl-4-nitro-1H-indol-3-yl)propanoic acid;
2-amino-3-(4-amino-1-methyl-7-nitro-1H-indol-3-yl)propanoic acid;
2-amino-3-(5-hydroxy-1-methyl-4-nitro-1H-indol-3-yl)propanoic acid;
2-amino-3-(6-hydroxy-1-methyl-4-nitro-1H-indol-3-yl)propanoic acid;
2-amino-3-(7-hydroxy-1-methyl-4-nitro-1H-indol-3-yl)propanoic acid;
2-amino-3-(4-hydroxy-1-methyl-7-nitro-1H-indol-3-yl)propanoic acid;
2-amino-3-(1-methyl-4-nitro-5-phenyl-1H-indol-3-yl)propanoic acid;
2-amino-3-(1-methyl-4-nitro-6-phenyl-1H-indol-3-yl)propanoic acid;
2-amino-3-(1-methyl-4-nitro-7-phenyl-1H-indol-3-yl)propanoic acid;
2-amino-3-(1-methyl-7-nitro-4-phenyl-1H-indol-3-yl)propanoic acid;
2-amino-3-(5-cyclopropyl-1-methyl-4-nitro-1H-indol-3-yl)propanoic acid;
2-amino-3-(6-cyclopropyl-1-methyl-4-nitro-1H-indol-3-yl)propanoic acid;
2-amino-3-(7-cyclopropyl-1-methyl-4-nitro-1H-indol-3-yl)propanoic acid;
2-amino-3-(4-cyclopropyl-1-methyl-7-nitro-1H-indol-3-yl)propanoic acid;
2-amino-3-(1-methyl-4-nitro-5-vinyl-1H-indol-3-yl)propanoic acid;
2-amino-3-(1-methyl-4-nitro-6-vinyl-1H-indol-3-yl)propanoic acid;
2-amino-3-(1-methyl-4-nitro-7-vinyl-1H-indol-3-yl)propanoic acid;
2-amino-3-(1-methyl-7-nitro-4-vinyl-1H-indol-3-yl)propanoic acid;
2-amino-3-(5-ethynyl-1-methyl-4-nitro-1H-indol-3-yl)propanoic acid;
2-amino-3-(6-ethynyl-1-methyl-4-nitro-1H-indol-3-yl)propanoic acid;
2-amino-3-(7-ethynyl-1-methyl-4-nitro-1H-indol-3-yl)propanoic acid;
2-amino-3-(4-ethynyl-1-methyl-7-nitro-1H-indol-3-yl)propanoic acid;
2-amino-3-(1-methyl-5-morpholino-4-nitro-1H-indol-3-yl)propanoic acid;
2-amino-3-(1-methyl-6-morpholino-4-nitro-1H-indol-3-yl)propanoic acid;
2-amino-3-(1-methyl-7-morpholino-4-nitro-1H-indol-3-yl)propanoic acid;
2-amino-3-(1-methyl-4-morpholino-7-nitro-1H-indol-3-yl)propanoic acid;
2-amino-3-(1-methyl-5-(methylthio)-4-nitro-1H-indol-3-yl)propanoic acid;
2-amino-3-(1-methyl-6-(methylthio)-4-nitro-1H-indol-3-yl)propanoic acid;
2-amino-3-(1-methyl-7-(methylthio)-4-nitro-1H-indol-3-yl)propanoic acid;
2-amino-3-(1-methyl-4-(methylthio)-7-nitro-1H-indol-3-yl)propanoic acid;
2-amino-3-(1-methyl-4-nitro-5-(pyridin-4-yl)-1H-indol-3-yl)propanoic acid;
2-amino-3-(1-methyl-4-nitro-4-(pyridin-4-yl)-1H-indol-3-yl)propanoic acid;
2-amino-3-(1-methyl-4-nitro-7-(pyridin-4-yl)-1H-indol-3-yl)propanoic acid;
2-amino-3-(1-methyl-7-nitro-4-(pyridin-4-yl)-1H-indol-3-yl)propanoic acid;
2-amino-3-(2,5-dimethyl-4-nitro-1H-indol-3-yl)propanoic acid;
2-amino-3-(2,6-dimethyl-4-nitro-1H-indol-3-yl)propanoic acid;
2-amino-3-(2,7-dimethyl-4-nitro-1H-indol-3-yl)propanoic acid;
2-amino-3-(2,4-dimethyl-7-nitro-1H-indol-3-yl)propanoic acid;
2-amino-3-(6-fluoro-2-methyl-4-nitro-1H-indol-3-yl)propanoic acid;
2-amino-3-(7-fluoro-2-methyl-4-nitro-1H-indol-3-yl)propanoic acid;
2-amino-3-(4-fluoro-2-methyl-7-nitro-1H-indol-3-yl)propanoic acid;
2-amino-3-(5-chloro-2-methyl-4-nitro-1H-indol-3-yl)propanoic acid;
2-amino-3-(6-chloro-2-methyl-4-nitro-1H-indol-3-yl)propanoic acid;
2-amino-3-(7-chloro-2-methyl-4-nitro-1H-indol-3-yl)propanoic acid;
2-amino-3-(4-chloro-2-methyl-4-nitro-1H-indol-3-yl)propanoic acid;
2-amino-3-(5-bromo-2-methyl-4-nitro-1H-indol-3-yl)propanoic acid;
2-amino-3-(6-bromo-2-methyl-4-nitro-1H-indol-3-yl)propanoic acid;
2-amino-3-(7-bromo-2-methyl-7-nitro-1H-indol-3-yl)propanoic acid;
2-amino-3-(4-bromo-2-methyl-7-nitro-1H-indol-3-yl)propanoic acid;
2-amino-3-(5-methoxy-2-methyl-4-nitro-1H-indol-3-yl)propanoic acid;
2-amino-3-(6-methoxy-2-methyl-4-nitro-1H-indol-3-yl)propanoic acid;
2-amino-3-(7-methoxy-2-methyl-4-nitro-1H-indol-3-yl)propanoic acid;
2-amino-3-(4-methoxy-2-methyl-7-nitro-1H-indol-3-yl)propanoic acid;
2-amino-3-(5-amino-2-methyl-4-nitro-1H-indol-3-yl)propanoic acid;
2-amino-3-(6-amino-2-methyl-4-nitro-1H-indol-3-yl)propanoic acid;
2-amino-3-(7-amino-2-methyl-4-nitro-1H-indol-3-yl)propanoic acid;
2-amino-3-(4-amino-2-methyl-7-nitro-1H-indol-3-yl)propanoic acid;
2-amino-3-(5-hydroxy-2-methyl-4-nitro-1H-indol-3-yl)propanoic acid;
2-amino-3-(6-hydroxy-2-methyl-4-nitro-1H-indol-3-yl)propanoic acid;
2-amino-3-(7-hydroxy-2-methyl-4-nitro-1H-indol-3-yl)propanoic acid;
2-amino-3-(4-hydroxy-2-ethyl-7-nitro-1H-indol-3-yl)propanoic acid;
2-amino-3-(2-methyl-4-nitro-5-phenyl-1H-indol-3-yl)propanoic acid;
2-amino-3-(2-methyl-4-nitro-6-phenyl-1H-indol-3-yl)propanoic acid;
2-amino-3-(2-methyl-4-nitro-7-phenyl-1H-indol-3-yl)propanoic acid;
2-amino-3-(2-methyl-7-nitro-4-phenyl-1H-indol-3-yl)propanoic acid;
2-amino-3-(5-cyclopropyl-2-methyl-4-nitro-1H-indol-3-yl)propanoic acid;
2-amino-3-(6-cyclopropyl-2-methyl-4-nitro-1H-indol-3-yl)propanoic acid;
2-amino-3-(7-cyclopropyl-2-methyl-4-nitro-1H-indol-3-yl)propanoic acid;
2-amino-3-(4-cyclopropyl-2-methyl-7-nitro-1H-indol-3-yl)propanoic acid
2-amino-3-(2-methyl-4-nitro-5-vinyl-1H-indol-3-yl)propanoic acid;
2-amino-3-(2-methyl-4-nitro-6-vinyl-1H-indol-3-yl)propanoic acid;
2-amino-3-(2-methyl-4-nitro-7-vinyl-1H-indol-3-yl)propanoic acid;
2-amino-3-(2-methyl-7-nitro-4-vinyl-1H-indol-3-yl)propanoic acid;
2-amino-3-(6-ethynyl-2-methyl-4-nitro-1H-indol-3-yl)propanoic acid;
2-amino-3-(6-ethyl-2-methyl-4-nitro-1H-indol-3-yl)propanoic acid;
2-amino-3-(7-ethynyl-2-methyl-4-nitro-1H-indol-3-yl)propanoic acid;
2-amino-3-(4-ethynyl-2-methyl-7-nitro-1H-indol-3-yl)propanoic acid;
2-amino-3-(2-methyl-5-morpholino-4-nitro-1H-indol-3-yl)propanoic acid;
2-amino-3-(2-methyl-6-morpholino-4-nitro-1H-indol-3-yl)propanoic acid;
2-amino-3-(2-methyl-7-morpholino-4-nitro-1H-indol-3-yl)propanoic acid;
2-amino-3-(2-methyl-4-morpholino-7-nitro-1H-indol-3-yl)propanoic acid;
2-amino-3-(2-methyl-5-(methylthio)-4-nitro-1H-indol-3-yl)propanoic acid;
2-amino-3-(2-methyl-6-(methylthio)-4-nitro-1H-indol-3-yl)propanoic acid;
2-amino-3-(2-methyl-7-(methylthio)-4-nitro-1H-indol-3-yl)propanoic acid;
2-amino-3-(2-methyl-4-methylthio)-7-nitro-1H-indol-3-yl)propanoic acid;
2-amino-3-(2-methyl-4-nitro-5-(pyridin-4-yl)-1H-indol-3-yl)propanoic acid;
2-amino-3-(2-methyl-4-nitro-6-(pyridin-4-yl)-1H-indol-3-yl)propanoic acid;
2-amino-3-(2-methyl-4-nitro-7-(pyridin-4-yl)-1H-indol-3-yl)propanoic acid;
2-amino-3-(2-methyl-7-nitro-4-(pyridin-4-yl)-1H-indol-3-yl)propanoic acid;
2-amino-3-(1,2,5-trimethyl-4-nitro-1H-indol-3-yl)propanoic acid;
2-amino-3-(1,2,6-trimethyl-4-nitro-1H-indol-3-yl)propanoic acid;
2-amino-3-(1,2,7-trimethyl-4-nitro-1H-indol-3-yl)propanoic acid;
2-amino-3-(1,2,4-trimethyl-7-nitro-1H-indol-3-yl)propanoic acid;
2-amino-3-(6-fluoro-1,2-dimethyl-4-nitro-1H-indol-3-yl)propanoic acid;
2-amino-3-(7-fluoro-1,2-dimethyl-4-nitro-1H-indol-3-yl)propanoic acid;
2-amino-3-(4-fluoro-1,2-dimethyl-7-nitro-1H-indol-3-yl)propanoic acid;
2-amino-3-(5-chloro-1,2-dimethyl-4-nitro-1H-indol-3-yl)propanoic acid;
2-amino-3-(6-chloro-1,2-dimethyl-4-nitro-1H-indol-3-yl)propanoic acid;
2-amino-3-(7-chloro-1,2-dimethyl-4-nitro-1H-indol-3-yl)propanoic acid;
2-amino-3-(4-chloro-1,2-dimethyl-7-nitro-1H-indol-3-yl)propanoic acid;
2-amino-3-(5-bromo-1,2-dimethyl-4-nitro-1H-indol-3-yl)propanoic acid;
2-amino-3-(6-bromo-1,2-dimethyl-4-nitro-1H-indol-3-yl)propanoic acid;
2-amino-3-(7-bromo-1,2-dimethyl-4-nitro-1H-indol-3-yl)propanoic acid;
2-amino-3-(4-bromo-1,2-dimethyl-7-nitro-1H-indol-3-yl)propanoic acid;
2-amino-3-(5-methoxy-1,2-dimethyl-4-nitro-1H-indol-3-yl)propanoic acid;
2-amino-3-(6-methoxy-1,2-dimethyl-4-nitro-1H-indol-3-yl)propanoic acid;
2-amino-3-(7-methoxy-1,2-dimethyl-4-nitro-1H-indol-3-yl)propanoic acid;
2-amino-3-(4-methoxy-1,2-dimethyl-7-nitro-1H-indol-3-yl)propanoic acid;
2-amino-3-(5-amino-1,2-dimethyl-4-nitro-1H-indol-3-yl)propanoic acid;
2-amino-3-(6-amino-1,2-dimethyl-4-nitro-1H-indol-3-yl)propanoic acid;
2-amino-3-(7-amino-1,2-dimethyl-4-nitro-1H-indol-3-yl)propanoic acid;
2-amino-3-(4-amino-1,2-dimethyl-7-nitro-1H-indol-3-yl)propanoic acid;
2-amino-3-(5-hydroxy-1,2-dimethyl-4-nitro-1H-indol-3-yl)propanoic acid;
2-amino-3-(6-hydroxy-1,2-dimethyl-4-nitro-1H-indol-3-yl)propanoic acid;
2-amino-3-(7-hydroxy-1,2-dimethyl-4-nitro-1H-indol-3-yl)propanoic acid;
2-amino-3-(4-hydroxy-1,2-dimethyl-7-nitro-1H-indol-3-yl)propanoic acid;
2-amino-3-(1,2-dimethyl-4-nitro-5-phenyl-1H-indol-3-yl)propanoic acid;
2-amino-3-(1,2-dimethyl-4-nitro-6-phenyl-1H-indol-3-yl)propanoic acid;
2-amino-3-(1,2-dimethyl-4-nitro-7-phenyl-1H-indol-3-yl)propanoic acid;
2-amino-3-(1,2-dimethyl-7-nitro-4-phenyl-1H-indol-3-yl)propanoic acid;
2-amino-3-(5-cyclopropyl-1,2-dimethyl-4-nitro-1H-indol-3-yl)propanoic acid;
2-amino-3-(6-cyclopropyl-1,2-dimethyl-4-nitro-1H-indol-3-yl)propanoic acid;
2-amino-3-(7-cyclopropyl-1,2-dimethyl-4-nitro-1H-indol-3-yl)propanoic acid;
2-amino-3-(4-cyclopropyl-1,2-dimethyl-7-nitro-1H-indol-3-yl)propanoic acid;
2-amino-3-(1,2-dimethyl-4-nitro-5-vinyl-1H-indol-3-yl)propanoic acid;
2-amino-3-(1,2-dimethyl-4-nitro-6-vinyl-1H-indol-3-yl)propanoic acid;
2-amino-3-(1,2-dimethyl-4-nitro-7-vinyl-1H-indol-3-yl)propanoic acid;
2-amino-3-(1,2-dimethyl-7-nitro-4-vinyl-1H-indol-3-yl)propanoic acid;
2-amino-3-(5-ethynyl-1,2-dimethyl-4-nitro-1H-indol-3-yl)propanoic acid;
2-amino-3-(6-ethynyl-1,2-dimethyl-4-nitro-1H-indol-3-yl)propanoic acid;
2-amino-3-(7-ethynyl-1,2-dimethyl-4-nitro-1H-indol-3-yl)propanoic acid;
2-amino-3-(4-ethynyl-1,2-dimethyl-7-nitro-1H-indol-3-yl)propanoic acid;
2-amino-3-(1,2-dimethyl-5-morpholino-4-nitro-1H-indol-3-yl)propanoic acid;
2-amino-3-(1,2-dimethyl-6-morpholino-4-nitro-1H-indol-3-yl)propanoic acid;
2-amino-3-(1,2-dimethyl-7-morpholino-4-nitro-1H-indol-3-yl)propanoic acid;
2-amino-3-(1,2-dimethyl-4-morpholino-7-nitro-1H-indol-3-yl)propanoic acid;
2-amino-3-(1,2-dimethyl-5-(methylthio)-4-nitro-1H-indol-3-yl)propanoic acid;
2-amino-3-(1,2-dimethyl-6-(methylthio)-4-nitro-1H-indol-3-yl)propanoic acid;
2-amino-3-(1,2-dimethyl-7-(methylthio)-4-nitro-1H-indol-3-yl)propanoic acid;
2-amino-3-(1,2-dimethyl-4-(methylthio)-7-nitro-1H-indol-3-yl)propanoic acid;
2-amino-3-(1,2-dimethyl-4-nitro-5-(pyridin-4-yl)-1H-indol-3-yl)propanoic acid;
2-amino-3-(1,2-dimethyl-4-nitro-7-(pyridin-4-yl)-1H-indol-3-yl)propanoic acid;
2-amino-3-(1,2-dimethyl-4-nitro-7-(pyridin-4-yl)-1H-indol-3-yl)propanoic acid; or
2-amino-3-(1,2-dimethyl-7-nitro-4-(pyridin-4-yl)-1H-indol-3-yl)propanoic acid;
and a pharmaceutically acceptable salt, prodrug, hydrate, or solvate thereof.

In some embodiments, a composition has a temperature below 37° C. (e.g., the temperature of the bacterial culture media of a composition is below 37° C.). In some embodiments, a composition has a temperature between 10 to 30° C. (e.g., the temperature of the bacterial culture media of a composition is between 10 to 30° C.). In some embodiments, a composition at a temperature of 28° C. (e.g., the temperature of the bacterial culture media of a composition is 28° C.).

In some embodiments, the disclosure relates to methods of producing a recombinant bacterial cell as described by the disclosure, the comprising the steps of: transforming a bacterial cell with an isolated nucleic acid engineered to express a fusion protein comprising a TxtE enzyme linked to a catalytic domain of a CYPI2A1 (P450BM3) reductase enzyme via an amino acid linker sequence that can be varied in terms of identities and length, e.g., that is between 14 and 27 amino acids in length; and an isolated nucleic acid engineered to express a nitric oxide synthase (NOS) enzyme; and culturing (e.g., growing) the bacterial cell.

In some embodiments of methods described by the disclosure a bacterial cell is transformed with an isolated nucleic acid engineered to express a glucose-1 dehydrogenase (GHD) enzyme.

In some embodiments of methods described by the disclosure, a bacterial cell is transformed with one or more an isolated nucleic acids comprising the sequence set forth in any one of SEQ ID NOs: 8-13.

In some aspects, the disclosure relates to methods for producing a composition as described by the disclosure, comprising the step of inoculating a bacterial culture medium with a recombinant bacterial cell as described by the disclosure.

In some aspects, the disclosure relates to methods for producing a nitrated L-tryptophan or nitrated L-tryptophan analogue, comprising the steps of: introducing into a bacterial cell culture comprising a one or more of a recombinant bacterial cell as described by the disclosure one or more L-Trp molecules and/or one or more L-Trp analogue molecules; and growing the bacterial cell culture under conditions under which a fusion protein expressed by the recombinant bacterial cell catalyzes a nitration reaction which produces one or more nitrated L-Trp molecules and/or one or more nitrated L-Trp analog molecules. In some embodiments, methods further comprise the step of isolating nitrated L-Trp molecules and/or nitrated L-Trp analog molecules from the bacterial cell culture. In some embodiments, the nitrated tryptophan or nitrated tryptophane analogue is a compound of Formulae I-VI. In some embodiments, the nitrated tryptophan or nitrated tryptophane analogue is:
(S)-2-amino-3-(4-methyl-5-nitro-1H-indol-3-yl)propanoic acid;
(S)-2-amino-3-(5-methyl-4-nitro-1H-indol-3-yl)propanoic acid;
(S)-2-amino-3-(6-methyl-4-nitro-1H-indol-3-yl)propanoic acid;
(S)-2-amino-3-(7-methyl-4-nitro-1H-indol-3-yl)propanoic acid;
(S)-2-amino-3-(4-methyl-7-nitro-1H-indol-3-yl)propanoic acid;
(S)-2-amino-3-(5-fluoro-4-nitro-1H-indol-3-yl)propanoic acid;
(S)-2-amino-3-(6-fluoro-4-nitro-1H-indol-3-yl)propanoic acid;
(S)-2-amino-3-(7-fluoro-4-nitro-1H-indol-3-yl)propanoic acid;
(S)-2-amino-3-(4-fluoro-7-nitro-1H-indol-3-yl)propanoic acid;
(S)-2-amino-3-(5-chloro-4-nitro-1H-indol-3-yl)propanoic acid;
(S)-2-amino-3-(6-chloro-4-nitro-1H-indol-3-yl)propanoic acid;
(S)-2-amino-3-(7-chloro-4-nitro-1H-indol-3-yl)propanoic acid;
(S)-2-amino-3-(4-chloro-7-nitro-1H-indol-3-yl)propanoic acid;
(S)-2-amino-3-(5-bromo-4-nitro-1H-indol-3-yl)propanoic acid;
(S)-2-amino-3-(6-bromo-4-nitro-1H-indol-3-yl)propanoic acid;
(S)-2-amino-3-(7-bromo-4-nitro-1H-indol-3-yl)propanoic acid;
(S)-2-amino-3-(4-bromo-7-nitro-1H-indol-3-yl)propanoic acid;
(S)-2-amino-3-(5-methoxy-4-nitro-1H-indol-3-yl)propanoic acid;
(S)-2-amino-3-(6-methoxy-4-nitro-1H-indol-3-yl)propanoic acid;
(S)-2-amino-3-(7-methoxy-4-nitro-1H-indol-3-yl)propanoic acid;
(S)-2-amino-3-(4-methoxy-7-nitro-1H-indol-3-yl)propanoic acid;
(S)-2-amino-3-(5-amino-4-nitro-1H-indol-3-yl)propanoic acid;
(S)-2-amino-3-(6-amino-4-nitro-1H-indol-3-yl)propanoic acid;
(S)-2-amino-3-(7-amino-4-nitro-1H-indol-3-yl)propanoic acid;
(S)-2-amino-3-(4-amino-7-nitro-1H-indol-3-yl)propanoic acid;
(S)-2-amino-3-(5-hydroxy-4-nitro-1H-indol-3-yl)propanoic acid;
(S)-2-amino-3-(6-hydroxy-4-nitro-1H-indol-3-yl)propanoic acid;
(S)-2-amino-3-(7-hydroxy-4-nitro-1H-indol-3-yl)propanoic acid;
(S)-2-amino-3-(4-hydroxy-7-nitro-1H-indol-3-yl)propanoic acid;
(S)-2-amino-3-(4-nitro-5-phenyl-1H-indol-3-yl)propanoic acid;
(S)-2-amino-3-(4-nitro-6-phenyl-1H-indol-3-yl)propanoic acid;
(S)-2-amino-3-(7-nitro-7-phenyl-1H-indol-3-yl)propanoic acid;
(S)-2-amino-3-(7-nitro-4-phenyl-1H-indol-3-yl)propanoic acid;
(S)-2-amino-3-(5-cyclopropyl-4-nitro-1H-indol-3-yl)propanoic acid;
(S)-2-amino-3-(6-cyclopropyl-4-nitro-1H-indol-3-yl)propanoic acid;
(S)-2-amino-3-(7-cyclopropyl-4-nitro-1H-indol-3-yl)propanoic acid;
(S)-2-amino-3-(4-cyclopropyl-7-nitro-1H-indol-3-yl)propanoic acid;
(S)-2-amino-3-(4-nitro-5-vinyl-1H-indol-3-yl)propanoic acid;
(S)-2-amino-3-(4-nitro-6-vinyl-1H-indol-3-yl)propanoic acid;
(S)-2-amino-3-(4-nitro-7-vinyl-1H-indol-3-yl)propanoic acid;
(S)-2-amino-3-(7-nitro-4-vinyl-1H-indol-3-yl)propanoic acid;
(S)-2-amino-3-(5-ethynyl-4-nitro-1H-indol-3-yl)propanoic acid;
(S)-2-amino-3-(6-ethynyl-4-nitro-1H-indol-3-yl)propanoic acid;
(S)-2-amino-3-(7-ethynyl-4-nitro-1H-indol-3-yl)propanoic acid;
(S)-2-amino-3-(4-ethynyl-7-nitro-1H-indol-3-yl)propanoic acid;
(S)-2-amino-3-(5-morpholino-4-nitro-1H-indol-3-yl)propanoic acid;
(S)-2-amino-3-(6-morpholino-4-nitro-1H-indol-3-yl)propanoic acid;
(S)-2-amino-3-(7-morpholino-4-nitro-1H-indol-3-yl)propanoic acid;
(S)-2-amino-3-(4-morpholino-7-nitro-1H-indol-3-yl)propanoic acid;
(S)-2-amino-3-(5-(methylthio)-4-nitro-1H-indol-3-yl)propanoic acid;
(S)-2-amino-3-(6-(methylthio)-4-nitro-1H-indol-3-yl)propanoic acid;
(S)-2-amino-3-(7-(methylthio)-4-nitro-1H-indol-3-yl)propanoic acid;
(S)-2-amino-3-(4-(methylthio)-7-nitro-1H-indol-3-yl)propanoic acid;
(S)-2-amino-3-(4-nitro-5-(pyridin-4-yl)-1H-indol-3-yl)propanoic acid;
(S)-2-amino-3-(4-nitro-6-(pyridin-4-yl)-1H-indol-3-yl)propanoic acid;
(S)-2-amino-3-(7-nitro-7-(pyridin-4-yl)-1H-indol-3-yl)propanoic acid;
(S)-2-amino-3-(7-nit-4-pyridin-4-yl)-1H-indol-3-yl)propanoic acid;
2-amino-3-(4-methyl-5-nitro-1H-indol-3-yl)propanoic acid;
2-amino-3-(5-methyl-4-nitro-1H-indol-3-yl)propanoic acid;
2-amino-3-(6-methyl-4-nitro-1H-indol-3-yl)propanoic acid;
2-amino-3-(7-methyl-4-nitro-1H-indol-3-yl)propanoic acid;
2-amino-3-(4-methyl-7-nitro-1H-indol-3-yl)propanoic acid;
2-amino-3-(6-fluoro-4-nitro-1H-indol-3-yl)propanoic acid;
2-amino-3-(7-fluoro-4-nitro-1H-indol-3-yl)propanoic acid;
2-amino-3-(4-fluoro-7-nitro-1H-indol-3-yl)propanoic acid;
2-amino-3-(5-chloro-4-nitro-1H-indol-3-yl)propanoic acid;
2-amino-3-(6-chloro-4-nitro-1H-indol-3-yl)propanoic acid;
2-amino-3-(7-chloro-4-nitro-1H-indol-3-yl)propanoic acid;
2-amino-3-(4-chloro-7-nitro-1H-indol-3-yl)propanoic acid;
2-amino-3-(5-bromo-4-nitro-1H-indol-3-yl)propanoic acid;
2-amino-3-(6-bromo-4-nitro-1H-indol-3-yl)propanoic acid;
2-amino-3-(7-bromo-4-nitro-1H-indol-3-yl)propanoic acid;
2-amino-3-(4-bromo-7-nitro-1H-indol-3-yl)propanoic acid;
2-amino-3-(5-methoxy-4-nitro-1H-indol-3-yl)propanoic acid;
2-amino-3-(6-methoxy-4-nitro-1H-indol-3-yl)propanoic acid;
2-amino-3-(7-methoxy-4-nitro-1H-indol-3-yl)propanoic acid;
2-amino-3-(4-methoxy-7-nitro-1H-indol-3-yl)propanoic acid;
2-amino-3-(5-amino-4-nitro-1H-indol-3-yl)propanoic acid;
2-amino-3-(6-amino-4-nitro-1H-indol-3-yl)propanoic acid;
2-amino-3-(7-amino-4-nitro-1H-indol-3-yl)propanoic acid;
2-amino-3-(4-amino-7-nitro-1H-indol-3-yl)propanoic acid;
2-amino-3-(5-hydroxy-4-nitro-1H-indol-3-yl)propanoic acid;
2-amino-3-(6-hydroxy-4-nitro-1H-indol-3-yl)propanoic acid;
2-amino-3-(7-hydroxy-4-nitro-1H-indol-3-yl)propanoic acid;
2-amino-3-(4-hydroxy-7-nitro-1H-indol-3-yl)propanoic acid;
2-amino-3-(4-nitro-5-phenyl-1H-indol-3-yl)propanoic acid;
2-amino-3-(4-nitro-6-phenyl-1H-indol-3-yl)propanoic acid;
2-amino-3-(4-nitro-7-phenyl-1H-indol-3-yl)propanoic acid;
2-amino-3-(7-nitro-4-phenyl-1H-indol-3-yl)propanoic acid;
2-amino-3-(5-cyclopropyl-4-nitro-1H-indol-3-yl)propanoic acid;
2-amino-3-(6-cyclopropyl-4-nitro-1H-indol-3-yl)propanoic acid;
2-amino-3-(7-cyclopropyl-4-nitro-1H-indol-3-yl)propanoic acid;
2-amino-3-(4-cyclopropyl-7-nitro-1H-indol-3-yl)propanoic aid;
2-amino-3-(4-nitro-5-vinyl-1H-indol-3-yl)propanoic acid;
2-amino-3-(4-nitro-6-vinyl-1H-indol-3-yl)propanoic acid;
2-amino-3-(4-nitro-7-vinyl-1H-indol-3-yl)propanoic acid;
2-amino-3-(7-nitro-4-vinyl-1H-indol-3-yl)propanoic acid;
2-amino-3(5-ethynyl-4-nitro-1H-indol-3-yl)propanoic acid;
2-amino-3-(6-ethynyl-4-nitro-1H-indol-3-yl)propanoic acid;
2-amino-3-(7-ethynyl-4-nitro-1H-indol-3-yl)propanoic acid;
2-amino-3-(4-ethynyl-7-nitro-1H-indol-3-yl)propanoic acid;
2-amino-3-(5-morpholino-4-nitro-1H-indol-3-yl)propanoic acid;
2-amino-3-(6-morpholino-4-nitro-1H-indol-3-yl)propanoic acid;
2-amino-3-(7-morpholino-4-nitro-1H-indol-3-yl)propanoic acid;
2-amino-3-(5-morpholino-7-nitro-1H-indol-3-yl)propanoic acid;
2-amino-3-(5-(methylthio)-4-nitro-1H-indol-3-yl)propanoic acid;
2-amino-3-(6-(methylthio)-4-nitro-1H-indol-3-yl)propanoic acid;
2-amino-3-(7-(methylthio)-4-nitro-1H-indol-3-yl)propanoic acid;
2-amino-3-(4-(methylthio-nitro-1H-indol-3-yl)propanoic acid;
2-amino-3-(4-nitro-5-(pyridin-4-yl)-1H-indol-3-yl)propanoic acid;
2-amino-3-(4-nitro-6-pyridin-4-yl)-1H-indol-3-yl)propanoic acid;
2-amino-3-(4-nitro-7-(pyridin-4-yl)-1H-indol-3-yl)propanoic acid;
2-amino-3-(7-nitro-4-(pyridin-4-yl)-1H-indol-3-yl)propanoic acid;
2-amino-3-(1,5-dimethyl-4-nitro-1H-indol-3-yl)propanoic acid;
2-amino-3-(1,6-dimethyl-4-nitro-1H-indol-3-yl)propanoic acid;
2-amino-3-(1,7-dimethyl-4-nitro-1H-indol-3-yl)propanoic acid;
2-amino-3-(6,4-dimethyl-7-nitro-1H-indol-3-yl)propanoic acid;
2-amino-3-(6-fluoro-1-methyl-4-nitro-1H-indol-3-yl)propanoic acid;
2-amino-3-(7-fluoro-1-methyl-4-nitro-1H-indol-3-yl)propanoic acid;
2-amino-3-(4-fluoro-1-methyl-7-nitro-1H-indol-3-yl)propanoic acid;
2-amino-3-(5-chloro 1-methyl-4-nitro-1H-indol-3-yl)propanoic acid;
2-amino-3-(6-chloro-1-methyl-4-nitro-1H-indol-3-yl)propanoic acid;
2-amino-3-(7-chloro-1-methyl-4-nitro-1H-indol-3-yl)propanoic acid;
2-amino-3(4-chlor-1-methyl-7-nitro-1H-indol-3-yl)propanoic acid;
2-amino-3-(5-bromo-1-methyl-4-nitro-1H-indol-3-yl)propanoic acid;
2-amino-3-(6-bromo-1-methyl-4-nitro-1H-indol-3-yl)propanoic acid;
2-amino-3-(7-bromo-1-methyl-7-nitro-1H-indol-3-yl)propanoic acid;
2-amino-3-(4-bromo-1-methyl-7-nitro-1H-indol-3-yl)propanoic acid;
2-amino-3-(5-methoxy-1-methyl-4-nitro-1H-indol-3-yl)propanoic acid;
2-amino-3-(6-methoxy-1-methyl-4-nitro-1H-indol-3-yl)propanoic acid;
2-amino-3-(7-methoxy-1-methyl-4-nitro-1H-indol-3-yl)propanoic acid;
2-amino-3-(4-methoxy-1-methyl-7-nitro-1H-indol-3-yl)propanoic acid;
2-amino-3-(5-amino-1-methyl-4-nitro-1H-indol-3-yl)propanoic acid;
2-amino-3-(6-amino-1-methyl-4-nitro-1H-indol-3-yl)propanoic acid;
2-amino-3-(7-amino-1-methyl-4-nitro-1H-indol-3-yl)propanoic acid;
2-amino-3-(4-amino-1-methyl-7-nitro-1H-indol-3-yl)propanoic acid;
2-amino-3-(5-hydroxy-1-methyl-4-nitro-1H-indol-3-yl)propanoic acid;
2-amino-3-(6-hydroxy-1-methyl-4-nitro-1H-indol-3-yl)propanoic acid;
2-amino-3-(7-hydroxy-1-methyl-4-nitro-1H-indol-3-yl)propanoic acid;
2-amino-3-(4-hydroxy-1-methyl-7-nitro-1H-indol-3-yl)propanoic acid;
2-amino-3-(1-methyl-4-nitro-5-phenyl-1H-indol-3-yl)propanoic acid;
2-amino-3-(1-methyl-4-nitro-6-phenyl-1H-indol-3-yl)propanoic acid;
2-amino-3-(1-methyl-4-nitro-7-phenyl-1H-indol-3-yl)propanoic acid;
2-amino-3-(5-methyl-7-nitro-phenyl-1H-indol-3-yl)propanoic acid;
2-amino-3-(5-cyclopropyl-1-methyl-4-nitro-1H-indol-3-yl)propanoic acid;
2-amino-3-(6-cyclopropyl-1-methyl-4-nitro-1H-indol-3-yl)propanoic acid;
2-amino-3-(7-cyclopropyl-1-methyl-4-nitro-1H-indol-3-yl)propanoic acid;
2-amino-3-(4-cyclopropyl-1-methyl-7-nitro-1H-indol-3-yl)propanoic acid;
2-amino-3-(1-methyl-4-nitro-5-vinyl-1H-indol-3-yl)propanoic acid;
2-amino-3-(1-methyl-4-nitro-6-vinyl-1H-indol-3-yl)propanoic acid;
2-amino-3-(1-methyl-4-nitro-7-vinyl-1H-indol-3-yl)propanoic acid;
2-amino-3-(1-methyl-7-nitro-4-viny-1H-indol-3-yl)propanoic acid;
2-amino-3-(5-ethynyl-1-methyl-4-nitro-1H-indol-3-yl)propanoic acid;
2-amino-3-(6-ethynyl-1-methyl-4-nitro-1H-indol-3-yl)propanoic acid;
2-amino-3-(7-ethynyl-1-methyl-4-nitro-1H-indol-3-yl)propanoic acid;
2-amino-3-(4-ethynyl-1-methyl-7-nitro-1H-indol-3-yl)propanoic acid;
2-amino-3-(1-methyl-5-morpholino-4-nitro-1H-indol-3-yl)propanoic acid;
2-amino-3-(1-methyl-6-morpholino-4-nitro-1H-indol-3-yl)propanoic acid;
2-amino-3-(1-methyl-7-morpholino-4-nitro-1H-indol-3-yl)propanoic acid;
2-amino-3-(1-methyl-4-morpholino-7-nitro-1H-indol-3-yl)propanoic acid;
2-amino-3-(1-methyl-5-(methylthio)-4-nitro-1H-indol-3-yl)propanoic acid;
2-amino-3-(1-methy-6-(methylthio)-4-nitro-1H-indol-3-yl)propanoic acid;
2-amino-3-(1-methyl-7-(methylthio)-4-nitro-1H-indol-3-yl)propanoic acid;
2-amino-3-(1-methyl-4-(methylthio)-7-nitro-1H-indol-3-yl)propanoic acid;
2-amino-3-(1-methyl-4-nitro-5-(pyridin-4-yl)-1H-indol-3-yl)propanoic acid;
2-amino-3-(1-methyl-4-nitro-6-(pyridin-4-yl)-1H-indol-3-yl)propanoic acid;
2-amino-3-(1-methyl-4-nitro-7-(pyridin-4-yl)-1H-indol-3-yl)propanoic acid;
2-amino-3-(1-methyl-7-nitro-4-(pyridin-4-yl)-1H-indol-3-yl)propanoic acid;
2-amino-3-(2,5-dimethyl-4-nitro-1H-indol-3-yl)propanoic acid;
2-amino-3-(2,6-dimethyl-4-nitro-1H-indol-3-yl)propanoic acid;
2-amino-3-(2,7-dimethyl-4-nitro-1H-indol-3-yl)propanoic acid;
2-amino-3-(2,4-dimethyl-7-nitro-1H-indol-3-yl)propanoic acid;
2-amino-3-(6-fluoro-2-methyl-4-nitro-1H-indol-3-yl)propanoic acid;
2-amino-3-(7-fluoro-2-methyl-4-nitro-1H-indol-3-yl)propanoic acid;
2-amino-3-(4-fluoro-2-methyl-7-nitro-1H-indol-3-yl)propanoic acid;
2-amino-3-(5-chloro-2-methyl-4-nitro-1H-indol-3-yl)propanoic acid;
2-amino-3-(6-chloro-2-methyl-4-nitro-1H-indol-3-yl)propanoic acid;
2-amino-3-(7-chloro-2-methyl-4-nitro-1H-indol-3-yl)propanoic acid;
2-amino-3-(4-chloro-2-methyl-7-nitro-1H-indol-3-yl)propanoic acid;
2-amino-3-(5-bromo-2-methyl-4-nitro-1H-indol-3-yl)propanoic acid;
2-amino-3-(6-bromo-2-methyl-4-nitro-1H-indol-3-yl)propanoic acid;
2-amino-3-(7-bromo-2-methyl-4-nitro-1H-indol-3-yl)propanoic acid;
2-amino-3-(4-bromo-2-methyl-7-nitro-1H-indol-3-yl)propanoic acid;
2-amino-3-(5-methoxy-2-methyl-4-nitro-1H-indol-3-yl)propanoic acid;
2-amino-3-(6-methoxy-2-methyl-4-nitro-1H-indol-3-yl)propanoic acid;
2-amino-3-(7-methoxy-2-methyl-4-nitro-1H-indol-3-yl)propanoic acid;
2-amino-3-(4-methoxy-2-methyl-7-nitro-1H-indol-3-yl)propanoic acid;
2-amino-3-(5-amino-2-methyl-4-nitro-1H-indol-3-yl)propanoic acid;
2-amino-3-(6-amino-2-methyl-4-nitro-1H-indol-3-yl)propanoic acid;
2-amino-3-(7-amino-2-methyl-4-nitro-1H-indol-3-yl)propanoic acid;
2-amino-3-(4-amino-2-methyl-7-nitro-1H-indol-3-yl)propanoic acid;
2-amino-3-(5-hydroxy-2-methyl-4-nitro-1H-indol-3-ylpropanoic acid;
2-amino-3-(6-hydroxy-2-methyl-4-nitro-1H-indol-3-yl)propanoic acid;
2-amino-3-(7-hydroxy-2-methyl-4-nitro-1H-indol-3-yl)propanoic acid;
2-amino-3-(4-hydroxy-2-methyl-7-nitro-1H-indol-3-yl)propanoic acid;
2-amino-3-(2-methyl-4-nitro-5-phenyl-1H-indol-3-yl)propanoic acid;
2-amino-3-(2-methyl-4-nitro-4-phenyl-1H-indol-3-yl)propanoic acid;
2-amino-3-(2-methyl-4-nitro-7-phenyl-1H-indol-3-yl)propanoic acid;
2-amino-3-(2-methyl-7-nitro-4-phenyl-1H-indol-3-yl)propanoic acid;
2-amino-3-(5-cyclopropyl-2-methyl-4-nitro-1H-indol-3-yl)propanoic acid;
2-amino-3-(6-cyclopropyl-2-methyl-4-nitro-1H-indol-3-yl)propanoic acid;
2-amino-3-(7-cyclopropyl-2-methyl-4-nitro-1H-indol-3-yl)propanoic acid;
2-amino-3-(4-cyclopropyl-2-methyl-7-nitro-1H-indol-3-yl)propanoic acid;
2-amino-3-(2-methyl-4-nitro-5-vinyl-1H-indol-3-yl)propanoic acid;
2-amino-3-(2-methyl-4-nitro-6-vinyl-1H-indol-3-yl)propanoic acid;
2-amino-3-(2-methyl-4-nitro-7-vinyl-1H-indol-3-yl)propanoic acid;
2-amino-3-(2-methyl-7-nitro-4-vinyl-1H-indol-3-yl)propanoic acid;
2-amino-3-(5-ethynyl-2-methyl-4-nitro-1H-indol-3-yl)propanoic acid;
2-amino-3-(6-ethynyl-2-methyl-4-nitro-1H-indol-3-yl)propanoic acid;
2-amino-3-(7-ethynyl-2-methyl-4-nitro-1H-indol-3-yl)propanoic acid;
2-amino-3-(4-ethynyl-2-methyl-7-nitro-1H-indol-3-yl)propanoic acid;
2-amino-3-(2-methyl-5-morpholino-4-nitro-1H-indol-3-yl)propanoic acid;
2-amino-3-(2-methyl-6-morpholino-4-nitro-1H-indol-3-yl)propanoic acid;
2-amino-3-(2-methyl-7-morpholino-4-nitro-1H-indol-3-yl)propanoic acid;
2-amino-3-(2-methyl-4-morpholino-7-nitro-1H-indol-3-yl)propanoic acid;
2-amino-3-(2-methyl-5-(methylthio)-4-nitro-1H-indol-3-yl)propanoic acid;
2-amino-3-(2-methyl-6-(methylthio)-4-nitro-1H-indol-3-yl)propanoic acid;
2-amino-3-(2-methyl-7-(methylthio)-4-nitro-1H-indol-3-yl)propanoic acid;
2-amino-3(2-methyl-4-(methylthio)-7-nitro-1H-indol-3-yl)propanoic acid;
2-amino-3-(2-methyl-4-nitro-5-(pyridin-4-yl)-1H-indol-3-yl)propanoic acid;
2-amino-3-(2-methyl-4-nitro-6-(pyridin-4-yl)-1H-indol-3-yl)propanoic acid;
2-amino-3-(2-methyl-4-nitro-7-(pyridin-4-yl)-1H-indol-3-yl)propanoic acid;
2-amino-3-(2-methyl-7-nitro-4-(pyridin-4-yl)-1H-indol-3-yl)propanoic acid;
2-amino-3-(1,2,5-trimethyl-4-nitro-1H-indol-3-yl)propanoic acid;
2-amino-3-(1,2,6-trimethyl-4-nitro-1H-indol-3-yl)propanoic acid;
2-amino-3-(1,2,7-trimethyl-4-nitro-1H-indol-3-yl)propanoic acid;
2-amino-3-(1,2,4-trimethyl-7-nitro-1H-indol-3-yl)propanoic acid;
2-amino-3-(6-fluoro-1,2-dimethyl-4-nitro-1H-indol-3-yl)propanoic acid;
2-amino-3-(7-fluoro-1,2-dimethyl-4-nitro-1H-indol-3-yl)propanoic acid;
2-amino-3-(4-fluoro-1,2-dimethyl-7-nitro-1H-indol-3-yl)propanoic acid;
2-amino-3(5-chloro-1,2-dimethyl-4-nitro-1H-indol-3-yl)propanoic acid;
2-amino-3-(6-chloro-1,2-dimethyl-4-nitro-1H-indol-3-yl)propanoic acid;
2-amino-3-(7-chloro-1,2-dimethyl-4-nitro-1H-indol-3-yl)propanoic acid;
2-amino-3-(4-chloro-1,2-dimethyl-7-nitro-1H-indol-3-yl)propanoic acid;
2-amino-3-(5-bromo-1,2-dimethyl-4-nitro-1H-indol-3-yl)propanoic acid;
2-amino-3-(6-bromo-1,2-dimethyl-4-nitro-1H-indol-3-yl)propanoic acid;
2-amino-3-(7-bromo-1,2-dimethyl-4-nitro-1H-indol-3-yl)propanoic acid;
2-amino-3-(4-bromo-1,2-dimethyl-7-nitro-1H-indol-3-yl)propanoic acid;
2-amino-3-(5-methoxy-1,2-dimethyl-4-nitro-1H-indol-3-yl)propanoic acid;
2-amino-3-(6-methoxy-1,2-dimethyl-4-nitro-1H-indol-3-yl)propanoic acid;
2-amino-3-(7-methoxy-1,2-dimethyl-4-nitro-1H-indol-3-yl)propanoic acid;
2-amino-3-(4-methoxy-1,2-dimethyl-7-nitro-1H-indol-3-yl)propanoic acid;
2-amino-3-(5-amino-1,2-dimethyl-4-nitro-1H-indol-3-yl)propanoic acid;
2-amino-3-(6-amino-1,2-dimethyl-4-nitro-1H-indol-3-yl)propanoic acid;
2-amino-3-(7-amino-1,2-dimethyl-4-nitro-1H-indol-3-yl)propanoic acid;
2-amino-3-(4-amino-1,2-dimethyl-7-nitro-1H-indol-3-yl)propanoic acid;
2-amino-3-(5-hydroxy-1,2-dimethyl-4-nitro-1H-indol-3-yl)propanoic acid;
2-amino-3-(6-hydroxy-1,2-dimethyl-4-nitro-1H-indol-3-yl)propanoic acid;
2-amino-3-(7-hydroxy-1,2-dimethyl-4-nitro-1H-indol-3-yl)propanoic acid;
2-amino-3-(4-hydroxy-1,2-dimethyl-7-nitro-1H-indol-3-yl)propanoic acid;
2-amino-3-(1,2-dimethyl-4-nitro-5-phenyl-1H-indol-3-yl)propanoic acid;
2-amino-3-(1,2-dimethyl-4-nitro-6-phenyl-1H-indol-3-yl)propanoic acid;
2-amino-3-(1,2-dimethyl-4-nitro-7-phenyl-1H-indol-3-yl)propanoic acid;
2-amino-3-(1,2-dimethyl-7-nitro-4-phenyl-1H-indol-3-yl)propanoic acid;
2-amino-3-(5-cyclopropyl-1,2-dimethyl-4-nitro-1H-indol-3-yl)propanoic acid;
2-amino-3-(6-cyclopropyl-1,2-dimethyl-4-nitro-1H-indol-3-yl)propanoic acid;
2-amino-3-(7-cyclopropyl-1,2-dimethyl-4-nitro-1H-indol-3-yl)propanoic acid;
2-amino-3-(4-cyclopropyl-1,2-dimethyl-7-nitro-1H-indol-3-yl)propanoic acid;
2-amino-3-(1,2-dimethyl-4-nitro-5-vinyl-4H-indol-3-yl)propanoic acid;
2-amino-3-(1,2-dimethyl-4-nit)-6-vinyl-1H-indol-3-yl)propanoic acid;
2-amino-3-(1,2-dimethyl-4-nitro-7-vinyl-1H-indol-3-yl)propanoic acid;
2-amino-3-(1,2-dimethyl-7-nitro-4-vinyl-1H-indol-3-yl)propanoic acid;
2-amino-3-(5-ethynyl-1,2-dimethyl-4-nitro-1H-indol-3-yl)propanoic acid;
2-amino-3-(6-ethynyl-1,2-dimethyl-4-nitro-1H-indol-3-yl)propanoic acid;
2-amino-3-(7-ethynyl-1,2-dimethyl-4-nitro-1H-indol-3-yl)propanoic acid;
2-amino-3-(4-ethynyl-1,2-dimethyl-7-nitro-1H-indol-3-yl)propanoic acid;
2-amino-3-(1,2-dimethyl-5-morpholino-4-nitro-1H-indol-3-yl)propanoic acid;
2-amino-3-(1,2-dimethyl-6-morpholino-4-nitro-1H-indol-3-yl)propanoic acid;
2-amino-3-(1,2-dimethyl-7-morpholino-4-nitro-1H-indol-3-yl)propanoic acid;
2-amino-3-(1,2-dimethyl-4-morpholino-7-nitro-1H-indol-3-yl)propanoic acid;
2-amino-3-(1,2-dimethyl-5-(methylthio)-4-nitro-1H-indol-3-yl)propanoic acid;
2-amino-3-(1,2-dimethyl-6-(methylthio)-4-nitro-1H-indol-3-yl)propanoic acid;
2-amino-3-(1,2-dimethyl-7-(methylthio)-4-nitro-1H-indol-3-yl)propanoic acid;
2-amino-3-(1,2-dimethyl-4-(methylthio)-7-nitro-1H-indol-3-yl)propanoic acid;
2-amino-3-(1,2-dimethyl-4-nitro-5-(pyridin-4-yl)-1H-indol-3-yl)propanoic acid;
2-amino-3-(1,2-dimethyl-4-nitro-6-(pyridin-4-yl)-1H-indol-3-yl)propanoic acid;
2-amino-3-(1,2-dimethyl-4-nitro-7-(pyridin-4-yl)-1H-indol-3-yl)propanoic acid; or
2-amino-3-(1,2-dimethyl-7-nitro-4-(pyridin-4-yl)-1H-indol-3-yl)propanoic acid;
and a pharmaceutically acceptable salt, prodrug, hydrate, or solvate thereof.

In some embodiments of methods described by the disclosure, one or more compounds of Formula Ia or IVa. In some embodiments of methods described by the disclosure, one or more L-Trp analogue molecules are selected from the group consisting of α-Me-Trp, 4-F-Trp, 4-Me-Trp, 5-MeO-Trp, 5-Me-Trp, 5-F-Trp, 6-F-Trp, and 7-Me-Trp.

In some embodiments of methods described by the disclosure, the step of growing the bacterial cell culture comprises introducing one or more antibiotic and/or one or more inducer into the bacterial cell culture. In some embodiments, one or more antibiotic is selected from ampicillin and kanamycin. In some embodiments, one or more of the inducers is Isopropyl β-D-1-thiogalactopyranoside (IPTG).

In some embodiments of methods described by the disclosure, the step of growing a bacterial cell culture is performed at a temperature below 37° C. In some embodiments, the step of growing the bacterial cell culture is performed at a temperature between 10 to 30° C. optionally at a temperature of 28° C.

In some embodiments, a bacterial cell culture is grown for up to 25 hours (e.g., up to 25 hours post-transformation with one or more isolated nucleic acids).

In some embodiments, isolating nitrated L-Trp molecules and/or nitrated L-Trp analog molecules comprises lysing one or more recombinant bacterial cells. In some embodiments, isolating nitrated L-Trp molecules and/or nitrated L-Trp analog molecules further comprises performing high-pressure liquid chromatography (HPLC) on a bacterial cell lysate, or purifying a bacterial lysate by performing a liquid/solid (e.g., carbon-based, such as C18) purification technique.

### BRIEF DESCRIPTION OF DRAWINGS

FIG. 1 is schematic overview of bacterial cell factories for the production of nitro-chemicals.

FIGS. 2A-2B show representative data for activity testing of a whole cell nitration system. FIG. 2A shows SDS-PAGE analysis of a whole cell nitration system. M, marker; 1, pETDUET-TB14-BsNOS+pET28b-GDH; 2, pETDUET-GDH-BsNOS+pET28b-TB14; 3, pACYCDUET-TB14-BsNOS+pET28b-GDH; 4, pACYCDUET-GDH-BsNOS+pET28b-TB14. Three soluble recombinant proteins (TB14, BsNOS, and GDH) are identified with arrows. FIG. 2B shows LCMS analysis of products in the whole cell nitration.

FIGS. 3A-3B show analysis of different embodiments of a whole cell nitration system. FIG. 3A is a schematic representation of plasmids combination used in different embodiments of a whole cell nitration system. FIG. 3B shows nitrated tryptophan concentration produced by different embodiments of a whole cell nitration system. Bacterial growth was supported by M9 medium.

FIG. 4 shows nitrated tryptophan concentration produced by one embodiment of a whole cell nitration system supported by different types of growth medium (e.g., M9, LB, SOC, TB, each with or without supplemented L-Arg or L-Trp).

FIG. 5 shows nitrated tryptophan concentration produced by one embodiment of a whole cell nitration system fermented at different temperatures.

FIG. 6 shows production of nitrated tryptophan analogues by one embodiment of a whole cell nitration system.

FIG. 7 shows L-Trp, 4-N-L-Trp, and indole concentration as a function of incubation time during a whole cell transformation process.

FIG. 8 is a schematic showing biosynthesis pathways of aromatic amino acids and targeted genes (e.g., TyrA, PheA. TrpR, TnaA) in a host engineering study.

FIG. 9 shows a schematic depicting one embodiment of a Nitrotrp biosynthetic pathway that is integrated with select major metabolic pathways in E. coli. TB14, BsNOS, and GDH genes are shadowed. Cellular Nitrotrp and nitric oxide (NO) are also shown. Inhibition of the shikimate pathway and L-Trp biosynthesis by TrpR is shown in dashed lines. The X symbol indicates gene inactivation. Inactivation of trpR, pheA and tyrA increases synthesis of L-Trp shown as wider arrows, GLC glucose, G6P glucose-6-phosphate, F6P fructose-6-phosphate. G3P glyceraldehyde-3-phosphate, PEP phosphoenolpyruvate, PYR pyruvate, GLN glucono-1,5-lactone, 6PG 6-phosphogluconolactone, RSP ribose-5-phosphate, E4P erythrose-4-phosphate, DAHP 3-deoxy-D-arabino-heptulosonate, CHA chorismate, PPA prephenate. ANT anthranilate. IND indole, TCA tricarboxylic acid cycle, AKG α-ketoglutarate, Ctl L-citrulline, trpE component I of anthranilate synthase, trpR trp operon repressor, tnaA tryptophanase, pheA chorismate mutase/prephenate dehydratase and tyrA chorismate mutase/prephenate dehydrogenase.

FIGS. 10A-10C show one embodiments of an E. coli-based system to produce Nitrotrp. FIG. 10A shows design of four Nitrotrp biosynthetic pathways comprising TB14, BsNOS, and GDH. These genes were cloned in pETDuet-1 and pET28b or pACYCDuet-1 and pET28b. FIG. 10B shows SDS-PAGE analysis of soluble protein fractions of E. coli cells transformed with the pathway I-IV. Protein expression in E. coli-I to -IV was induced by 0.5 mM IPTG in TB at 18° C., 250 rpm for 20 h. An equal volume of soluble protein fractions prepared from the same concentrations of cell resuspension solutions was used for SDS-PAGE analysis. Bands of three soluble recombinant proteins were indicated with arrows. FIG. 10C shows production of Nitrotrp by E. coli-I to -IV in the M9 medium at 20° C., 250 rpm. Aliquots (0.1 mL) of the fermentation culture were taken at days 1 to 4 and the concentration of Nitrotrp in the samples was measured in HPLC analysis. The data represent means±s. d. of at least two independent experiments.

FIG. 11 shows SDS-PAGE analysis of purified recombinant TB14, BsNOS, and GDH. All three proteins showed expected molecular weights, the same as those in E. coli soluble lysates.

FIGS. 12A-12B show E. coli cells carrying the pathway I produced Nitrotrp. FIG. 12A shows HPLC analysis of authentic Nitrotrp (1) and clear fermentation medium (II) demonstrated the production of Nitrotrp. FIG. 12B shows ESI-MS spectrum of Nitrotrp produced by E. coli. The calculated m/z of [M+H]+ is 250.1, identical to determined value.

FIG. 13 shows HPLC analysis of authentic 4-nitroindole (1) and clear fermentation medium prepared at day 4 (II) demonstrated no production of 4-nitroindole. The calculated m/z of [M+H]+ is 250.1, identical to determined value.

FIGS. 14A-14B show TnaA does not convert Nitrotrp into 4-nitroindole. FIG. 14A shows SDS-PAGE analysis of purified recombinant TnaA (around 56 kD). FIG. 14B shows HPLC analysis of TnaA reactions with L-Trp (I) and Nitrotrp (II) as substrates. The reactions contained 0.1 μM purified TnaA and 0.5 mM substrate and incubated at 37° C. for 10 min. No 4-nitro indole was produced in the enzyme reaction.

FIG. 15 shows E. coli BL21-GOLD (DE3) transformed with the pathway I-IV showed similar growth rates in TB medium. Cultures were grown at 37° C., 250 rpm for 4 hours and protein expression was induced with 0.5 mM IPTG at 18° C., 250 rpm for 12 hours. OD600 were measured at serial time points. The data represent means±s. d. of at least two independent experiments.

FIGS. 16A-16B show improvement of Nitrotrp production by varying fermentation media and temperature. FIG. 16A shows the titers of Nitrotrp were varied when E. coli-II was fermented in M9, LB and TB media in the presence or absence of 5 mM L-Trp or L-Arg at 20° C., 250 rpm. FIG. 16B shows the titers of Nitrotrp were influenced by fermentation temperature. The fermentation was performed in TB medium at 15° C., 20° C., 28° C. or 37° C. and 250 rpm. Aliquots (0.1 mL) of the fermentation culture were taken at various time points and the concentration of Nitrotrp in the samples was measured in HPLC analysis. The data represent means±s. d, of at least two independent experiments.

FIG. 17 shows relative titers of Nitrotrp by five E. coli strains. All strains were cultured in TB at 28° C., 250 rpm. Aliquots (0.1 mL) of the fermentation culture were taken after 24 h and the concentration of Nitrotrp in the samples was measured in HPLC analysis. The amount of Nitrotrp by E. coli-II was set as 100% for normalizing the relative titer of other strains. The data represent means±s. d. of at least two independent experiments.

### DETAILED DESCRIPTION

The disclosure relates, in some aspects. to compositions and methods useful for production of nitrated aromatic molecules. The disclosure is based, in part, on whole cell systems expressing artificial fusion proteins comprising cytochrome P450 enzymes linked to reductase enzymes. A significant advantage of whole cell nitration systems described by the disclosure compared to in vitro nitration reactions is the in situ production of NO from L-Arg, which enables recombinant bacterial cells to produce NO from L-Arg, which is synthesized by the bacterial cells from cheap carbon and nitrogen sources. Thus, it is believed that whole cell nitration systems described by the disclosure greatly lower the cost of biocatalytic nitration processes relative to currently utilized methods.

### Recombinant Bacterial Cells

in some aspects, the disclosure relates to a recombinant bacterial cell comprising one or more isolated nucleic acids engineered to express: a fusion protein comprising a TxtE enzyme linked to a catalytic domain of a CYP102A1 (P450BM3) reductase enzyme via an amino acid linker sequence that is between 14 and 27 amino acids in length; and a nitric oxide synthase (NOS) enzyme.

As used herein “nucleic acid” refers to a DNA or RNA molecule. An “isolated nucleic acid” refers to a nucleic acid (e.g., DNA or RNA) that has been prepared in vitro, for example by recombinant technology. Nucleic acids are polymeric macromolecules comprising a plurality of nucleotides. In some embodiments, the nucleotides are deoxyribonucleotides or ribonucleotides. In some embodiments, the nucleotides comprising the nucleic acid are selected from the group consisting of adenine, guanine, cytosine, thymine, uracil and inosine. In some embodiments, the nucleotides comprising the nucleic acid are modified nucleotides. Non-limiting examples of natural nucleic acids include genomic DNA and plasmid DNA. In some embodiments, the nucleic acids of the instant disclosure are synthetic. As used herein, the term “synthetic nucleic acid” refers to a nucleic acid molecule that is constructed via joining nucleotides by a synthetic or non-natural method. One non-limiting example of a synthetic method is solid-phase oligonucleotide synthesis. In some embodiments, the nucleic acids of the instant disclosure are isolated.

In some aspects, the disclosure relates to bacterial cells (e.g., populations of bacterial cells) that have been genetically engineered to express biocatalysts useful for aromatic nitration in situ (e.g., aromatic nitration inside the bacterial cell(s)). Generally, a bacterial cell of the disclosure may be any Gram-negative bacterial cell, including but not limited to bacteria of the genus Eschereria sp. (e.g., E. coli), Pseudomonas sp., Xunthomonas sp., Rhizobium sp., Azotobacter sp., Acetobacter sp., Gluconobacter sp., Methylococcus sp., Klebsiella sp., Bacteroides sp., Yersinia sp., Vibrio sp., Bacillus sp., Clostridium sp., Lactococcus sp., Lactobacillus sp., Staphylococcus sp., and Streptococcus sp. In some embodiments, a bacterial cell is an E. coli cell. Examples of E. coli bacterial strains include E. coli Dh5α, E. coli BL21, E. coli DE3, E. coli Lemo21, E. coli NiCo21, E. coli Rosetta, etc. In some embodiments, a bacterial cell is an E. coli BL21-Gold cell.

In some embodiments. bacterial cells are genetically engineered to express certain biocatalysts (e.g., proteins, such as enzymes) capable of aromatic nitration. Typically, the biocatalyst comprises a self-sufficient cytochrome P450 enzyme, or a portion thereof, Previously described self-sufficient cytochrome P450 enzymes typically comprise (i) a cytochrome P450 enzyme which catalyzes transfer of a nitro functional group to aromatic moieties (e.g., indole); (ii) an amino acid linker; and, (iii) a catalytic domain of a reductase enzyme. For example, TxtE-P450BM3 (also referred to as TB13-Q) is described in PCT Publication WO 2016/134145, the entire contents of which are incorporated by reference herein. In another example a TxtE-450BM3 variant having a 14-amino acid linker (also referred to herein as TB14) is described in PCT International Application Number PCT/US2017/058579, the entire contents of which are incorporated herein by reference. In some embodiments, a fusion protein expressed by a recombinant bacterial cell described by the disclosure is a TB14 fusion protein having the sequence set forth in SEQ ID NO: 1.

As used herein, the term “TxtE enzyme” refers to a (i) polypeptide comprising the entire amino acid sequence of TxtE, (ii) a portion of TxtE which maintains the function of catalyzing transfer of a nitro functional group to aromatic moieties (e.g., indole), or (iii) an enzyme which catalyzes transfer of a nitro functional group to aromatic moieties (e.g., indole) and is at least 95% homologous to the amino acid sequence of TxtE. For example, in some embodiments, a TxtE enzyme comprises or consists of a sequence set forth in Genbank Accession No. CBG70284.1 or a portion thereof (e.g., SEQ ID NO: 14).

The skilled artisan recognizes that for a portion of TxtE to maintain the nitration function, the portion must include active site residues of TxtE, for example Arg59, Asn293, Thr296 and Glu394. However, genetic modification of residues at a location of the TxtE polypeptide remote from the active site may maintain the activity of the enzyme. As used herein, the term “genetic modification” refers to amino acid substitution (conservative, missense and/or non-sense), deletion and/or insertion. Thus in some embodiments, a portion of TxtE comprises at least 1, at least 2, at least 3, at least 4, at least 5, at least 10, at least 20, at least 30, at least 40, at least 50, at least 75. or at least 100 genetic modifications relative to wild-type TxtE. In some embodiments a portion of TxtE is truncated relative to wild-type TxtE. Truncations may occur at the N-terminus or C-terminus of the portion of TxtE. For example, a portion of TxtE may be truncated by 1, 2, 3, 4, 5, 10, 20, 30, 40, 50, 75, 100 or 200 amino acids at it N-terminus or C-terminus relative to wild-type TxtE.

In some embodiments, the disclosure provides a TxtE enzyme in which the loop region corresponding to residues A274 to V279 of GenBank Accession No. CBG70284.1 are replaced by the heme domain (e.g. loops “j” and “k”) of CYP102A1 (e.g., P450BM3, PDB ID: 1BVY). In some embodiments, a TxtE enzyme (e.g., TxtE-P450BM3) comprises the sequence set forth in SEQ ID NO: 14 or 15.

Methods of genetically modifying TxtE or portions thereof and screening for retention of functional activity are known in the art and available to the skilled artisan. For example, TxtE may be modified by directed evolution or random mutagenesis and biochemically assayed for the capability to transfer a nitro group to aromatic moieties (e.g., indole). In some embodiments, a TxtE enzyme may be an enzyme which catalyzes transfer of a nitro functional group to aromatic moieties (e.g., indole) and has less than 95% homologous to the amino acid sequence of TxtE. In some embodiments, the enzyme has about 90%, about 80%, about 70%, about 60% or about 50% homology to the amino acid sequence of TxtE.

In some aspects, the disclosure provides fusion proteins comprising a catalytic domain of a reductase enzyme. As used herein, the term “reductase enzyme” refers to an enzyme that catalyzes a reduction reaction. Non-limiting examples of reductase enzymes include thioredoxin reductase, cytochrome P450 reductase and flavin adenine dinucleotide (FAD) reductase. In some embodiments, the reductase enzyme is a prokaryotic reductase enzyme. In some embodiments, the reductase enzyme is a bacterial reducatase enzyme. In some embodiments, the bacterial reductase enzyme naturally occurs in a self-sufficient cytochrome P450, for example CYP102A1 (P450BM3) reductase or a P450RhF reductase. In some embodiments, the catalytic domain of a reductase enzyme comprises or consists of the sequence set forth in SEQ ID NO: 15.

In some embodiments, the fusion protein comprises an amino acid linker. As used herein, the term “linker” refers to an amino acid sequence that joins two larger polypeptide domains to form a single fusion polypeptide. Amino acid linkers are well known to those skilled in the art and include flexible linkers (e.g. glycine rich linkers such as [GGGS]ₙ where n>2), rigid linkers (e.g. poly-proline rich linkers) and cleavable linkers (e.g. photocleavable and enzyme-sensitive linkers). In some embodiments, an amino acid linker is derived from a TxtE enzyme or a reductase enzyme (e.g., CYP102A1). For example, in some embodiments, an amino acid linker may comprise between about 3 and about 27 continuous (e.g., covalently linked) amino acids of a reductase enzyme (e.g., between about 3 and about 27 contiguous amino acids the sequence set forth in UniProtKB/Swiss-Prot Accession No. P14779.2. In some embodiments, an amino acid linker comprises between about 3 and about 27 contiguous amino acids, for example between about 3 and about 25 contiguous amino acids of SEQ ID NO: 16.

In some embodiments, amino acid linker length affects the folding and orientation of fusion polypeptides. For example, a linker that is too long can prevent the interaction of a reductase domain with the cytochrome P450 enzyme to which it is linked. (It is also known that long linkers can fold and take on specific orientations that can be desirable.) Conversely, a linker that is too short can cause a reductase enzyme to sterically inhibit binding of substrate to the active site of the P450 enzyme to which it is linked. In some embodiments, TxtE-BM3 fusion proteins comprising linkers having a certain length (e.g., 11, 12, 14, 15, 16, 17, etc. amino acids in length) exhibit improved function (e.g., increased nitration activity, coupling efficiency, total turnover number (TTN), etc.) compared to previously described self-sufficient cytochrome p450 enzymes. Accordingly, in some embodiments, a fusion protein described by the disclosure comprises an amino acid linker between about 3 and about 27 amino acids in length. In some embodiments, an amino acid linker is between about 11 and about 17 amino acids in length. In some embodiments, an amino acid linker is between about 14 and 16 amino acids in length. In some embodiments, the length of the linker is 1, 12, 14, 15, 16, 17, 18, 19, 20, 21, 22, 23, 24, 25, 26, or 27 amino acids in length.

In some embodiments, the amino acid linker joins a catalytic domain of a reductase enzyme to a terminus of a cytochrome P450 enzyme. As used herein, the term “terminus” refers to the ends of a polypeptide sequence relative to the start codon of said polypeptide. For example, the N-terminus of a polypeptide is the end of the polypeptide containing the start codon (AUG) of the polypeptide. whereas the C-terminus of the polypeptide is the end of the polypeptide opposite of the start codon. In some embodiments, the amino acid linker joins the 11 catalytic domain of a reductase enzyme to the C-terminus of a cytochrome P450 enzyme. In some embodiments, the amino acid linker joins CYP102A1 (P450BM3) reductase or P450RhF reductase to the C-terminus of a TxtE enzyme.

Generally, fusion proteins described by the disclosure can be produced by any suitable means known in the art. For example, in some embodiments, a fusion protein is produced by an overlap PCR method. As used herein, “overlap PCR” refers to the splicing (e.g., joining together) of two or more oligonucleotides by polymerase chain reaction employing primers that sham complementarity with the terminus of each of the oligonucleotides, for example as described by Higuchi et al. (1988) Nucleic Acids Res. 16 (15): 7351-67. In some embodiments, fusion proteins described by the disclosure are not produced by overlap PCR. In some embodiments, fusion proteins described by the disclosure are produced by a restriction digest-based method (e.g., traditional cloning), for example as described in Molecular Cloning: A Laboratory Manual, J. Sambrook. et al., eds., Second Edition. Cold Spring Harbor Laboratory Press, Cold Spring Harbor, New York, 1989.

The disclosure relates, in some aspects, to recombinant bacterial cells comprising an isolated nucleic acid engineered to express a nitric oxide synthase (NOS) enzyme. Generally, nitric oxide synthase (NOS) is a protein that catalyzes production of nitric oxide (NO) from L-arginine. Without wishing to be bound by any particular theory. NO is an important co-substrate for TxtE-based nitration reactions, and thus in some embodiments it is desirable to increase NO production in recombinant bacterial cells for the purpose of increasing nitration reaction yields. Generally, a NOS enzyme can be a prokaryotic or eukaryotic NOS enzyme. In some embodiments, a NOS enzyme is a bacterial NOS enzyme. Bacterial NOS enzymes are described, for example in Crane et al. (2010) Anna Rev Biochem. 79:455-70. In some embodiments, a NOS enzyme is a Bacillus subtilis NOS enzyme. In some embodiments, a Bacillus subtilis NOS enzyme is encoded by the sequence set forth in SEQ ID NO: 3. In some embodiments, a Bacillus subtilis NOS enzyme comprises the amino acid sequence set forth in SEQ 1) NO: 5. BsNOS enzymes are described, for example by Commichau et al. (2008) J Bacteriol 190(10):3557-3564.

In some aspects, the disclosure relates to recombinant bacterial cells comprising an isolated nucleic acid engineered to express a glucose dehydrogenase (GHD) enzyme. Glucose dehydrogenase (GDH) is an enzyme that catalyzes the reversible conversion of D-glucose to D-glucono-1,5-lactone while reducing NAD(P)+ to NAD(P)H. Without wishing to be bound by 11 any particular theory, overexpression of GDH in recombinant bacterial cells (e.g., as part of a whole cell nitration system) may, in some embodiments, increase yield of nitration reactions by providing a sufficient supply of NADPH to fuel NOS-mediated conversion of L-Arg to L-citrulline. In some embodiments, a GDH enzyme is a bacterial GDH enzyme. In some embodiments, a bacterial GDH enzyme is a Bacillus megaterium GDH enzyme.

As used herein, the term “engineered to express” refers to an isolated nucleic acid that comprises a gene to be expressed (e.g., TB14, BsNOS, GDH, etc.) and, optionally, one or more expression control sequences. Examples of expression control sequences include but are not limited to promoter sequences, enhancer sequences, repressor sequences, poly A tail sequences, internal ribosomal entry sites, Kozak sequences, antibiotic resistance genes (e.g., ampR, kanR, a chloramphenicol resistance gene, a β-lactamase resistance gene, etc.), an origin of replication (ori), etc.

In some embodiments, one or more isolated nucleic acid is operably linked to a promoter sequence. A promoter can be a constitutive promoter or an inducible promoter. In some embodiments, a promoter is a constitutive promoter. Examples of constitutive promoters include but are not limited to constitutive E. coli π⁷⁰ promoters, constitutive E. coli σ^{S} promoters, constitutive E. coli σ³² promoters, constitutive E. coli σ^{B} promoters, constitutive B. subtilis σ^{A} promoters, constitutive B, subtilis σ^{B} promoters, constitutive bacteriophage T7 promoters, constitutive bacteriophage SP6 promoters, constitutive yeast promoters, etc.

In some embodiments, a promoter is an inducible promoter (e.g., induced in the presence of a small molecule, such as IPTG or tetracycline). Examples of inducible promoters include but are not limited to a promoter comprising a tetracycline responsive element (TRE), a pLac promoter, a pBad promoter, alcohol-regulated promoters (e.g., AlcA promoter). steroid-regulated promoters (e.g., LexA promoter), temperature-inducible promoters (e.g., Hsp70- or Hsp90-derived promoters, light-inducible promoters (e.g., YFI), etc.

In some embodiments. an isolated nucleic acid engineered to express a fusion protein is operably linked to a first promoter, an isolated nucleic acid engineered to express a NOS enzyme is operably linked to a second promoter, and an isolated nucleic acid engineered to express a GDH enzyme is operably linked to a third promoter. In some embodiments, a first promoter, a second promoter, and/or a third promoter is a T7 promoter.

In some embodiments, an isolated nucleic acid engineered to express a protein is a component of a vector. Examples of vectors include plasmids, viral vectors, cosmids, and artificial chromosomes. In some aspects, one or more isolated nucleic acids engineered to express a protein (e.g., TB14, NOS, GDH, etc.) are located (e.g., situated) on a plasmid, for example a bacterial plasmid. In some embodiments, the vector is a high-copy plasmid. In some embodiments, the vector is a low-copy plasmid. In some embodiments, a bacterial cell comprises one or more plasmids comprising the one or more isolated nucleic acids. For example, a plasmid may comprise 1, 2, 3, 4, 5, 6, 7, 8, 9, or 10 isolated nucleic acids. In some embodiments, a plasmid comprises 1, 2, or 3 isolated nucleic acids. In some embodiments, an isolated nucleic acid engineered to express the NOS enzyme and an isolated nucleic acid engineered to express the GDH enzyme are located on the same plasmid. in some embodiments. an isolated nucleic acid engineered to express the fusion protein is located on a plasmid that does not contain an isolated nucleic acid engineered to express the NOS enzyme and/or an isolated nucleic acid engineered to express the GDH enzyme. In some embodiments, a recombinant bacterial cell as described by the disclosure comprises a first plasmid comprising an isolated nucleic acid engineered to express a TxtE fusion protein (e.g. TB14), a second plasmid comprising an isolated nucleic acid engineered to express a NOS enzyme (e.g., BsNOS), and a third plasmid comprising an isolated nucleic acid engineered to express a GDH enzyme.

In some embodiments, one or more isolated nucleic acids (e.g., one or more isolated nucleic acids encoding a fusion protein, a NOS enzyme, and/or a GDH enzyme) are integrated into a chromosome of a bacterial cell. Methods of integrating exogenous (e.g., foreign) DNA into a bacterial chromosome are known in the art and are described, for example, by Gu et al. (2015) Scientific Reports 5; Article number 9684.

The disclosure is based, in part, on recombinant bacterial cells that are capable of producing nitrated aromatic compounds. In some embodiments. recombinant bacterial cells are produced from bacterial strains that have been metabolically modified. As used herein, “metabolically modified” refers to a bacterial cell (or strain) that has been manipulated using recombinant DNA technology or other genome engineering methodologies to lack one or more genes in a particular metabolic pathway. For example, in some embodiments, a recombinant bacterial cell may be produced using a bacterial strain that has been engineered to lack one or more genes relating to tryptophan metabolism, tryptophan biosynthesis, L-tyrosine biosynthesis, phenylalanine biosynthesis, or any combination of the foregoing. In some embodiments, a bacterial cell is genetically modified to lack expression of one or more of the following genes: traA (tryptophanase), trpR (tryptophan repressor), tyrA (T protein), and pheA (P protein). In some embodiments, a bacterial cell comprises the genotype ΔtrpRΔtyrAΔpheA (e.g., is a triple deletion mutant for trpR, tyrA, and pheA). In some embodiments, a bacterial cell comprises a tnaA gene, or a gene product (e.g., protein, enzyme, etc.) expressed from a tnaA gene.

### Compositions Comprising Recombinant Bacterial Cells

In some aspects, the disclosure relates to a composition comprising one or more of a recombinant bacterial cell as described by the disclosure, and a bacterial culture media.

As used herein, a “bacterial culture media” is a nutrient rich composition that supports growth and reproduction of bacterial cells. Generally, bacterial culture media can be liquid or solid (e.g., culture media mixed with agar to form a gel). In some embodiments, bacterial culture media is a liquid. Examples of bacterial culture media include but are not limited to M9, Lysogeny Broth (LB), SOC media, Terrific Broth (TB), etc.

The volume of bacterial culture media in a composition comprising a recombinant bacterial cell can vary depending upon several factors including but not limited to the desired amount of nitrated aromatic compounds to be produced. the concentration (densityl) of bacterial cells desired in the composition, the volume of the container housing the composition, etc. In some embodiments, a composition comprises between about 10 μl and 1 L bacterial culture media. In some embodiments, a composition comprises between about 10 μl and about 1 mL bacterial culture media, for example about 10 μl, about 50 μl, about 100 μl, about 500 μl, about 750 μl, or about 1 mL (e.g., any volume between 10 μl and 1 mL, inclusive). In some embodiments, a composition comprises between about 750 μl and 5 mL (e.g., any volume between 750 μl and 5 mL, inclusive). In some embodiments, a composition comprises between about 2 mL and about 20 mL bacterial culture media (e.g., any volume between 2 mL and 20 mL, inclusive). In some embodiments, a composition comprises between about 10 mL and about 20 mL bacterial culture media (e.g., any volume between 10 mL and 200 mL inclusive). In some embodiments, a composition comprises between about 100 mL and about 500 mL bacterial culture media (e.g., any volume between 100 mL and 500 mL, inclusive). In some embodiments, a composition comprises between about 250 mL and about 1 L bacterial culture media (e.g., any volume between 250 mL and 1 L. inclusive). In some embodiments, a composition comprises more than 1 L (e.g., 5 L, 10 L, 100 L, 200 L, 1000 L, 10,000 L, 50,000 L, etc.) bacterial culture media.

In some embodiments, a composition further comprises one or more antibiotic agents. In in some embodiments, one or more antibiotic agent is ampicillin or kanamycin. A composition may comprise 1, 2, 3, 4, 5, 6, 7, 8, 9, 10, or more antibiotic agents. The concentration of an antibiotic agent can vary. In some embodiments, the concentration of an antibiotic agent ranges from about 0 (e.g., lacking antibiotic) to about 125 μg/ml.

Aspects of the disclosure relate to uptake and subsequent nitration of L-tryptophan (and L-tryptophan analogues) by recombinant bacterial cells described herein. Without wishing to be bound by any particular theory, compositions comprising recombinant bacterial cells described herein and bacterial culture media may be “fed” with exogenous L-tryptophan or analogues thereof, which are internalized by the bacteria (e.g., via permease transport across the bacterial cell membrane) and subsequently nitrated by a fusion protein (e.g., a TxtE fusion protein, such as TB14). Thus, in some embodiments, a composition further comprises one or more of the following: L-tryptophan (L-Trp), L-arginine (L Arg), or an analogue of L-tryptophan. In some embodiments, a composition further comprises one or more compounds of Formula Ia or IVa. In some embodiments, an analogue of L-tryptophan is selected from the group consisting of α-Me-Trp, 4-F-Trp, 4-Me-Trp, 5-MeO-Trp, 5-Me-Trp, 5-F-Trp, 6-F-Trp, and 7-Me-Trp.

In some embodiments, a composition further comprises one or more compounds of Formulae I-VI. In some embodiments, a composition further comprises one or more of the following: 4-NO₂-L-Trp, α-Me-4-NO₂Trp, 4-F-7-NO₂-Trp, 4-Me-7-NO-Trp, 5-MeO-4-NO₂-Trp, 5-Me-4-NO₂-Trp, 5-F-4-NO₂-Trp, 6-F-4-NO₂-Trp, or 7-Me-4-NO₂-Trp. In some embodiments, the compound of Formulae I-VI is selected from:
(S)-2-amino-3-(4-methyl-5-nitro-1H-indol-3-yl)propanoic acid;
(S)-2-amino-3-(5-methyl-4-nitro-1H-indol-3-yl)propanoic acid;
(S)-2-amino-3-(6-methyl-4-nitro-1H-indol-3-yl)propanoic acid;
(S)-2-amino-3-(4-methyl-4-nitro-1H-indol-3-yl)propanoic acid;
(S)-2-amino-3-(4-methyl-7-nitro-1H-4-indol-3-yl)propanoic acid;
(S)-2-amino-3-(5-fluoro-4-nitro-1H-indol-3-yl)propanoic acid;
(S)-2-amino-3-(6-fluoro-4-nitro-1H-indol-3-yl)propanoic acid;
(S)-2-amino-3-(7-fluoro-4-nitro-1H-indol-3-yl)propanoic acid;
(S)-2-amino-3-(4-fluoro-7-nitro-1H-indol-3-yl)propanoic acid;
(S)-2-amino-3-(5-chloro-4-nitro-1H-indol-3-yl)propanoic acid;
(S)-2-amino-3-(6-chloro-4-nitro-1H-indol-3-yl)propanoic acid;
(S)-2-amino-3-(7-chloro-4-nitro-1H-indol-3-yl)propanoic acid;
(S)-2-amino-3-(4-bromo-7-nitro-1H-indol-3-yl)propanoic acid;
(S)-2-amino-3-(5-bromo-4-nitro-1H-indol-3-yl)propanoic acid;
(S)-2-amino-3-(6-bromo-4-nitro-1H-indol-3-yl)propanoic acid;
(S)-2-amino-3-(7-bromo-4-nitro-1H-indol-3-yl)propanoic acid;
(S)-2-amino-3-(5-bromo-7-nitro-1H-indol-3-yl)propanoic acid;
(S)-2-amino-3-(5-methoxy-4-nitro-1H-indol-3-yl)propanoic acid;
(S)-2-amino-3-(6-methoxy-4-nitro-1H-indol-3-yl)propanoic acid;
(S)-2-amino-3-(7-methoxy-4-nitro-1H-indol-3-yl)propanoic acid;
(S)-2-amino-3-(4-methoxy-7-nitro-1H-indol-3-yl)propanoic acid;
(S)-2-amino-3-(5-amino-4-nitro-1H-indol-3-yl)propanoic acid;
(S)-2-amino-3-(6-amino-4-nitro-1H-indol-3-yl)propanoic acid;
(S)-2-amino-3-(7-amino-4-nitro-1H-indol-3-yl)propanoic acid;
(S)-2-amino-3-(4-amino-7-nitro-1H-indol-3-yl)propanoic acid;
(S)-2-amino-3-(5-hydroxy-4-nitro-1H-indol-3-yl)propanoic acid;
(S)-2-amino-3-(6-hydroxy-4-nitro-1H-indol-3-yl)propanoic acid;
(S)-2-amino-3-(7-hydroxy-4-nitro-1H-indol-3-yl)propanoic acid;
(S)-2-amino-3-(4-hydroxy-7-nitro-1H-indol-3-yl)propanoic acid;
(S)-2-amino-3-(4-nitro-5-phenyl-1H-indol-3-yl)propanoic acid;
(S)-2-amino-3-(4-nitro-6-phenyl-1H-indol-3-yl)propanoic acid;
(S)-2-amino-3-(4-nitro-7-phenyl-1H-indol-3-yl)propanoic acid;
(S)-2-amino-3-(7-nitro-4-phenyl-1H-indol-3-yl)propanoic acid;
(S)-2-amino-3-(5-cyclopropyl-4-nitro-1H-indol-3-yl)propanoic acid;
(S)-2-amino-3-(6-cyclopropyl-4-nitro-1H-indol-3-yl)propanoic acid;
(S)-2-amino-3-(7-cyclopropyl-4-nitro-1H-indol-3-yl)propanoic acid;
(S)-2-amino-3-(4-cyclopropyl-7-nitro-1H-indol-3-yl)propanoic acid;
(S)-2-amino-3-(4-nitro-5-vinyl-1H-indol-3-yl)propanoic acid;
(S)-2-amino-3-(4-nitro-6-vinyl-1H-indol-3-yl)propanoic acid;
(S)-2-amino-3-(4-nitro-7-vinyl-1H-indol-3-yl)propanoic acid;
(S)-2-amino-3-(7-nitro-4-vinyl-1H-indol-3-yl)propanoic acid;
(S)-2-amino-3-(5-ethynyl-4-nitro-1H-indol-3-yl)propanoic acid;
(S)-2-amino-3-(6-ethynyl-4-nitro-1H-indol-3-yl)propanoic acid;
(S)-2-amino-3-(7-ethynyl-4-nitro-1H-indol-3-yl)propanoic acid;
(S)-2-amino-3-(4-ethynyl-7-nitro-1H-indol-3-yl)propanoic acid;
(S)-2-amino-3-(5-morpholino-4-nitro-1H-indol-3-yl)propanoic acid;
(S)-2-amino-3-(6-morpholino-4-nitro-1H-indol-3-yl)propanoic acid;
(S)-2-amino-3-(7-morpholino-4-nitro-1H-indol-3-yl)propanoic acid;
(S)-2-amino-3-(4-morpholino-7-nitro-1H-indol-3-yl)propanoic acid;
(S)-2-amino-3-(5-(methylthio)-4-nitro-1H-indol-3-yl)propanoic acid;
(S)-2-amino-3-(6-(methylthio)-4-nitro-1H-indol-3-yl)propanoic acid;
(S)-2-amino-3-(7-(methylthio)-4-nitro-1H-indol-3-yl)propanoic acid;
(S)-2-amino-3-(4-(methylthio)-7-nitro-1H-indol-3-yl)propanoic acid;
(S)-2-amino-3-(4-nitro-5-(pyridin-4-yl)-1H-indol-3-yl)propanoic acid;
(S)-2-amino-3-(4-nitro-6-(pyridin-4-yl)-1H-indol-3-yl)propanoic acid;
(S)-2-amino-3-(4-nitro-7-(pyridin-4-yl)-1H-indol-3-yl)propanoic acid;
(S)-2-amino-3-(7-nitro-4-(pyridin-4-yl)-1H-indol-3-yl)propanoic acid;
2-amino-3-(4-methyl-5-nitro-1H-indol-3-yl)propanoic acid;
2-amino-3-(5-methyl-4-nitro-1H-indol-3-yl)propanoic acid;
2-amino-3-(6-methyl-4-nitro-1H-indol-3-yl)propanoic acid;
2-amino-3-(7-methyl-4-nitro-1H-indol-3-yl)propanoic acid;
2-amino-3-(4-methyl-7-nitro-1H-indol-3-yl)propanoic acid;
2-amino-3-(6-fluoro-4-nitro-1H-indol-3-yl)propanoic acid;
2-amino-3-(7-fluoro-4-nitro-1H-indol-3-yl)propanoic acid;
2-amino-3-(4-fluoro-7-nitro-1H-indol-3-yl)propanoic acid;
2-amino-3-(5-chloro-4-nitro-1H-indol-3-yl)propanoic acid;
2-amino-3-(6-chloro-4-nitro-1H-indol-3-yl)propanoic acid;
2-amino-3-(7-chloro-4-nitro-1H-indol-3-yl)propanoic acid;
2-amino-3-(4-choo-7-nitro-1H-indol-3-yl)propanoic acid;
2-amino-3-(5-bromo-4-nitro-1H-indol-3-yl)propanoic acid;
2-amino-3-(6-bromo-4-nitro-1H-indol-3-yl)propanoic acid;
2-amino-3-(7-bromo-4-nitro-1H-indol-3-yl)propanoic acid;
2-amino-3-(4-bromo-7-nitro-1H-indol-3-yl)propanoic acid;
2-amino-3-(5-methoxy-4-nitro-1H-indol-3-yl)propanoic acid;
2-amino-3-(6-methoxy-4-nitro-1H-indol-3-yl)propanoic acid;
2-amino-3-(7-methoxy-4-nitro-1H-indol-3-yl)propanoic acid;
2-amino-3-(4-methoxy-7-nitro-1H-indol-3-yl)propanoic acid;
2-amino-3-(5-amino-4-nitro-1H-indol-3-yl)propanoic acid;
2-amino-3-(6-amino-4-nitro-1H-indol-3-yl)propanoic acid;
2-amino-3-(7-amino-4-nitro-1H-indol-3-yl)propanoic acid;
2-amino-3-(4-amino-7-nitro-1H-indol-3-yl)propanoic acid;
2-amino-3-(5-hydroxy-4-nitro-1H-indol-3-yl)propanoic acid;
2-amino-3-(6-hydroxy-4-nitro-1H-indol-3-yl)propanoic acid;
2-amino-3-(7-hydroxy-4-nitro-1H-indol-3-yl)propanoic acid;
2-amino-3-(4-hydroxy-7-nitro-1H-indol-3-yl)propanoic acid;
2-amino-3-(4-nitro-5-phenyl-1H-indol-3-yl)propanoic acid;
2-amino-3-(4-nitro-6-phenyl-1H-indol-3-yl)propanoic acid;
2-amino-3-(4-nitro-7-phenyl-1H-indol-3-yl)propanoic acid;
2-amino-3-(7-nitro-4-phenyl-1H-indol-3-yl)propanoic acid;
2-amino-3-(5-cyclopropyl-4-nitro-1H-indol-3-yl)propanoic acid;
2-amino-3-(6-cyclopropyl-4-nitro-1H-indol-3-yl)propanoic acid;
2-amino-3-(7-cyclopropyl-4-nitro-1H-indol-3-yl)propanoic acid;
2-amino-3-(4-cyclopropyl-7-nitro-1H-indol-3-yl)propanoic acid;
2-amino-3-(4-nitro-5-vinyl-1H-indol-3-yl)propanoic acid;
2-amino-3-(4-nitro-6-vinyl-1H-indol-3-yl)propanoic acid;
2-amino-3-(4-nitro-7-vinyl-1H-indol-3-yl)propanoic acid;
2-amino-3-(7-nitro-4-vinyl-1H-indol-3-yl)propanoic acid;
2-amino-3-(5-ethynyl-4-nitro-1H-indol-3-yl)propanoic acid;
2-amino-3-(6-ethynyl-4-nitro-1H-indol-3-yl)propanoic acid;
2-amino-3-(7-ethynyl-4-nitro-1H-indol-3-yl)propanoic acid;
2-amino-3-(4-ethynyl-7-nitro-1H-indol-3-yl)propanoic acid;
2-amino-3-(5-morpholino-4-nitro-1H-indol-3-yl)propanoic acid;
2-amino-3-(6-morpholino-4-nitro-1H-indol-3-yl)propanoic acid;
2-amino-3-(7-morpholino-4-nitro-1H-indol-3-yl)propanoic acid;
2-amino-3-(4-morpholino-7-nitro-1H-indol-3-yl)propanoic acid;
2-amino-3-(5-(methylthio)-4-nitro-1H-indol-3-yl)propanoic acid;
2-amino-3-(6-(methylthio)-4-nitro-1H-indol-3-yl)propanoic acid;
2-amino-3-(7-(methylthio)-4-nitro-1H-indol-3-yl)propanoic acid;
2-amino-3-(4-(methylthio)-7-nitro-1H-indol-3-yl)propanoic acid;
2-amino-3-(4-nitro-5-(pyridin-4-yl)-1H-indol-3-yl)propanoic acid;
2-amino-3-(4-nitro-6-(pyridin-4-yl)-1H-indol-3-yl)propanoic acid;
2-amino-3-(4-nitro-7-(pyridin-4-yl)-1H-indol-3-yl)propanoic acid;
2-amino-3-(7-nitro-4-(pyridin-4-yl)-1H-indol-3-yl)propanoic acid;
2-amino-3-(1,5-dimethyl-4-nitro-1H-indol-3-yl)propanoic acid;
2-amino-3-(1,6-dimethyl-4-nitro-1H-indol-3-yl)propanoic acid;
2-amino-3-(1,7-dimethyl-4-nitro-1H-indol-3-yl)propanoic acid;
2-amino-3-(1,4-dimethyl-7-nitro-1H-indol-3-yl)propanoic acid;
2-amino-3-(6-fluoro-1-methyl-4-nitro-1H-indol-3-yl)propanoic acid;
2-amino-3-(7-fluoro-1-methyl-4-nitro-1H-indol-3-yl)propanoic acid;
2-amino-3-(4-fluoro-1-methyl-7-nitro-1H-indol-3-yl)propanoic acid;
2-amino-3-(5-chloro-1-methyl-4-nitro-1H-indol-3-yl)propanoic acid;
2-amino-3-(6-chloro-1-methyl-4-nitro-1H-indol-3-yl)propanoic acid;
2-amino-3-(7-chloro-1-methyl-4-nitro-1H-indol-3-yl)propanoic acid;
2-amino-3-(4-chloro-1-methyl-7-nitro-1H-indol-3-yl)propanoic acid;
2-amino-3-(5-bromo-1-methyl-4-nitro-1H-indol-3-yl)propanoic acid;
2-amino-3-(6-bromo-1-methyl-4-nitro-1H-indol-3-yl)propanoic acid;
2-amino-3-(7-bromo-1-methyl-4-nitro-1H-indol-3-yl)propanoic acid;
2-amino-3-(4-bromo-1-methyl-7-nitro-1H-indol-3-yl)propanoic acid;
2-amino-3-(5-methoxy-1-methyl-4-nitro-1H-indol-3-yl)propanoic acid;
2-amino-3-(6-methoxy-1-methyl-4-nitro-1H-indol-3-yl)propanoic acid;
2-amino-3-(7-methoxy-1-methyl-4-nitro-1H-indol-3-yl)propanoic acid;
2-amino-3-(4-methoxy-1-methyl-7-nitro-1H-indol-3-yl)propanoic acid;
2-amino-3-(5-amino-1-methyl-4-nitro-1H-indol-3-yl)propanoic acid;
2-amino-3-(6-amino-1-methyl-4-nitro-1H-indol-3-yl)propanoic acid;
2-amino-3-(7-amino-1-methyl-4-nitro-1H-indol-3-yl)propanoic acid;
2-amino-3-(4-amino-1-methyl-7-nitro-1H-indol-3-yl)propanoic acid;
2-amino-3-(5-hydroxy-1-methyl-4-nitro-1H-indol-3-yl)propanoic acid;
2-amino-3-(6-hydroxy-1-methyl-4-nitro-1H-indol-3-yl)propanoic acid;
2-amino-3-(7-hydroxy-1-methyl-4-nitro-1H-indol-3-yl)propanoic acid;
2-amino-3-(4-hydroxy-1-methyl-7-nitro-1H-indol-3-yl)propanoic acid;
2-amino-3-(1-methyl-4-nitro-5-phenyl-1H-indol-3-yl)propanoic acid;
2-amino-3-(1-methyl-4-nitro-6-phenyl-1H-indol-3-yl)propanoic acid;
2-amino-3-(1-methyl-4-nitro-7-phenyl-1H-indol-3-yl)propanoic acid;
2-amino-3-(1-methyl-7-nitro-4-phenyl-1H-indol-3-yl)propanoic acid;
2-amino-3-(5-cyclopropyl-1-methyl-4-nitro-1H-indol-3-yl)propanoic acid;
2-amino-34(6-cyclopropyl-1-methyl-4-nitro-1H-indol-3-yl)propanoic acid;
2-amino-3-(7-cyclopropyl-1-methyl-4-nitro-1H-indol-3-yl)propanoic acid;
2-amino-3-(4-cyclopropyl-1-methyl-7-nitro-1H-indol-3-yl)propanoic acid;
2-amino-3-(1-methyl-4-nitro-5-vinyl-1H-indol-3-yl)propanoic acid;
2-amino-3-(1-methyl-4-nitro-6-vinyl-1H-indol-3-yl)propanoic acid;
2-amino-3-(1-methyl-4-nitro-7-vinyl-1H-indol-3-yl)propanoic acid;
2-amino-3-(1-methyl-7-nitro-4-vinyl-1H-indol-3-yl)propanoic acid;
2-amino-3-(5-ethynyl-1-methyl-4-nitro-1H-indol-3-yl)propanoic acid;
2-amino-3-(6-ethynyl-1-methyl-4-nitro-1H-indol-3-yl)propanoic acid;
2-amino-3-(7-ethynyl-1-methyl-4-nitro-1H-indol-3-yl)propanoic acid;
2-amino-3-(4-ethynyl-1-methyl-7-nitro-1H-indol-3-yl)propanoic acid;
2-amino-3-(1-methyl-5-morpholino-4-nitro-1H-indol-3-yl)propanoic acid;
2-amino-3-(1-methyl-6-morpholino-4-nitro-1H-indol-3-yl)propanoic acid;
2-amino-3-(1-methyl-7-morpholino-4-nitro-1H-indol-3-yl)propanoic acid;
2-amino-3-(1-methyl-4-morpholino-7-nitro-1H-indol-3-yl)propanoic acid;
2-amino-3-(1-methyl-5-(methylthio)-4-nitro-1H-indol-3-yl)propanoic acid;
2-amino-3-(1-methyl-6-(methylthio)-4-nitro-1H-indol-3-yl)propanoic acid;
2-amino-3-(1-methyl-7-(methylthio)-4-nitro-1H-indol-3-yl)propanoic acid;
2-amino-3-(1-methyl-4-(methylthio)-7-nitro-1H-indol-3-yl)propanoic acid;
2-amino-3-(1-methyl-4-nitro-5-(pyridin-4-yl)-1H-indol-3-yl)propanoic acid;
2-amino-3-(1-methyl-4-nitro-6-(pyridin-4-yl)-1H-indol-3-yl)propanoic acid;
2-amino-3-(1-methyl-4-nitro-7-(pyridin-4-yl)-1H-indol-3-yl)propanoic acid;
2-amino-3-(1-methyl-7-nitro-4-(pyridin-4-yl)-1H-indol-3-yl)propanoic acid;
2-amino-3-(2,5-dimethyl-4-nitro-1H-indol-3-yl)propanoic acid;
2-amino-3-(2,6-dimethyl-4-nitro-1H-indol-3-yl)propanoic acid;
2-amino-3-(2,7-dimethyl-4-nitro-1H-indol-3-yl)propanoic acid;
2-amino-3-(2,4-dimethyl-7-nitro-1H-indol-3-yl)propanoic acid;
2-amino-3-(6-fluoro-2-methyl-4-nitro-1H-indol-3-yl)propanoic acid;
2-amino-3-(7-fluoro-2-methyl-4-nitro-1H-indol-3-yl)propanoic acid;
2-amino-3-(4-fluoro-2-methyl-7-nitro-1H-indol-3-yl)propanoic acid;
2-amino-3-(5-chloro-2-methyl-4-nitro-1H-indol-3-yl)propanoic acid;
2-amino-3-(6-chloro-2-methyl-4-nitro-1H-indol-3-yl)propanoic acid;
2-amino-3-(7-chloro-2-methyl-4-nitro-1H-indol-3-yl)propanoic acid;
2-amino-3-(4-chloro-2-methyl-7-nitro-1H-indol-3-yl)propanoic acid;
2-amino-3-(5-bromo-2-methyl-4-nitro-1H-indol-3-yl)propanoic acid;
2-amino-3-(6-bromo-2-methyl-4-nitro-1H-indol-3-yl)propanoic acid;
2-amino-3-(7-bromo-2-methyl-4-nitro-1H-indol-3-yl)propanoic acid;
2-amino-3-(4-bromo-2-methyl-7-nitro-1H-indol-3-yl)propanoic acid;
2-amino-3-(5-methoxy-2-methyl-4-nitro 1H-indol-3-yl)propanoic acid;
2-amino-3-(6-methoxy-2-methyl-4-nitro-1H-indol-3-yl)propanoic acid;
2-amino-3-(7-methoxy-2-methyl-4-nitro-1H-indol-3-yl)propanoic acid;
2-amino-3-(4-methoxy-2-methyl-7-nitro-1H-indol-3-yl)propanoic acid;
2-amino-3-(5-amino-2-methyl-4-nitro-1H-indol-3-yl)propanoic acid;
2-amino-3-(6-amino-2-methyl-4-nitro-1H-indol-3-yl)propanoic acid;
2-amino-3-(7-amino-2-methyl-4-nitro-1H-indol-3-yl)propanoic acid;
2-amino-3-(4-amino-2-methyl-7-nitro-1H-indol-3-yl)propanoic acid;
2-amino-3-(5-hydroxy-2-methyl-4-nitro-1H-indol-3-yl)propanoic acid;
2-amino-3-(6-hydroxy-2-methyl-4-nitro-1H-indol-3-yl)propanoic acid;
2-amino-3-(7-hydroxy-2-methyl-4-nitro-1H-indol-3-yl)propanoic acid;
2-amino-3-(4-hydroxy-2-methyl-7-nitro-1H-indol-3-yl)propanoic acid;
2-amino-3-(2-methyl-4-nitro-5-phenyl-1H-indol-3-yl)propanoic acid;
2-amino-3-(2-methyl-4-nitro-6-phenyl-1H-indol-3-yl)propanoic acid;
2-amino-3-(2-methyl-4-nitro-7-phenyl-1H-indol-3-yl)propanoic acid;
2-amino-3-(2-methyl-7-nitro-4-phenyl-1H-indol-3-yl)propanoic acid;
2-amino-3-(5-cyclopropyl-2-methyl-4-nitro-1H-indol-3-yl)propanoic acid;
2-amino-3-(6-cyclopropyl-2-methyl-4-nitro-1H-indol-3-yl)propanoic acid;
2-amino-3-(7-cyclopropyl-2-methyl-4-nitro-1H-indol-3-yl)propanoic acid;
2-amino-3-(4-cyclopropyl-2-methyl-7-nitro-1H-indol-3-yl)propanoic acid
2-amino-3-(2-methyl-4-nitro-5-vinyl-1H-indol-3-yl)propanoic acid;
2-amino-3-(2-methyl-4-nitro-6-vinyl-1H-indol-3-yl)propanoic acid;
2-amino-3-(2-methyl-4-nitro-7-vinyl-1H-indol-3-yl)propanoic acid;
2-amino-3-(2-methyl-7-nitro-4-vinyl-1H-indol-3-yl)propanoic acid;
2-amino-3-(5-ethynyl-2-methyl-4-nitro-1H-indol-3-yl)propanoic acid;
2-amino-3-(6-ethynyl-2-methyl-4-nitro-1H-indol-3-yl)propanoic acid;
2-amino-3-(4-ethynyl-2-methyl-4-nitro-1H-indol-3-yl)propanoic acid;
2-amino-3-(4-ethynyl-5-methyl-7-nitro-1H-indol-3-yl)propanoic acid;
2-amino-3-(2-methyl-5-morpholino-4-nitro-1H-indol-3-yl)propanoic acid;
2-amino-3-(2-methyl-6-morpholino-4-nitro-1H-indol-3-yl)propanoic acid;
2-amino-3-(2-methyl-7-morpholino-4-nitro-1H-indol-3-yl)propanoic acid;
2-amino-3-(2-methyl-4-morpholino-7-nitro-1H-indol-3-yl)propanoic acid;
2-amino-3-(2-methyl-5-(methylthio)-4-nitro-1H-indol-3-yl)propanoic acid;
2-amino-3-(2-methyl-6-(methylthio)-4-nitro-1H-indol-3-yl)propanoic acid;
2-amino-3-(2-methyl-7-(methylthio)-4-nitro-1H-indol-3-yl)propanoic acid;
2-amino-3-(2-methyl-4-(methylthio)-7-nitro-1H-indol-3-yl)propanoic acid;
2-amino-3-(2-methyl-4-nitro-5-(pyridin-4-yl)-1H-indol-3-yl)propanoic acid;
2-amino-3-(2-methyl-4-nitro-6-(pyridin-4-yl)-1H-indol-3-yl)propanoic acid;
2-amino-3-(2-methyl-4-nitro-7-(pyridin-4-yl)-1H-indol-3-yl)propanoic acid;
2-amino-3-(2-methyl-7-nitro-4-(pyridin-4-yl)-1H-indol-3-yl)propanoic acid;
2-amino-3-(1,2,5-trimethyl-4-nitro-1H-indol-3-yl)propanoic acid;
2-amino-3-(1,2,6-trimethyl-4-nitro-1H-indol-3-yl)propanoic acid;
2-amino-3-(1,2,7-trimethyl-4-nitro-1H-indol-3-yl)propanoic acid;
2-amino-3-(1,2,4-trimethyl-7-nitro-1H-indol-3-yl)propanoic acid;
2-amino-3-(6-fluoro-1,2-dimethyl-4-nitro-1H-indol-3-yl)propanoic acid;
2-amino-3-(7-fluoro-1,2-dimethyl-4-nitro-1H-indol-3-yl)propanoic acid;
2-amino-3-(4-fluoro-1,2-dimethyl-7-nitro-1H-indol-3-yl)propanoic acid;
2-amino-3-(5-chloro-1,2-dimethyl-4-nitro-1H-indol-3-yl)propanoic acid;
2-amino-3-(6-chloro-1,2-dimethyl-4-nitro-1H-indol-3-yl)propanoic acid;
2-amino-3-(7-chloro-1,2-dimethyl-4-nitro-1H-indol-3-yl)propanoic acid;
2-amino-3-(4-chloro-1,2-dimethyl-7-nitro-1H-indol-3-yl)propanoic acid;
2-amino-3(5-bromo-1,2-dimethyl-4-nitro-1H-indol-3-yl)propanoic acid;
2-amino-3-(6-bromo-1,2-dimethyl-4-nitro-1H-indol-3-yl)propanoic acid;
2-amino-3-(7-bromo-1,2-dimethyl-4-nitro-1H-indol-3-yl)propanoic acid;
2-amino-3-(4-bromo-1,2-dimethyl-7-nitro-1H-indol-3-yl)propanoic acid;
2-amino-3-(5-methoxy-1,2-dimethyl-4-nitro-1H-indol-3-yl)propanoic acid;
2-amino-3-(6-methoxy-1,2-dimethyl-4-nitro-1H-indol-3-yl)propanoic acid;
2-amino-3-(7-methoxy-1,2-dimethyl-4-nitro-1H-indol-3-yl)propanoic acid;
2-amino-3-(4-methoxy-1,2-dimethyl-7-nitro-1H-indol-3-yl)propanoic acid;
2-amino-3-(5-amino-1,2-dimethyl-4-nitro-1H-indol-3-yl)propanoic acid;
2-amino-3-(6-amino-1,2-dimethyl-4-nitro-1H-indol-3-yl)propanoic acid;
2-amino-3-(7-amino-1,2-dimethyl-4-nitro-1H-indol-3-yl)propanoic acid;
2-amino-3-(4-amino-1,2-dimethyl-7-nitro-1H-indol-3-yl)propanoic acid;
2-amino-3-(5-hydroxy-1,2-dimethyl-4-nitro-1H-indol-3-yl)propanoic acid;
2-amino-3-(6-hydroxy-1,2-dimethyl-4-nitro-1H-indol-3-yl)propanoic acid;
2-amino-3-(7-hydroxy-1,2-dimethyl-4-nitro-1H-indol-3-yl)propanoic acid;
2-amino-3-(4-hydroxy-1,2-dimethyl-7-nitro-1H-indol-3-yl)propanoic acid;
2-amino-3-(1,2-dimethyl-4-nitro-5-phenyl-1H-indol-3-yl)propanoic acid;
2-amino-3-(1,2-dimethyl-4-nitro-6-phenyl-1H-indol-3-yl)propanoic acid;
2-amino-3-(1,2-dimethyl-4-nitro-7-phenyl-1H-indol-3-yl)propanoic acid;
2-amino-3-(1,2-dimethyl-7-nitro-4-phenyl-1H-indol-3-yl)propanoic acid;
2-amino-3-(5-cyclopropyl-1,2-dimethyl-4-nitro-1H-indol-3-yl)propanoic acid;
2-amino-3-(6-cyclopropyl-1,2-dimethyl-4-nitro-1H-indol-3-yl)propanoic acid;
2-amino-3-(7-cyclopropyl-1,2-dimethyl-4-nitro-1H-indol-3-yl)propanoic acid;
2-amino-3-(4-cyclopropyl-1,2-dimethyl-7-nitro-1H-indol-3-yl)propanoic acid;
2-amino-3-(1,2-dimethyl-4-nitro-5-vinyl-1H-indol-3-yl)propanoic acid;
2-amino-3-(1,2-dimethyl-4-nitro-6-vinyl-1H-indol-3-yl)propanoic acid;
2-amino-3-(1,2-dimethyl-4-nitro-7-vinyl-1H-indol-3-yl)propanoic acid;
2-amino-3-(5,2-dimethyl-7-nitro-4-vinyl-1H-indol-3-yl)propanoic acid;
2-amino-3-(5-ethynyl-1,2-dimethyl-4-nitro-1H-indol-3-yl)propanoic acid;
2-amino-3-(6-ethynyl-1,2-dimethyl-4-nitro-1H-indol-3-yl)propanoic acid;
2-amino-3-(7-ethynyl-1,2-dimethyl-4-nitro-1H-indol-3-yl)propanoic acid;
2-amino-3-(4-ethynyl-1,2-dimethyl-7-nitro-1H-indol-3-yl)propanoic acid;
2-amino-3-(1,2-dimethyl-5-morpholino-4-nitro-1H-indol-3-yl)propanoic acid;
2-amino-3-(1,2-dimethyl-6-morpholino-4-nitro-1H-indol-3-yl)propanoic acid;
2-amino-3-(1,2-dimethyl-7-morpholino-4-nitro-1H-indol-3-yl)propanoic acid;
2-amino-3-(1,2-dimethyl-4-morpholino-7-nitro-1H-indol-3-yl)propanoic acid;
2-amino-3-(1,2-dimethyl-5-(methylthio)-4-nitro-1H-indol-3-yl)propanoic acid;
2-amino-3-(1,2-dimethyl-6-(methylthio)-4-nitro-1H-indol-3-yl)propanoic acid;
2-amino-3-(1,2-dimethyl-7-(methylthio)-4-nitro-1H-indol-3-yl)propanoic acid;
2-amino-3-(1,2-dimethyl-4-(methylthio)-7-nitro-1H-indol-3-yl)propanoic acid;
2-amino-3-(1,2-dimethyl-4-nitro-5-(pyridin-4-yl)-1H-indol-3-yl)propanoic acid;
2-amino-3-(1,2-dimethyl-4-nitro-6-(pyridin-4-yl)-1H-indol-3-yl)propanoic acid;
2-amino-3-(1,2-dimethyl-4-nitro-7-(pyridin-4-yl)-1H-indol-3-yl)propanoic acid; or
2-amino-3-(1,2-dimethyl-7-nitro-4-(pyridin-4-yl)-1H-indol-3-yl)propanoic acid;
and a pharmaceutically acceptable salt, prodrug, hydrate. or solvate thereof.

The skilled artisan recognizes that the conditions under which a composition as described herein is maintained may affect the production and/or stability of nitrated aromatic compounds by the recombinant bacterial cell(s). The disclosure is based, in part, on the recognition that production of nitrated aromatic compounds is reduced or absent at temperatures at which bacterial cells are generally cultured (e.g., 37° C.). In some embodiments, a composition has a temperature below 37° C. (e.g., the temperature of the bacterial culture media of a composition is below 37° C.). The disclosure is based, in part, on the recognition that production of nitrated aromatic compounds is increased at temperatures between 10 to 30° C. (e.g., 10° C., 1° IC, 12° C., 13° C., 4° C., 15° C., 16° C., 17° C., 18° C., 19° C., 20° C., 21° C., 22° C., 23° C., 24° C., 25° C., 26° C., 27° C., 28° C., 29° C., or 30° C.). In some embodiments, a composition has a temperature of 28° C. (e.g., the temperature of the bacterial culture media of a composition is 28° C.).

In some embodiments, a composition as described by the disclosure comprises additional components. for example one or more cryopreservatives (e.g., glycol. DMSO. PEG, glycerol, etc.), antifungals, etc.

### Methods of Producing Recombinant Bacterial Cells

In some embodiments, the disclosure relates to methods of producing a recombinant bacterial cell as described by the disclosure. Typically, the methods comprise the steps of: transforming a bacterial cell with an isolated nucleic acid engineered to express a fusion protein comprising a TxtE enzyme linked to a catalytic domain of a CYP102A1 (P450BM3) reductase enzyme via an amino acid linker sequence that is between 14 and 27 amino acids in length; and an isolated nucleic acid engineered to express a nitric oxide synthase (NOS) enzyme; and culturing (e.g., growing) the bacterial cell.

Methods of introducing vectors into bacteria are well known in the art and described, for example, in Current Protocols in Molecular Biology. Ausubel et al. (Eds), John Wiley and Sons, New York, 2007.

In some embodiments of methods described by the disclosure. a bacterial cell is transformed with one or more an isolated nucleic acids comprising the sequence set furth in any one of SEQ ID NOs: 8-13.

### Whole Cell-Based Nitration of L-Tryptophan and L-Tryptophan Analogues

In some aspects, the disclosure relates to methods for producing a nitrated L-tryptophan or nitrated L-tryptophan analogue, comprising the steps of: introducing into a bacterial cell culture comprising a one or more of a recombinant bacterial cell as described by the disclosure one or more L-Trp molecules and/or one or more L-Trp analogue molecules; and growing the bacterial cell culture under conditions under which a fusion protein expressed by the recombinant bacterial cell catalyzes a nitration reaction which produces one or more nitrated L-Trp molecules and/or one or more nitrated L-Trp analog molecules. In some embodiments, methods further comprise the step of isolating nitrated L-Trp molecules and/or nitrated L-Trp analog molecules from the bacterial cell culture.

In some embodiments of methods described by the disclosure, one or more L-Trp analogue molecules are selected from the group consisting of α-Me-Trp, 4-F-Trp, 4-Me-Trp, 5-MeO-Trp, 5-Me-Trp, 5-F-Trp, 6-F-Trp, and 7-Me-Trp. The concentration of L-Trp or L-Trp analog added (introduced into) to a bacterial cell culture can vary. In some embodiments. L-Trp or an L-Trp analogue is added to a bacterial cell culture in an amount such that the final concentration of L-Trp or L-Trp analogue in the bacterial cell culture ranges from about 1 μM to a saturation concentration or above (e.g., 1 μM, 10 μM, 50 μM, 100 μM, 500 μM, 750 μM, 1 mM, 10 mM, 100 mM, etc.).

In some embodiments, the order in which the proteins encoded by the isolated nucleic acids of the recombinant bacterial cell are expressed affects the production of nitrated aromatic compounds. In some embodiments, a TxtE fusion protein, NOS enzyme, and optionally GDH, are simultaneously expressed in a recombinant bacterial cell. In some embodiments, a TxtE fusion protein. NOS enzyme, and optionally GDH, are expressed sequentially in a recombinant bacterial cell. The order of sequential expression of the proteins can vary. For example a TxtE fusion protein may be expressed first prior to NOS and/or GDH. In some embodiments, a NOS enzyme is expressed prior to expression of a TxtE fusion protein. In some embodiments, a TxtE fusion protein. NOS enzyme, and optionally GDH, are expressed in a recombinant bacterial cell prior to addition of L-Trp or L-Trp analogue. In some embodiments, a TxtE fusion protein, NOS enzyme, and optionally GDH, are expressed in a recombinant bacterial cell after addition of L-Trp or L-Trp analogue.

In some embodiments of methods described by the disclosure, the step of growing the bacterial cell culture comprises introducing one or more antibiotic and/or one or more inducer into the bacterial cell culture. In some embodiments, one or more antibiotic is selected from ampicillin and kanamycin. In some embodiments, one or more of the inducers is Isopropyl β-D-1-thiogalactopyranoside (IPTG).

The skilled artisan recognizes that the conditions under which a composition as described herein is maintained may affect the production and/or stability of nitrated aromatic compounds by the recombinant bacterial cell(s). The disclosure is based, in part, on the recognition that production of nitrated aromatic compounds is reduced or absent at temperatures at which bacterial cells are generally cultured (e.g., 37° C.). In some embodiments, a composition has a temperature below 37° C. (e.g., the temperature of the bacterial culture media of a composition is below 37° C.). The disclosure is based, in part, on the recognition that production of nitrated aromatic compounds is increased at temperatures between 10 to 30° C. (e.g., 10° C., 11° C., 12° C. 13° C., 14° C. 15° C. 16° C., 17° C., 18° C., 19° C., 20° C., 2P° C., 22° C., 23° C., 24° C., 25° C., 26° C., 27° C., 28° C. 29° C., or 30° C.). In some embodiments, a composition has a temperature of 28° C. (e.g., the temperature of the bacterial culture media of a composition is 28° C.).

The length of time a bacterial cell culture is grown after addition of L-Trp or a L-Trp analogue can vary. In some embodiments, a bacterial cell culture is grown for about 1 hour to about 30 hours (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 hours) after the addition of L-Trp or L-Trp analogue. In some embodiments, a bacterial cell culture is grown for about 1 hour to about 30 hours (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 hours) post-transformation with one or more isolated nucleic acids. In some embodiments, a bacterial cell culture is grown for up to 25 hours (e.g., up to 25 hours post-transformation with one or more isolated nucleic acids).

In some embodiments, isolating nitrated L-Trp molecules and/or nitrated L-Trp analog molecules comprises lysing one or more recombinant bacterial cells. Lysis of bacterial cells is generally known in the art and may be achieved, for example, by incubating bacterial cells in a lysis buffer (e.g., a hypertonic solution, a solution containing lysozyme, a solution containing detergent, etc.) or by centrifugation.

In some embodiments. nitrated 1-Trp molecules and/or nitrated L-Trp analog molecules are isolated from a bacterial cell lysate by performing high-pressure liquid chromatography (HPLC) or other liquid extraction methods known in the art. Additional methods for purification and/or analysis of nitrated aromatic compounds (e.g., 4-NO₂-L-Trp, etc.) include mass spectroscopy and nuclear magnetic resonance (NMR) analysis.

The present invention is further illustrated by the following Examples, which in no way should be construed as further limiting.

### EXAMPLES

### Example 1: Materials and Methods

General Chemicals, DNA sub-cloning. and Bacterial Strains

Molecular biology reagents and enzymes were purchased from Fisher Scientific. Primers (Table 1) were ordered from Sigma-Aldrich, 4-Me-DLTrp was from MP Biomedical (Santa Ana. CA), while NOC-5 (3-(Aminopropyl)-1-hydroxy-3-isopropyl-2-oxo-1-triazene) was purchased from EMD Millipore. Other chemicals and solvents were purchased from Sigma-Aldrich and Fisher Scientific. Escherichia coli DH5a (Life Technologies) was used for cloning and plasmid harvesting. E. coli BL21-GOLD (DE3) (Agilent) was used for protein overexpression. E. coli strains were grown in Luria-Bertani broth or Terrific broth. DNA sequencing was performed at Eurofins. A Shimadzu Prominence UHPLC system (Kyoto, Japan) fitted with an Agilent Poroshell 120 EC-C18 column (2.7 μm, 3.0×50 mm), coupled with a PDA detector was used for HPLC analysis.

### Construction of Plasmids for Whole Cell Transformation

TB14 gene was amplified from TB14/pET28b using a pair of TB14FN and TB14RH primers in PCR reactions (Table 1). The PCR product was analyzed by agarose gel and extracted with a GeneJET Gel Extraction Kit (Thermo). Purified PCR products. pACYCDuet and pETDuet were digested with the restriction enzymes NcoI and HindIII, and corresponding linear DNAs were ligated to generate expression constructs. GDH gene was amplified from GDH/pET21b using a pair of GDHFB and GDHRH primers in PCR reactions (Table 1). The PCR product was analyzed by agarose gel and extracted with a GeneJET Gel Extraction Kit (Thermo). Purified PCR products, pET28b, pACYCDuet and pETDuet were digested with the restriction enzymes BamHI and HindIII, and corresponding linear DNAs were ligated to generate expression constructs. BsNOS gene was amplified from BsNOS/pET15b using a pair of BsNOSFN and BsNOSRH primers in PCR reactions (Table 1). The PCR product was analyzed by agarose gel and extracted with a GeneJET Gel Extraction Kit (Thermo). Purified PCR products, pET28b, pACYCDuet and pETDuet were digested with the restriction enzymes NdeI and HindIII, and corresponding linear DNAs were ligated to generate expression constructs. SsTxtD and StTxtD were amplified from genomic DNA of S. scabies 87.22 (NRRL B-24449) and S. turgidiscabies Car8 using a pair of SsTxtDFN/SsTxtDRH and StTxtDFN/StTxtDRH primers in PCR reactions (Table 1). The PCR product was analyzed by agarose gel and extracted with a GeneJET Gel Extraction Kit (Thermo). Purified PCR products and pET28b were digested with the restriction enzymes NdeI and HindIII, and corresponding linear DNAs were ligated to generate expression constructs. All inserts in the constructs were sequenced to exclude mutations introduced during PCR amplification and gene manipulation,

### Heterologous Expression and Purification of Recombinant Proteins

Protein expression and purification followed established protocols. The purified proteins were exchanged into storage buffer (25 mM Tris-HCl, pH 8.0, 100 mM NaCl, 3 mM βME. and 10% glycerol) by PD-10 column, aliquoted and stored at −80° C. until needed. CO difference spectroscopy was used to measure the concentrations of functional P450s.

### Analytical HPLC Analysis

For analytical analysis, an HPLC column was kept at 40° C., water with 0.1% formic acid was used as solvent A and acetonitrile with 0.1% formic acid was used as solvent B. The column was eluted first with 1% solvent B for 1 min and then with a linear gradient of 1-20% solvent B in 8 min, followed by another linear gradient of 20-99% solvent B in 2 min. The column was further cleaned with 99% solvent B for 2 min and then re-equilibrated with 1% solvent B for 2 min. The flow rate was set as 1 mL/min, and the products were detected at 211 nm with a PDA detector.

### Whole-Cell Biotransformation

E. coli BL21 Gold cells containing pETDuet and pET28b derived plasmids were grown from glycerol stock overnight in 5 mL Luria broth with 0.1 mg/mL ampicillin and 0.05 mg/mL kanamycin (37° C., 250 rpm). The pre-culture was used to inoculate 100 mL of Terrific broth medium (0.1 mg/mL ampicillin and 0.05 mg/mL kanamycin) in a 500 mL flask; this culture was incubated at 37° C., 250 rpm to OD₆₀₀=0.6-0.8. The cultures were cooled on water-ice mixture and induced with 0.5 mM IPTG. Expression was conducted at 18° C., 250 rpm. for 20 h. For the culture of E. coli BL2l Gold cells containing pACYCDuet and pET28b derived plasmids, 0.05 mg/mL chloramphenicol was used instead of 0.05 mg/mL kanamycin. The cultures were then harvested and resuspended to OD₆₀₀=30 in test medium. Aliquots of the cell suspension were used in the whole cell transformation. To a test tube was added 5 mL cell suspension, 25 μL 100 mM L-Trp or L-Trp analogues, and 25 μL 100 mM L-Arg when necessary. The mixture was then incubated at different conditions. The reactions were quenched by adding equal volume of methanol and the resulting mixture was aliquoted and transferred to a microcentrifuge tube and centrifuged at 14,000 rpm for 10 minutes. The supernatant was transferred to an HPLC vial and analyzed by LC-MS.

TABLE 1
Primers for construction of whole cell transformation plasmids.

| Name | Sequence (5′→3′) | Function |
|---|---|---|
| TB14FN | ATACCATGGTGACCGTCCCCTCGCCG (SEQ ID NO: 17) | TB14 cloning |
| TB14RH | ATCAAGCTTCCCAGCCCACACGTCTTTTGC (SEQ ID NO: 18) | TB14 cloning |
| GDHFB | CAGGATCCGATGTATAAAGATCTGGAAGGTAAAGTGGTG | GDH cloning |
| | (SEQ ID NO: 19) | |
| GDHRH | CAAAGCTTTTAGCCACGACCTGCCTGAAAG (SEQ ID NO: 20) | GDH cloning |
| BsNOSFN | ACTCATATGATGGAAGAAAAAGAAATC (SEQ ID NO: 21) | BsNOS cloning |
| BsNOSRH | ACTAAGCTTCTATTCATACGGTTTGTC (SEQ ID NO: 22) | BsNOS cloning |
| SsTxtDFN | CTACATATGGTGACTTTCGAAGTCGC (SEQ ID NO: 23) | SsTxtD cloning |
| SsTxtDRH | CTCAAGCTTCTGATGAGGGTAAAAGTTG (SEQ ID NO: 24) | SsTxtD cloning |
| StTxtDFN | ACTCATATGGTGACTTTCGAAGTCGCCCTG (SEQ ID NO: 25) | StTxtD cloning |
| StTxtDRH | ACTAAGCTTCTGATGAGGGTAAAAGTTGGGG (SEQ ID NO: 26) | StTxtD cloning |

### Example 2: Whole Cell Nitration System

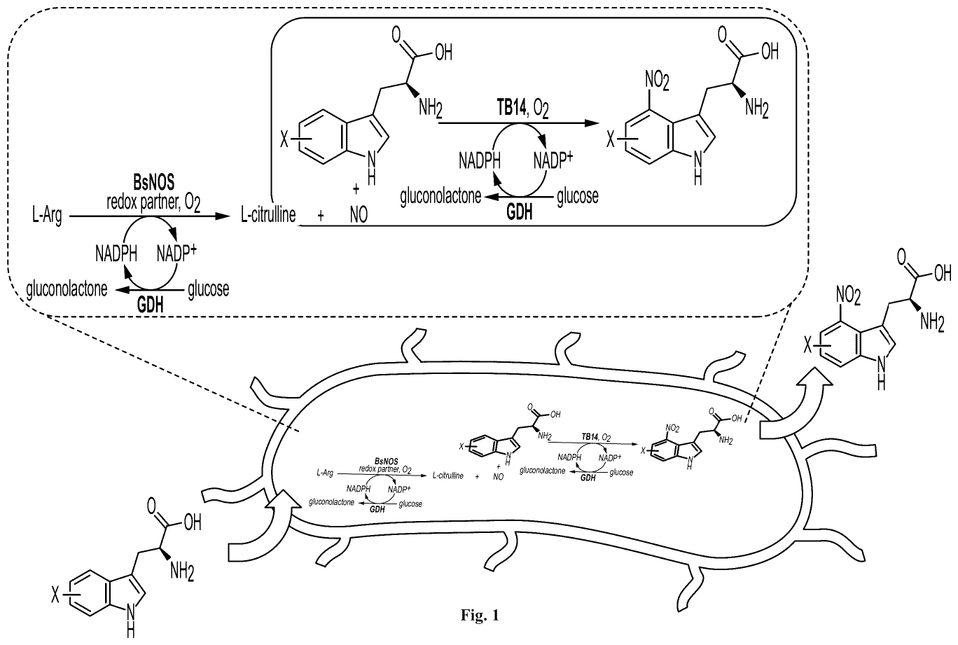

This example describes an E. coli-based biotransformation system for the production of nitrated L-Trp was developed (FIG. 1). The engineered E. coli strain contained three functional genes, TB14 (TB14). nitro oxide synthase (NOS). and Glucose Dehydrogenase 1 (GDH). TB14 is a self-sufficient nitration biocatalyst that is a fusion protein comprising a cytochrome P450 (e.g., a Streptomyces TxtE enzyme) and a catalytic domain of a reductase enzyme (e.g., a prokaryotic reductase enzyme, such as a CYP102A1 (P450BM3) reductase). TB14 is soluble in E. coli. In some embodiments, TB14 is represented by SEQ ID NO: 1.

The co-substrate NO is indispensable for a TxtE nitration reaction. In the in vitro assays. NO was derived from the NO precursor NOC-5 that is expensive, has a short-life, and is often incompatible with bacterial cells (e.g., NO at high concentration is toxic to bacterial cells). The thaxtomin biosynthetic gene cluster in Streptomyces scabies contains a TxtD gene encoding a nitric oxide synthase that converts L-Arg into L-citrulline and NO along with the consumption of NADPH. The expression of the NOS gene in E. coli can, in some embodiments, provide a sustainable and environment-friendly approach to eliminate the dependence of the high-cost and unstable NO precursors in whole cell nitration biotransformation.

It was observed that expression of the TxtD gene from two thaxtomin-producing Streptomyces strains (Streptomyces scabies and Streptomyces turgidiscabies) and yielded only insoluble proteins after optimizing expression conditions. However, expression of a codon-optimized NOS gene from Bacillus subtilis resulted in production of soluble NOS protein in E. coli and was used in the subsequent experiments.

The reaction of NOS requires redox partners for transferring electrons from NADPH. It was observed that non-specific redox partners of E. coli effectively support the BsNOS reaction, making BsNOS containing E. coli strain a viable biosystem to supply NO for the nitration reaction. In some embodiments, insufficient supply of NADPH limits the productivity of biotransformation. Thus, in some embodiments of whole cell nitration systems described in this example, the GDH gene from Bacillus megaterium was also engineered into E. coli to regenerate NADPH that is consumed in both TB14 and BsNOS reactions. GDH catalyzes the oxidation of β-D-glucose to β-D-glucono-1,5-lactone with simultaneous reduction of the cofactor NADP⁺ to NADPH, and may be applied in biocatalysis procedures to regenerate NADPH.

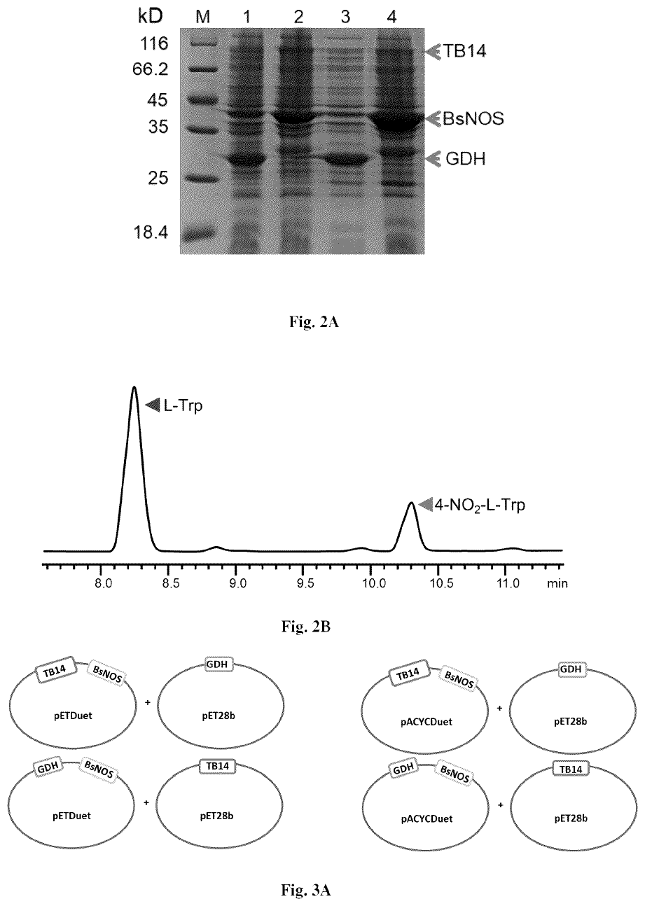

In some experiments. TB14 and BsNOS genes were co-expressed using vector pETDuet, while the GDH gene was separately expressed in the vector pET28b. Both vectors have the same, medium copy numbers (15 to 60) in the host and drive the expression of each gene with a strong inducible promoter T7. In addition, the different antibiotic resistant markers (ampicillin R and kanamycin R) in these two vectors make them suitable for simultaneous expression of three genes in the same host. The two constructs described above were transformed into E. coli BL21-GOLD strain. The overexpression of the three enzymes was induced by IPTG. SDS-PAGE analysis of the soluble crude extract (FIG. 2A, lane 1) indicated successful overexpression of BsNOS (42 kD) and GDH (28 kD). Soluble TB14 (110 kD) expression was also observed. In some embodiments, co-expression of BsNOS and/or GDH negatively influences the expression of TB14. Nevertheless, this engineered E. coli strain was used in whole-cell biotransformation to produce 4-NO₂-L-Trp from fed L-Trp. After 20-h incubation, the successful production of 4-NO₂-L-Trp was confirmed by LC-MS (FIG. 2B).

### Optimization of Heterologous Enzyme Expression

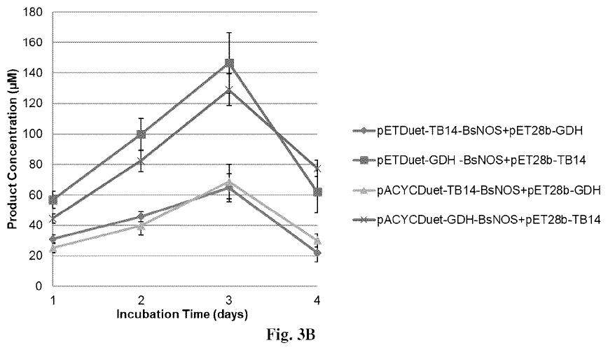

Next, the copy numbers of the three genes (e.g., TB14, BsNOS, GDH) were varied in order to improve TB14 expression and to improve plasmid stability. Plasmid pACYCDuet was used for the co-expression of two target genes. The pACYCDuet plasmid includes two T7 promoters to drive the proteins expression carries the P15A replicon instead of pBR322-derived ColE1 replicon as in pETDuet and pET28b. which can provide higher plasmid stability when two plasmids are used. Three new pairs of expression constructs were created, pETDuet-GDH-BsNOS+pET28b-TB14, pACYCDuet-TB14-BsNOS+pET28b-GDH, and pACYCDuet-GDH-BsNOS+pET28b-TB14, and the corresponding engineered E. coli strains (FIG. 3A). Protein expression levels in these strains was then examined by SDS-PAGE (FIG. 2A). The bacterial strain transformed with the pair of pETDuet-GDH-BsNOS and pET28b-TB14 plasmids showed significantly increased TB14 expression level, and it also demonstrated the high nitration activity (FIG. 3B). This strain was used in the following experiments.

### Optimization of Fermentation Conditions

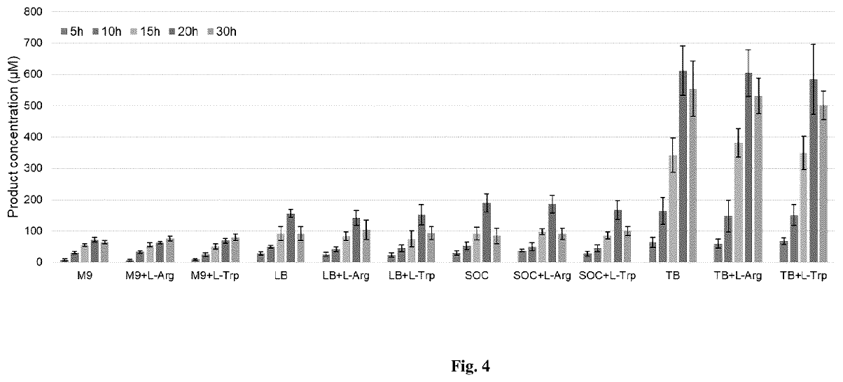

Fermentation conditions, including medium, temperature, substrate supplement, and harvesting time were then investigated. Minimal medium M9 was used in previously described experiments. As M9 medium is nutritiously poor, it was investigated whether nutrition availability could influence the whole cell nitration efficiency. Three nutrition rich media, including LB medium, SOC medium and TB medium, were tested along with the M9 medium in a whole cell nitration system. As shown in (FIG. 4), transformations supported by nutrition richer media generally had higher efficiency than those supported by the M9. Notably. TB medium supported transformation yielded as high as 600 μM of nitrated tryptophan (149 mg/L) after 20-hour fermentation.

The time profile of the product formation (FIG. 4) was then tested. In all the nutrition rich media, the highest productivity was observed at approximately 20 hours. By contrast, it required 3 days in the M9 medium. Notably, the concentration of nitrated tryptophan in the rich media started to decrease after 30-hour fermentation. In some embodiments, E. coli endogenous tryptophanase (EC 4.1.99.1), which converts tryptophan to indole, pyruvate and NH₃, mediates production decomposition. E. coli tryptophanase (tnaA) was cloned and recombinant enzyme was prepared. In vitro biochemical assay data indicated that TnaA was not able to decompose 4-NO₂-Trp.

In TB14 reactions, the co-substrate NO is generated from L-arginine by BsNOS. The effect of increasing the concentration of L-Arg was then tested (FIG. 4). No significant change in nitro-tryptophan production was observed when 5 mM of L-Arg was added to each of the transformation systems tested. This result indicates that L-Arg or NO is not the limiting factor in the whole cell transformation. The effect of increased concentration of the substrate L-Trp was also tested (FIG. 4). No significant change of the production was observed when 5 mM of L-Trp was added to the transformation system.

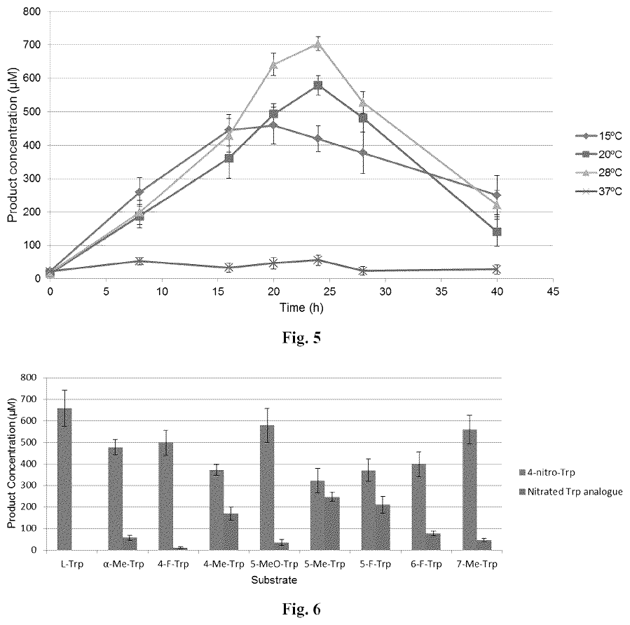

The temperature effects on the whole cell nitration were also investigated. In vitro studies indicated TB14 was active at temperatures between 10 to 30° C. All previous experiments were performed at 20° C., Productivity of the whole cell system at four different fermentation temperatures (15° C. 20° C., 28° C. and 37° C.) at different time points was investigated (FIG. 5). The transformations at 15° C., 20° C. and 28° C. each resulted in product yield. At 28° C., product concentration was observed to be 700 μM after 24-hour fermentation. Interestingly, the optimal growth temperature of E. coli, 37° C., almost completely abolished the nitration transformation.

### Production of Nitrated Tryptophan Analogues by Whole Cell Nitration System

A series of tryptophan analogues that can be nitrated by TxtE and its variants in vitro have been identified. In this example, the substrate scope of whole cell systems was investigated using these tryptophan analogues. These unnatural analogues generally compete with the native substrate L-Trp abundant in the TB medium in the whole cell transformation. However, data indicate that α-Me-Trp, 4-F-Trp, 4-Me-Trp, 5-MeO-Trp, 5-Me-Trp, 5-F-Trp, 6-F-Trp, and 7-Me-Trp all were successfully nitrated using the whole cell nitration system. Similar to observations in the in vitro enzymatic reactions, whole cell-based nitration demonstrated the highest conversion rates with 4-Me-Trp, 5-Me-Trp and 5-F-Trp (FIG. 6). The substrate 5-Me-Trp product concentration reached approximately 250 μM after 24-hour fermentation, along with approximately 320 μM of nitrated tryptophan (FIG. 6). The following nitro-tryptophan and nitro-tryptophan analogs can be synthesized using any of the methods delineated herein:

### Example 3: Preparation of (S)-2-amino-3-(5-methyl-4-nitro-1H-iodol-3-yl)propanoic acid (3)

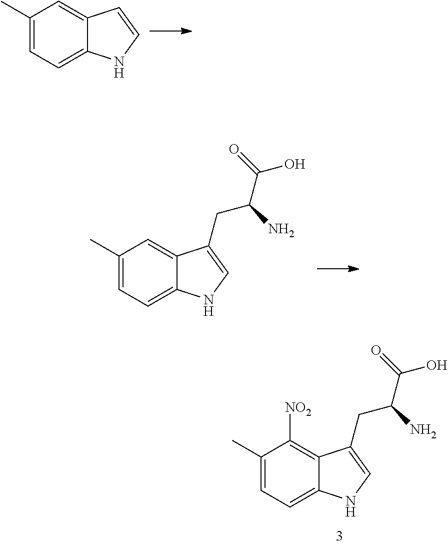

Example 3 can be prepared from 5-methylindole as shown above.

### Example 4: Preparation of (S)-2-amino-3-(6-methyl-4-nit-1H-indol-3-yl)propanoic acid (4)

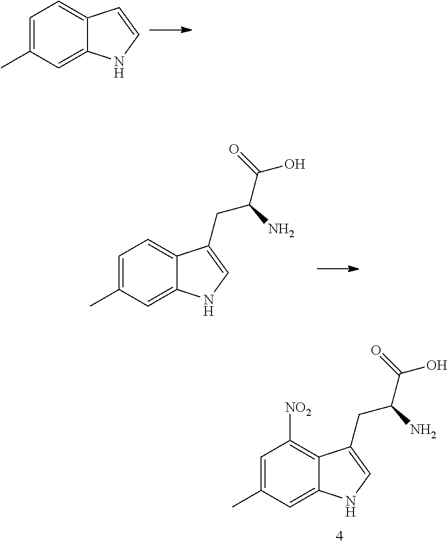

Example 4 can be prepared from 6-methylindole as shown above.

### Example 5: Preparation of (S)-2-amino-3-(7-methyl-4-nitro-1H-indol-3-yl)propanoic acid (5)

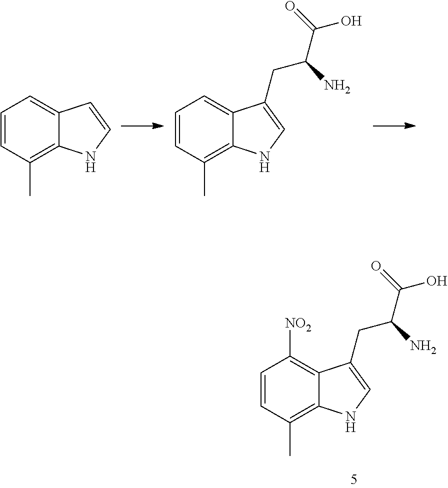

Example 5 can be prepared from 7-methylindole as shown above.

### Example 6: Preparation of (S)-2-amino-3-(5-fluoro-4-nitro-1H-indol-3-yl)propanoic acid (6)

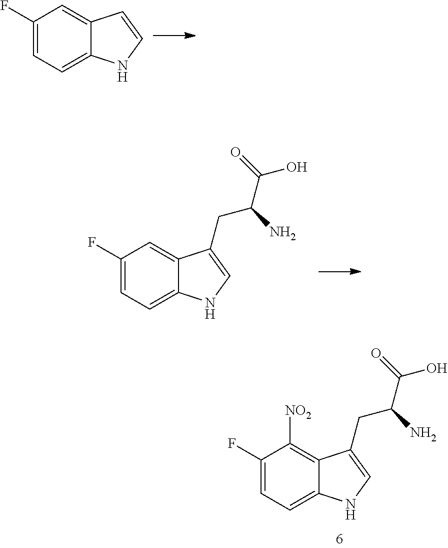 [SMILES: Fc1ccc2[nH]ccc2c1 ; N[C@@H](Cc1c[nH]c2ccc(F)cc12)C(=O)O ; N[C@@H](Cc1c[nH]c2ccc(F)c([N+](=O)[O-])c12)C(=O)O]

Example 6 can be prepared from 5-fluoroindole as shown above.

### Example 7: Preparation of (S)-2-amino-3-(6-fluoro-4-nitro-1H-indol-3-yl)propanoic acid (7)

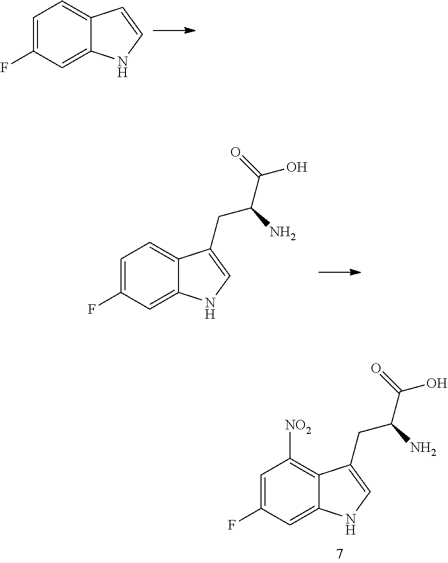 [SMILES: Fc1ccc2cc[nH]c2c1 ; N[C@@H](Cc1c[nH]c2cc(F)ccc12)C(=O)O ; N[C@@H](Cc1c[nH]c2cc(F)cc([N+](=O)[O-])c12)C(=O)O]

Example 7 can be prepared from 6-fluoroindole.

### Example 8: Preparation of (S)-2-amino-3-(7-fluoro-4-nitro-1H-indol-3-yl)propanoic acid (S)

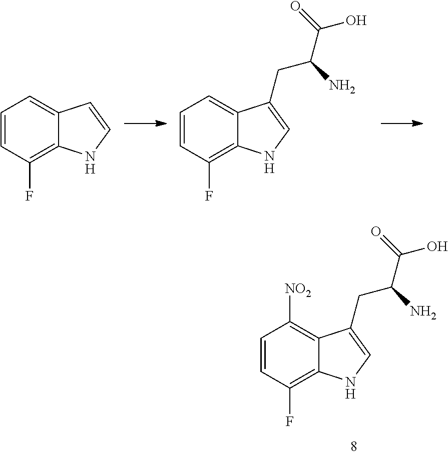 [SMILES: Fc1cccc2cc[nH]c12 ; N[C@@H](Cc1c[nH]c2c(F)cccc12)C(=O)O ; N[C@@H](Cc1c[nH]c2c(F)ccc([N+](=O)[O-])c12)C(=O)O]

Example 8 can be prepared from 7-fluoroindole as shown above.

### Example 9: Preparation of (S)-2-amino-3-(4-fluoro-7-nitro-1H-indol-3-yl)propanoic acid (9)

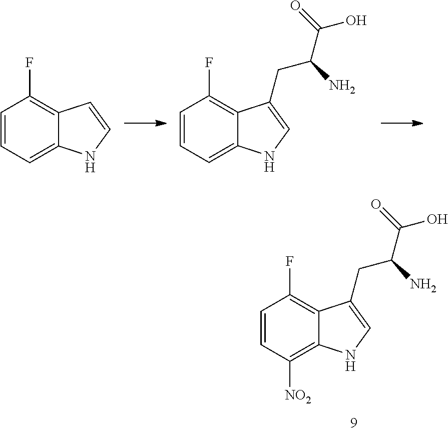 [SMILES: Fc1cccc2[nH]ccc12 ; N[C@@H](Cc1c[nH]c2cccc(F)c12)C(=O)O ; N[C@@H](Cc1c[nH]c2c([N+](=O)[O-])ccc(F)c12)C(=O)O]

Example 9 can be prepared from 4-fluoroindole as shown above.

### Example 10: Preparation of (S)-2-amino-3-(5-chloro-4-nitro-1H-indol-3-yl)propanoic acid

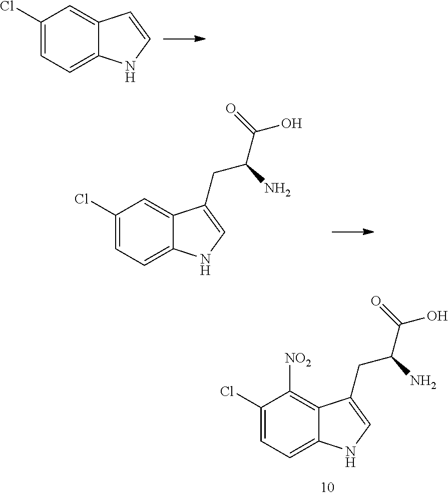 [SMILES: Clc1ccc2[nH]ccc2c1 ; N[C@@H](Cc1c[nH]c2ccc(Cl)cc12)C(=O)O ; N[C@@H](Cc1c[nH]c2ccc(Cl)c([N+](=O)[O-])c12)C(=O)O]

Example 10 can be prepared from 5-chloroindole as shown above.

### Example 11: Preparation of (S)-2-amino-3-(6-chloro-4-nitro-1H-indol-3-yl)propanoic acid (11)

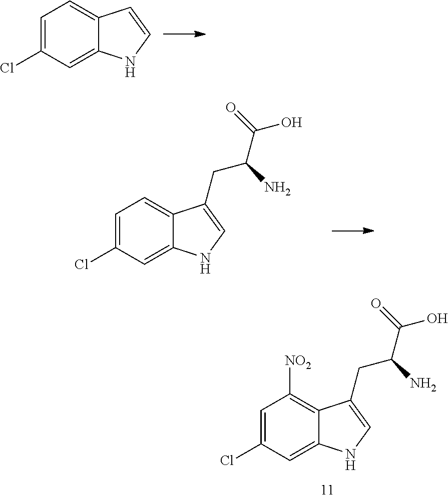 [SMILES: Clc1ccc2cc[nH]c2c1 ; N[C@@H](Cc1c[nH]c2cc(Cl)ccc12)C(=O)O ; N[C@@H](Cc1c[nH]c2cc(Cl)cc([N+](=O)[O-])c12)C(=O)O]

Example 11 can be prepared from 6-chloroindole as shown above.

### Example 12: Preparation of (S)-2-amino-3-(7-chloro-4-nitro-1H-indol-3-yl)propanoic acid (12)

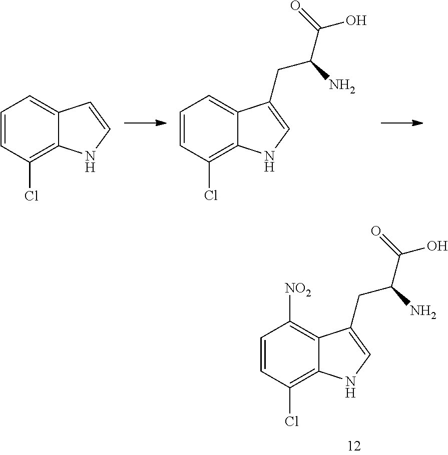 [SMILES: Clc1cccc2cc[nH]c12 ; N[C@@H](Cc1c[nH]c2c(Cl)cccc12)C(=O)O ; N[C@@H](Cc1c[nH]c2c(Cl)ccc([N+](=O)[O-])c12)C(=O)O]

Example 12 can be prepared from 7-chloroindole as shown above.

### Example 13: Preparation of (S)-2-amino-3-(4-chloro-7-nitro-1H-indol-3-yl)propanoic acid (13)

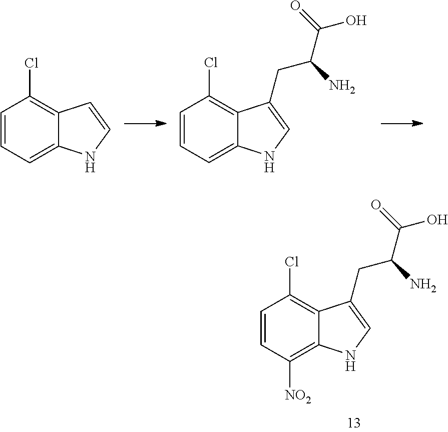 [SMILES: Clc1cccc2[nH]ccc12 ; N[C@@H](Cc1c[nH]c2cccc(Cl)c12)C(=O)O ; N[C@@H](Cc1c[nH]c2c([N+](=O)[O-])ccc(Cl)c12)C(=O)O]

Example 13 can be prepared from 4-chloroindole as shown above.

### Example 14: Preparation of (S)-2-amino-3-(5-bromo-4-nitro-1H-indol-3-yl)propanoic acid (14)

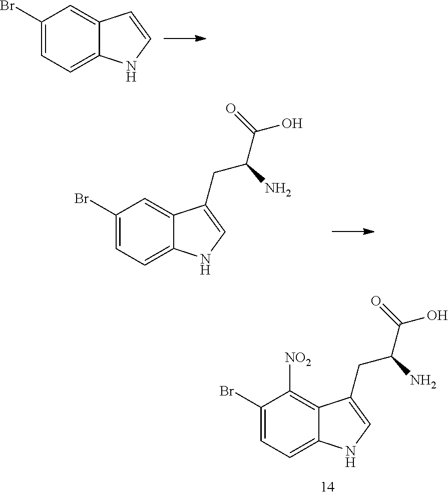 [SMILES: Brc1ccc2[nH]ccc2c1 ; N[C@@H](Cc1c[nH]c2ccc(Br)cc12)C(=O)O ; N[C@@H](Cc1c[nH]c2ccc(Br)c([N+](=O)[O-])c12)C(=O)O]

Example 14 can be prepared from 5-bromoindole as shown above.

### Example 15: Preparation of (S)-2-amino-3-(6-bromo-4-nitro-1H-indol-3-yl)propanoic acid (15)

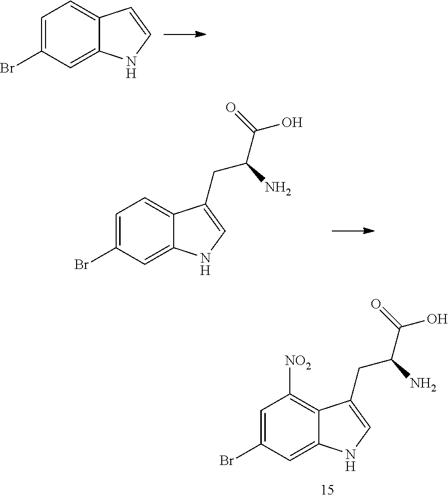 [SMILES: Brc1ccc2cc[nH]c2c1 ; N[C@@H](Cc1c[nH]c2cc(Br)ccc12)C(=O)O ; N[C@@H](Cc1c[nH]c2cc(Br)cc([N+](=O)[O-])c12)C(=O)O]

Example 15 can be prepared from 6-bromoindole as shown.

### Example 16: Preparation of (S)-2-amino-3-(7-bromo-4-nitro-1H-indol-3-yl)propanoic acid (16)

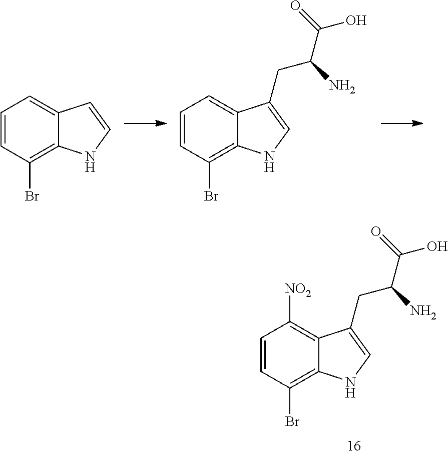 [SMILES: Brc1cccc2cc[nH]c12 ; N[C@@H](Cc1c[nH]c2c(Br)cccc12)C(=O)O ; N[C@@H](Cc1c[nH]c2c(Br)ccc([N+](=O)[O-])c12)C(=O)O]

Example 16 can be prepared from 7-bromoindole as shown above.

### Example 17: Preparation of (S)-2-amino-3-(4-bromo-7-nitro-1H-indol-3-yl)propanoic acid (17)

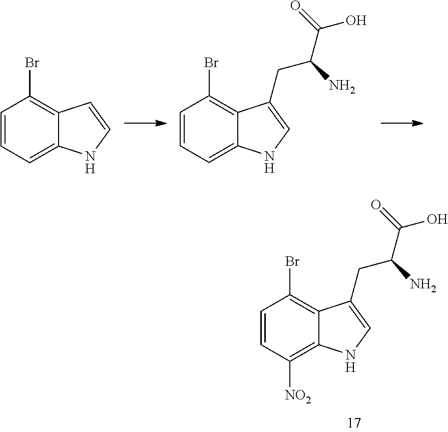 [SMILES: Brc1cccc2[nH]ccc12 ; N[C@@H](Cc1c[nH]c2cccc(Br)c12)C(=O)O ; N[C@@H](Cc1c[nH]c2c([N+](=O)[O-])ccc(Br)c12)C(=O)O]

Example 17 can be prepared from 4-bromoindole as shown above.

### Example 18: Preparation of (S)-2-amino-3-(5-methoxy-4-nitro-1H-indol-3-yl)propanoic acid (18)

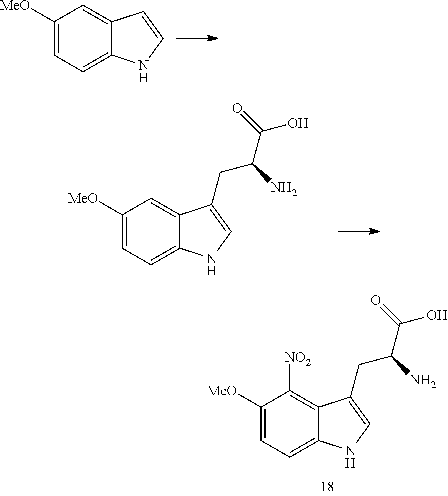 [SMILES: COc1ccc2[nH]ccc2c1 ; COc1ccc2[nH]cc(C[C@H](N)C(=O)O)c2c1 ; COc1ccc2[nH]cc(C[C@H](N)C(=O)O)c2c1[N+](=O)[O-]]

Example 18 can be prepared from 5-methoxyindole as shown above.

### Example 19: Preparation of (S)-2-amino-3-(6-methoxy-4-nitro-1H-indol-3-yl)propanoic acid (19)

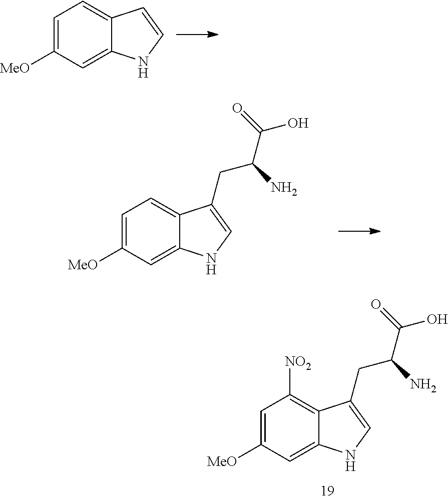 [SMILES: COc1ccc2cc[nH]c2c1 ; COc1ccc2c(C[C@H](N)C(=O)O)c[nH]c2c1 ; COc1cc([N+](=O)[O-])c2c(C[C@H](N)C(=O)O)c[nH]c2c1]

Example 19 can be prepared from 6-methoxyindole as shown above.

### Example 20: Preparation of (S)-2-amino-3-(7-methoxy-4-nitro-1H indol-3-yl)propanoic acid (20)

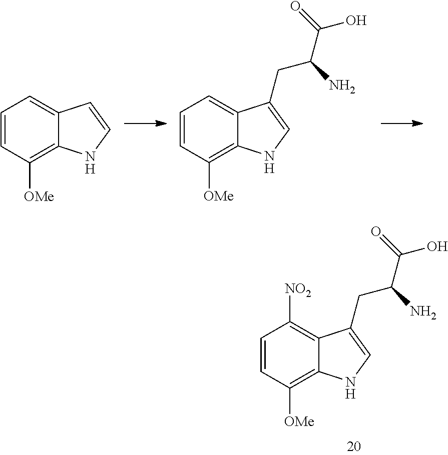 [SMILES: COc1cccc2cc[nH]c12 ; COc1cccc2c(C[C@H](N)C(=O)O)c[nH]c12 ; COc1ccc([N+](=O)[O-])c2c(C[C@H](N)C(=O)O)c[nH]c12]

Example 20 can be prepared from 7-methoxyindole as shown above.

### Example 21: Preparation of (S)-2-amino-3-(4-methoxy-7-nitro-1H-indol-3-yl)propanoic acid (21)

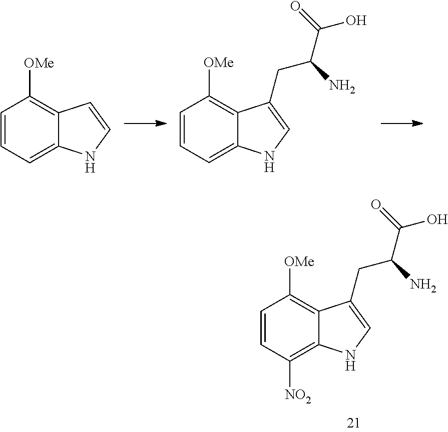 [SMILES: COc1cccc2[nH]ccc12 ; COc1cccc2[nH]cc(C[C@H](N)C(=O)O)c12 ; COc1ccc([N+](=O)[O-])c2[nH]cc(C[C@H](N)C(=O)O)c12]

Example 21 can be prepared from 4-methoxyindole as shown above.

### Example 22: Preparation of (S)-2-amino-3-(5-amino-4-nitro-1H-indol-3-yl)propanoic acid (22)

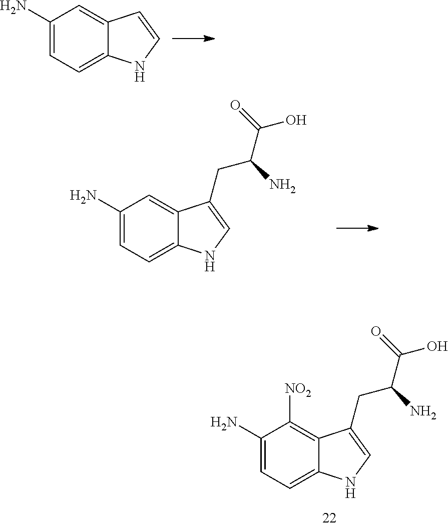 [SMILES: Nc1ccc2[nH]ccc2c1 ; Nc1ccc2[nH]cc(C[C@H](N)C(=O)O)c2c1 ; Nc1ccc2[nH]cc(C[C@H](N)C(=O)O)c2c1[N+](=O)[O-]]

Example 22 can be prepared from 5-aminoindole as shown above.

### Example 23: Preparation of (S)-2-amino-3-(6-amino-4-nitro-1H-indol-3-yl)propanoic acid (23)

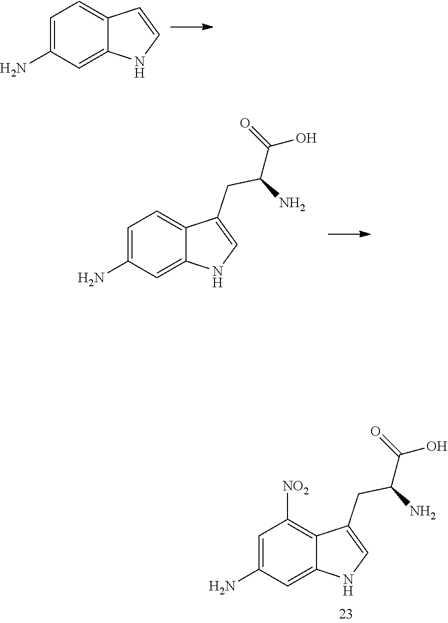 [SMILES: Nc1ccc2cc[nH]c2c1 ; Nc1ccc2c(C[C@H](N)C(=O)O)c[nH]c2c1 ; Nc1cc([N+](=O)[O-])c2c(C[C@H](N)C(=O)O)c[nH]c2c1]

Example 23 can be prepared from 6-aminoindole as shown above.

### Example 24: Preparation of (S)-2-amino-3-(7-amino-4-nitro-1H-indol-3-yl)propanoic acid (24)

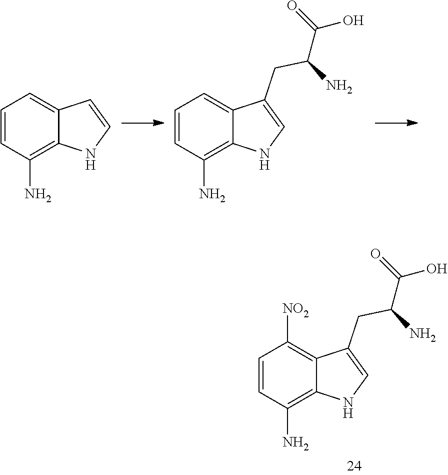 [SMILES: Nc1cccc2cc[nH]c12 ; Nc1cccc2c(C[C@H](N)C(=O)O)c[nH]c12 ; Nc1ccc([N+](=O)[O-])c2c(C[C@H](N)C(=O)O)c[nH]c12]

Example 24 can be prepared from 7-aminoindole as shown above.

### Example 25: Preparation of (S)-2-amino-3-(4-amino-7-nitro-1H-indol-3-yl)propanoic acid (25)

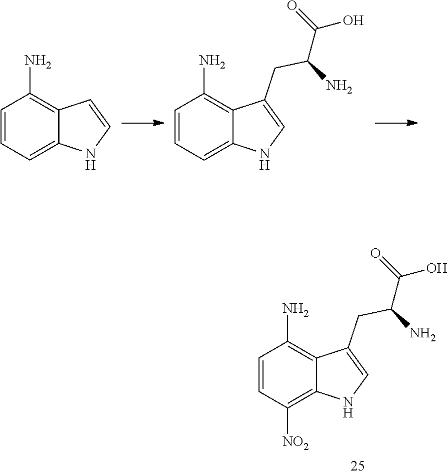 [SMILES: Nc1cccc2[nH]ccc12 ; Nc1cccc2[nH]cc(C[C@H](N)C(=O)O)c12 ; Nc1ccc([N+](=O)[O-])c2[nH]cc(C[C@H](N)C(=O)O)c12]

Example 25 can be prepared from 4-aminoindole as shown above.

### Example 26: Preparation of (S)-2-amino-3-(5-hydroxy-4-nitro-1H-indol-3-yl)propanoic acid (26)

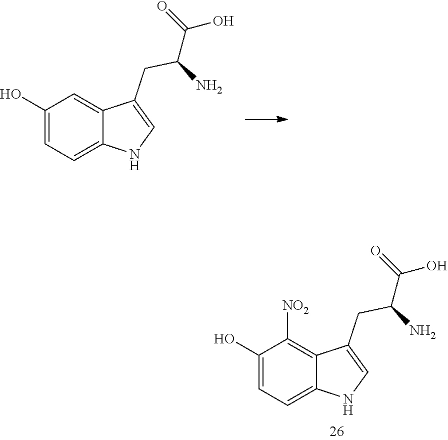 [SMILES: N[C@@H](Cc1c[nH]c2ccc(O)cc12)C(=O)O ; N[C@@H](Cc1c[nH]c2ccc(O)c([N+](=O)[O-])c12)C(=O)O]

Example 26 can be prepared from (S)-2-amino-3-(5-hydroxy-1H-indol-3-yl)propanoic acid as shown above.

### Example 27: Preparation of (S)-2-amino-3-(6-hydroxy-4-nitro-1H-4-indol-3-yl)propanoic acid (27)

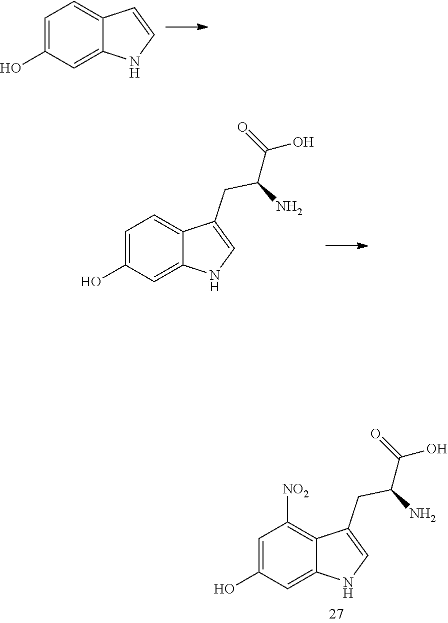 [SMILES: Oc1ccc2cc[nH]c2c1 ; N[C@@H](Cc1c[nH]c2cc(O)ccc12)C(=O)O ; N[C@@H](Cc1c[nH]c2cc(O)cc([N+](=O)[O-])c12)C(=O)O]

Example 27 can be prepared from 6-hydroxyindole as shown above.

### Example 28: Preparation of (S)-2-amino-3-(7-hydroxy-4-nitro-1H-indol-3-yl)propanoic acid (25)

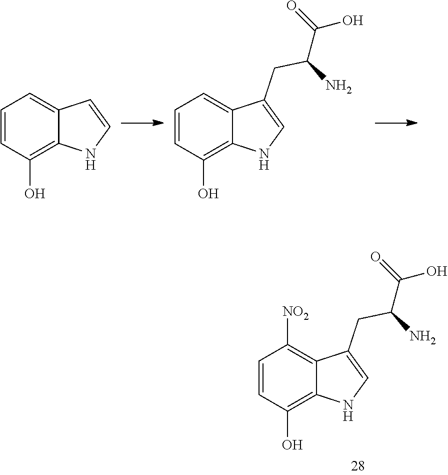 [SMILES: Oc1cccc2cc[nH]c12 ; N[C@@H](Cc1c[nH]c2c(O)cccc12)C(=O)O ; N[C@@H](Cc1c[nH]c2c(O)ccc([N+](=O)[O-])c12)C(=O)O]

Example 28 can be prepared from 7-hydroxyindole as shown above.

### Example 29: Preparation of (S)-2-amino-3-(4-hydroxy-7-nitro-1H-indol-3-yl)propanoc acid (29)

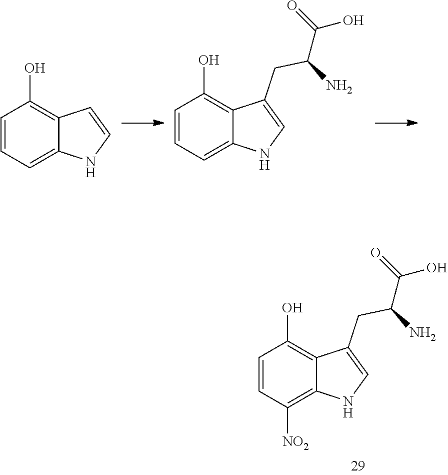 [SMILES: Oc1cccc2[nH]ccc12 ; N[C@@H](Cc1c[nH]c2cccc(O)c12)C(=O)O ; N[C@@H](Cc1c[nH]c2c([N+](=O)[O-])ccc(O)c12)C(=O)O]

Example 29 can be prepared from 4-hydroxyindole as shown above.

### Example 30: Preparation of (S)-2-amino-3-(4-nitro-5-phenyl-1H-indol-3-yl)propanoic acid (30)

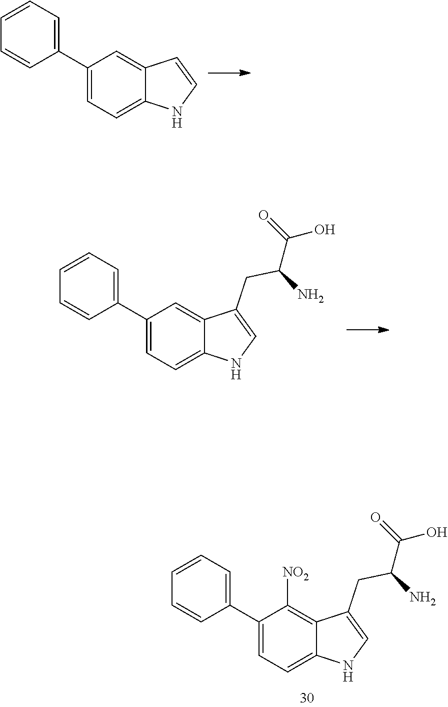 [SMILES: c1ccc(-c2ccc3[nH]ccc3c2)cc1 ; N[C@@H](Cc1c[nH]c2ccc(-c3ccccc3)cc12)C(=O)O ; N[C@@H](Cc1c[nH]c2ccc(-c3ccccc3)c([N+](=O)[O-])c12)C(=O)O]

Example 30 can be prepared from 5-phenylindole as shown above.

### Example 31: Preparation of (S)-2-amino-3-(4-nitro-6-phenyl-1H-indol-3-yl)propanoic acid (31)

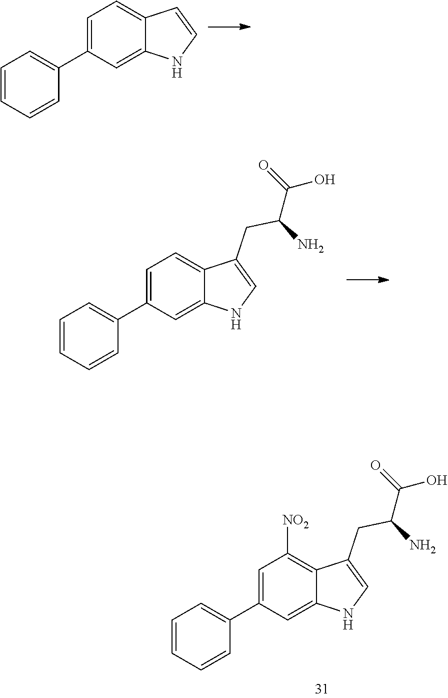 [SMILES: c1ccc(-c2ccc3cc[nH]c3c2)cc1 ; N[C@@H](Cc1c[nH]c2cc(-c3ccccc3)ccc12)C(=O)O ; N[C@@H](Cc1c[nH]c2cc(-c3ccccc3)cc([N+](=O)[O-])c12)C(=O)O]

Example 31 can be prepared from 6-phenylindole as shown above.

### Example 32: Preparation of (S)-2-amino-3-(4-nitro-7-phenyl-1H-indol-3-yl)propanoic acid (32)

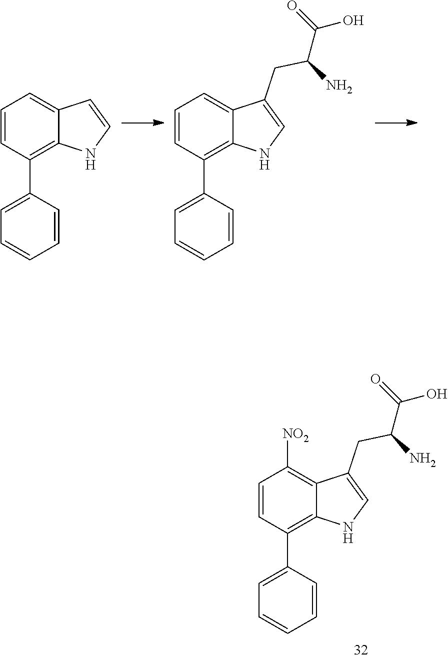 [SMILES: c1ccc(-c2cccc3cc[nH]c23)cc1 ; N[C@@H](Cc1c[nH]c2c(-c3ccccc3)cccc12)C(=O)O ; N[C@@H](Cc1c[nH]c2c(-c3ccccc3)ccc([N+](=O)[O-])c12)C(=O)O]

Example 32 can be prepared from 7-phenylindole as shown above.

### Example 33: Preparation of (S)-2-amino-3-(7-nitro-4-phenyl-1H-indol-3-yl)propanoic acid (33)

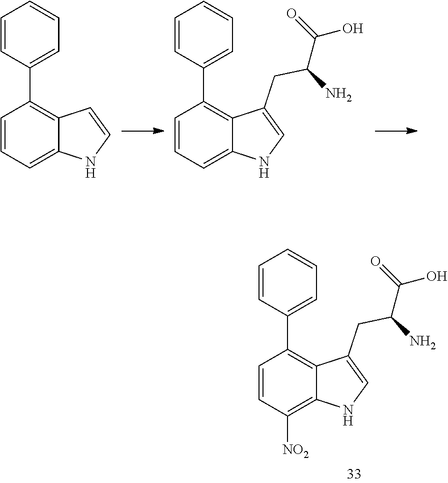 [SMILES: c1ccc(-c2cccc3[nH]ccc23)cc1 ; N[C@@H](Cc1c[nH]c2cccc(-c3ccccc3)c12)C(=O)O ; N[C@@H](Cc1c[nH]c2c([N+](=O)[O-])ccc(-c3ccccc3)c12)C(=O)O]

Example 33 can be prepared from 4-phenylindole as shown above.

### Example 34: Preparation of (S)-2-amino-3-(5-cyclopropyl-4-nitro-1H-idol-3-yl)propanoic acid (34)

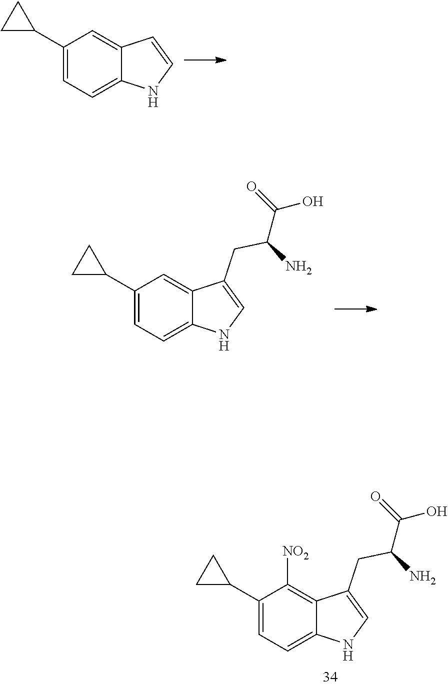 [SMILES: c1cc2cc(C3CC3)ccc2[nH]1 ; N[C@@H](Cc1c[nH]c2ccc(C3CC3)cc12)C(=O)O ; N[C@@H](Cc1c[nH]c2ccc(C3CC3)c([N+](=O)[O-])c12)C(=O)O]

Example 34 can be prepared from 5-cyclopropylindole as shown above.

### Example 35: Preparation of(S)-2-amino-3-(6-cyclopropyl-4-nitro-1H-indol-3-yl)propanoic acid (35)

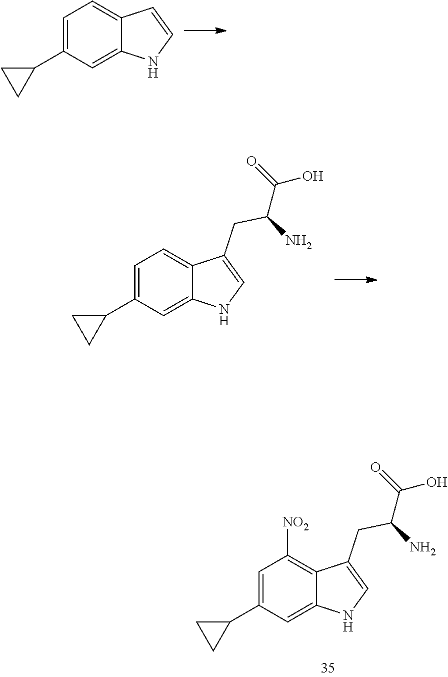 [SMILES: c1cc2ccc(C3CC3)cc2[nH]1 ; N[C@@H](Cc1c[nH]c2cc(C3CC3)ccc12)C(=O)O ; N[C@@H](Cc1c[nH]c2cc(C3CC3)cc([N+](=O)[O-])c12)C(=O)O]

Example 35 can be prepared from 6-cyclopropylindole as shown above.

### Example 36: Preparation of (S)-2-amino-3-(7-cyclopropyl-4-nitro-1H-indol-3-yl)propanoic acid (36)

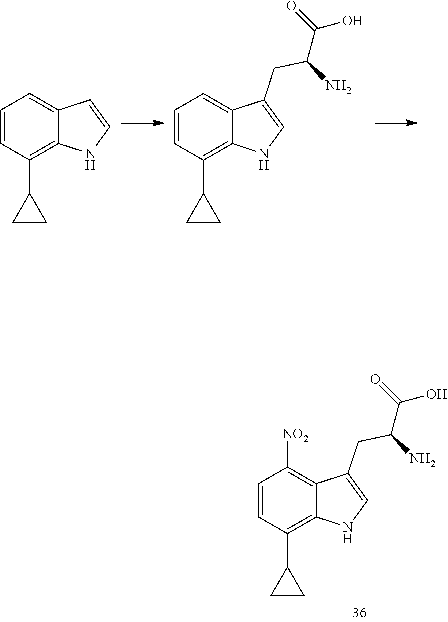 [SMILES: c1cc(C2CC2)c2[nH]ccc2c1 ; N[C@@H](Cc1c[nH]c2c(C3CC3)cccc12)C(=O)O ; N[C@@H](Cc1c[nH]c2c(C3CC3)ccc([N+](=O)[O-])c12)C(=O)O]

Example 36 can be prepared from 7-cyclopropylindole as shown above.

### Example 37: Preparation of (S)-2-amino-3-(4-cyclopropyl-7-nitro-1H-indol-3-yl)propanoic acid (37)

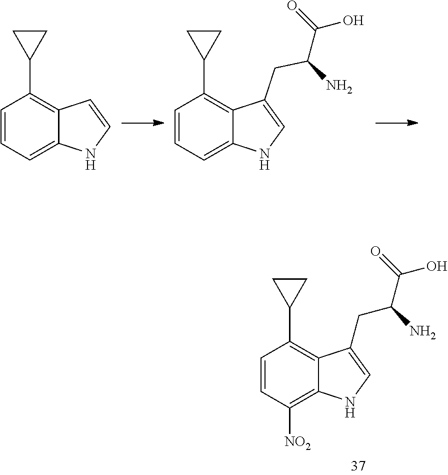 [SMILES: c1cc(C2CC2)c2cc[nH]c2c1 ; N[C@@H](Cc1c[nH]c2cccc(C3CC3)c12)C(=O)O ; N[C@@H](Cc1c[nH]c2c([N+](=O)[O-])ccc(C3CC3)c12)C(=O)O]

Example 37 can be prepared from 4-cyclopropylindole as shown above.

### Example 38: Preparation of (S)-2-amino-3-(4-nitro-5-vinyl-1H-indol-3-yl)propanoic acid (38)

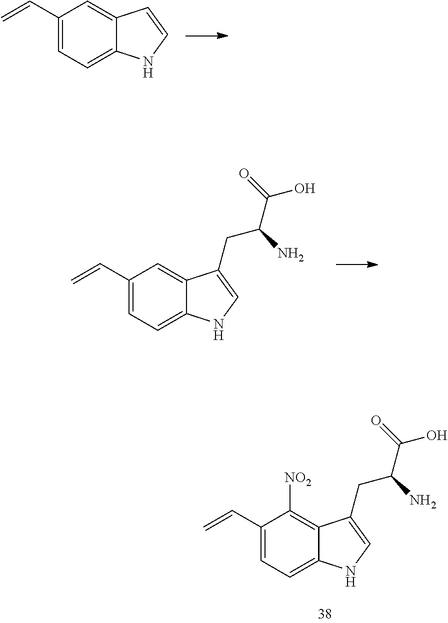 [SMILES: C=Cc1ccc2[nH]ccc2c1 ; C=Cc1ccc2[nH]cc(C[C@H](N)C(=O)O)c2c1 ; C=Cc1ccc2[nH]cc(C[C@H](N)C(=O)O)c2c1[N+](=O)[O-]]

Example 38 can be prepared from 5-vinylindole as shown above.

### Example 39: Preparation of (S)-2-amino-3-(4-nitro-6-vinyl-1H-indol-3-yl)propanoic acid (39)

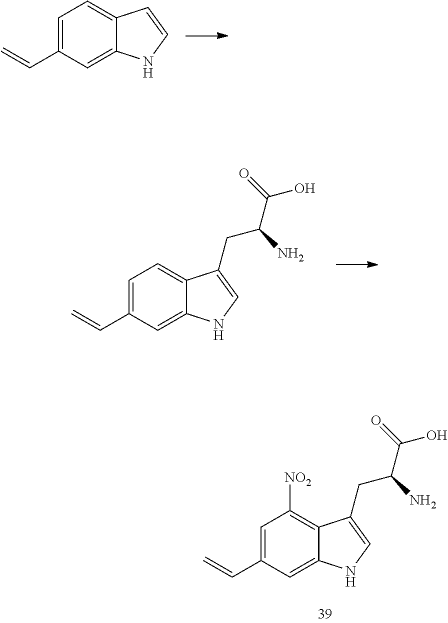 [SMILES: C=Cc1ccc2cc[nH]c2c1 ; C=Cc1ccc2c(C[C@H](N)C(=O)O)c[nH]c2c1 ; C=Cc1cc([N+](=O)[O-])c2c(C[C@H](N)C(=O)O)c[nH]c2c1]

Example 39 can be prepared from 6-vinylindole as shown above.

### Example 40: Preparation of (S)-2-amino-3-(4-nitro-7-vinyl-1H-indol-3-yl)propanoic acid (40)

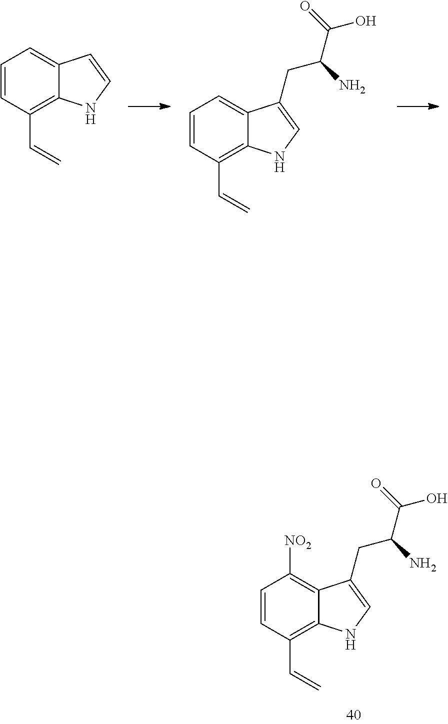 [SMILES: C=Cc1cccc2cc[nH]c12 ; C=Cc1cccc2c(C[C@H](N)C(=O)O)c[nH]c12 ; C=Cc1ccc([N+](=O)[O-])c2c(C[C@H](N)C(=O)O)c[nH]c12]

Example 40 can be prepared from 7-vinylindole as shown above.

### Example 41: Preparation of (S)-2-amino-3-(7-nitro-4-vinyl-1H-indol-3-yl)propanoic acid (41)

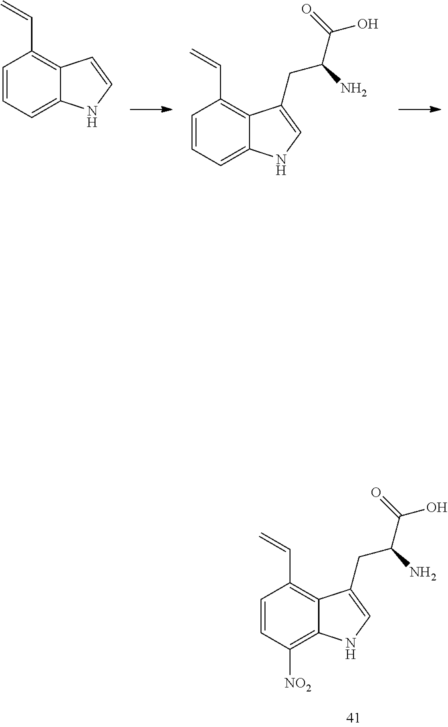 [SMILES: C=Cc1cccc2[nH]ccc12 ; C=Cc1cccc2[nH]cc(C[C@H](N)C(=O)O)c12 ; C=Cc1ccc([N+](=O)[O-])c2[nH]cc(C[C@H](N)C(=O)O)c12]

Example 41 can be prepared from 4-vinylindole as shown above.

### Example 42: Preparation of (S)-2-amino-3-(5-ethynyl-4-nitro-1H-indol-3-yl)propanoic acid (42)

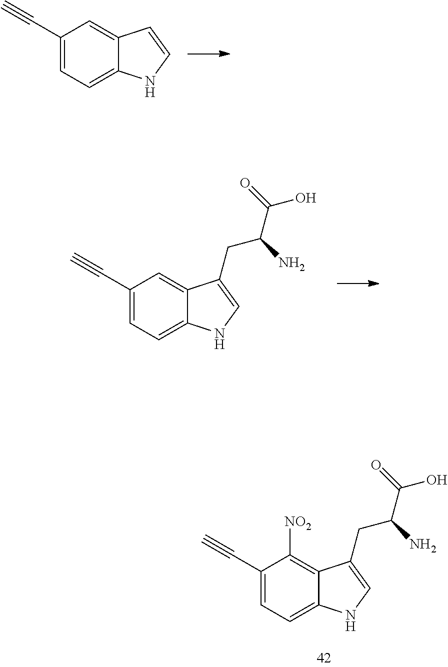 [SMILES: C#Cc1ccc2[nH]ccc2c1 ; C#Cc1ccc2[nH]cc(C[C@H](N)C(=O)O)c2c1 ; C#Cc1ccc2[nH]cc(C[C@H](N)C(=O)O)c2c1[N+](=O)[O-]]

Example 42 can be prepared from 5-ethylindole as shown above.

### Example 43: Preparation of (S)-2-amine-3-(6-ethynyl-4-nitro-1H-indol-3-yl)propanoic acid (43)

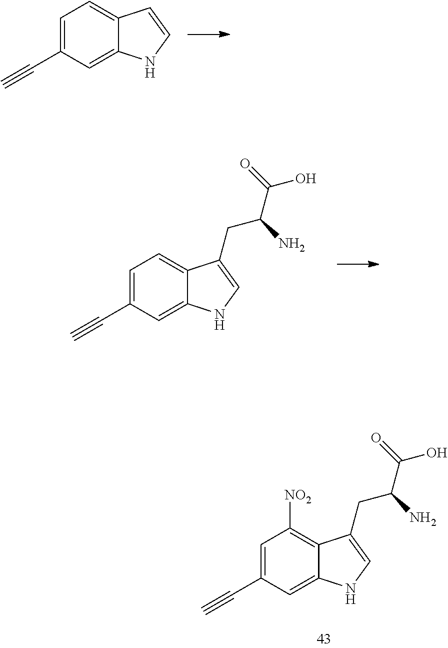 [SMILES: C#Cc1ccc2cc[nH]c2c1 ; C#Cc1ccc2c(C[C@H](N)C(=O)O)c[nH]c2c1 ; C#Cc1cc([N+](=O)[O-])c2c(C[C@H](N)C(=O)O)c[nH]c2c1]

Example 43 can be prepared from 6-ethylindole as shown above.

### Example 44: Preparation a((S)-2-amino-3-(7-ethynyl-4-nitro-1H-indol-3-yl)propanoic acid (44)

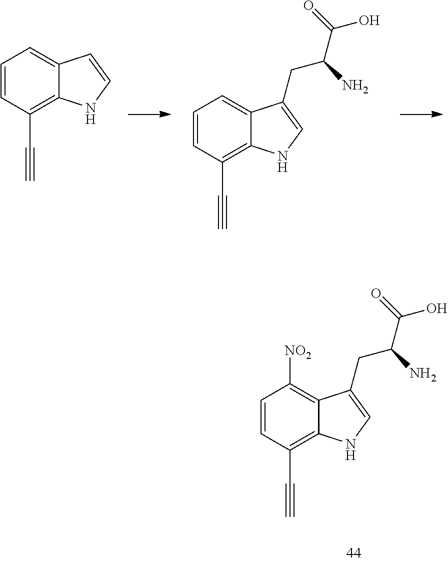 [SMILES: C#Cc1cccc2cc[nH]c12 ; C#Cc1cccc2c(C[C@H](N)C(=O)O)c[nH]c12 ; C#Cc1ccc([N+](=O)[O-])c2c(C[C@H](N)C(=O)O)c[nH]c12]

Example 44 can be prepared from 7-ethylindole as shown above.

### Example 45: Preparation of (S)-2-amino-3-(4-ethynyl-7-nitro-1H-indol-3-yl)propanoic acid (45)

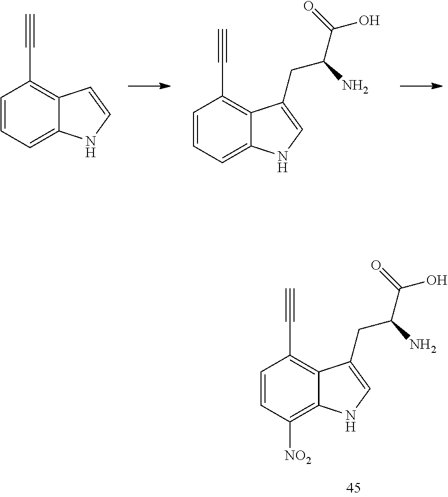 [SMILES: C#Cc1cccc2[nH]ccc12 ; C#Cc1cccc2[nH]cc(C[C@H](N)C(=O)O)c12 ; C#Cc1ccc([N+](=O)[O-])c2[nH]cc(C[C@H](N)C(=O)O)c12]

Example 45 can be prepared from 4-ethylindole as shown above.

### Example 46: Preparation of (S)-2-amino-3-(5-morpholino-4-nitro-1H-indol-3-yl)propanoic acid (46)

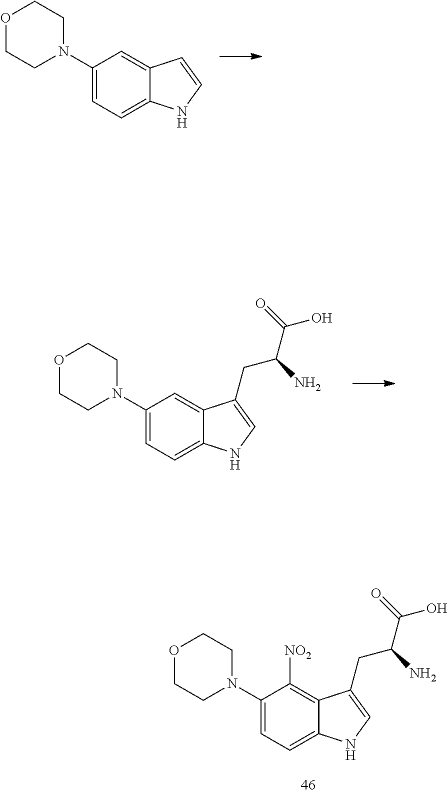 [SMILES: c1cc2cc(N3CCOCC3)ccc2[nH]1 ; N[C@@H](Cc1c[nH]c2ccc(N3CCOCC3)cc12)C(=O)O ; N[C@@H](Cc1c[nH]c2ccc(N3CCOCC3)c([N+](=O)[O-])c12)C(=O)O]

Example 46 can be prepared from 5-morpholinoindole as shown above.

### Example 47: Preparation of (S)-2-amino-3-(6-morpholino-4-nitro-1H-indol-3-yl)propanoic acid (47)

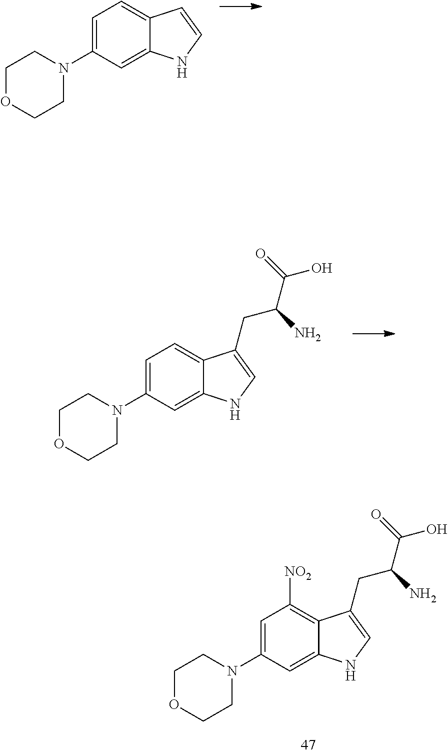 [SMILES: c1cc2ccc(N3CCOCC3)cc2[nH]1 ; N[C@@H](Cc1c[nH]c2cc(N3CCOCC3)ccc12)C(=O)O ; N[C@@H](Cc1c[nH]c2cc(N3CCOCC3)cc([N+](=O)[O-])c12)C(=O)O]

Example 47 can be prepared from 6-morpholinoindole as shown above.

### Example 48: Preparation of (S)-2-amino-3-(7-morpholino-4-nitro-1H-indol-3-yl)propanoic acid (48)

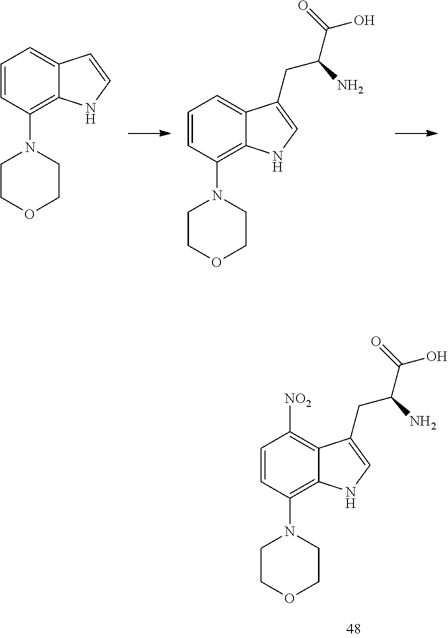 [SMILES: c1cc(N2CCOCC2)c2[nH]ccc2c1 ; N[C@@H](Cc1c[nH]c2c(N3CCOCC3)cccc12)C(=O)O ; N[C@@H](Cc1c[nH]c2c(N3CCOCC3)ccc([N+](=O)[O-])c12)C(=O)O]

Example 48 can be prepared from 7-morpholinoindole as shown above.

### Example 49: Preparation of (S)-2-amin-3-(4-morpholino-7-nitro-1H-indol-3-yl)propanoic acid (49)

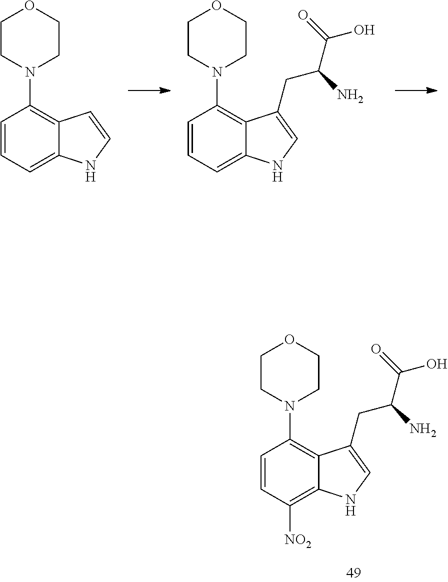 [SMILES: c1cc(N2CCOCC2)c2cc[nH]c2c1 ; N[C@@H](Cc1c[nH]c2cccc(N3CCOCC3)c12)C(=O)O ; N[C@@H](Cc1c[nH]c2c([N+](=O)[O-])ccc(N3CCOCC3)c12)C(=O)O]

Example 49 can be prepared from 4-morpholinoindole as shown above.

### Example 50: Preparation of (S)-2-amino-3-(5-(methylthio)-4-nitro-1H-indol-3-yl)propanoic acid (50)

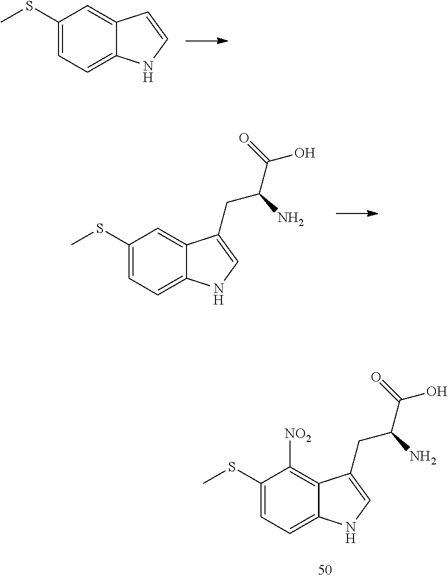 [SMILES: CSc1ccc2[nH]ccc2c1 ; CSc1ccc2[nH]cc(C[C@H](N)C(=O)O)c2c1 ; CSc1ccc2[nH]cc(C[C@H](N)C(=O)O)c2c1[N+](=O)[O-]]

Example 50 can be prepared from 5-(methylthio)indole as shown above.

### Example 51: Preparation of (S)-2-amino-3-(6-(methylthio)-4-nitro-1H-indol-3-yl)propanoic acid (51)

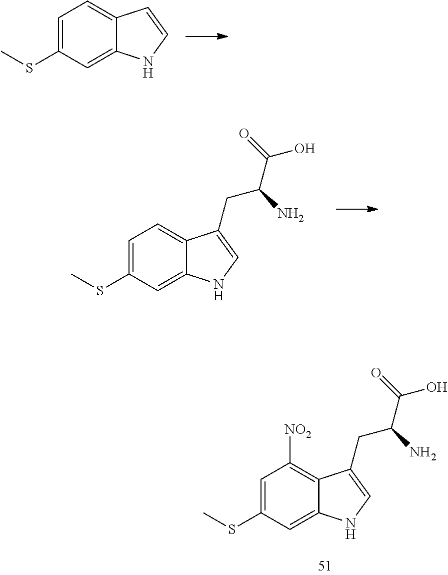 [SMILES: CSc1ccc2cc[nH]c2c1 ; CSc1ccc2c(C[C@H](N)C(=O)O)c[nH]c2c1 ; CSc1cc([N+](=O)[O-])c2c(C[C@H](N)C(=O)O)c[nH]c2c1]

Example 51 can be prepared from 6-(methylthio)indole as shown above.

### Example 52: Preparation of (S)-2-amino-3-(7-(methylthio)-4-nitro-1H-indol-3-yl)propanoic acid (52)

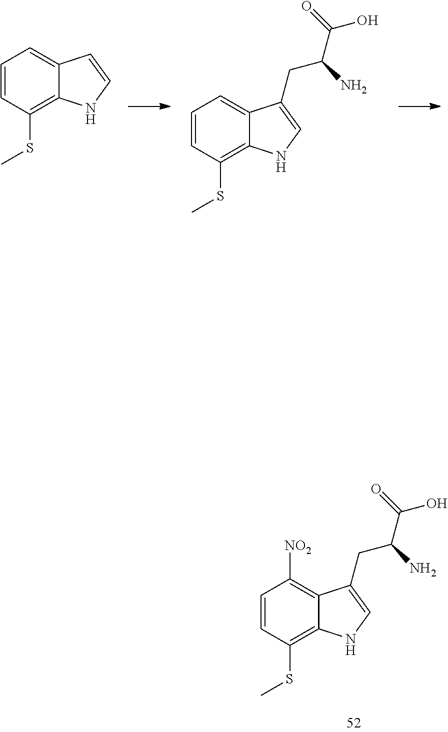 [SMILES: CSc1cccc2cc[nH]c12 ; CSc1cccc2c(C[C@H](N)C(=O)O)c[nH]c12 ; CSc1ccc([N+](=O)[O-])c2c(C[C@H](N)C(=O)O)c[nH]c12]

Example 52 can be prepared from 7-(methylthio)indole as shown above.

### Example 53: Preparation of (S)-2-amino-3-(4-(methylthio)-7-nitro-1H-indol-3-yl)propanoic acid (53)

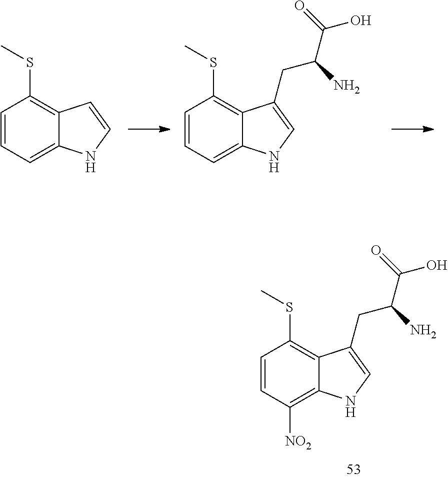 [SMILES: CSc1cccc2[nH]ccc12 ; CSc1cccc2[nH]cc(C[C@H](N)C(=O)O)c12 ; CSc1ccc([N+](=O)[O-])c2[nH]cc(C[C@H](N)C(=O)O)c12]

Example 53 can be prepared from 4-(methylthio)indole as shown above.

### Example 54: Preparation of (S)-2-amino-3-(4-nitro-5-(pyridin-4-yl)-1H-indol-3-yl)propanoic acid (54)

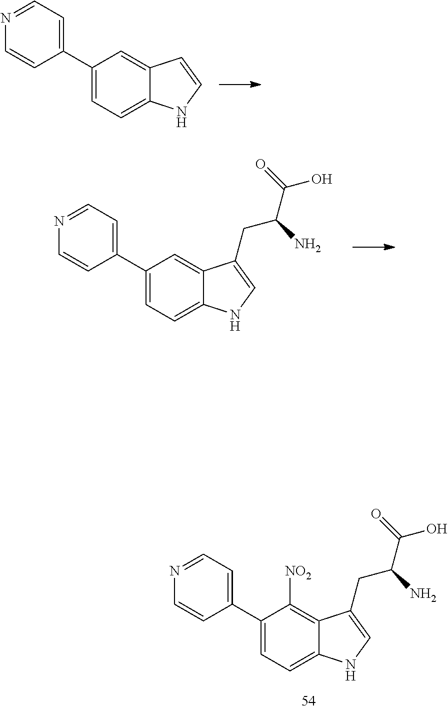 [SMILES: c1cc(-c2ccc3[nH]ccc3c2)ccn1 ; N[C@@H](Cc1c[nH]c2ccc(-c3ccncc3)cc12)C(=O)O ; N[C@@H](Cc1c[nH]c2ccc(-c3ccncc3)c([N+](=O)[O-])c12)C(=O)O]

Example 54 can be prepared from 5-(pyridin-4-yl)indole as shown above.

### Example 55: Preparation of (S)-2-amino-3-(4-nitro-6-(pyrin-4-yl)-1H-indol-3-yl)propanoic acid (55)

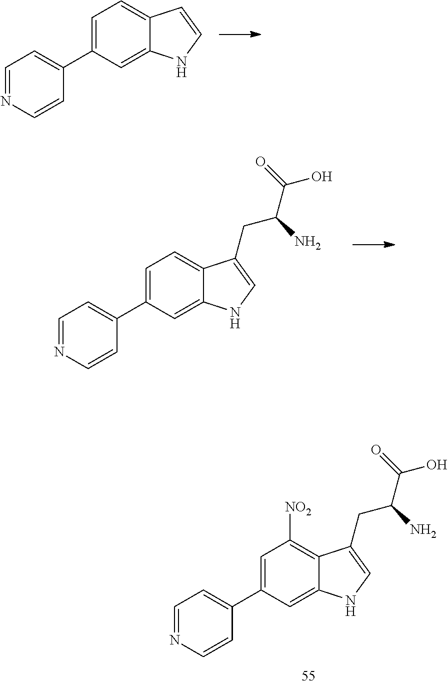 [SMILES: c1cc(-c2ccc3cc[nH]c3c2)ccn1 ; N[C@@H](Cc1c[nH]c2cc(-c3ccncc3)ccc12)C(=O)O ; N[C@@H](Cc1c[nH]c2cc(-c3ccncc3)cc([N+](=O)[O-])c12)C(=O)O]

Example 55 can be prepared from 6-(pyridin-4-yl)indole as shown above.

### Example 56: Preparation of (S)-2-amino-3-(4-nitro-7-(pyridin-4-yl)-1H-indol-3-yl)propanoic acid (56)

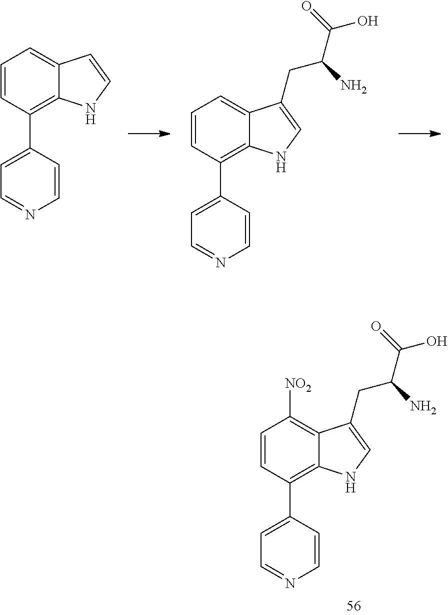 [SMILES: c1cc(-c2ccncc2)c2[nH]ccc2c1 ; N[C@@H](Cc1c[nH]c2c(-c3ccncc3)cccc12)C(=O)O ; N[C@@H](Cc1c[nH]c2c(-c3ccncc3)ccc([N+](=O)[O-])c12)C(=O)O]

Example 56 can be prepared from 7-(pyridin-4-yl)indole as shown above.

### Example 57: Preparation of (S)-2-amino-3-(7-nitro-4-(pyridin-4-yl)-1H-indol-3-yl)propanoic acid (57)

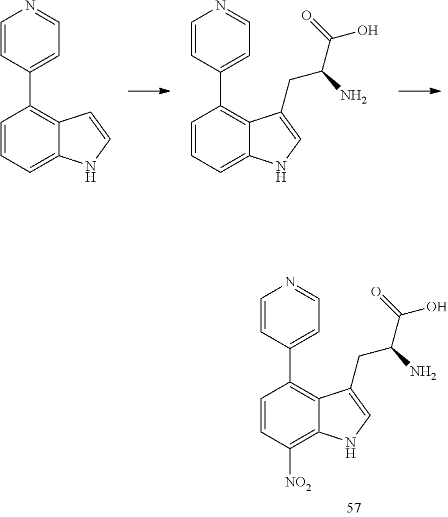 [SMILES: c1cc(-c2ccncc2)c2cc[nH]c2c1 ; N[C@@H](Cc1c[nH]c2cccc(-c3ccncc3)c12)C(=O)O ; N[C@@H](Cc1c[nH]c2c([N+](=O)[O-])ccc(-c3ccncc3)c12)C(=O)O]

Example 57 can be prepared from 4-(pyridin-4-yl)indole as shown above.

### Example 58: Preparation of 2-amino-3-(5-methyl-4-nitro-1H-indol-3-yl)propanoic acid (58)

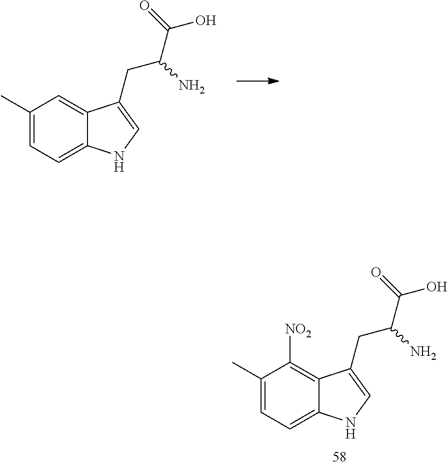 [SMILES: Cc1ccc2[nH]cc(CC(N)C(=O)O)c2c1 ; Cc1ccc2[nH]cc(CC(N)C(=O)O)c2c1[N+](=O)[O-]]

Example 58 can be prepared from 2-amino-3-(5-methyl-1H-indol-3-yl)propanoic acid as shown above.

### Example 59: Preparation of 2-amino-3-(6-methyl-4-nitro-1H-indol-3-yl)propanoic acid (59)

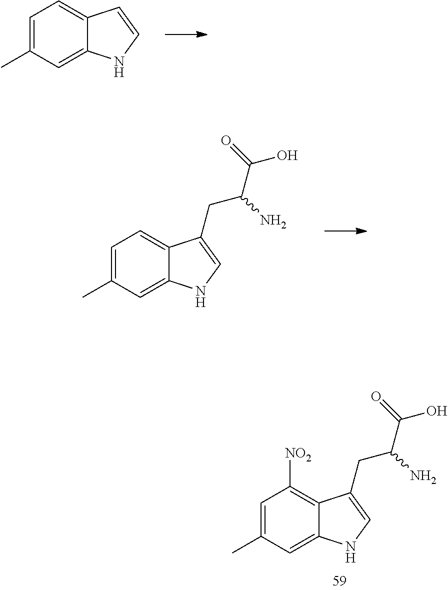

Example 59 can be prepared from 6-methylindole as shown above.

### Example 60: Preparation of 2-amino-3-(7-methyl-4-nitro-1H-indol-3-yl)propanoic acid (60)

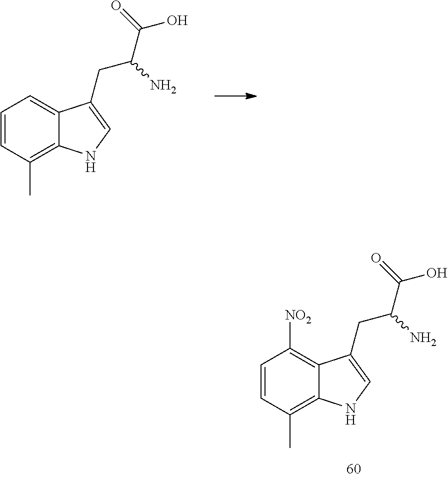 [SMILES: Cc1cccc2c(CC(N)C(=O)O)c[nH]c12 ; Cc1ccc([N+](=O)[O-])c2c(CC(N)C(=O)O)c[nH]c12]

Example 60 can be prepared from 2-amino-3-(7-methyl-1H-indol-3-yl)propanoic acid as shown above.

### Example 61: Preparation of 2-amino-3-(4-methyl-7-nitro-1H-indol-3-yl)propanoic acid (61)

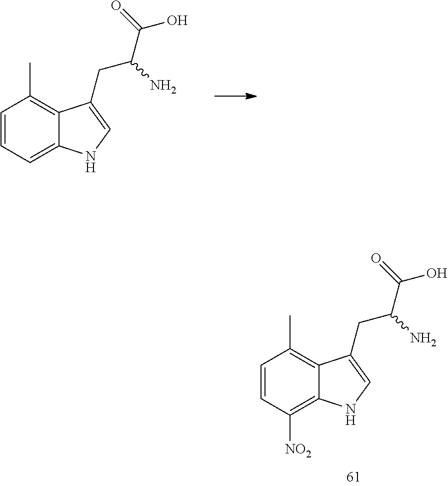 [SMILES: Cc1cccc2[nH]cc(CC(N)C(=O)O)c12 ; Cc1ccc([N+](=O)[O-])c2[nH]cc(CC(N)C(=O)O)c12]

Example 61 was prepared from 2-amino-3-(4-methyl-1H-indol-3-yl)propanoic acid as shown above.

### Example 62: Preparation of 2-amino-3-(6-fluoro-4-nitro-1H-indol-3-yl)propanoic acid (62)

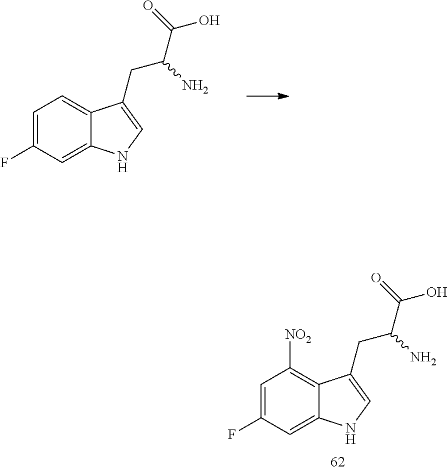 [SMILES: NC(Cc1c[nH]c2cc(F)ccc12)C(=O)O ; NC(Cc1c[nH]c2cc(F)cc([N+](=O)[O-])c12)C(=O)O]

Example 62 can be prepared from 2-amino-3-(6-fluoro-1H-indol-3-yl)propanoic acid as shown above.

### Example 63: Preparation of 2-amino-3-(7-fluoro-4-nitro-1H-indol-3-yl)propanoic acid (63)

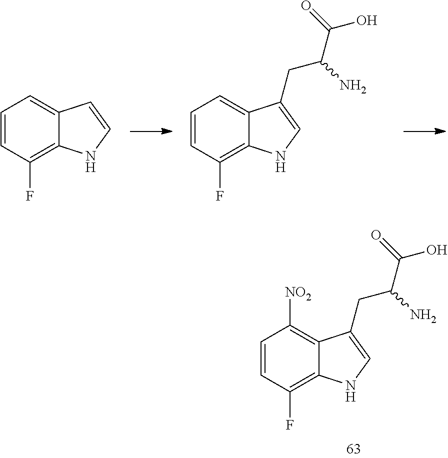 [SMILES: Fc1cccc2cc[nH]c12 ; NC(Cc1c[nH]c2c(F)cccc12)C(=O)O ; NC(Cc1c[nH]c2c(F)ccc([N+](=O)[O-])c12)C(=O)O]

Example 63 can be prepared from 7-fluoroindole as shown above.

### Example 64: Preparation of 2-amino-3-(4-fluor-7-nitro-1H-indol-3-yl)propanoic acid (64)

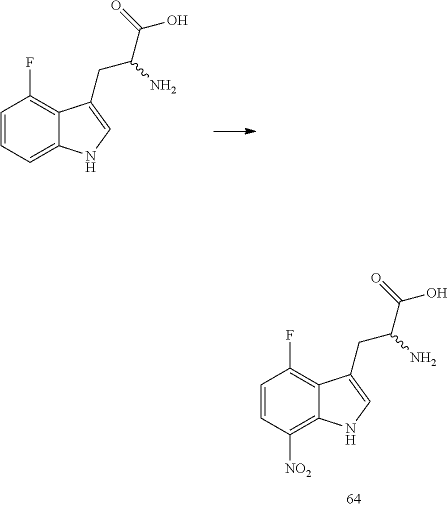 [SMILES: NC(Cc1c[nH]c2cccc(F)c12)C(=O)O ; NC(Cc1c[nH]c2c([N+](=O)[O-])ccc(F)c12)C(=O)O]

Example 64 can be prepared from 2-amino-3-(4-fluoro-1H-indol-3-yl)propanoic acid as shown above.

### Example 65: Preparation of 2-amino-3-(5-chloro-4-nitro-1H-indol-3-yl)propanoic acid (65)

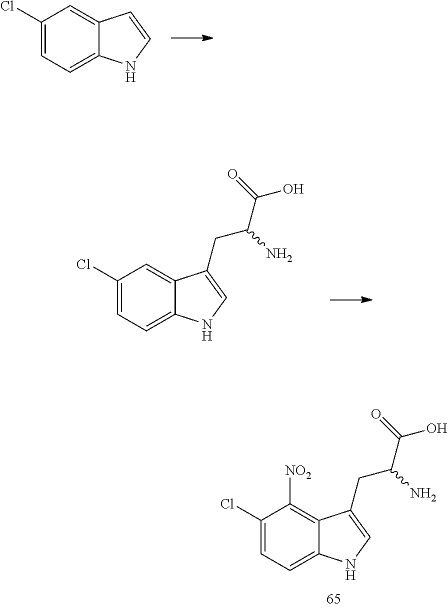 [SMILES: Clc1ccc2[nH]ccc2c1 ; NC(Cc1c[nH]c2ccc(Cl)cc12)C(=O)O ; NC(Cc1c[nH]c2ccc(Cl)c([N+](=O)[O-])c12)C(=O)O]

Example 65 can be prepared from 5-chromandiol as shown above.

### Example 66: Preparation of 2-amino-3-(6-chloro-4-nitro-1H-indol-3-yl)propanoic acid (66)

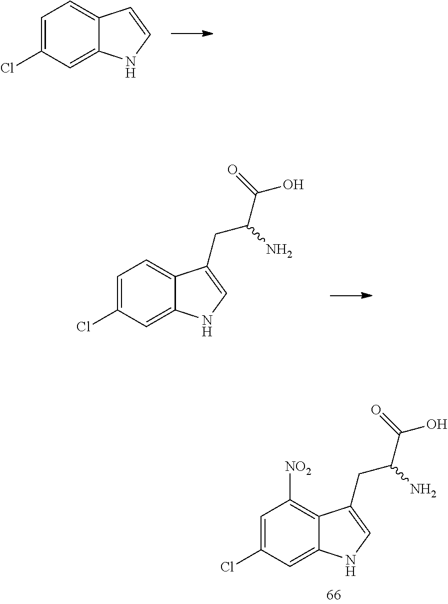 [SMILES: Clc1ccc2cc[nH]c2c1 ; NC(Cc1c[nH]c2cc(Cl)ccc12)C(=O)O ; NC(Cc1c[nH]c2cc(Cl)cc([N+](=O)[O-])c12)C(=O)O]

Example 66 can be prepared from 6-chloroindole as shown above.

### Example 67: Preparation of 2-amino-3-(7-chloro-4-nitro-1H-indol-3-yl)propanoic acid (67)

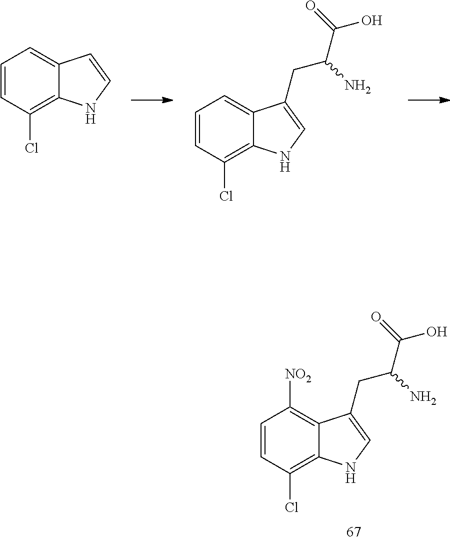 [SMILES: Clc1cccc2cc[nH]c12 ; NC(Cc1c[nH]c2c(Cl)cccc12)C(=O)O ; NC(Cc1c[nH]c2c(Cl)ccc([N+](=O)[O-])c12)C(=O)O]

Example 67 can be prepared from 7-chloroindole as shown above.

### Example 68: Preparation of 2-amino-3-(4-chloro-7-nitro-1H-indol-3-yl)propanoic acid (68)

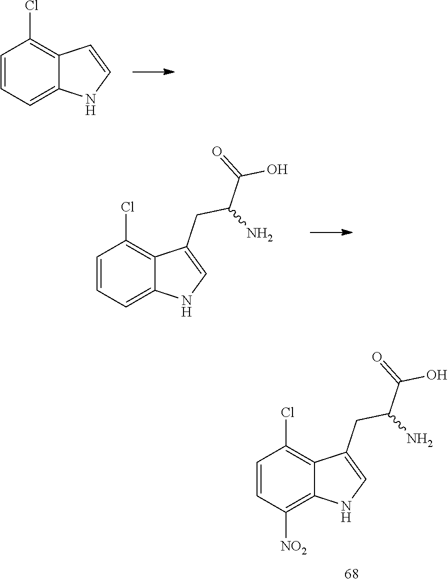 [SMILES: Clc1cccc2[nH]ccc12 ; NC(Cc1c[nH]c2cccc(Cl)c12)C(=O)O ; NC(Cc1c[nH]c2c([N+](=O)[O-])ccc(Cl)c12)C(=O)O]

Example 68 can be prepared from 4-chloroindol as shown above.

### Example 69: Preparation of 2-amino-3-(5-bromo-4-nitro-1H-indol-3-yl)propanoic acid (69)

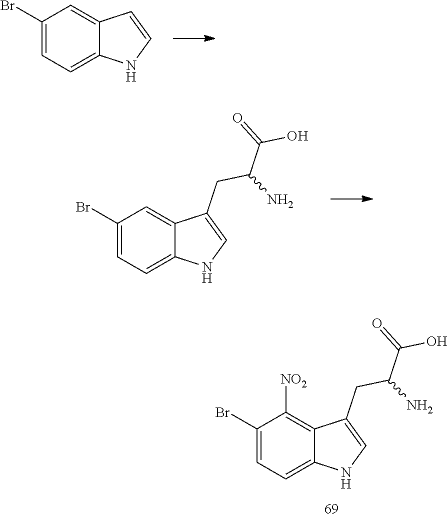 [SMILES: Brc1ccc2[nH]ccc2c1 ; NC(Cc1c[nH]c2ccc(Br)cc12)C(=O)O ; NC(Cc1c[nH]c2ccc(Br)c([N+](=O)[O-])c12)C(=O)O]

Example 69 can be prepared from 5-bromoindole as shown above.

### Example 70: Preparation of 2-amino-3-(6-bromo-4-nitro-1H-indol-3-yl)propanoic acid (70)

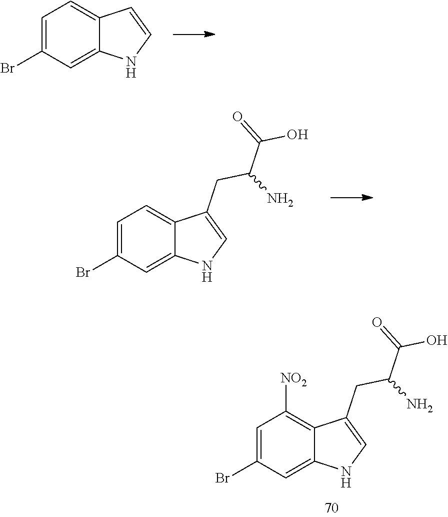 [SMILES: Brc1ccc2cc[nH]c2c1 ; NC(Cc1c[nH]c2cc(Br)ccc12)C(=O)O ; NC(Cc1c[nH]c2cc(Br)cc([N+](=O)[O-])c12)C(=O)O]

Example 70 can be prepared from 6-bromoindole as shown above.

### Example 71: Preparation of 2-nitro-3-(7-bromo-4-nitro-1H-indol-3-yl)propanoic acid (71)

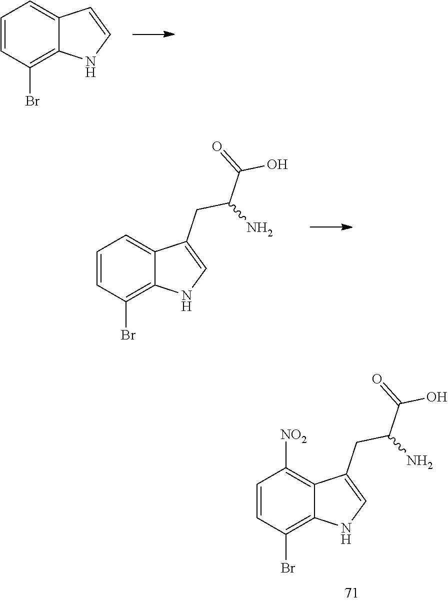 [SMILES: Brc1cccc2cc[nH]c12 ; NC(Cc1c[nH]c2c(Br)cccc12)C(=O)O ; NC(Cc1c[nH]c2c(Br)ccc([N+](=O)[O-])c12)C(=O)O]

Example 71 can be prepared from 7-bromoindole as shown above.

### Example 72: Preparation of 2-amino-3-(4-bromo-7-nitro-1H-indol-3-yl)propanoic acid (72)

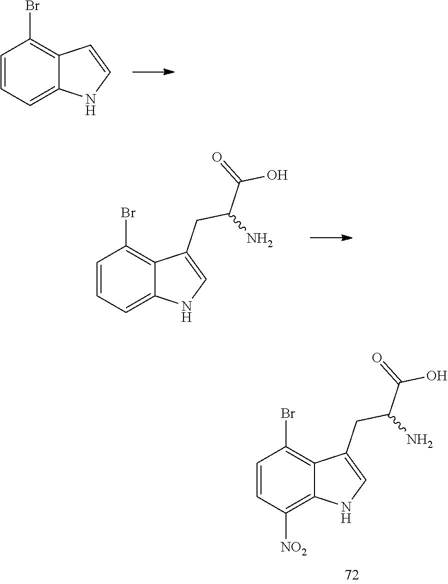 [SMILES: Brc1cccc2[nH]ccc12 ; NC(Cc1c[nH]c2cccc(Br)c12)C(=O)O ; NC(Cc1c[nH]c2c([N+](=O)[O-])ccc(Br)c12)C(=O)O]

Example 72 can be prepared from 4-bromoindole as shown above.

### Example 73: Preparation of 2-amino-3-(5-methoxy-4-nitro-1H-indol-3-yl)propanoic acid (73)

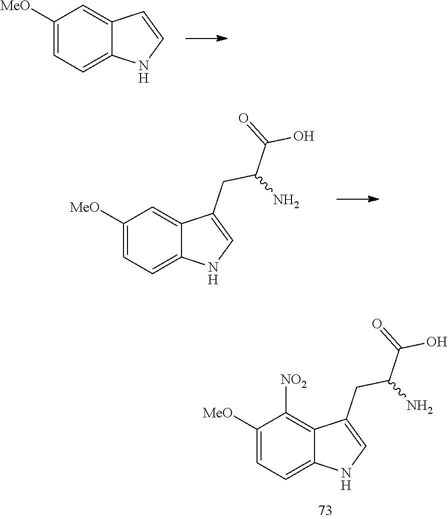 [SMILES: COc1ccc2[nH]ccc2c1 ; COc1ccc2[nH]cc(CC(N)C(=O)O)c2c1 ; COc1ccc2[nH]cc(CC(N)C(=O)O)c2c1[N+](=O)[O-]]

Example 73 can be prepared from 5-methoxyindole as shown above.

### Example 74: Preparation of 2-amino-3-(6-methoxy-4-nitro-1H-indol-3-yl)propanoic acid (74)

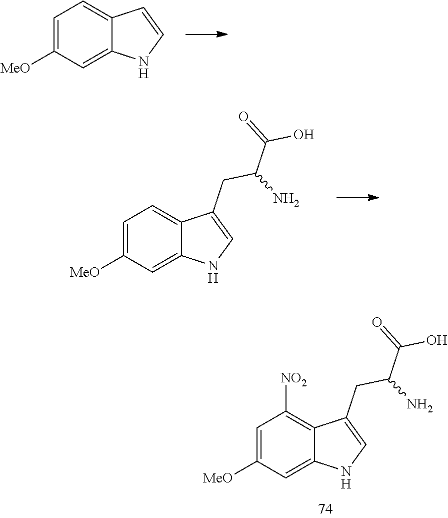 [SMILES: COc1ccc2cc[nH]c2c1 ; COc1ccc2c(CC(N)C(=O)O)c[nH]c2c1 ; COc1cc([N+](=O)[O-])c2c(CC(N)C(=O)O)c[nH]c2c1]

Example 74 can be prepared from 6-methoxyindole as shown above.

### Example 75: Preparation of 2-nitro-3-(7-methoxy-4-nitro-1H-indol-3-yl)propanoic acid (75)

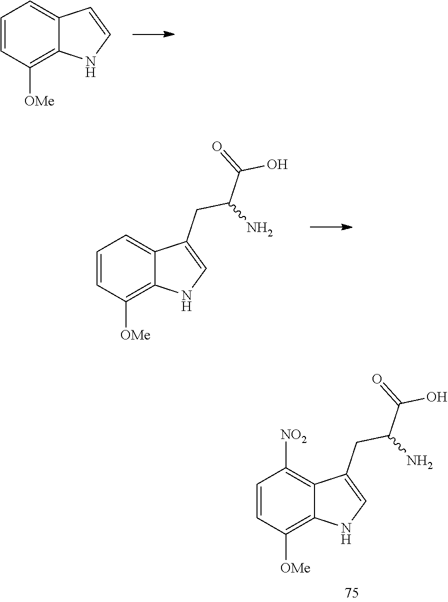 [SMILES: COc1cccc2cc[nH]c12 ; COc1cccc2c(CC(N)C(=O)O)c[nH]c12 ; COc1ccc([N+](=O)[O-])c2c(CC(N)C(=O)O)c[nH]c12]

Example 75 can be prepared from 7-methoxyindole as shown above.

### Example 76: Preparation of 2-amino-3-(4-methoxy-7-nitro-1H-Indol-3-yl)propanoic acid (76)

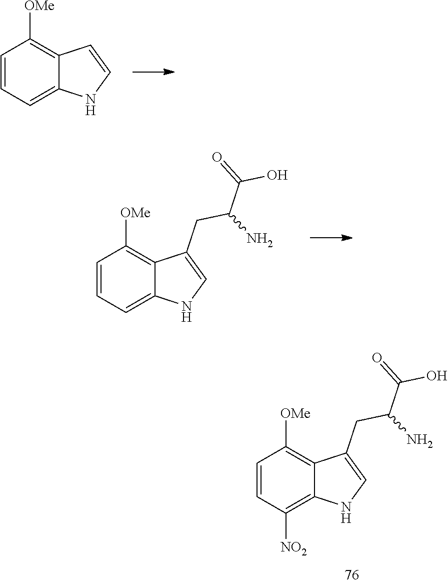 [SMILES: COc1cccc2[nH]ccc12 ; COc1cccc2[nH]cc(CC(N)C(=O)O)c12 ; COc1ccc([N+](=O)[O-])c2[nH]cc(CC(N)C(=O)O)c12]

Example 76 can be prepared from 4-methoxyindole as shown above.

### Example 77: Preparation of 2-amino-3-(5-amino-4-nitro-1H-indol-3-yl)propanoic acid (77)

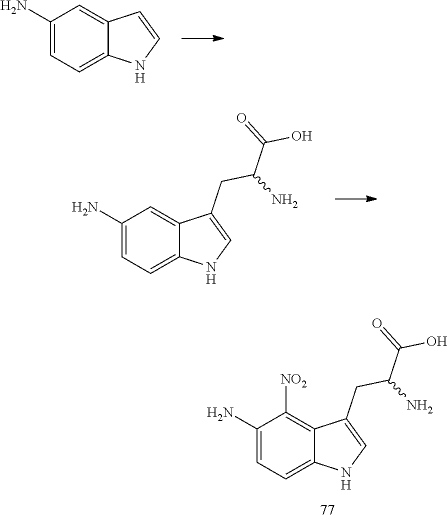 [SMILES: Nc1ccc2[nH]ccc2c1 ; Nc1ccc2[nH]cc(CC(N)C(=O)O)c2c1 ; Nc1ccc2[nH]cc(CC(N)C(=O)O)c2c1[N+](=O)[O-]]

Example 77 can be prepared from 5-aminoindole as shown above.

### Example 78: Preparation of 2-amino-3-(6-amino-4-nitro-1H-indol-3-yl)propanoic acid (78)

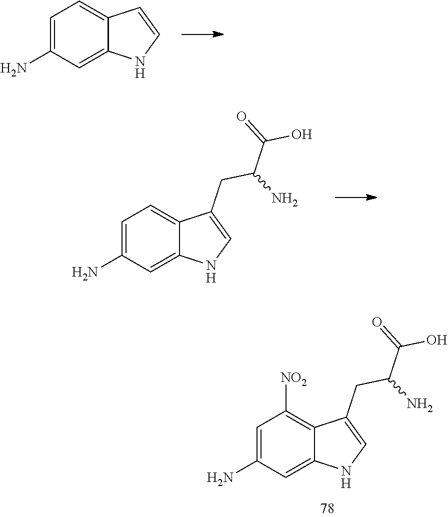 [SMILES: Nc1ccc2cc[nH]c2c1 ; Nc1ccc2c(CC(N)C(=O)O)c[nH]c2c1 ; Nc1cc([N+](=O)[O-])c2c(CC(N)C(=O)O)c[nH]c2c1]

Example 78 can be prepared from 6-aminoindole as shown above.

### Example 79: Preparation of 2-amino-3-(7-amino-4-nitro-1H-indol-3-yl)propanoic acid (79)

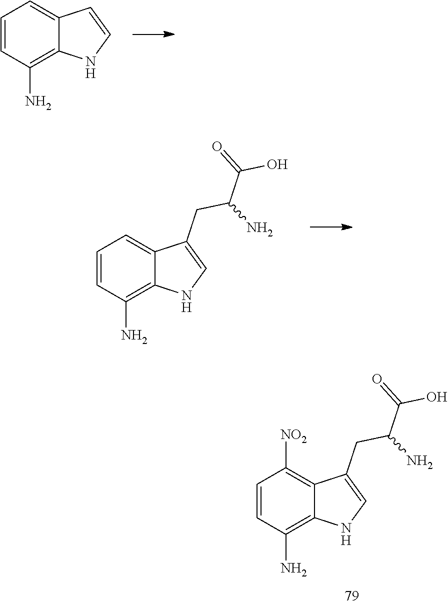 [SMILES: Nc1cccc2cc[nH]c12 ; Nc1cccc2c(CC(N)C(=O)O)c[nH]c12 ; Nc1ccc([N+](=O)[O-])c2c(CC(N)C(=O)O)c[nH]c12]

Example 79 can be prepared from 7-aminoindole as shown above.

### Example 80: Preparation of 2-amino-3-(4-amino-7-nitro-1H-indol-3-yl)propanoic acid (80)

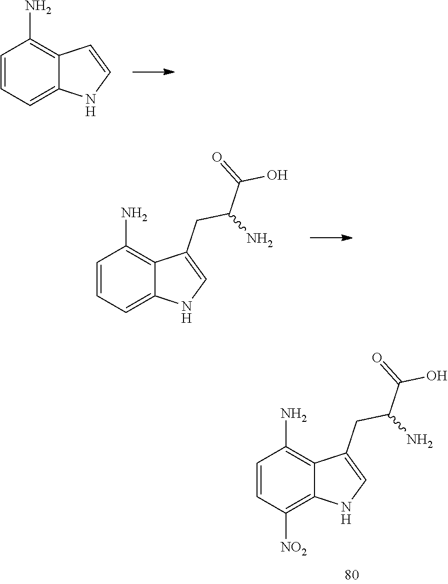 [SMILES: Nc1cccc2[nH]ccc12 ; Nc1cccc2[nH]cc(CC(N)C(=O)O)c12 ; Nc1ccc([N+](=O)[O-])c2[nH]cc(CC(N)C(=O)O)c12]

Example 80 can be prepared from 4-aminoindole as shown above.

### Example 81: Preparation of 2-amino-3-(5-hydroxy-4-nitro-1H-indol-3-yl)propanoic acid (81)

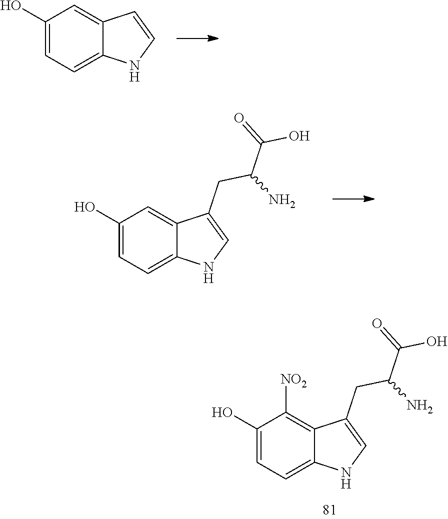 [SMILES: Oc1ccc2[nH]ccc2c1 ; NC(Cc1c[nH]c2ccc(O)cc12)C(=O)O ; NC(Cc1c[nH]c2ccc(O)c([N+](=O)[O-])c12)C(=O)O]

Example 81 can be prepared from 5-hydroxyindole as shown above.

### Example 82: Preparation of 2-amino-3-(6-hydroxy-4-nitro-1H-indol-3-yl) propanoic acid (82)

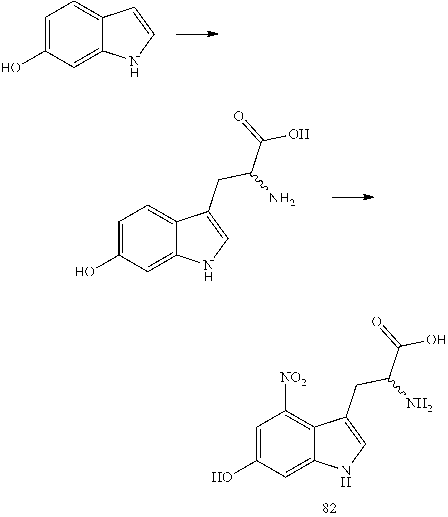 [SMILES: Oc1ccc2cc[nH]c2c1 ; NC(Cc1c[nH]c2cc(O)ccc12)C(=O)O ; NC(Cc1c[nH]c2cc(O)cc([N+](=O)[O-])c12)C(=O)O]

Example 82 can be prepared from 6-hydroxyindole as shown above.

### Example 83: Preparation of 2-amino-3-(7-hydroxy-4-nitro-1H-indol-3-yl)propanoic acid (83)

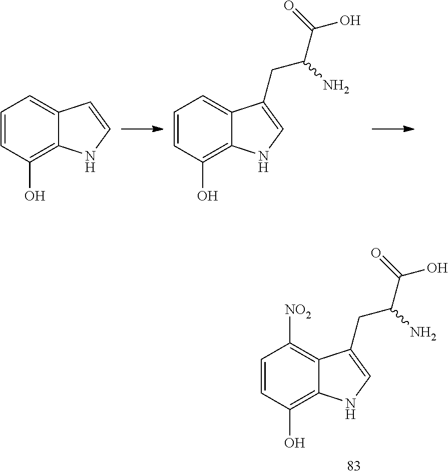 [SMILES: Oc1cccc2cc[nH]c12 ; NC(Cc1c[nH]c2c(O)cccc12)C(=O)O ; NC(Cc1c[nH]c2c(O)ccc([N+](=O)[O-])c12)C(=O)O]

Example 83 can be prepared from 7-hydroxyindole as shown above.

### Example 84: Preparation of 2-amino-3-(4-hydroxy-7-nitro-1H-indol-3-yl)propanoic acid (84)

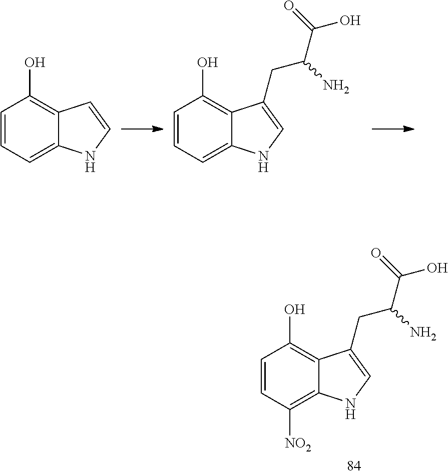 [SMILES: Oc1cccc2[nH]ccc12 ; NC(Cc1c[nH]c2cccc(O)c12)C(=O)O ; NC(Cc1c[nH]c2c([N+](=O)[O-])ccc(O)c12)C(=O)O]

Example 84 can be prepared from 4-hydroxyindole as shown above.

Example 85: Preparation of 2-amino-3-(4-nitro-5-phenyl-1H-indol-3-yl)propanoic acid (85)

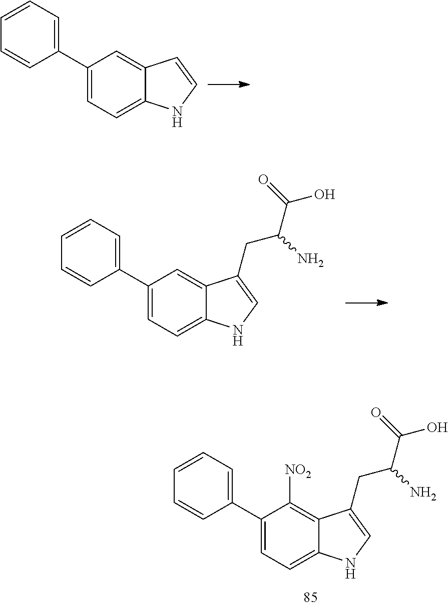 [SMILES: c1ccc(-c2ccc3[nH]ccc3c2)cc1 ; NC(Cc1c[nH]c2ccc(-c3ccccc3)cc12)C(=O)O ; NC(Cc1c[nH]c2ccc(-c3ccccc3)c([N+](=O)[O-])c12)C(=O)O]

Example 85 can be prepared from 5-phenylindole as shown above.

### Example 86: Preparation of 2-amino-3-(4-nitro-6-phenyl-1H-indol-3-yl)propanoic acid (86)

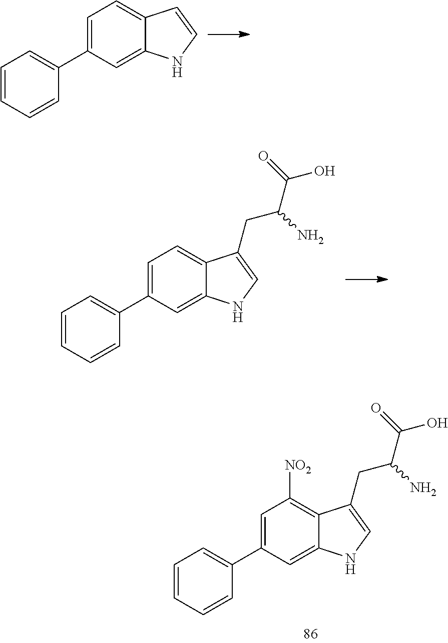 [SMILES: c1ccc(-c2ccc3cc[nH]c3c2)cc1 ; NC(Cc1c[nH]c2cc(-c3ccccc3)ccc12)C(=O)O ; NC(Cc1c[nH]c2cc(-c3ccccc3)cc([N+](=O)[O-])c12)C(=O)O]

Example 86 can be prepared from 6-phenylindole as shown above.

### Example 87: Preparation of 2-amino-3-(4-nitro-7-phenyl-1H-indol-3-yl)propanoic acid (87)

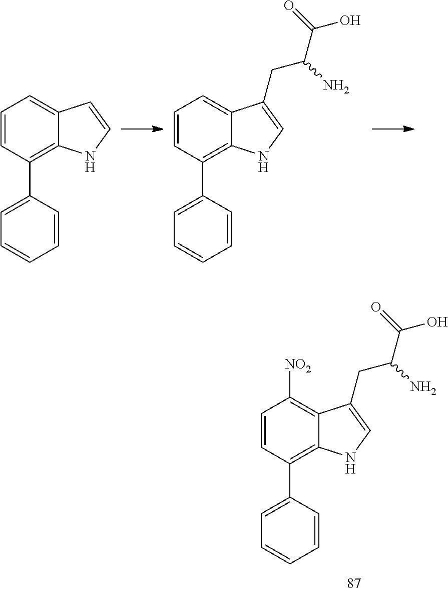 [SMILES: c1ccc(-c2cccc3cc[nH]c23)cc1 ; NC(Cc1c[nH]c2c(-c3ccccc3)cccc12)C(=O)O ; NC(Cc1c[nH]c2c(-c3ccccc3)ccc([N+](=O)[O-])c12)C(=O)O]

Example 87 can be prepared from 7-phenylindole as shown above.

### Example 88: Preparation of 2-amino-3-(7-nitro-4-phenyl-1H-indol-3-yl)propanoic acid (88)

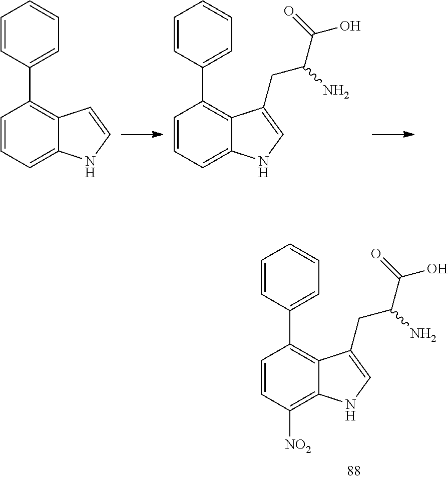 [SMILES: c1ccc(-c2cccc3[nH]ccc23)cc1 ; NC(Cc1c[nH]c2cccc(-c3ccccc3)c12)C(=O)O ; NC(Cc1c[nH]c2c([N+](=O)[O-])ccc(-c3ccccc3)c12)C(=O)O]

Example 88 can be prepared from 4-phenylindole as shown above.

### Example 89: Preparation of 2-amino-3-(5-cyclopropyl-4-nitro-1H-indol-3-yl)propanoic acid (89)

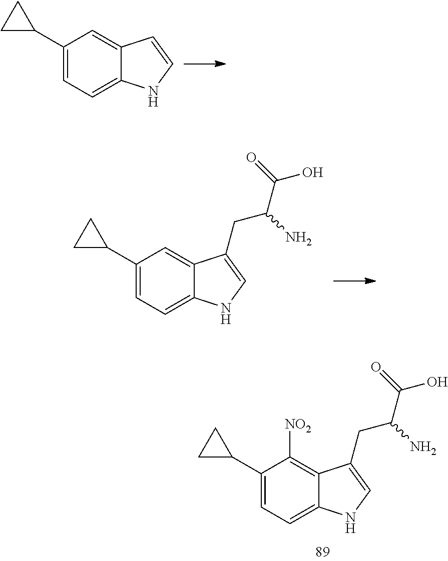 [SMILES: c1cc2cc(C3CC3)ccc2[nH]1 ; NC(Cc1c[nH]c2ccc(C3CC3)cc12)C(=O)O ; NC(Cc1c[nH]c2ccc(C3CC3)c([N+](=O)[O-])c12)C(=O)O]

Example 89 can be prepared from 5-cyclopropylindole as shown above.

### Example 90: Preparation of 2-amino-3-(6-cyclopropyl-4-nitro-1H-indol-3-yl)propanoic acid (90)

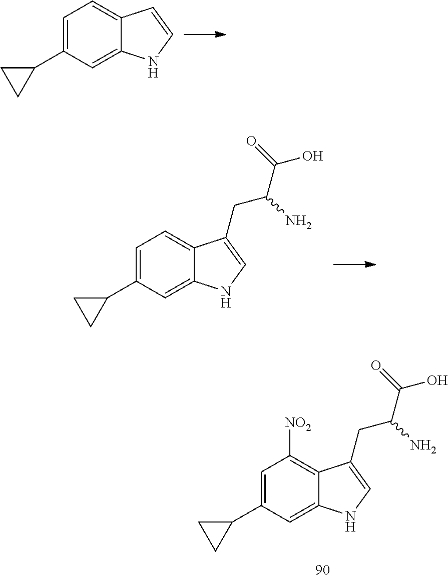 [SMILES: c1cc2ccc(C3CC3)cc2[nH]1 ; NC(Cc1c[nH]c2cc(C3CC3)ccc12)C(=O)O ; NC(Cc1c[nH]c2cc(C3CC3)cc([N+](=O)[O-])c12)C(=O)O]

Example 90 can be prepared from 6-cyclopropylindole as shown above.

### Example 91: Preparation of 2-amino-3-(7-cyclopropyl-4-nitro-1H-indol-3-yl)propanoic acid (91)

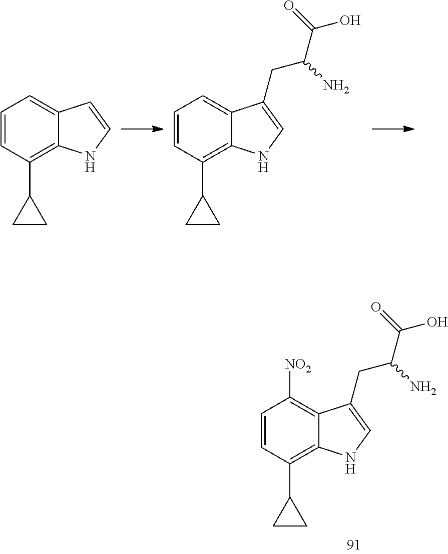 [SMILES: c1cc(C2CC2)c2[nH]ccc2c1 ; NC(Cc1c[nH]c2c(C3CC3)cccc12)C(=O)O ; NC(Cc1c[nH]c2c(C3CC3)ccc([N+](=O)[O-])c12)C(=O)O]

Example 91 can be prepared from 7-cyclopropylindole as shown above.

### Example 92: Preparation of 2-amino-3-(4-cyclopropyl-7-nitro-1H-indol-3-yl)propanoic acid (92)

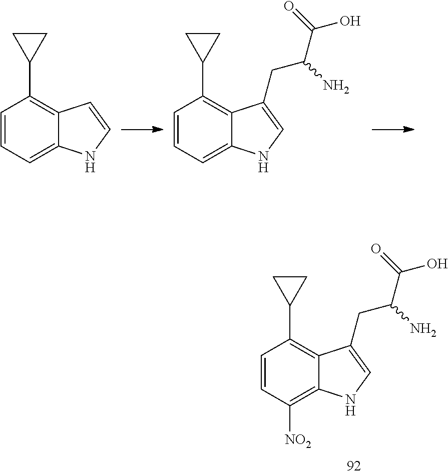 [SMILES: c1cc(C2CC2)c2cc[nH]c2c1 ; NC(Cc1c[nH]c2cccc(C3CC3)c12)C(=O)O ; NC(Cc1c[nH]c2c([N+](=O)[O-])ccc(C3CC3)c12)C(=O)O]

Example 92 can be prepared from 4-cyclopropylindole as shown above.

### Example 93: Preparation of 2-amino-3-(4-nitro-5-vinyl-1H-indol-3-yl)propanoic acid (93)

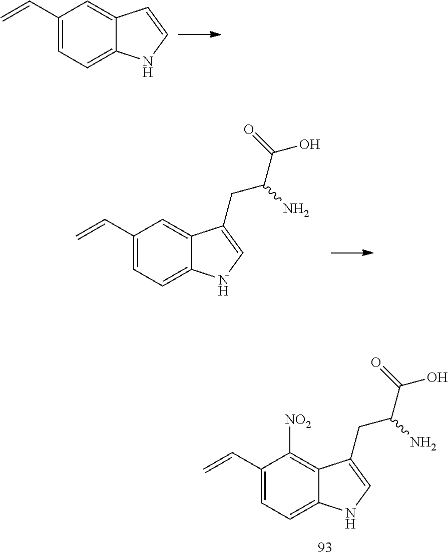 [SMILES: C=Cc1ccc2[nH]ccc2c1 ; C=Cc1ccc2[nH]cc(CC(N)C(=O)O)c2c1 ; C=Cc1ccc2[nH]cc(CC(N)C(=O)O)c2c1[N+](=O)[O-]]

Example 93 can be prepared from 5-vinylindole as shown above.

### Example 94: Preparation of 2-amino-3-(4-nitro-6-vinyl-1H-indol-3-yl)propanoic acid (94)

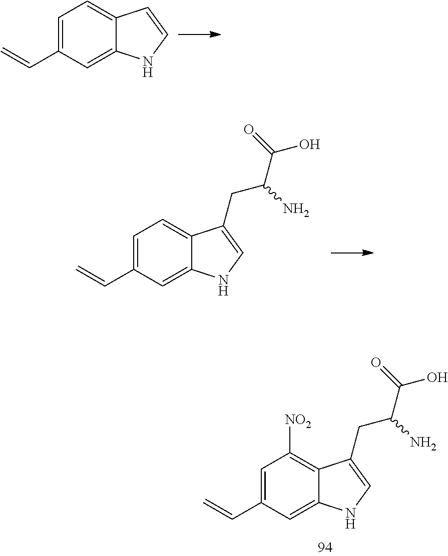 [SMILES: C=Cc1ccc2cc[nH]c2c1 ; C=Cc1ccc2c(CC(N)C(=O)O)c[nH]c2c1 ; C=Cc1cc([N+](=O)[O-])c2c(CC(N)C(=O)O)c[nH]c2c1]

Example 94 can be prepared from 6-vinylindole as shown above.

### Example 95: Preparation of 2-amino-3-(4-nitro-7-phenyl-1H-indol-3-yl)propanoic acid (95)

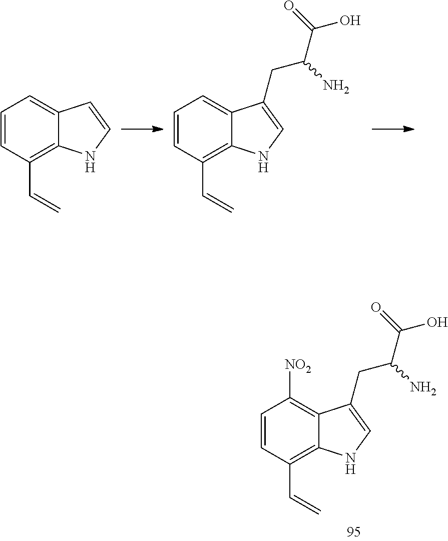 [SMILES: C=Cc1cccc2cc[nH]c12 ; C=Cc1cccc2c(CC(N)C(=O)O)c[nH]c12 ; C=Cc1ccc([N+](=O)[O-])c2c(CC(N)C(=O)O)c[nH]c12]

Example 95 can be prepared from 7-vinylindole as shown above.

### Example 96: Preparation of 2-amino-3-(7-nitro-4-vinyl-1H-indol-3-yl)propanoic acid (96)

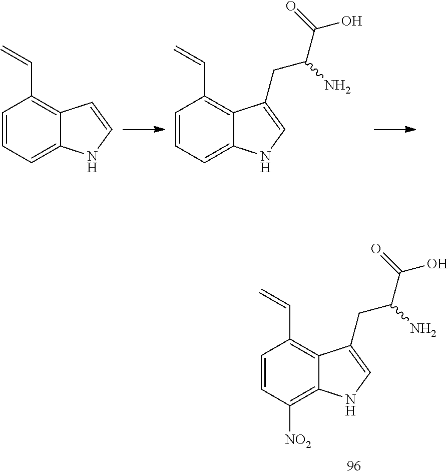 [SMILES: C=Cc1cccc2[nH]ccc12 ; C=Cc1cccc2[nH]cc(CC(N)C(=O)O)c12 ; C=Cc1ccc([N+](=O)[O-])c2[nH]cc(CC(N)C(=O)O)c12]

Example % can be prepared from 4-vinylindole as shown above.

### Example 97: Preparation of 2-amino-3-(5-ethynyl-4-nitro-1H-indol-3-yl)propanoic acid (97)

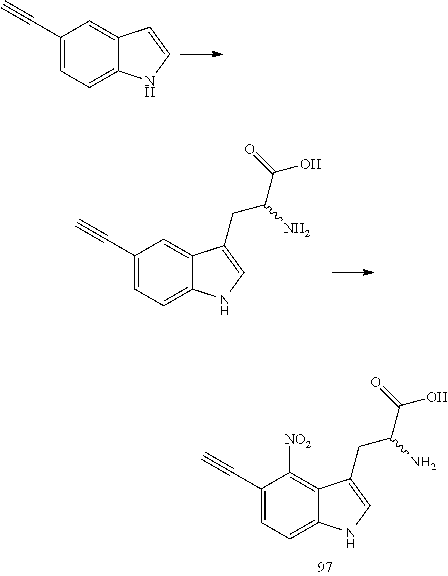 [SMILES: C#Cc1ccc2[nH]ccc2c1 ; C#Cc1ccc2[nH]cc(CC(N)C(=O)O)c2c1 ; C#Cc1ccc2[nH]cc(CC(N)C(=O)O)c2c1[N+](=O)[O-]]

Example 97 can be prepared from 5-ethylindole as shown above.

### Example 97: Preparation of 2-amino-3-(6-ethynyl-4-nitro-1H-indol-3-yl)propanoic acid (98)

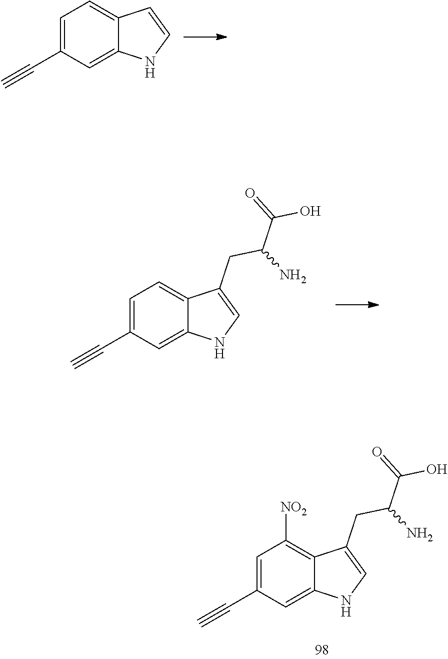 [SMILES: C#Cc1ccc2cc[nH]c2c1 ; C#Cc1ccc2c(CC(N)C(=O)O)c[nH]c2c1 ; C#Cc1cc([N+](=O)[O-])c2c(CC(N)C(=O)O)c[nH]c2c1]

Example 98 can be prepared from 6-ethylindole as shown above.

### Example 99: Preparation of 2-amino-3-(7-ethynyl-4-nitro-1H-indol-3-yl)propanoic acid (99)

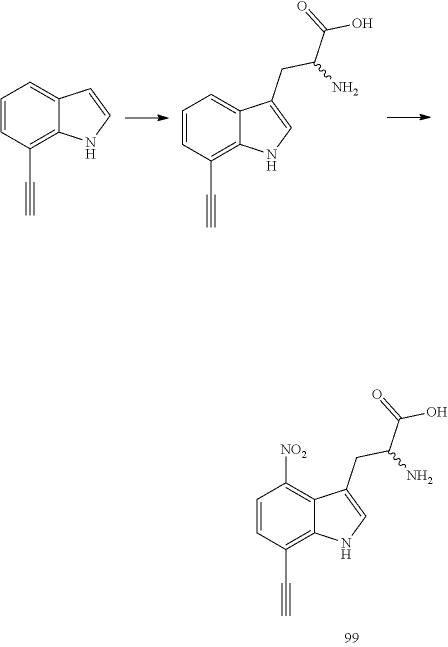 [SMILES: C#Cc1cccc2cc[nH]c12 ; C#Cc1cccc2c(CC(N)C(=O)O)c[nH]c12 ; C#Cc1ccc([N+](=O)[O-])c2c(CC(N)C(=O)O)c[nH]c12]

Example 99 can be prepared from 7-ethylindole as shown above.

### Example 100: Preparation of 2-amino-3-(4-phenyl-7-nitro-1H-indol-3-yl)propanoic acid (100)

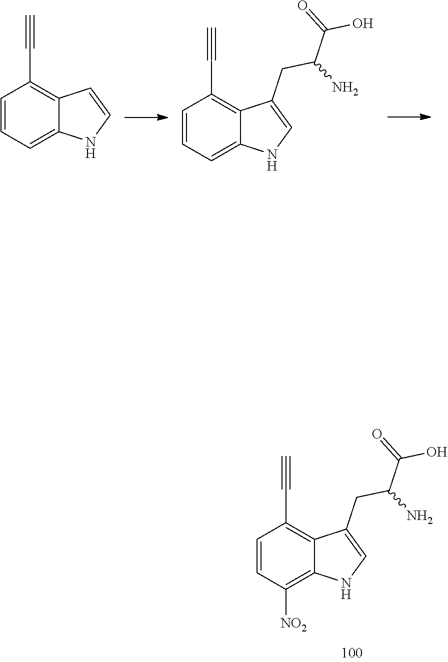 [SMILES: C#Cc1cccc2[nH]ccc12 ; C#Cc1cccc2[nH]cc(CC(N)C(=O)O)c12 ; C#Cc1ccc([N+](=O)[O-])c2[nH]cc(CC(N)C(=O)O)c12]

Example 100 can be prepared from 4-ethylindole as shown above.

### Example 101: Preparation of 2-amino-3-(5-morpholino-4-nitro-1H-indol-3-yl)propanoic acid (101)

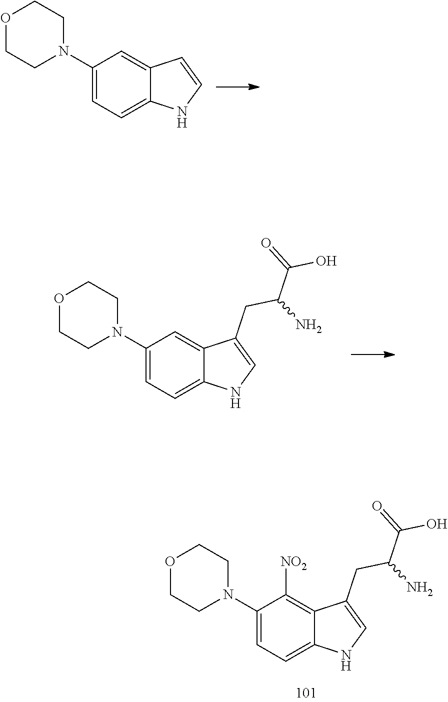 [SMILES: c1cc2cc(N3CCOCC3)ccc2[nH]1 ; NC(Cc1c[nH]c2ccc(N3CCOCC3)cc12)C(=O)O ; NC(Cc1c[nH]c2ccc(N3CCOCC3)c([N+](=O)[O-])c12)C(=O)O]

Example 101 can be prepared from 5-morpholinoindole as shown above,

### Example 102: Preparation of 2-amino-3-(6-morpholino-4-nitro-1H-indol-3-yl)propanoic acid (102)

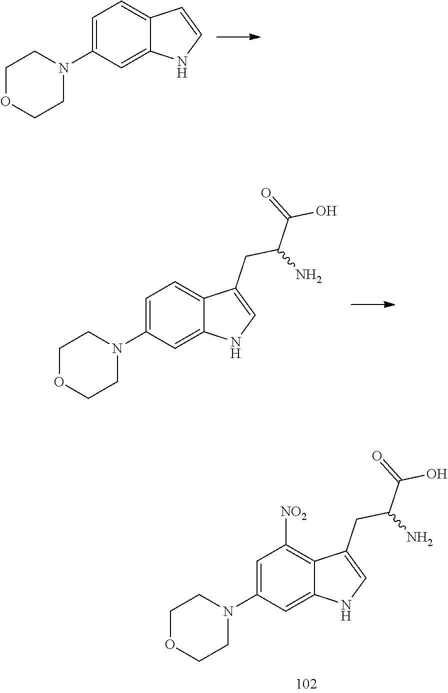 [SMILES: c1cc2ccc(N3CCOCC3)cc2[nH]1 ; NC(Cc1c[nH]c2cc(N3CCOCC3)ccc12)C(=O)O ; NC(Cc1c[nH]c2cc(N3CCOCC3)cc([N+](=O)[O-])c12)C(=O)O]

Example 102 can be prepared from 6-morpholinoindole as shown above.

### Example 103: Preparation of 2-amino-3-(7-morpholino-4-nitro-1H-indol-3-yl)propanoic acid (103)

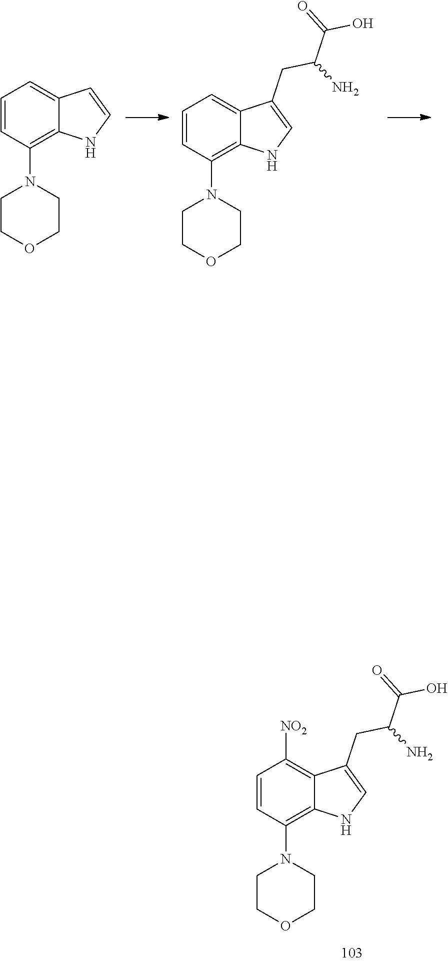 [SMILES: c1cc(N2CCOCC2)c2[nH]ccc2c1 ; NC(Cc1c[nH]c2c(N3CCOCC3)cccc12)C(=O)O ; NC(Cc1c[nH]c2c(N3CCOCC3)ccc([N+](=O)[O-])c12)C(=O)O]

Example 103 can be prepared from 7-morpholinoindole as shown above.

### Example 104: Preparation of 2-amino-3-(4-morpholino-7-nitro-1H-indol-3-yl)propanoic acid (104)

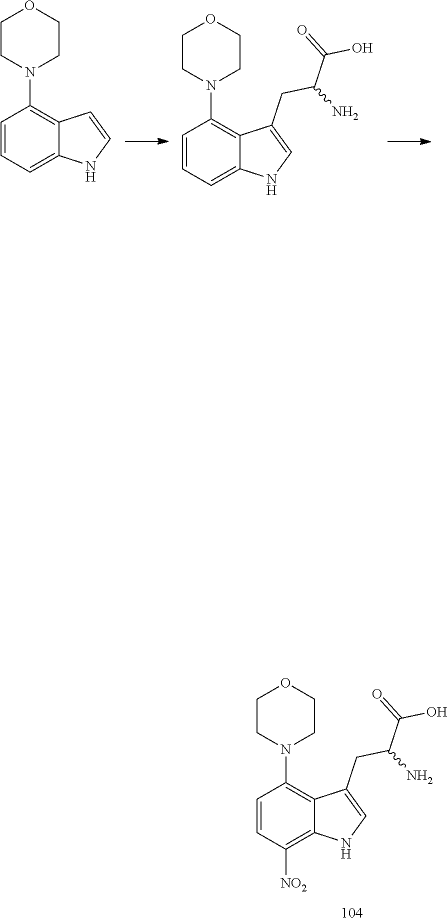 [SMILES: c1cc(N2CCOCC2)c2cc[nH]c2c1 ; NC(Cc1c[nH]c2cccc(N3CCOCC3)c12)C(=O)O ; NC(Cc1c[nH]c2c([N+](=O)[O-])ccc(N3CCOCC3)c12)C(=O)O]

Example 104 can be prepared from 4-morpholinoindole as shown above.

### Example 105: Preparation of 2-amino-3-(5-(methylthio)-4-nitro-1H-indol-3-yl)propanoic acid (105)

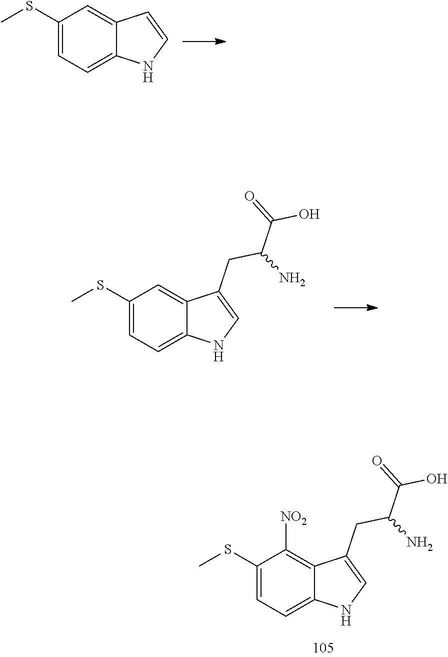 [SMILES: CSc1ccc2[nH]ccc2c1 ; CSc1ccc2[nH]cc(CC(N)C(=O)O)c2c1 ; CSc1ccc2[nH]cc(CC(N)C(=O)O)c2c1[N+](=O)[O-]]

Example 105 can be prepared from 5-(methylthio)indole as shown above.

### Example 106: Preparation of 2-amino-3-(6-(methylthio)-4-nitro-1H-indol-3-yl)propanoic acid (106)

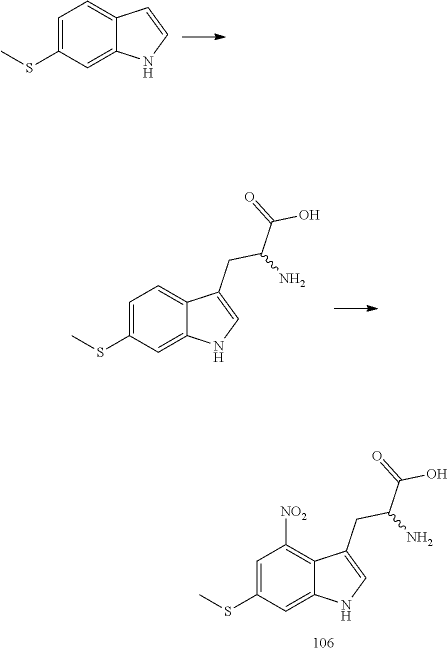 [SMILES: CSc1ccc2cc[nH]c2c1 ; CSc1ccc2c(CC(N)C(=O)O)c[nH]c2c1 ; CSc1cc([N+](=O)[O-])c2c(CC(N)C(=O)O)c[nH]c2c1]

Example 106 can be prepared from 6-(methylthio)indole as shown above.

### Example 107: Preparation of 2-amino-3-(7-(methylthio)-4-nitro-1H-indol-3-yl)propanoic acid (107)

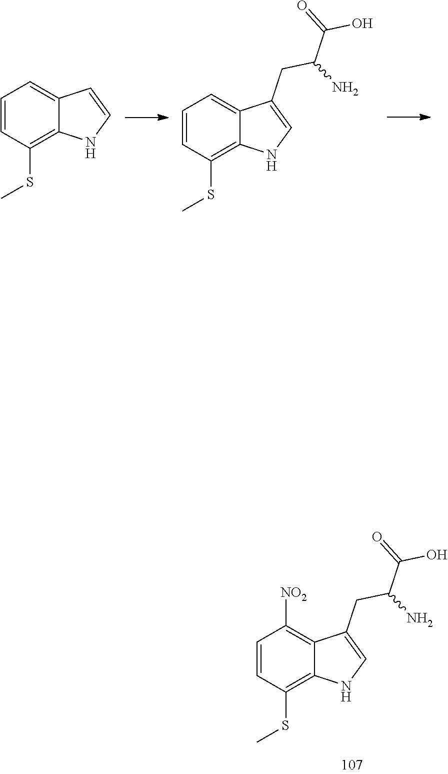 [SMILES: CSc1cccc2cc[nH]c12 ; CSc1cccc2c(CC(N)C(=O)O)c[nH]c12 ; CSc1ccc([N+](=O)[O-])c2c(CC(N)C(=O)O)c[nH]c12]

Example 107 can be prepared from 7-(methylthio)indole as shown above.

### Example 108: Preparation of 2-amino-3-(4-(methylthio)-7-nitro-1H-indol-3-yl)propanoic acid (108)

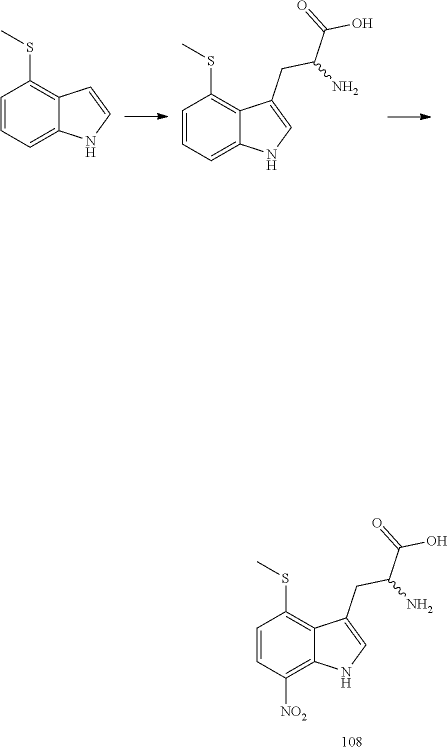 [SMILES: CSc1cccc2[nH]ccc12 ; CSc1cccc2[nH]cc(CC(N)C(=O)O)c12 ; CSc1ccc([N+](=O)[O-])c2[nH]cc(CC(N)C(=O)O)c12]

Example 108 can be prepared from 4-(methylthio)indole as shown above.

### Example 109: Preparation of 2-amino-3-(4-nitro-5-(pyridin-4-yl)-1H-indol-3-yl)propanoic acid (109)

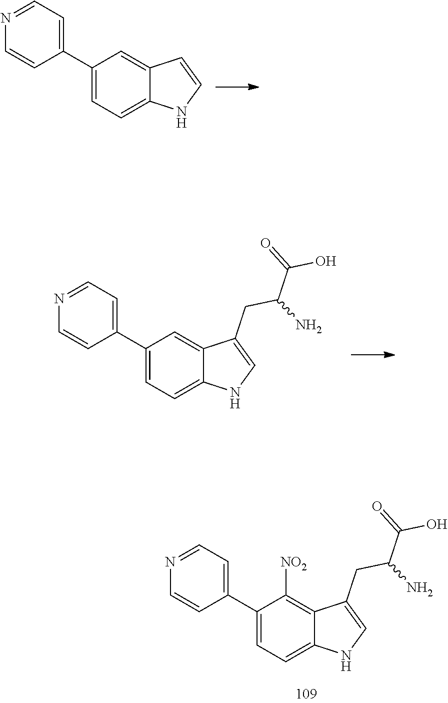 [SMILES: c1cc(-c2ccc3[nH]ccc3c2)ccn1 ; NC(Cc1c[nH]c2ccc(-c3ccncc3)cc12)C(=O)O ; NC(Cc1c[nH]c2ccc(-c3ccncc3)c([N+](=O)[O-])c12)C(=O)O]

Example 109 can be prepared from 5-(pyridin-4-yl)indole as shown above.

### Example 110: Preparation of 2-amino-3-(4-nitro-6-(pyridin-4-yl)-1H-indol-3-yl)propanoic acid (110)

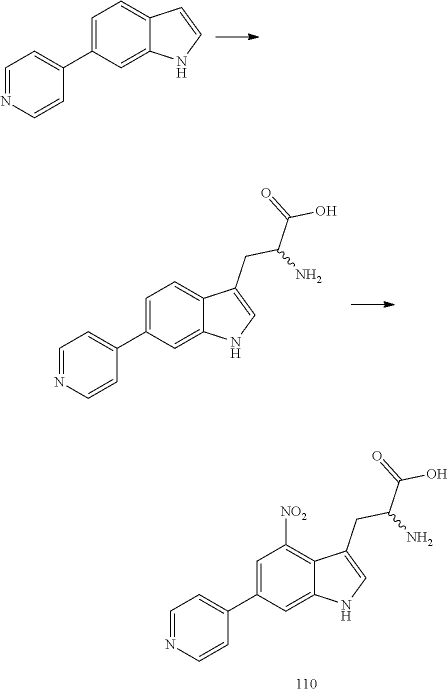 [SMILES: c1cc(-c2ccc3cc[nH]c3c2)ccn1 ; NC(Cc1c[nH]c2cc(-c3ccncc3)ccc12)C(=O)O ; NC(Cc1c[nH]c2cc(-c3ccncc3)cc([N+](=O)[O-])c12)C(=O)O]

Example 110 can be prepared from 6-(pyridin-4-yl)indole as shown above.

### Example 111: Preparation of 2-amino-3-(4-nitro-7-(pyridin-4-yl)-1H-indol-3-yl)propanoic acid (111)

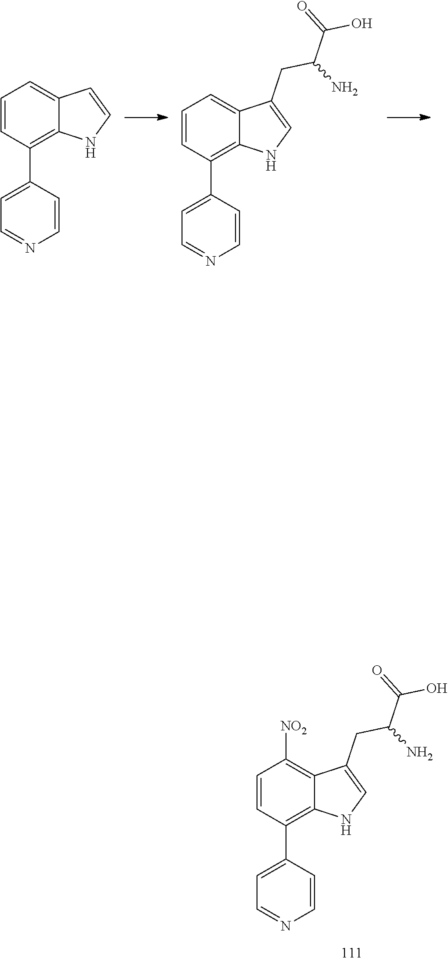 [SMILES: c1cc(-c2ccncc2)c2[nH]ccc2c1 ; NC(Cc1c[nH]c2c(-c3ccncc3)cccc12)C(=O)O ; NC(Cc1c[nH]c2c(-c3ccncc3)ccc([N+](=O)[O-])c12)C(=O)O]

Example 111 can be prepared from 7-(pyridin-4-yl)indole as shown above.

### Example 112: Preparation of 2-amino-3-(7-nitro-4-(pyridin-4-yl)-1H-indol-3-yl)propanoic acid (112)

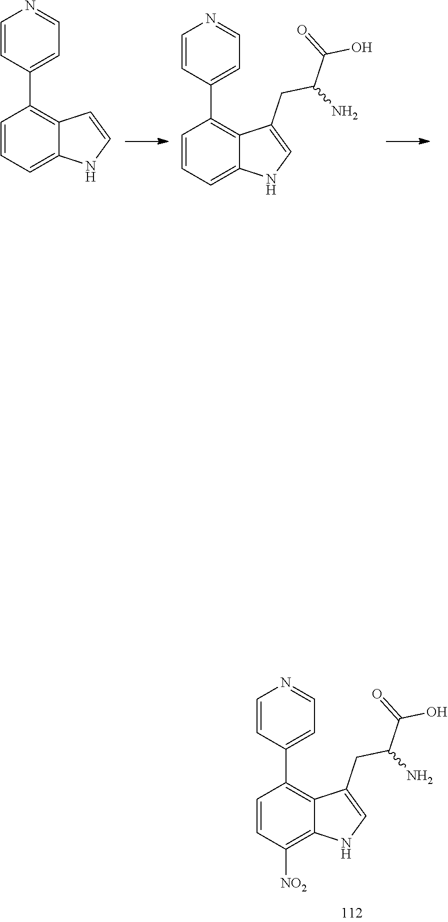 [SMILES: c1cc(-c2ccncc2)c2cc[nH]c2c1 ; NC(Cc1c[nH]c2cccc(-c3ccncc3)c12)C(=O)O ; NC(Cc1c[nH]c2c([N+](=O)[O-])ccc(-c3ccncc3)c12)C(=O)O]

Example 112 can be prepared from 4-(pyridin-4-yl)indol as shown above.

### Example 113: Preparation of 2-amino-3-(1,5-dimethyl-4-nitro-1H-indol-3-yl)propanoic acid (113)

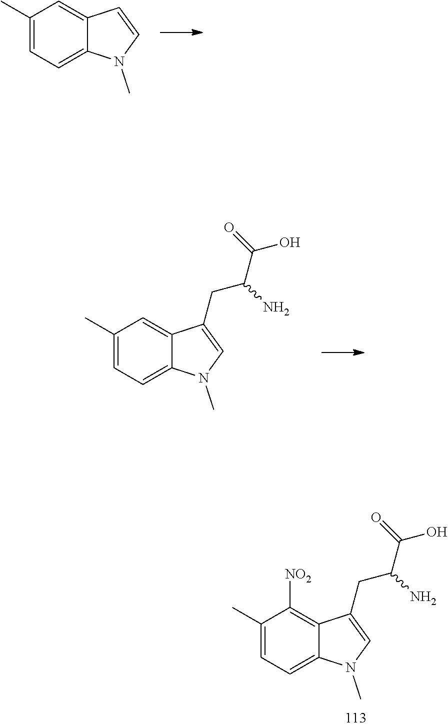 [SMILES: Cc1ccc2c(ccn2C)c1 ; Cc1ccc2c(c1)c(CC(N)C(=O)O)cn2C ; Cc1ccc2c(c(CC(N)C(=O)O)cn2C)c1[N+](=O)[O-]]

Example 113 can be prepared from 1,5-dimethyl-1H-indole as shown above.

### Example 114: Preparation of 2-amino-3-(1,6-dimethyl-4-nitro-1H-indol-3-yl)propanoic acid (114)

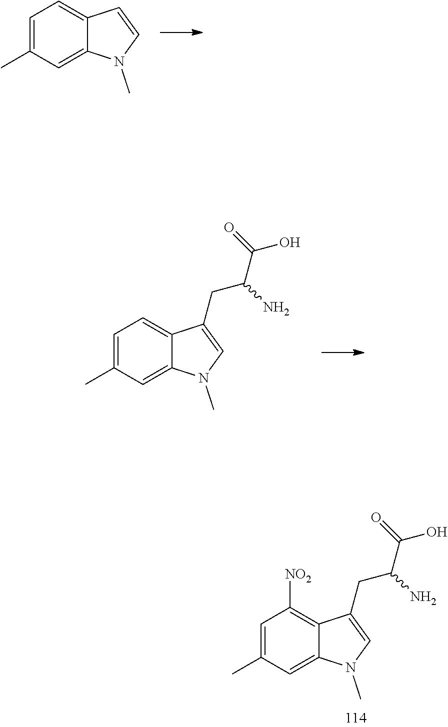 [SMILES: Cc1ccc2ccn(C)c2c1 ; Cc1ccc2c(CC(N)C(=O)O)cn(C)c2c1 ; Cc1cc([N+](=O)[O-])c2c(CC(N)C(=O)O)cn(C)c2c1]

Example 114 can be prepared from 1,6-dimethyl-1H-indole as shown above.

### Example 115: Preparation of 2-amino-3-(1,7-dimethyl-4-nitro-1H-indol-3-yl)propanoic acid (115)

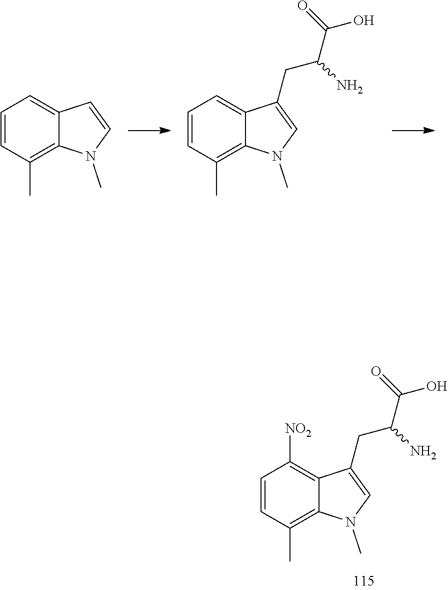 [SMILES: Cc1cccc2ccn(C)c12 ; Cc1cccc2c(CC(N)C(=O)O)cn(C)c12 ; Cc1ccc([N+](=O)[O-])c2c(CC(N)C(=O)O)cn(C)c12]

Example 115 can be prepared from 1,7-dimethyl-1H-indole as shown above.

### Example 116: Preparation of 2-amino-3-(1,4-dimethyl-7-nitro-1H-indol-3-yl)propanoic acid (116)

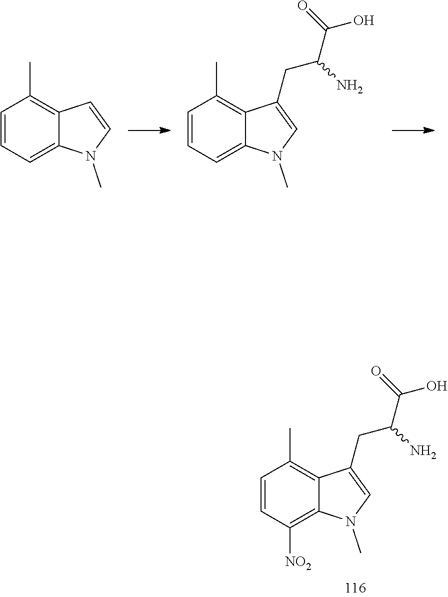 [SMILES: Cc1cccc2c1ccn2C ; Cc1cccc2c1c(CC(N)C(=O)O)cn2C ; Cc1ccc([N+](=O)[O-])c2c1c(CC(N)C(=O)O)cn2C]

Example 116 can be prepared from 1,4-dimethyl-1H-indole as shown above.

### Example 117: Preparation of 2-amino-3-(6-fluoro-1-methyl-4-nitro-1H-indol-3-yl)propanoic acid (117)

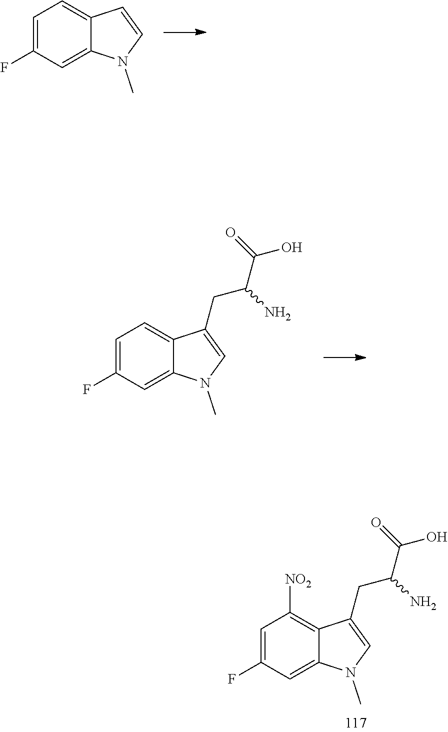 [SMILES: Cn1ccc2ccc(F)cc21 ; Cn1cc(CC(N)C(=O)O)c2ccc(F)cc21 ; Cn1cc(CC(N)C(=O)O)c2c([N+](=O)[O-])cc(F)cc21]

Example 117 can be prepared from 6-fluoro-1-methyl-1H-indole as shown above.

### Example 118: Preparation of 2-amino-3-(7-fluoro-1-methyl-4-nitro-1H-indol-3-yl)propanoic acid (118)

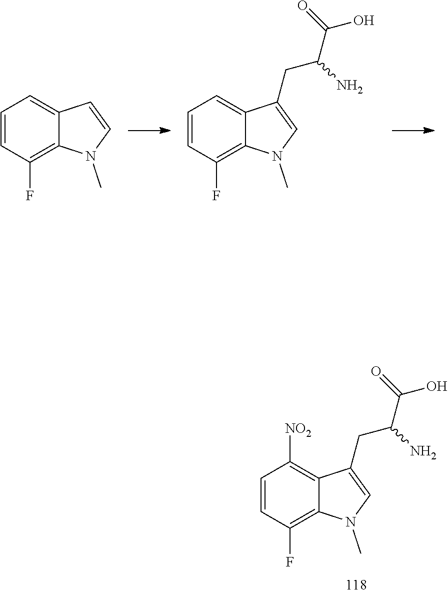 [SMILES: Cn1ccc2cccc(F)c21 ; Cn1cc(CC(N)C(=O)O)c2cccc(F)c21 ; Cn1cc(CC(N)C(=O)O)c2c([N+](=O)[O-])ccc(F)c21]

Example 118 can be prepared from 7-fluoro-1-methyl-indole as shown above.

### Example 119: Preparation of 2-amino-3-(4-fluoro-1-methyl-7-nitro-1H-indol-3-yl)propanoic acid (119)

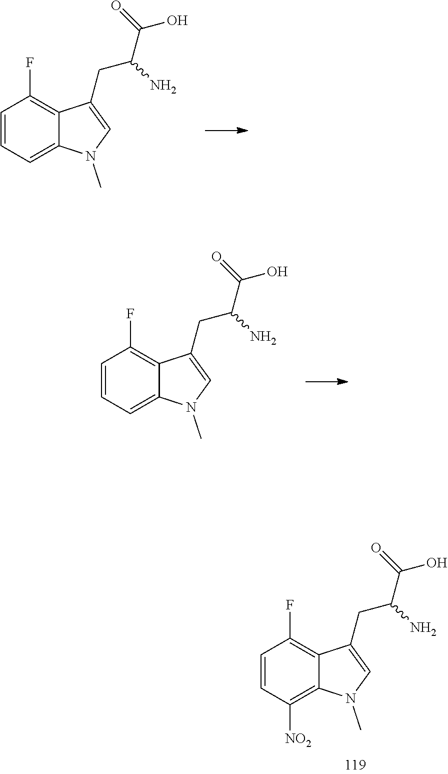 [SMILES: Cn1cc(CC(N)C(=O)O)c2c(F)cccc21 ; Cn1cc(CC(N)C(=O)O)c2c(F)cccc21 ; Cn1cc(CC(N)C(=O)O)c2c(F)ccc([N+](=O)[O-])c21]

Example 119 can be prepared from 4-fluoro-1-methyl-indole as shown above.

### Example 120: Preparation of 2-amino-3-(5-chloro-1-methyl-4-nitro-1H-indol-3-yl)propanoic acid (120)

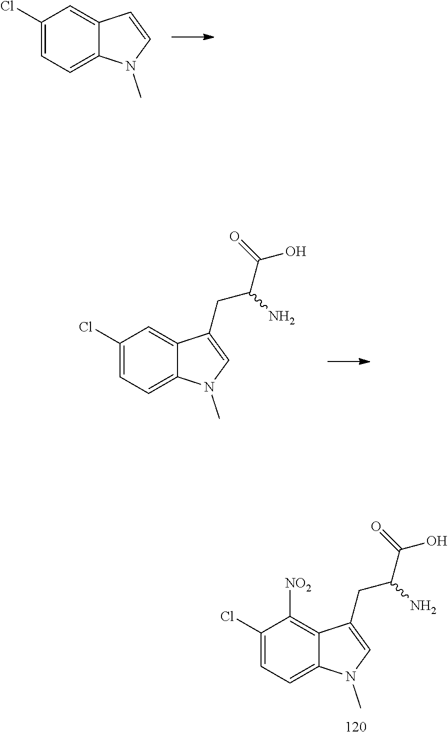 [SMILES: Cn1ccc2cc(Cl)ccc21 ; Cn1cc(CC(N)C(=O)O)c2cc(Cl)ccc21 ; Cn1cc(CC(N)C(=O)O)c2c([N+](=O)[O-])c(Cl)ccc21]

Example 120 can be prepared from 5-chloro-1-methyl-indole as shown above.

### Example 121: Preparation of 2-amino-3-(6-choro-1-methyl-4-nitro-1H-indol-3-yl)propanoic acid (121)

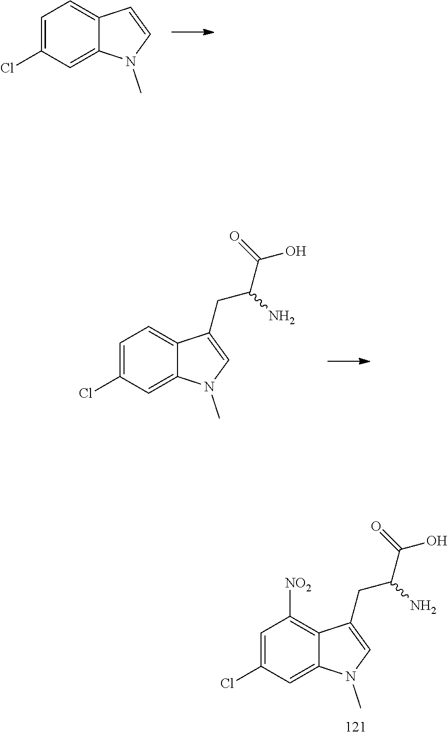 [SMILES: Cn1ccc2ccc(Cl)cc21 ; Cn1cc(CC(N)C(=O)O)c2ccc(Cl)cc21 ; Cn1cc(CC(N)C(=O)O)c2c([N+](=O)[O-])cc(Cl)cc21]

Example 121 can be prepared from 6-chloro-1-methyl-indole as shown above.

### Example 122: Preparation of 2-amino-3-(7-chloro-1-methyl-4-nitro-1H-indol-3-yl)propanoic acid (122)

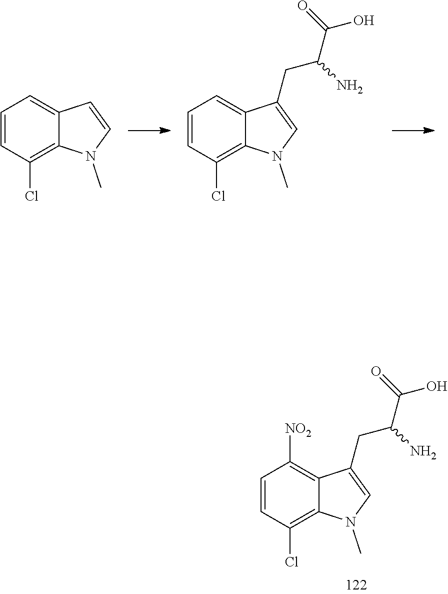 [SMILES: Cn1ccc2cccc(Cl)c21 ; Cn1cc(CC(N)C(=O)O)c2cccc(Cl)c21 ; Cn1cc(CC(N)C(=O)O)c2c([N+](=O)[O-])ccc(Cl)c21]

Example 122 can be prepared from 7-chloro-1-methyl-indole as shown above.

### Example 123: Preparation of 2-amino-3-(4-chloro-1-methyl-7-nitro-1H-indol-3-yl)propanoic acid (123)

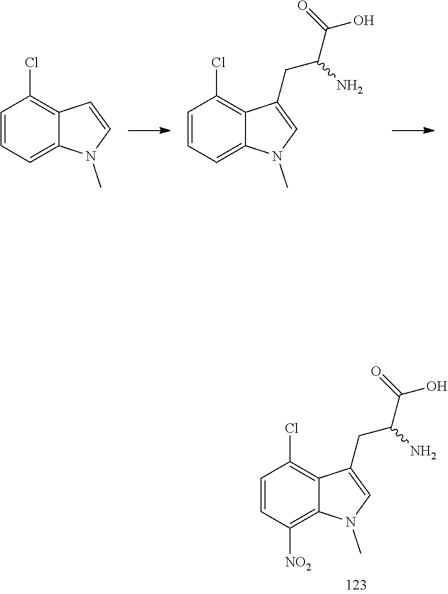 [SMILES: Cn1ccc2c(Cl)cccc21 ; Cn1cc(CC(N)C(=O)O)c2c(Cl)cccc21 ; Cn1cc(CC(N)C(=O)O)c2c(Cl)ccc([N+](=O)[O-])c21]

Example 123 can be prepared from 4-chloro-1-methyl-indole as shown above.

### Example 124: Preparation of 2-amino-3-(5-bromol-methyl-4-nitro-1H-indol-3-yl)propanoic acid (124)

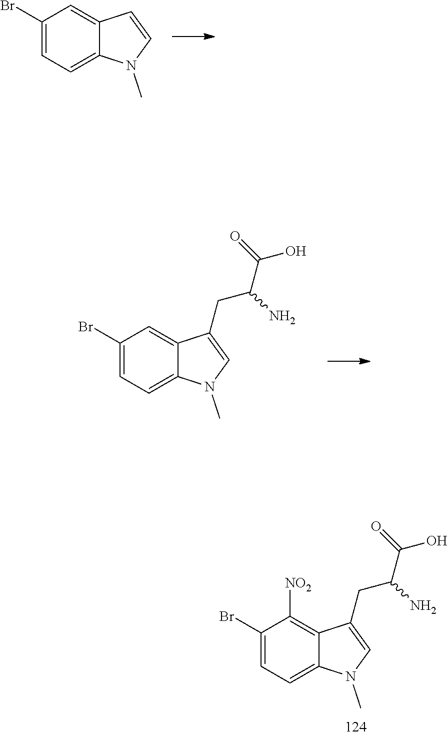 [SMILES: Cn1ccc2cc(Br)ccc21 ; Cn1cc(CC(N)C(=O)O)c2cc(Br)ccc21 ; Cn1cc(CC(N)C(=O)O)c2c([N+](=O)[O-])c(Br)ccc21]

Example 124 can be prepared from 5-bromo-1-methyl-indole as shown above.

### Example 125: Preparation of 2-amino-3-(6-bromo-1-methyl-4-nitro-1H-indol-3-yl)propanoic acid (125)

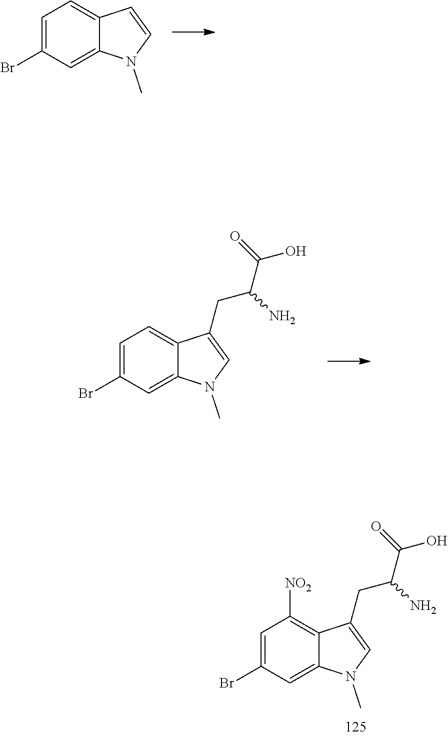 [SMILES: Cn1ccc2ccc(Br)cc21 ; Cn1cc(CC(N)C(=O)O)c2ccc(Br)cc21 ; Cn1cc(CC(N)C(=O)O)c2c([N+](=O)[O-])cc(Br)cc21]

Example 125 can be prepared from 6-bromo-1-methyl-indole as shown above.

### Example 126: Preparation of 2-amino-3-(7-bromo-1-methyl-4-nitro-1H-indol-3-yl)propanoic acid (126)

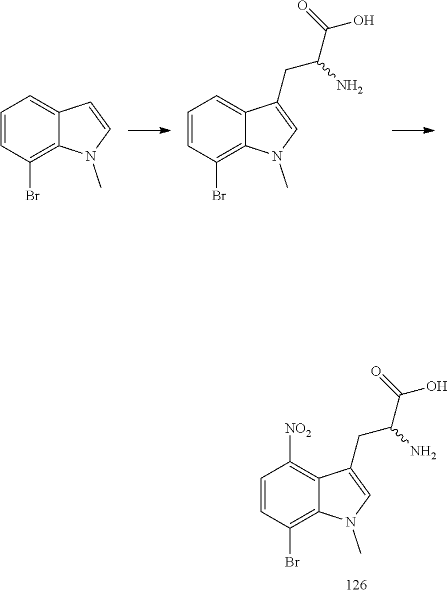 [SMILES: Cn1ccc2cccc(Br)c21 ; Cn1cc(CC(N)C(=O)O)c2cccc(Br)c21 ; Cn1cc(CC(N)C(=O)O)c2c([N+](=O)[O-])ccc(Br)c21]

Example 126 can be prepared from 7-bromo-1-methyl-indole as shown above.

### Example 127: Preparation of 2-amino-3-(4-bromo-1-methyl-7-nitro-1H-indol-3-yl)propanoic acid (127)

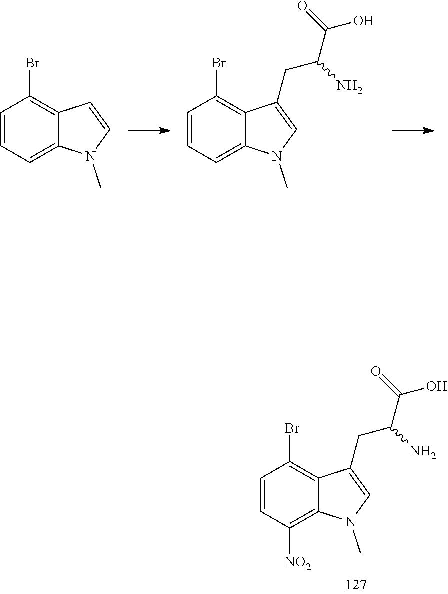 [SMILES: Cn1ccc2c(Br)cccc21 ; Cn1cc(CC(N)C(=O)O)c2c(Br)cccc21 ; Cn1cc(CC(N)C(=O)O)c2c(Br)ccc([N+](=O)[O-])c21]

Example 127 can be prepared from 4-bromo-1-methyl-indole as shown above.

### Example 128: Preparation of 2-amino-3-(5-methoxy-1-methyl-4-nitro-1H-indol-3-yl)propanoic acid (128)

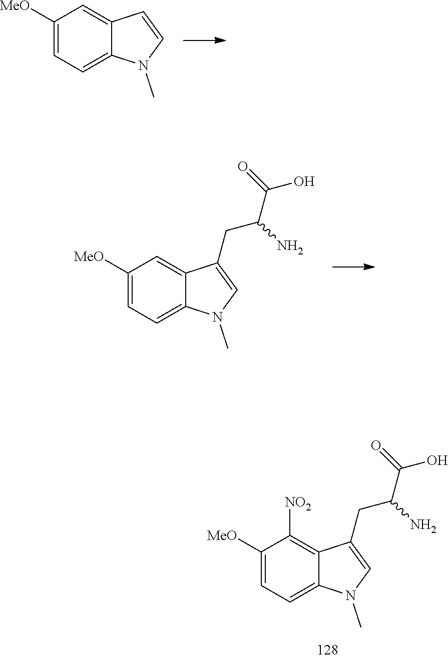 [SMILES: COc1ccc2c(ccn2C)c1 ; COc1ccc2c(c1)c(CC(N)C(=O)O)cn2C ; COc1ccc2c(c(CC(N)C(=O)O)cn2C)c1[N+](=O)[O-]]

Example 128 can be prepared from 5-methoxy-1-methyl-indole as shown above.

### Example 129: Preparation of 2-amino-3-(6-methoxy-1-methyl-4-nitro-1H-indol-3-yl)propanoic acid (129)

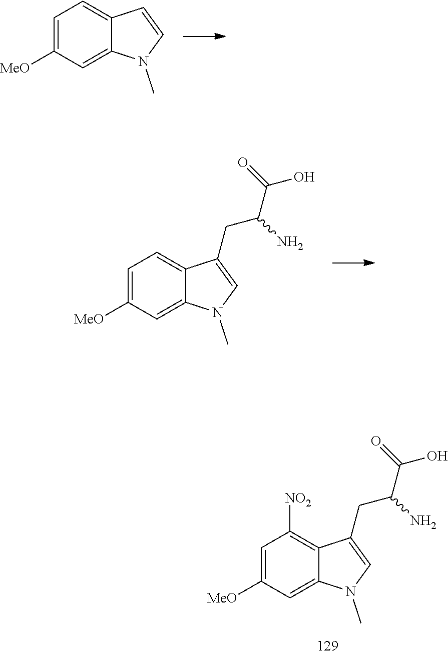 [SMILES: COc1ccc2ccn(C)c2c1 ; COc1ccc2c(CC(N)C(=O)O)cn(C)c2c1 ; COc1cc([N+](=O)[O-])c2c(CC(N)C(=O)O)cn(C)c2c1]

Example 129 can be prepared from 6-methoxy-1-methyl-indole as shown above.

### Example 130: Preparation of 2-amino-3-(7-methoxy-1-methyl-4-nitro-1H-indol-3-yl)propanoic acid (130)

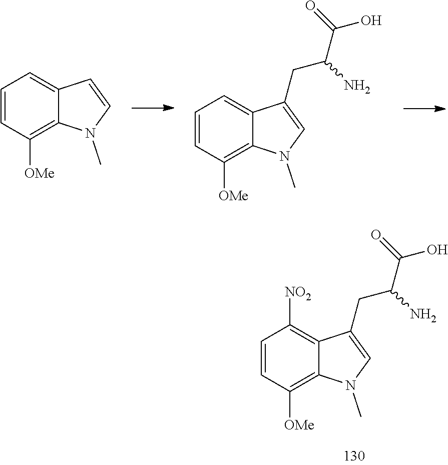 [SMILES: COc1cccc2ccn(C)c12 ; COc1cccc2c(CC(N)C(=O)O)cn(C)c12 ; COc1ccc([N+](=O)[O-])c2c(CC(N)C(=O)O)cn(C)c12]

Example 130 can be prepared from 7-methoxy-1-methyl-indole as shown above.

### Example 131: Preparation of 2-amino-3-(4-methoxy-1-methyl-7-nitro-1H-indol-3-yl)propanoic acid (131)

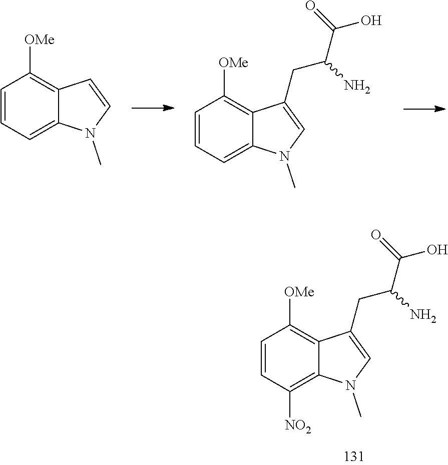 [SMILES: COc1cccc2c1ccn2C ; COc1cccc2c1c(CC(N)C(=O)O)cn2C ; COc1ccc([N+](=O)[O-])c2c1c(CC(N)C(=O)O)cn2C]

Example 131 can be prepared from 4-methoxy-1-methyl-indole as shown above.

### Example 132: Preparation of 2-amino-3-(5-amino-1-methyl-4-nitro-1H-indol-3-yl)propanoic acid (132)

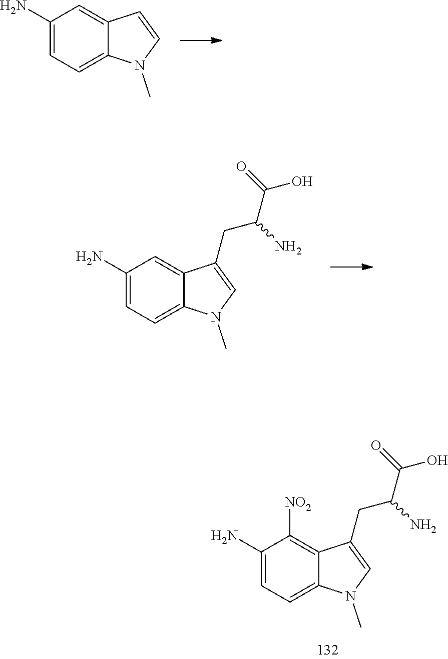 [SMILES: Cn1ccc2cc(N)ccc21 ; Cn1cc(CC(N)C(=O)O)c2cc(N)ccc21 ; Cn1cc(CC(N)C(=O)O)c2c([N+](=O)[O-])c(N)ccc21]

Example 132 can be prepared from 5-amino-1-methyl-indole as shown above.

### Example 133: Preparation of 2-amino-3-(6-amino-1-methyl-4-nitro-1H-indol-3-yl)propanoic acid (133)

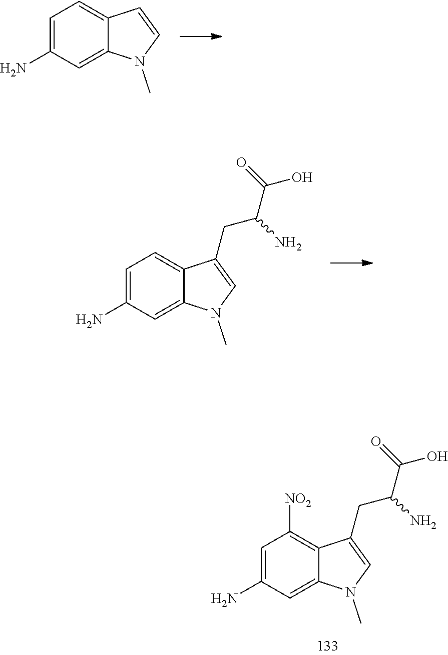 [SMILES: Cn1ccc2ccc(N)cc21 ; Cn1cc(CC(N)C(=O)O)c2ccc(N)cc21 ; Cn1cc(CC(N)C(=O)O)c2c([N+](=O)[O-])cc(N)cc21]

Example 133 can be prepared from 6-amino-1-methyl-indole as shown above.

### Example 134: Preparation of 2-amino-3-(7-amino-1-methyl-4-nitro-1H-indol-3-yl)propanoic acid (134)

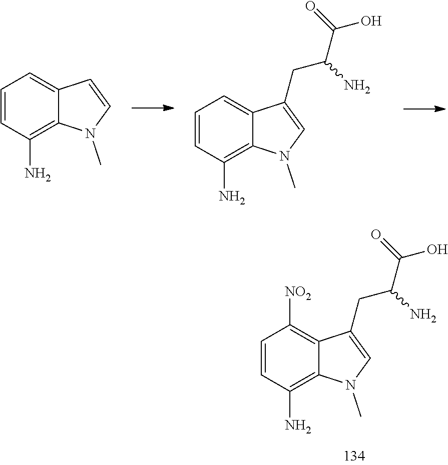 [SMILES: Cn1ccc2cccc(N)c21 ; Cn1cc(CC(N)C(=O)O)c2cccc(N)c21 ; Cn1cc(CC(N)C(=O)O)c2c([N+](=O)[O-])ccc(N)c21]

Example 134 can be prepared from 7-amino-1-methyl-indole as shown above.

### Example 135: Preparation of 2-amino-3-(4-amino-1-methyl-7-nitro-1H-indol-3-yl)propanoic acid (135)

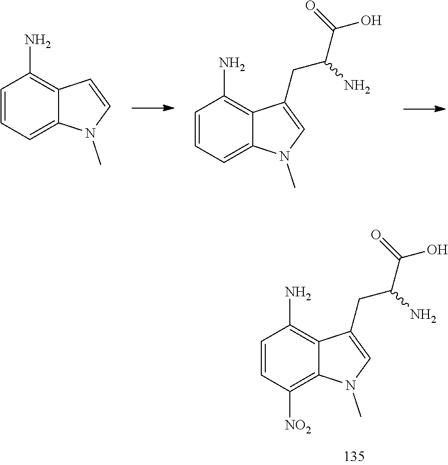 [SMILES: Cn1ccc2c(N)cccc21 ; Cn1cc(CC(N)C(=O)O)c2c(N)cccc21 ; Cn1cc(CC(N)C(=O)O)c2c(N)ccc([N+](=O)[O-])c21]

Example 135 can be prepared from 4-amino-1-methyl-indole as shown above.

### Example 136: Preparation of 2-amino-3-(5-hydroxy-1-methyl-4-nitro-1H-indol-3-yl)propanoic acid (136)

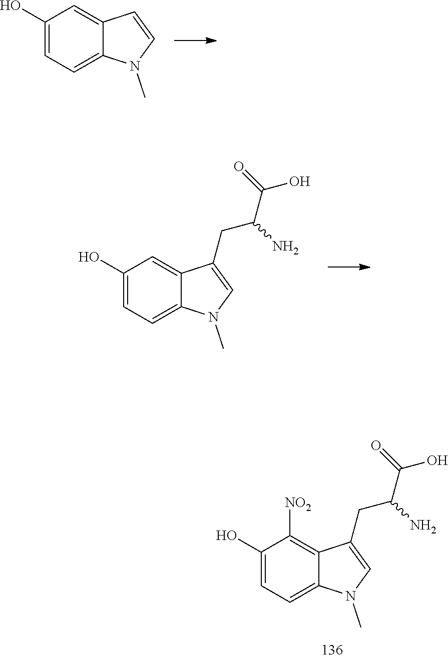 [SMILES: Cn1ccc2cc(O)ccc21 ; Cn1cc(CC(N)C(=O)O)c2cc(O)ccc21 ; Cn1cc(CC(N)C(=O)O)c2c([N+](=O)[O-])c(O)ccc21]

Example 136 can be prepared from 5-hydroxy-1-methyl-indole as shown above.

### Example 137: Preparation of 2-amino-3-(6-hydroxy-1-methyl-4-nitro-1H-indol-3-yl)propanoic acid (137)

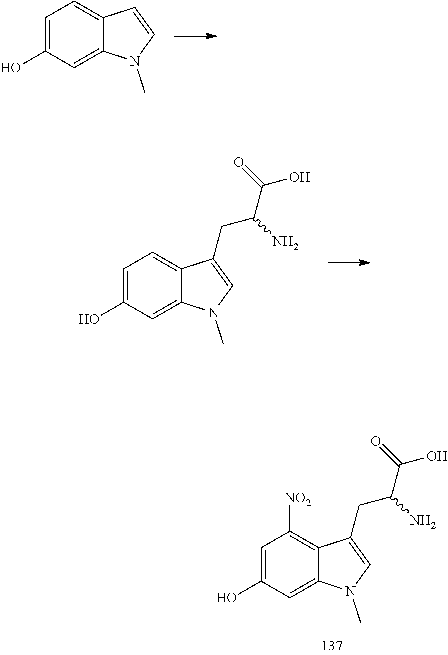 [SMILES: Cn1ccc2ccc(O)cc21 ; Cn1cc(CC(N)C(=O)O)c2ccc(O)cc21 ; Cn1cc(CC(N)C(=O)O)c2c([N+](=O)[O-])cc(O)cc21]

Example 137 can be prepared from 6-hydroxy-1-methyl-indole as shown above.

### Example 138: Preparation of 2-amino-3-(7-hydroxy-1-methyl-4-nit %-1H-indol-3-yl)propanoic acid (138)

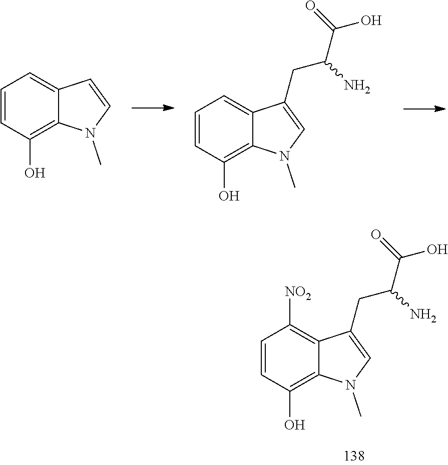 [SMILES: Cn1ccc2cccc(O)c21 ; Cn1cc(CC(N)C(=O)O)c2cccc(O)c21 ; Cn1cc(CC(N)C(=O)O)c2c([N+](=O)[O-])ccc(O)c21]

Example 138 can be prepared from 7-hydroxy-1-methyl-indole as shown above.

### Example 139: Preparation of 2-amino-3-(4-hydroxy-1-methyl-7-nitro-1H-indol-3-yl)propanoic acid (139)

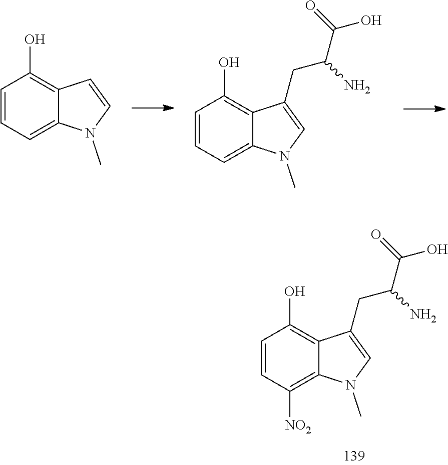 [SMILES: Cn1ccc2c(O)cccc21 ; Cn1cc(CC(N)C(=O)O)c2c(O)cccc21 ; Cn1cc(CC(N)C(=O)O)c2c(O)ccc([N+](=O)[O-])c21]

Example 139 can be prepared from 4-hydroxy-1-methyl-indole as shown above.

### Example 140: Preparation of 2-amino-3-(1-methyl-4-nitro-5-phenyl-1H-indol-3-yl)propanoic acid (140)

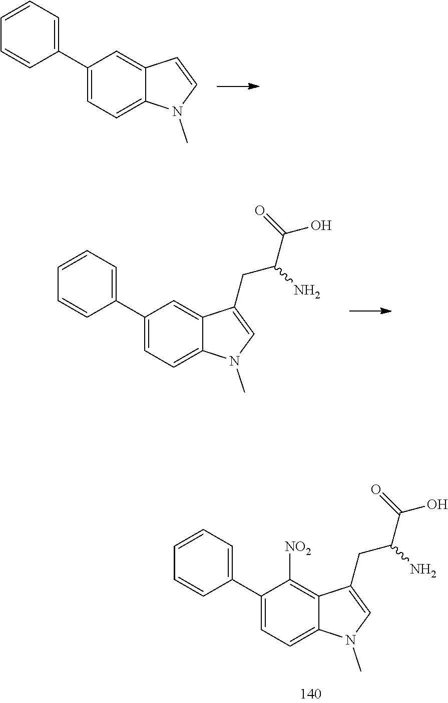 [SMILES: Cn1ccc2cc(-c3ccccc3)ccc21 ; Cn1cc(CC(N)C(=O)O)c2cc(-c3ccccc3)ccc21 ; Cn1cc(CC(N)C(=O)O)c2c([N+](=O)[O-])c(-c3ccccc3)ccc21]

Example 140 can be prepared from 5-phenyl-1-methyl-indole as shown above.

### Example 141: Preparation of 2-amino-3-(1-methyl-4-nitro-6-phenyl-1H-indol-3-yl)propanoic acid (141)

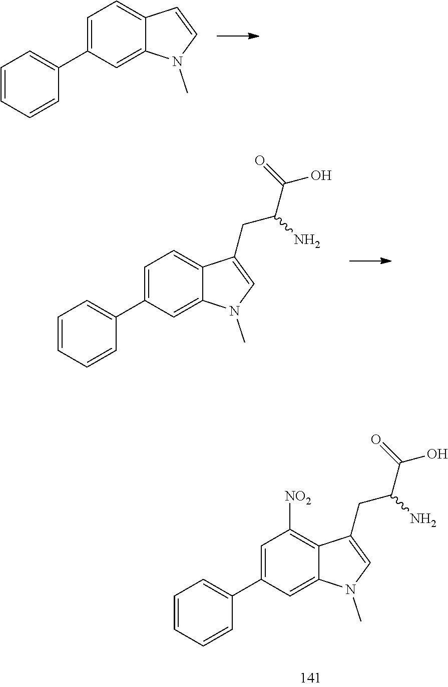 [SMILES: Cn1ccc2ccc(-c3ccccc3)cc21 ; Cn1cc(CC(N)C(=O)O)c2ccc(-c3ccccc3)cc21 ; Cn1cc(CC(N)C(=O)O)c2c([N+](=O)[O-])cc(-c3ccccc3)cc21]

Example 141 can be prepared from 6-phenyl-1-methyl-indole as shown above.

### Example 142: Preparation of 2-amino-3-(1-methy-4-nitro-7-phenyl-1H-indol-3-yl)propanoic acid (142)

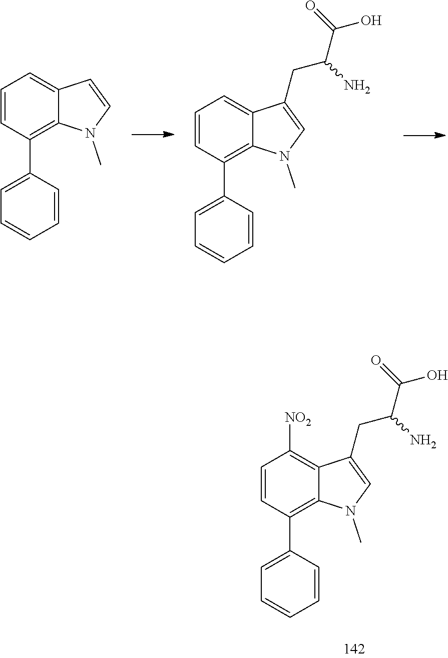 [SMILES: Cn1ccc2cccc(-c3ccccc3)c21 ; Cn1cc(CC(N)C(=O)O)c2cccc(-c3ccccc3)c21 ; Cn1cc(CC(N)C(=O)O)c2c([N+](=O)[O-])ccc(-c3ccccc3)c21]

Example 142 can be prepared from 7-phenyl-1-methyl-indole as shown above.

### Example 143: Preparation of 2-amino-3-(1-methyl-7-nitro-4-phenyl-1H-indol-3-yl)propanoic acid (143)

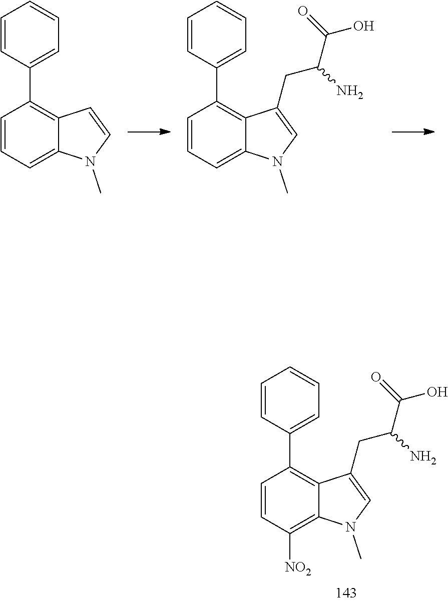 [SMILES: Cn1ccc2c(-c3ccccc3)cccc21 ; Cn1cc(CC(N)C(=O)O)c2c(-c3ccccc3)cccc21 ; Cn1cc(CC(N)C(=O)O)c2c(-c3ccccc3)ccc([N+](=O)[O-])c21]

Example 143 can be prepared from 4-phenyl-1-methyl-indole as shown above.

### Example 144: Preparation of 2-amino-3-(5-cyclopropyl-1-methyl-4-nitro-1H-indol-3-yl)propanoic acid (144)

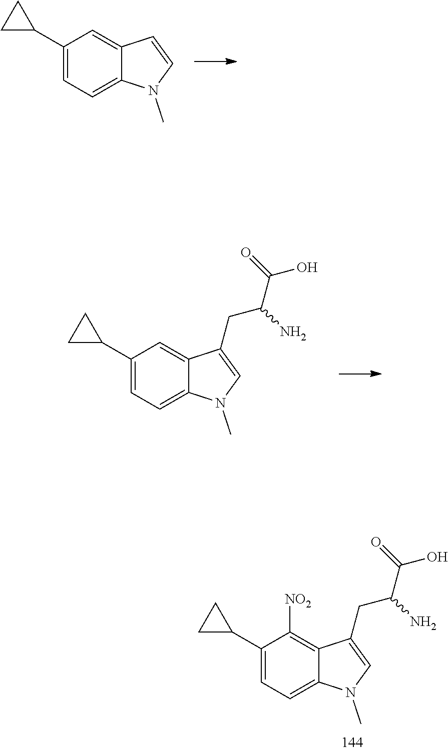 [SMILES: Cn1ccc2cc(C3CC3)ccc21 ; Cn1cc(CC(N)C(=O)O)c2cc(C3CC3)ccc21 ; Cn1cc(CC(N)C(=O)O)c2c([N+](=O)[O-])c(C3CC3)ccc21]

Example 144 can be prepared from 5-cyclopropyl-1-methyl-indole as shown above.

### Example 145: Preparation of 2-amino-3-(6-cyclopropyl-1-methyl-4-nitro-1H-indol-3-yl)propanoic acid (145)

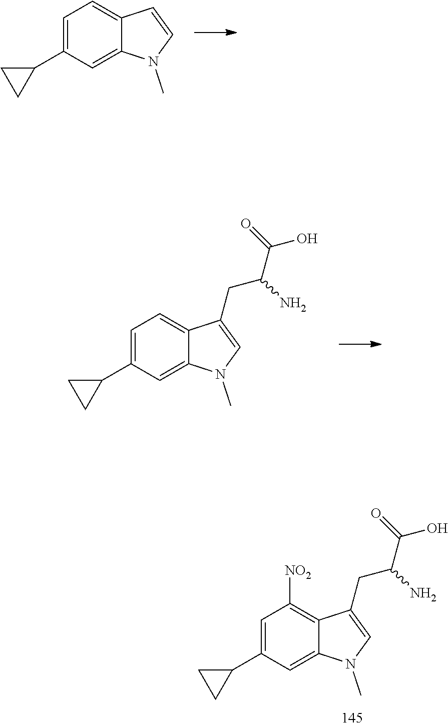 [SMILES: Cn1ccc2ccc(C3CC3)cc21 ; Cn1cc(CC(N)C(=O)O)c2ccc(C3CC3)cc21 ; Cn1cc(CC(N)C(=O)O)c2c([N+](=O)[O-])cc(C3CC3)cc21]

Example 145 can be prepared from 6-cyclopropyl-1-methyl-indole as shown above.

### Example 146: Preparation of 2-amino-3-(7-cyclopropyl-1-methyl-4-nitro-1H-indol-3-yl)propanoic acid (146)

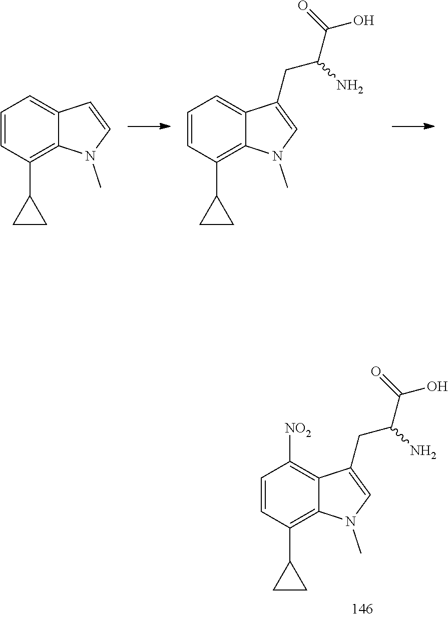 [SMILES: Cn1ccc2cccc(C3CC3)c21 ; Cn1cc(CC(N)C(=O)O)c2cccc(C3CC3)c21 ; Cn1cc(CC(N)C(=O)O)c2c([N+](=O)[O-])ccc(C3CC3)c21]

Example 146 can be prepared from 7-cyclopropyl-1-methyl-indole as shown above.

### Example 147: Preparation of 2-amino-3-(4-cyclopropyl-1-methyl-7-nitro-1H-indol-3-yl)propanoic acid (147)

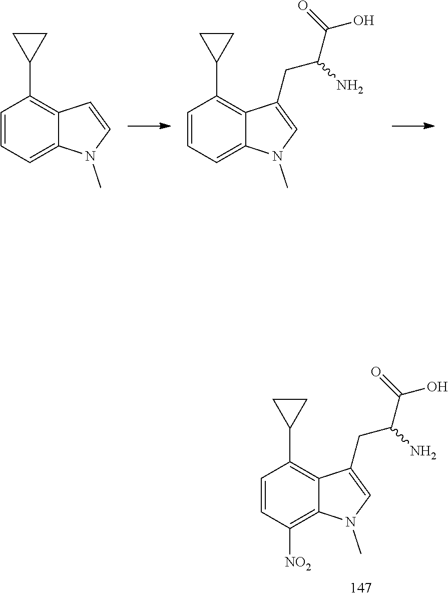 [SMILES: Cn1ccc2c(C3CC3)cccc21 ; Cn1cc(CC(N)C(=O)O)c2c(C3CC3)cccc21 ; Cn1cc(CC(N)C(=O)O)c2c(C3CC3)ccc([N+](=O)[O-])c21]

Example 147 can be prepared from 4-cyclopropyl-1-methyl-indole as shown above.

### Example 148: Preparation of 2-amino-3-(1-methy-4-nitro-5-viny-1H-indol-3-yl)propanoic acid (148)

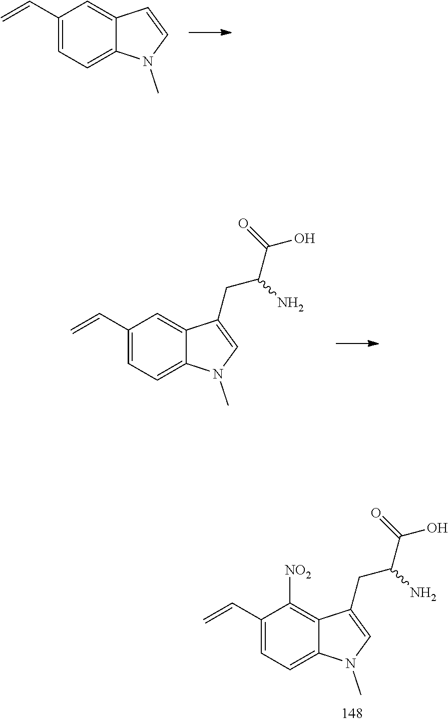 [SMILES: C=Cc1ccc2c(ccn2C)c1 ; C=Cc1ccc2c(c1)c(CC(N)C(=O)O)cn2C ; C=Cc1ccc2c(c(CC(N)C(=O)O)cn2C)c1[N+](=O)[O-]]

Example 148 can be prepared from 5-vinyl-1-methyl-indole as shown above.

### Example 149: Preparation of 2-amino-3-(1-methyl-4-nitro-6-vinyl-1H-indol-3-yl)propanoic acid (149)

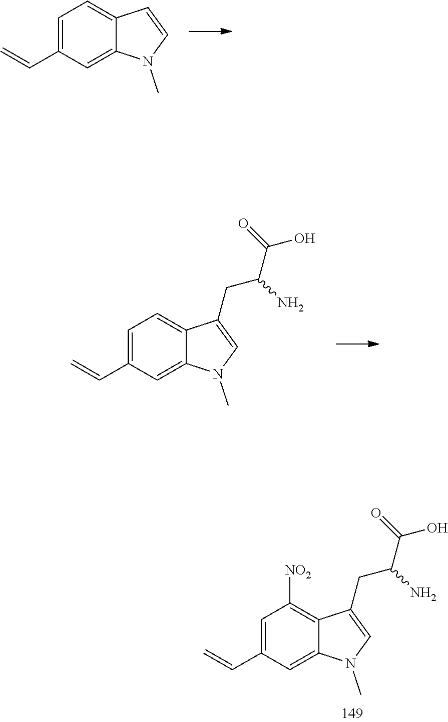 [SMILES: C=Cc1ccc2ccn(C)c2c1 ; C=Cc1ccc2c(CC(N)C(=O)O)cn(C)c2c1 ; C=Cc1cc([N+](=O)[O-])c2c(CC(N)C(=O)O)cn(C)c2c1]

Example 149 can be prepared from 6-vinyl-1-methyl-indole as shown above.

### Example 150: Preparation of 2-amino-3-(1-methyl-4-nitro-7-vinyl-1H-indol-3-yl)propanoic acid (150)

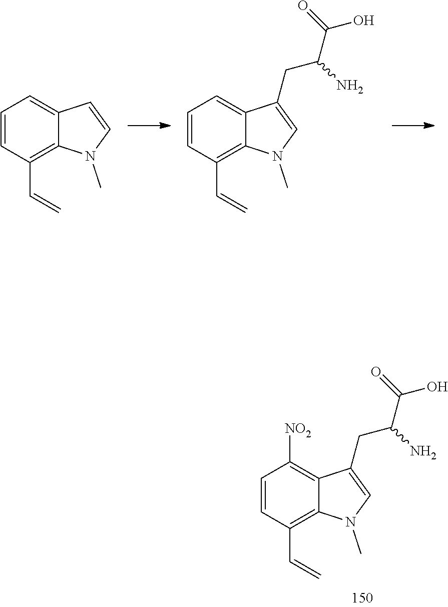 [SMILES: C=Cc1cccc2ccn(C)c12 ; C=Cc1cccc2c(CC(N)C(=O)O)cn(C)c12 ; C=Cc1ccc([N+](=O)[O-])c2c(CC(N)C(=O)O)cn(C)c12]

Example 150 can be prepared from 7-vinyl-1-methyl-indole as shown above.

### Example 151: Preparation of 2-amino-3-(1-methyl-7-nitro-4-vinyl-1H-indol-3-yl)propanoic acid (151)

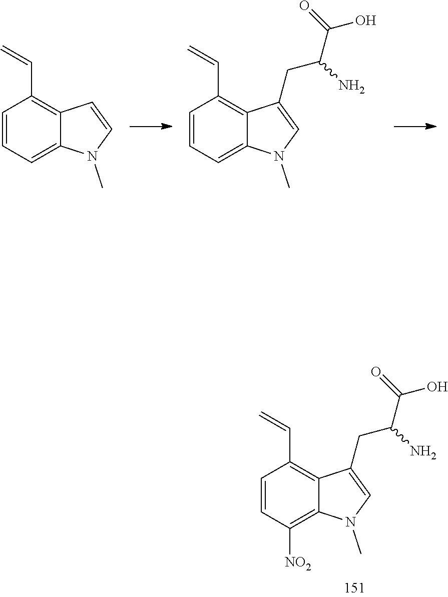 [SMILES: C=Cc1cccc2c1ccn2C ; C=Cc1cccc2c1c(CC(N)C(=O)O)cn2C ; C=Cc1ccc([N+](=O)[O-])c2c1c(CC(N)C(=O)O)cn2C]

Example 151 can be prepared from 4-vinyl-1-methyl-indole as shown above.

### Example 152: Preparation of 2-amino-3-(5-ethynyl-1-methyl-4-nitro-1H-indol-3-yl)propanoic acid (152)

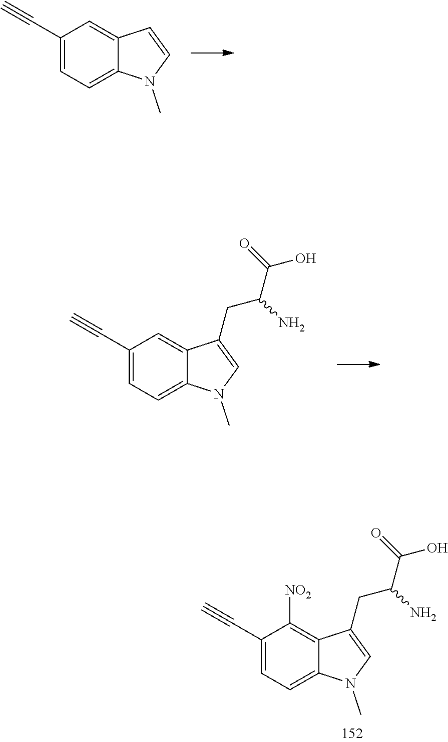 [SMILES: C#Cc1ccc2c(ccn2C)c1 ; C#Cc1ccc2c(c1)c(CC(N)C(=O)O)cn2C ; C#Cc1ccc2c(c(CC(N)C(=O)O)cn2C)c1[N+](=O)[O-]]

Example 152 can be prepared from 5-ethynyl-1-methyl-indole as shown above.

### Example 153: Preparation of 2-amino-3-(6-ethynyl-1-methyl-4-nitro-1H-indol-3-yl)propanoic acid (153)

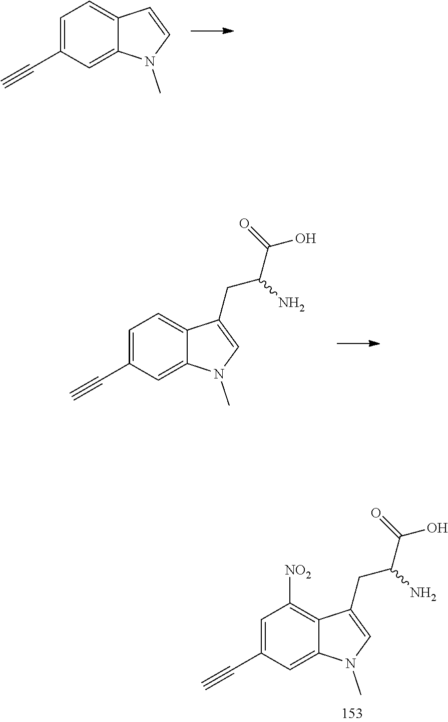 [SMILES: C#Cc1ccc2ccn(C)c2c1 ; C#Cc1ccc2c(CC(N)C(=O)O)cn(C)c2c1 ; C#Cc1cc([N+](=O)[O-])c2c(CC(N)C(=O)O)cn(C)c2c1]

Example 153 can be prepared from 6-ethynyl-1-methyl-indole as shown above.

### Example 154: Preparation of 2-amino-3-(7-ethynyl-1-methyl-4-nitro-1H-indol-3-yl)propanoic acid (154)

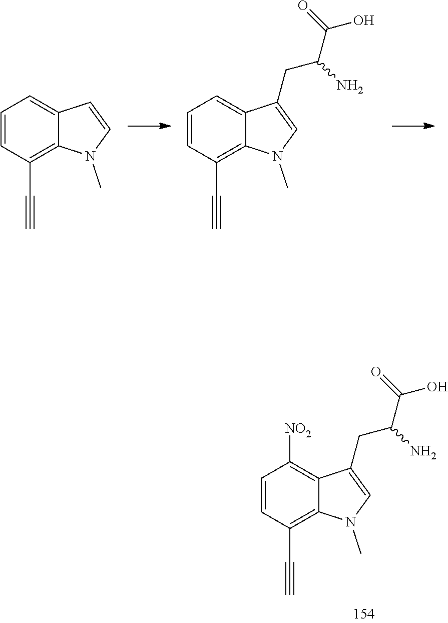 [SMILES: C#Cc1cccc2ccn(C)c12 ; C#Cc1cccc2c(CC(N)C(=O)O)cn(C)c12 ; C#Cc1ccc([N+](=O)[O-])c2c(CC(N)C(=O)O)cn(C)c12]

Example 154 can be prepared from 7-ethynyl-1-methyl-indole as shown above.

### Example 155: Preparation of 2-amino-3-(4-ethynyl-1-methyl-7-nitro-1H-indol-3-yl)propanoic acid (155)

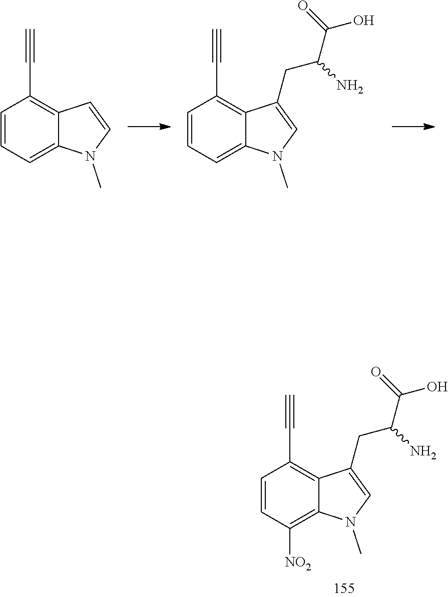 [SMILES: C#Cc1cccc2c1ccn2C ; C#Cc1cccc2c1c(CC(N)C(=O)O)cn2C ; C#Cc1ccc([N+](=O)[O-])c2c1c(CC(N)C(=O)O)cn2C]

Example 155 can be prepared from 4-ethynyl-J-methyl-indole as shown above.

### Example 156: Preparation of 2-amino-3-(1-methyl-5-morpholino-4-nitro-1H-indol-3-yl)propanoic acid (156)

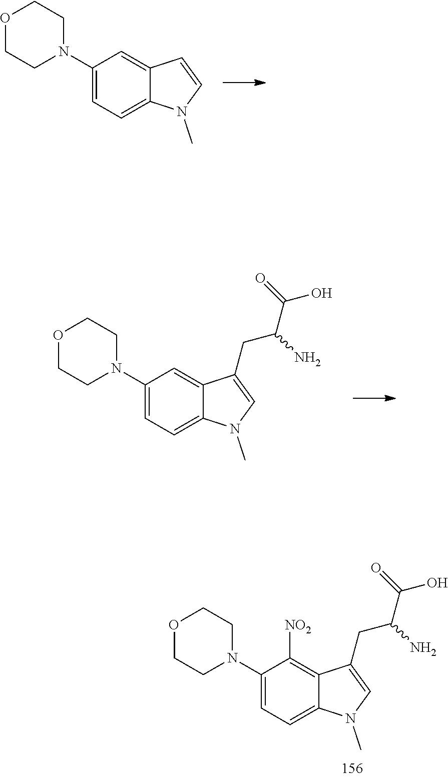 [SMILES: Cn1ccc2cc(N3CCOCC3)ccc21 ; Cn1cc(CC(N)C(=O)O)c2cc(N3CCOCC3)ccc21 ; Cn1cc(CC(N)C(=O)O)c2c([N+](=O)[O-])c(N3CCOCC3)ccc21]

Example 156 can be prepared from 5-morpholino-1-methyl-indole as shown above.

### Example 157: Preparation of 2-amino-3-(1-methyl-6-morpholino-4-nitro-1H-indol-3-yl)propanoic acid (157)

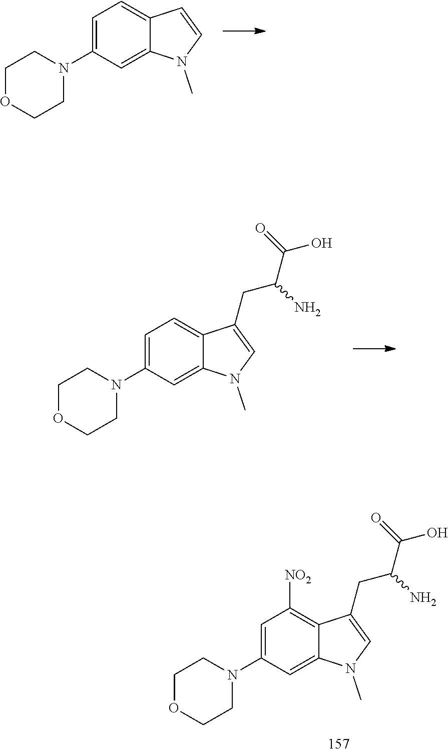 [SMILES: Cn1ccc2ccc(N3CCOCC3)cc21 ; Cn1cc(CC(N)C(=O)O)c2ccc(N3CCOCC3)cc21 ; Cn1cc(CC(N)C(=O)O)c2c([N+](=O)[O-])cc(N3CCOCC3)cc21]

Example 157 can be prepared from 6-morpholino-1-methyl-indole as shown above.

### Example 158: Preparation of 2-amino-3-(1-methyl-7-morpholino-4-nitro-1H-indol-3-yl)propanoic acid (158)

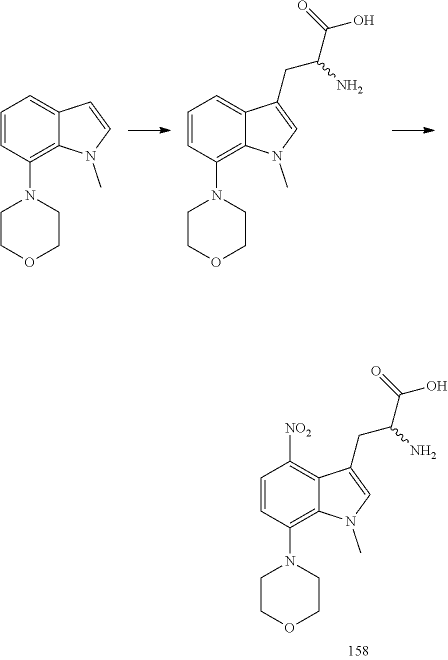 [SMILES: Cn1ccc2cccc(N3CCOCC3)c21 ; Cn1cc(CC(N)C(=O)O)c2cccc(N3CCOCC3)c21 ; Cn1cc(CC(N)C(=O)O)c2c([N+](=O)[O-])ccc(N3CCOCC3)c21]

Example 158 can be prepared from 7-morpholino-1-methyl-indole as shown above.

### Example 159: Preparation of 2-amino-3-(1-methyl-4-morpholino-7-nitro-1H-indol-3-yl)propanoic acid (159)

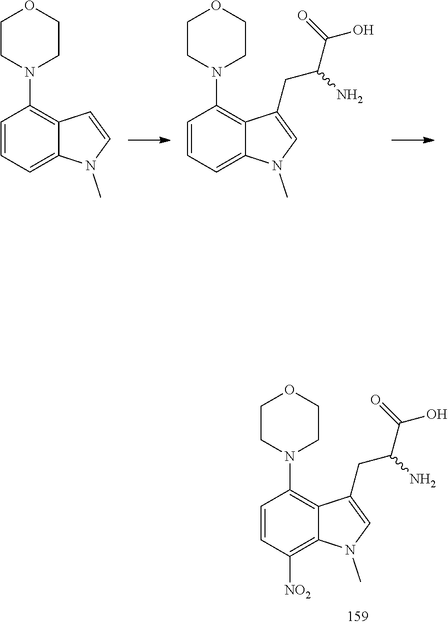 [SMILES: Cn1ccc2c(N3CCOCC3)cccc21 ; Cn1cc(CC(N)C(=O)O)c2c(N3CCOCC3)cccc21 ; Cn1cc(CC(N)C(=O)O)c2c(N3CCOCC3)ccc([N+](=O)[O-])c21]

Example 159 can be prepared from 4-morpholino-1-methyl-indole as shown above.

### Example 160: Preparation of 2-amino-3-(1-methyl-5-(methylthio)-4-nitro-1H-indol-3-yl)propanoic acid (160)

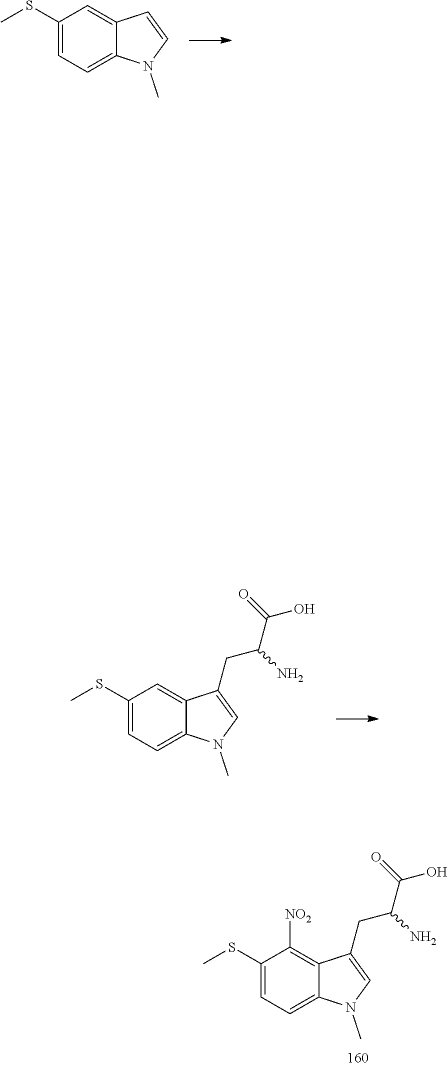 [SMILES: CSc1ccc2c(ccn2C)c1 ; CSc1ccc2c(c1)c(CC(N)C(=O)O)cn2C ; CSc1ccc2c(c(CC(N)C(=O)O)cn2C)c1[N+](=O)[O-]]

Example 160 can be prepared from 5-(methylthio)-1-methyl-indole as shown above.

### Example 161: Preparation of 2-amino-3-(1-methyl-6-(methylthio)-4-nitro-1H-indol-3-yl)propanoic acid (161)

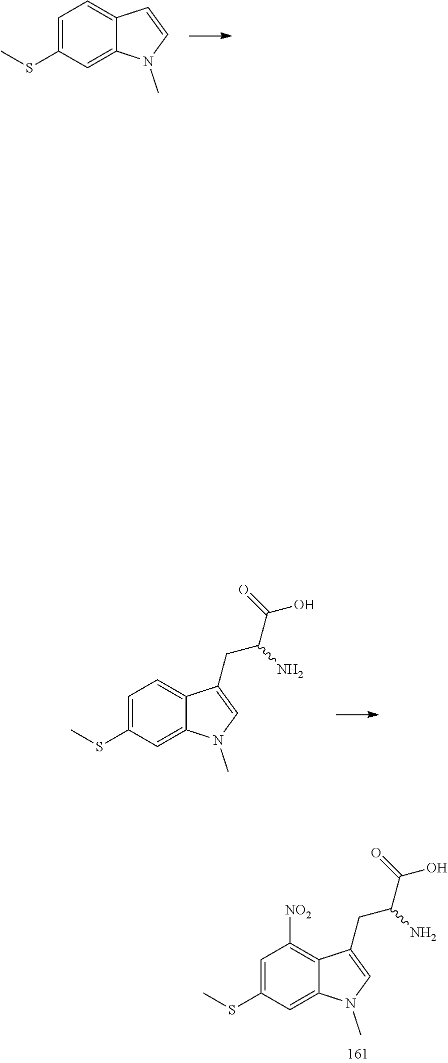 [SMILES: CSc1ccc2ccn(C)c2c1 ; CSc1ccc2c(CC(N)C(=O)O)cn(C)c2c1 ; CSc1cc([N+](=O)[O-])c2c(CC(N)C(=O)O)cn(C)c2c1]

Example 161 can be prepared from 6-(methylthio)-1-methyl-indole as shown above.

### Example 162: Preparation of 2-amino-3-(1-methyl-7-(methylthio)-4-nitro-1H-indol-3-yl)propanoic acid (162)

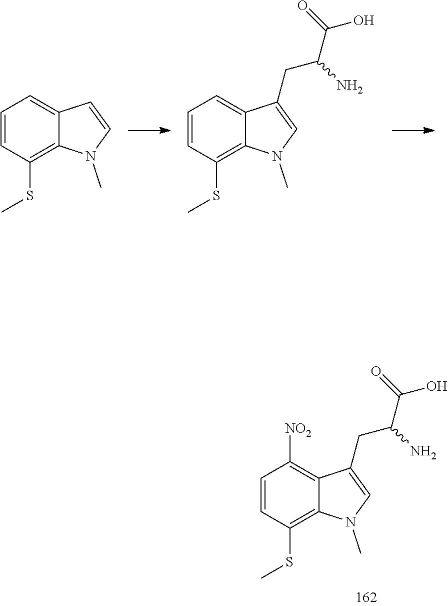 [SMILES: CSc1cccc2ccn(C)c12 ; CSc1cccc2c(CC(N)C(=O)O)cn(C)c12 ; CSc1ccc([N+](=O)[O-])c2c(CC(N)C(=O)O)cn(C)c12]

Example 162 can be prepared from 7-(methylthio)-1-methyl-indole as shown above.

### Example 163: Preparation of 2-amino-3-(1-methyl-4-(methylthio)-7-nitro-1H-indol-3-yl)propanoic acid (163)

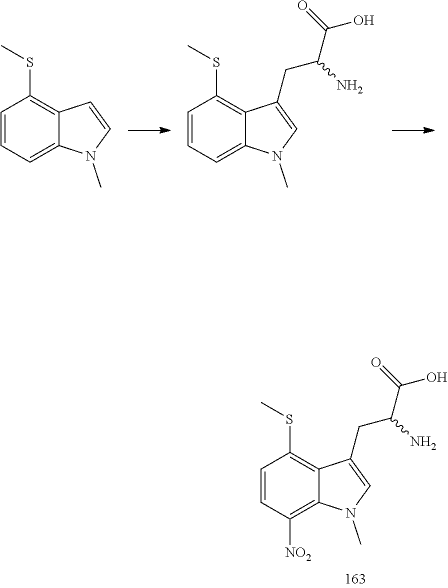 [SMILES: CSc1cccc2c1ccn2C ; CSc1cccc2c1c(CC(N)C(=O)O)cn2C ; CSc1ccc([N+](=O)[O-])c2c1c(CC(N)C(=O)O)cn2C]

Example 163 can be prepared from 4-(methylthio)-1-methyl-indole as shown above.

### Example 164: Preparation of 2-amino-3-(1-methyl-4-nitro-5-(pyridin-4-yl)-1H-indol-3-yl)propanoic acid (164)

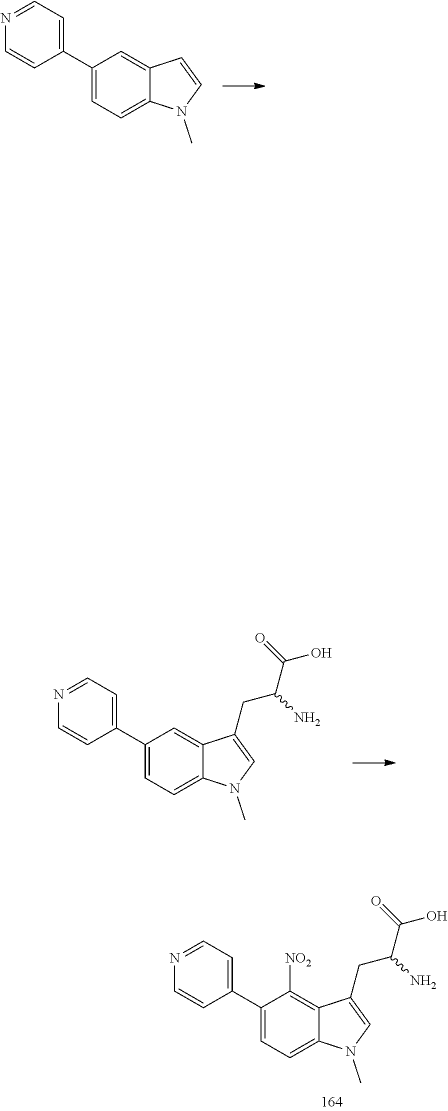 [SMILES: Cn1ccc2cc(-c3ccncc3)ccc21 ; Cn1cc(CC(N)C(=O)O)c2cc(-c3ccncc3)ccc21 ; Cn1cc(CC(N)C(=O)O)c2c([N+](=O)[O-])c(-c3ccncc3)ccc21]

Example 164 can be prepared from 5-(pyridin-4-yl)-1-methyl-indole as shown above.

### Example 165: Preparation of 2-amin-3-(1-methyl-4-nitro-6-(pyridin-4-yl)-1H-indol-3-yl)propanoic acid (165)

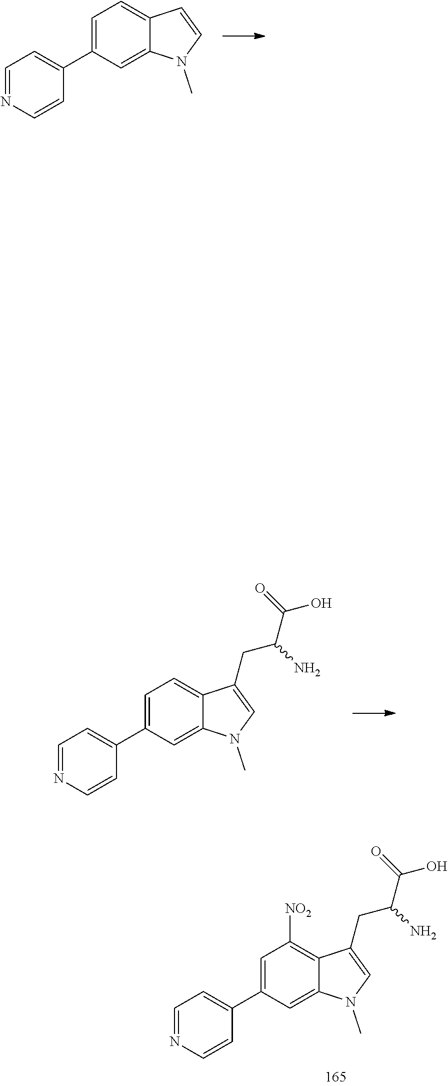 [SMILES: Cn1ccc2ccc(-c3ccncc3)cc21 ; Cn1cc(CC(N)C(=O)O)c2ccc(-c3ccncc3)cc21 ; Cn1cc(CC(N)C(=O)O)c2c([N+](=O)[O-])cc(-c3ccncc3)cc21]

Example 165 can be prepared from 6-(pyridin-4-yl)-1-methyl-indole as shown above.

### Example 166: Preparation of 2-amino-3-(1-methyl-4-nitro-7-(pyridin-4-yl)-1H-indol-3-yl)propanoic acid (166)

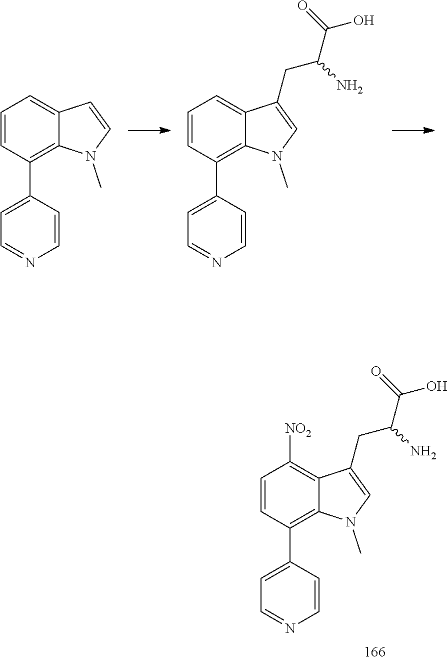 [SMILES: Cn1ccc2cccc(-c3ccncc3)c21 ; Cn1cc(CC(N)C(=O)O)c2cccc(-c3ccncc3)c21 ; Cn1cc(CC(N)C(=O)O)c2c([N+](=O)[O-])ccc(-c3ccncc3)c21]

Example 166 can be prepared from 7-(pyridin-4-yl)-1-methyl-indole as shown above.

### Example 167: Preparation of 2-amino-3-(1-methyl-7-nitro-4-(pyridin-4-yl)-1H-indol-3-yl)propionic acid (167)

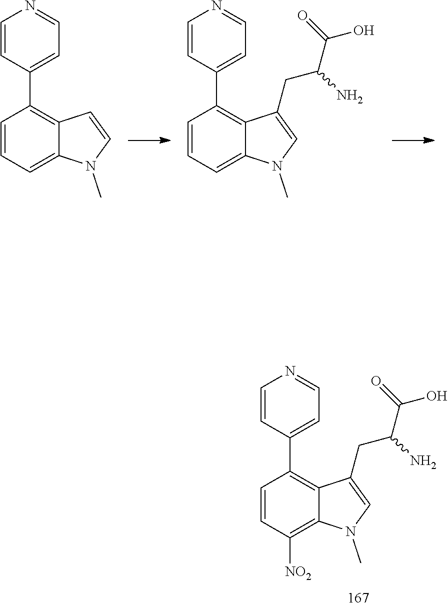 [SMILES: Cn1ccc2c(-c3ccncc3)cccc21 ; Cn1cc(CC(N)C(=O)O)c2c(-c3ccncc3)cccc21 ; Cn1cc(CC(N)C(=O)O)c2c(-c3ccncc3)ccc([N+](=O)[O-])c21]

Example 167 can be prepared from 4-(pyridin-4-yl)-1-methyl-indole as shown above.

### Example 168: Preparation of 2-amino-3-(2,5-dimethyl-4-nitro-1H-indol-3-yl)propanoic acid (168)

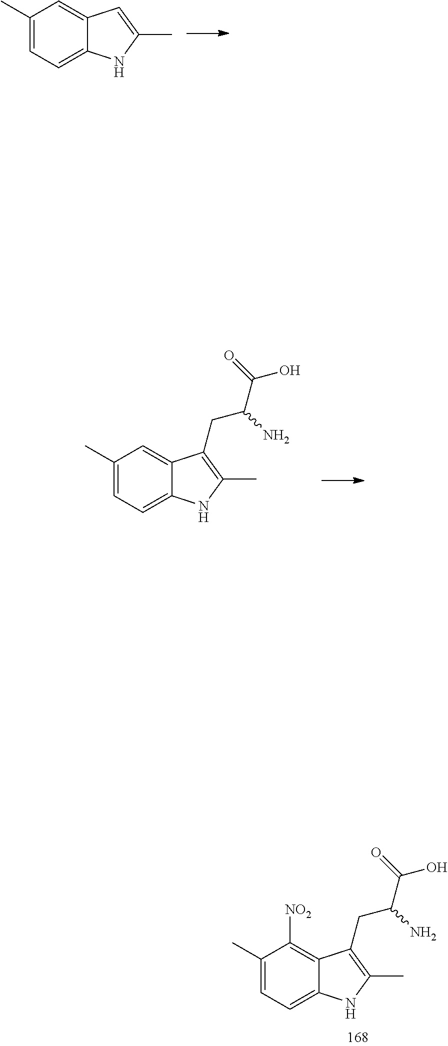

Example 168 can be prepared from 2,5-dimethyl-1H-indole as shown above.

### Example 169: Preparation of 2-amino-3-(2,6-dimethyl-4-nitro-1H-indol 3 yl)propanoic acid (169)

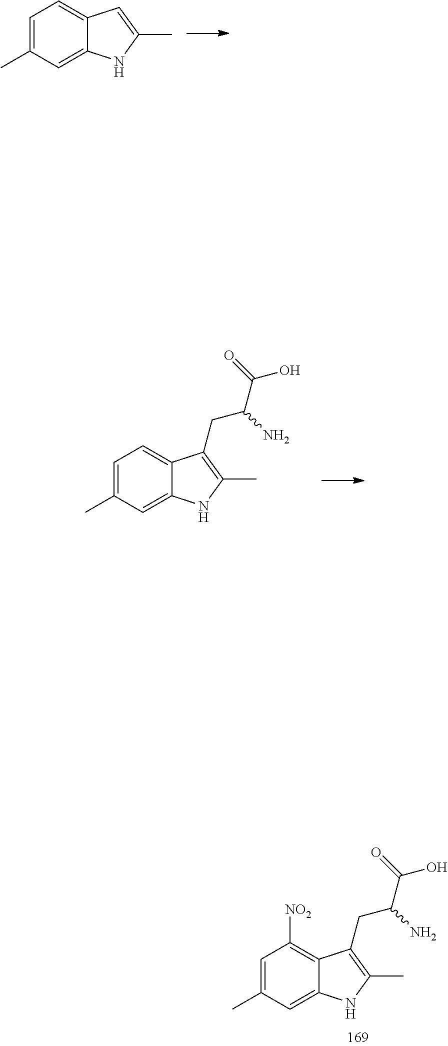

Example 169 can be prepared from 2,6-dimethyl-1H-indole as shown above.

### Example 170: Preparation of 2-wino-3-(2,7-dimethyl-4-nitro-1H-indol-3-yl)propanoic acid (170)

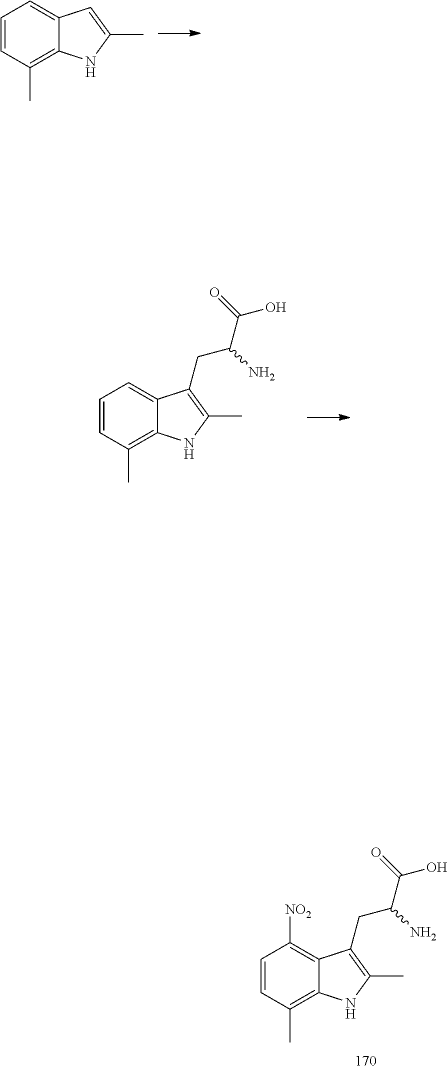 [SMILES: Cc1cc2cccc(C)c2[nH]1 ; Cc1[nH]c2c(C)cccc2c1CC(N)C(=O)O ; Cc1[nH]c2c(C)ccc([N+](=O)[O-])c2c1CC(N)C(=O)O]

Example 170 can be prepared from 2,7-dimethyl-1H-indole as shown above.

### Example 171: Preparation of 2-amino-3-(2,4-dimethyl-7-nitro-1H-indol-3-yl)propanoic acid (171)

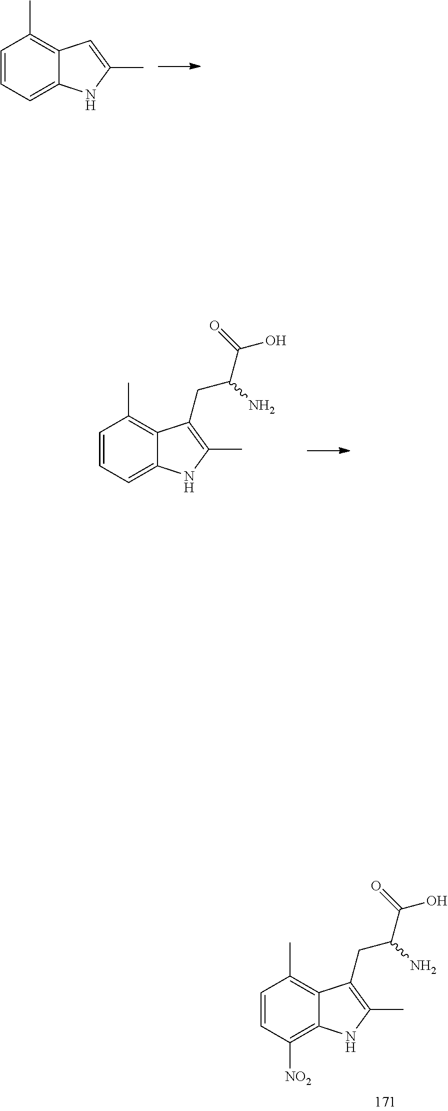 [SMILES: Cc1cc2c(C)cccc2[nH]1 ; Cc1[nH]c2cccc(C)c2c1CC(N)C(=O)O ; Cc1[nH]c2c([N+](=O)[O-])ccc(C)c2c1CC(N)C(=O)O]

Example 171 can be prepared from 2,4-dimethyl-1H-indole as shown above.

### Example 172: Preparation of 2-amino-3-(6-fluoro-2-methyl-4-nitro-1H-indol-3-yl)propanoic acid (172)

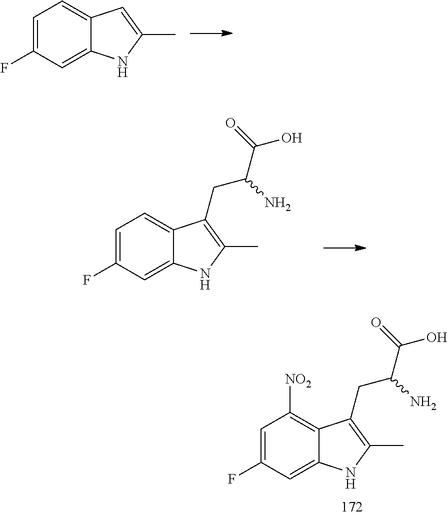 [SMILES: Cc1cc2ccc(F)cc2[nH]1 ; Cc1[nH]c2cc(F)ccc2c1CC(N)C(=O)O ; Cc1[nH]c2cc(F)cc([N+](=O)[O-])c2c1CC(N)C(=O)O]

Example 172 can be prepared from 6-fluoro-2-methyl-1H-indole as shown above.

### Example 173: Preparation of 2-amino-3-(7-fluoro-2-methyl-4-nitro-1H-indole-3-yl)propanoic acid (173)

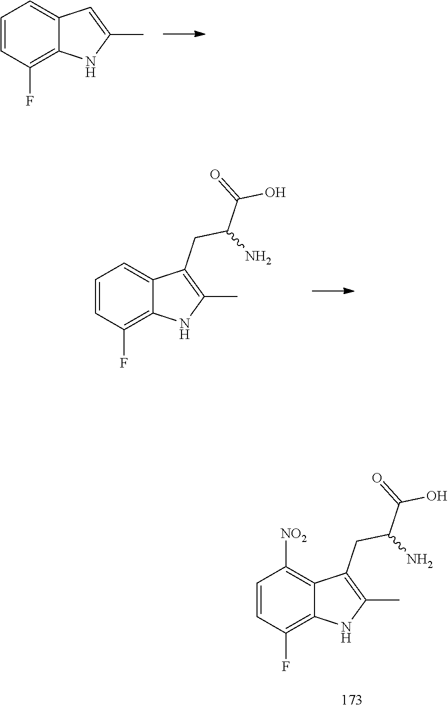 [SMILES: Cc1cc2cccc(F)c2[nH]1 ; Cc1[nH]c2c(F)cccc2c1CC(N)C(=O)O ; Cc1[nH]c2c(F)ccc([N+](=O)[O-])c2c1CC(N)C(=O)O]

Example 173 can be prepared from 7-fluoro-2-methyl-indole as shown above.

### Example 174: Preparation of 2-amino-3-(4-fluoro-2-methyl-7-nitro-1H-indole-3-yl)propanoic acid (174)

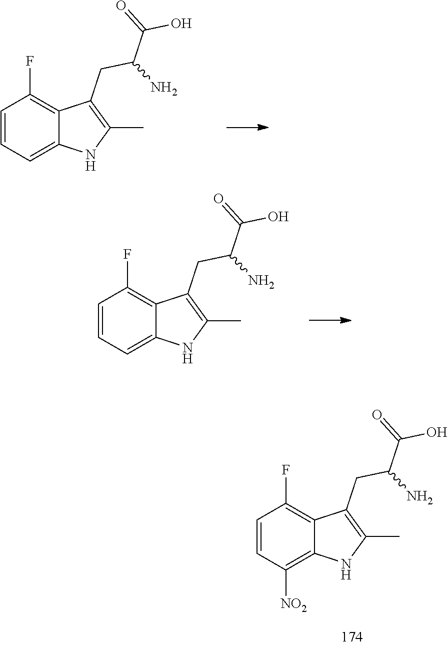 [SMILES: Cc1[nH]c2cccc(F)c2c1CC(N)C(=O)O ; Cc1[nH]c2cccc(F)c2c1CC(N)C(=O)O ; Cc1[nH]c2c([N+](=O)[O-])ccc(F)c2c1CC(N)C(=O)O]

Example 174 can be prepared from 4-fluoro-2-methyl-indole as shown above.

### Example 175: Preparation of 2-amino-3-(5-chloro-2-methyl-4-nitro-1H-indol-3-yl)propanoic acid (175)

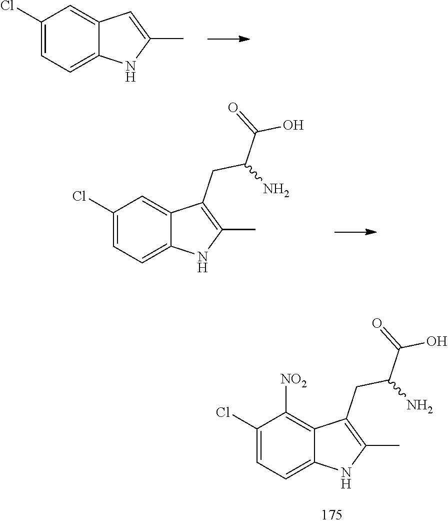 [SMILES: Cc1cc2cc(Cl)ccc2[nH]1 ; Cc1[nH]c2ccc(Cl)cc2c1CC(N)C(=O)O ; Cc1[nH]c2ccc(Cl)c([N+](=O)[O-])c2c1CC(N)C(=O)O]

Example 175 can be prepared from 5-chloro-2-methyl-indole as shown above.

### Example 176: Preparation of 2-amino-3-(6-chloro-2-methyl-4-nitro-1H-indol-3-yl)propanoic acid (176)

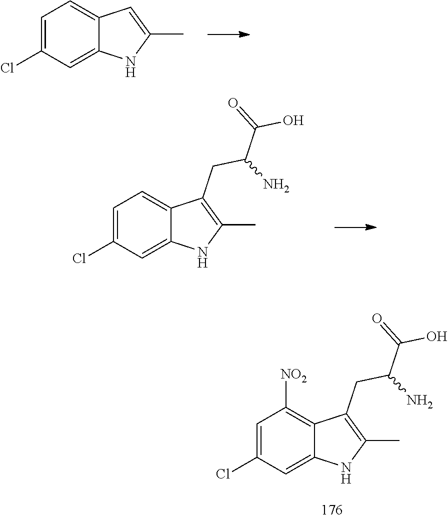 [SMILES: Cc1cc2ccc(Cl)cc2[nH]1 ; Cc1[nH]c2cc(Cl)ccc2c1CC(N)C(=O)O ; Cc1[nH]c2cc(Cl)cc([N+](=O)[O-])c2c1CC(N)C(=O)O]

Example 176 can be prepared from 6-chloro-2-methyl-indole as shown above.

### Example 177: Preparation of 2-amino-3-(7-chloro-2-methyl-4-nitro-1H-indol-3-yl)propanoic acid (177)

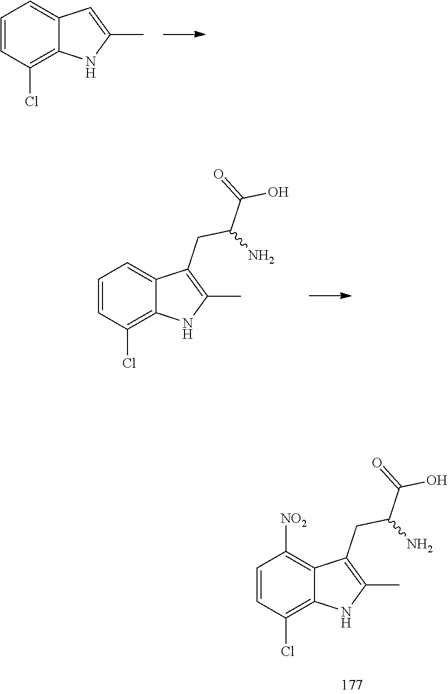 [SMILES: Cc1cc2cccc(Cl)c2[nH]1 ; Cc1[nH]c2c(Cl)cccc2c1CC(N)C(=O)O ; Cc1[nH]c2c(Cl)ccc([N+](=O)[O-])c2c1CC(N)C(=O)O]

Example 177 can be prepared from 7-chloro-2-methyl-indole as shown above.

### Example 178: Preparation of 2-amino-3-(4-chloro-2-methyl-7-nitro-1H-indol-3-yl)propanoic acid (178)

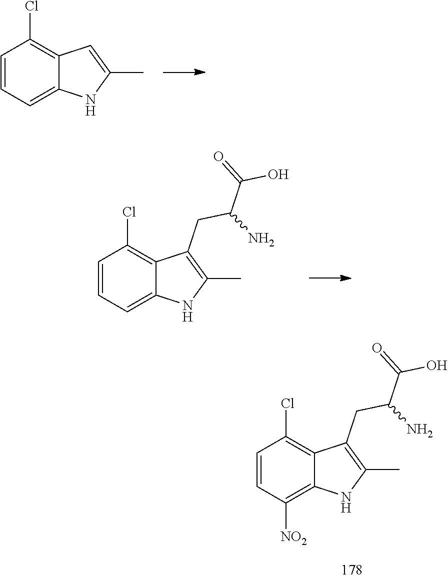 [SMILES: Cc1cc2c(Cl)cccc2[nH]1 ; Cc1[nH]c2cccc(Cl)c2c1CC(N)C(=O)O ; Cc1[nH]c2c([N+](=O)[O-])ccc(Cl)c2c1CC(N)C(=O)O]

Example 178 can be prepared from 4-chloro-2-methyl-indole as shown above.

### Example 179: Preparation of 2-amino-3-(5-bromo-2-methyl-4-nitro-1H-indol-3-yl)propanoic acid (179)

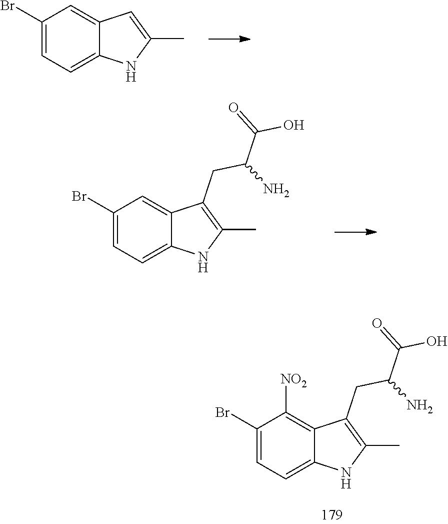 [SMILES: Cc1cc2cc(Br)ccc2[nH]1 ; Cc1[nH]c2ccc(Br)cc2c1CC(N)C(=O)O ; Cc1[nH]c2ccc(Br)c([N+](=O)[O-])c2c1CC(N)C(=O)O]

Example 179 can be prepared from 5-bromo-2-methyl-indole as shown above.

### Example 180: Preparation of 2-amino-3-(6-bromo-2-methyl-4-nitro-1H-indol-3-yl)propanoic acid (180)

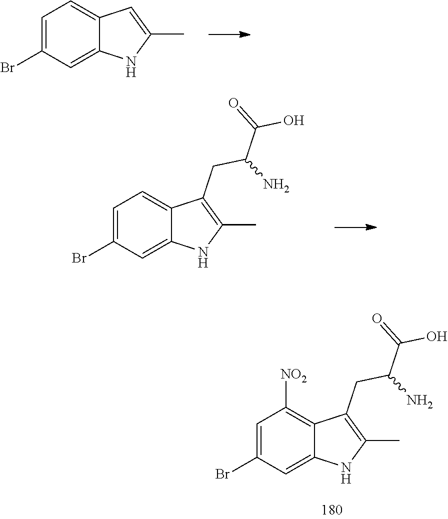 [SMILES: Cc1cc2ccc(Br)cc2[nH]1 ; Cc1[nH]c2cc(Br)ccc2c1CC(N)C(=O)O ; Cc1[nH]c2cc(Br)cc([N+](=O)[O-])c2c1CC(N)C(=O)O]

Example 180 can be prepared from 6-bromo-2-methyl-indole as shown above.

### Example 181: Preparation of 2-amino-3-(7-bromo-2-methyl-4-nitro-1H-indol-3-yl)propanoic acid (191)

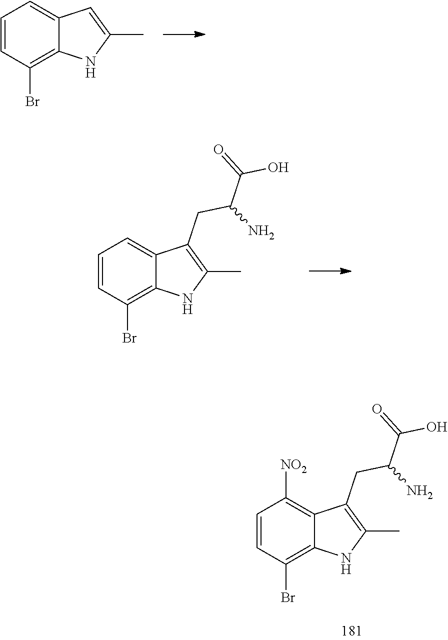 [SMILES: Cc1cc2cccc(Br)c2[nH]1 ; Cc1[nH]c2c(Br)cccc2c1CC(N)C(=O)O ; Cc1[nH]c2c(Br)ccc([N+](=O)[O-])c2c1CC(N)C(=O)O]

Example 181 can be prepared from 7-bromo-2-methyl-indole as shown above.

### Example 182: Preparation of 2-amino-3-(4-bromo-2-methyl-7-nitro-1H-indol-3-yl)propanoic acid (182)

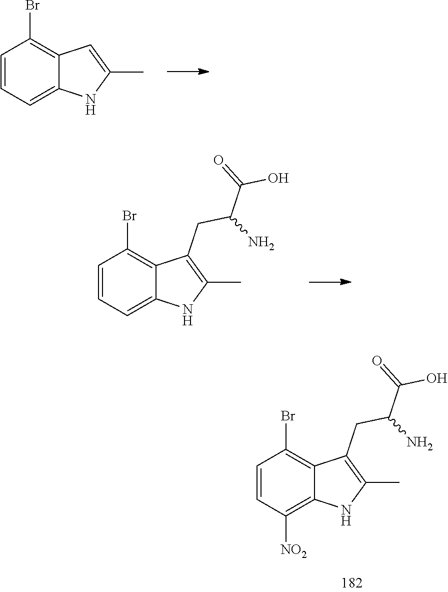 [SMILES: Cc1cc2c(Br)cccc2[nH]1 ; Cc1[nH]c2cccc(Br)c2c1CC(N)C(=O)O ; Cc1[nH]c2c([N+](=O)[O-])ccc(Br)c2c1CC(N)C(=O)O]

Example 182 can be prepared from 4-bromo-2-methyl-indole as shown above.

### Example 183: Preparation of 2-amino-3-(5-methoxy-2-methyl-4-nitro-1H-indol-3-yl)propanoic acid (183)

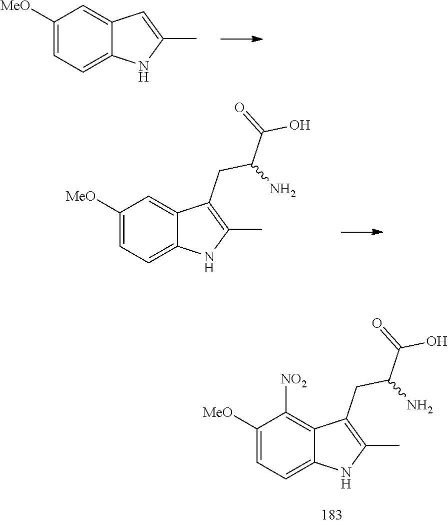 [SMILES: COc1ccc2[nH]c(C)cc2c1 ; COc1ccc2[nH]c(C)c(CC(N)C(=O)O)c2c1 ; COc1ccc2[nH]c(C)c(CC(N)C(=O)O)c2c1[N+](=O)[O-]]

Example 183 can be prepared from 5-methoxy-2-methyl-indole as shown above.

### Example 184: Preparation of 2-amino-3-(6-methoxy-2-methyl-4-nitro-1H-indel-3-yl)propanoic acid (184)

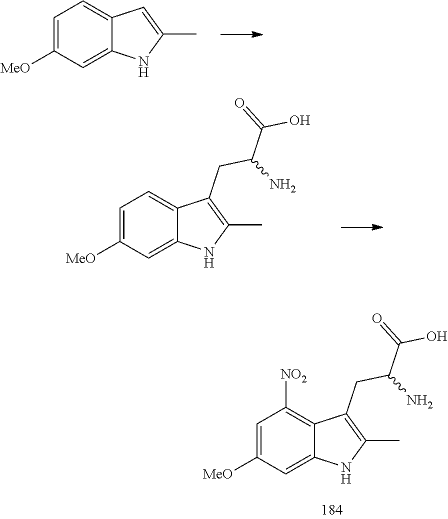 [SMILES: COc1ccc2cc(C)[nH]c2c1 ; COc1ccc2c(CC(N)C(=O)O)c(C)[nH]c2c1 ; COc1cc([N+](=O)[O-])c2c(CC(N)C(=O)O)c(C)[nH]c2c1]

Example 184 can be prepared from 6-methoxy-2-methyl-indole as shown above.

### Example 185: Preparation of 2-amino-3-(7-methoxy-2-methyl-4-nitro-1H-indol-3-yl)propanoic acid (185)

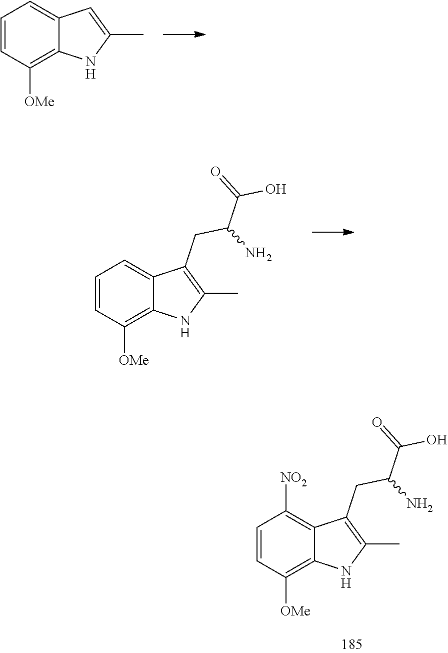 [SMILES: COc1cccc2cc(C)[nH]c12 ; COc1cccc2c(CC(N)C(=O)O)c(C)[nH]c12 ; COc1ccc([N+](=O)[O-])c2c(CC(N)C(=O)O)c(C)[nH]c12]

Example 185 can be prepared from 7-methoxy-2-methyl-indole as shown above.

### Example 186: Preparation of 2-amino-3-(4-methoxy-2-methyl-7-nitro-1H-indol-3-yl)propanoic acid (186)

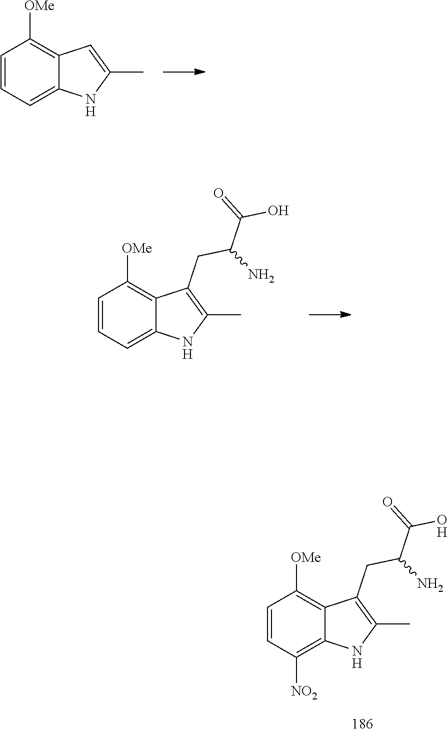 [SMILES: COc1cccc2[nH]c(C)cc12 ; COc1cccc2[nH]c(C)c(CC(N)C(=O)O)c12 ; COc1ccc([N+](=O)[O-])c2[nH]c(C)c(CC(N)C(=O)O)c12]

Example 186 can be prepared from 4-methoxy-2-methyl-indole as shown above.

### Example 187: Preparation of 2-amino-3-(5-amino-2-methyl-4-nitro-1H-indol-3-yl)propanoic acid (187)

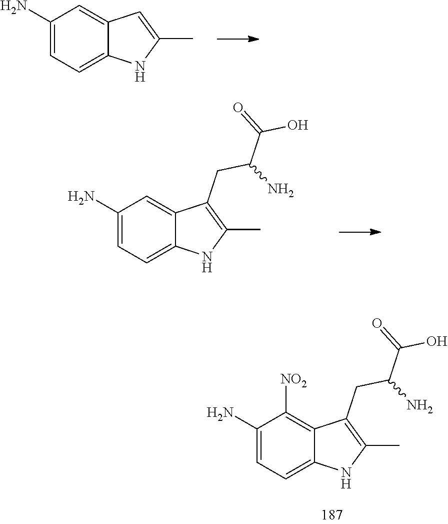 [SMILES: Cc1cc2cc(N)ccc2[nH]1 ; Cc1[nH]c2ccc(N)cc2c1CC(N)C(=O)O ; Cc1[nH]c2ccc(N)c([N+](=O)[O-])c2c1CC(N)C(=O)O]

Example 187 can be prepared from 5-amino-2-methyl-indole as shown above.

### Example 188: Preparation of 2-amino-3-(6-amino-2-methyl-4-nitro-1H-indol-3-yl)propanoic acid (188)

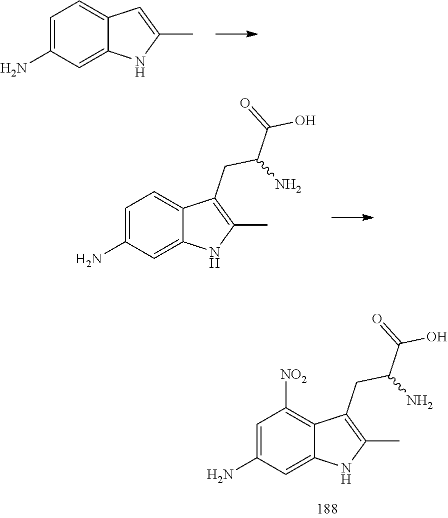 [SMILES: Cc1cc2ccc(N)cc2[nH]1 ; Cc1[nH]c2cc(N)ccc2c1CC(N)C(=O)O ; Cc1[nH]c2cc(N)cc([N+](=O)[O-])c2c1CC(N)C(=O)O]

Example 188 can be prepared from 6-amino-2-methyl-indole as shown above.

### Example 189: Preparation of 2-amino-3-(7-amino-2-methyl-4-nitro-1H-indol-3-yl)propanoic acid (189)

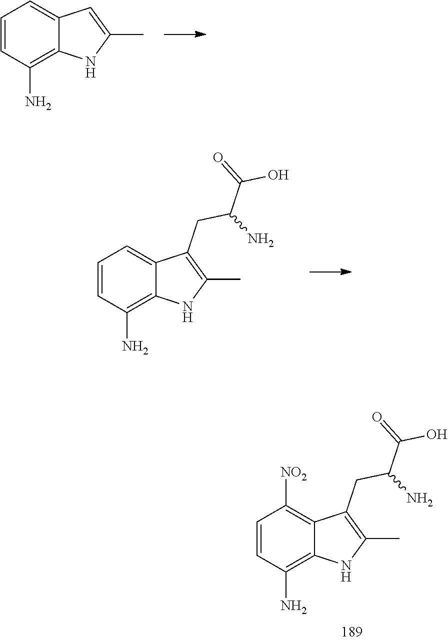 [SMILES: Cc1cc2cccc(N)c2[nH]1 ; Cc1[nH]c2c(N)cccc2c1CC(N)C(=O)O ; Cc1[nH]c2c(N)ccc([N+](=O)[O-])c2c1CC(N)C(=O)O]

Example 189 can be prepared from 7-amino-2-methyl-indole as shown above.

### Example 190: Preparation of 2-amino-3-(4-amino-2-methyl-7-nitro-1H-indol-3-yl)propanoic acid (190)

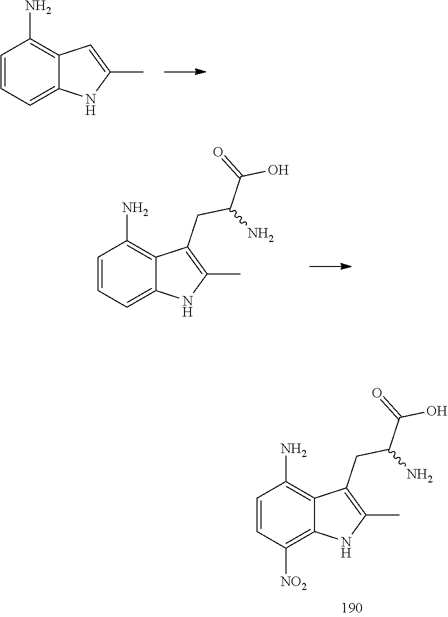 [SMILES: Cc1cc2c(N)cccc2[nH]1 ; Cc1[nH]c2cccc(N)c2c1CC(N)C(=O)O ; Cc1[nH]c2c([N+](=O)[O-])ccc(N)c2c1CC(N)C(=O)O]

Example 190 can be prepared from 4-amino-2-methyl-indole as shown above.

### Example 191: Preparation of 2-amino-3-(5-hydroxy-2-methyl-4-nitro-1H-Indol-3-yl)propanoic acid (191)

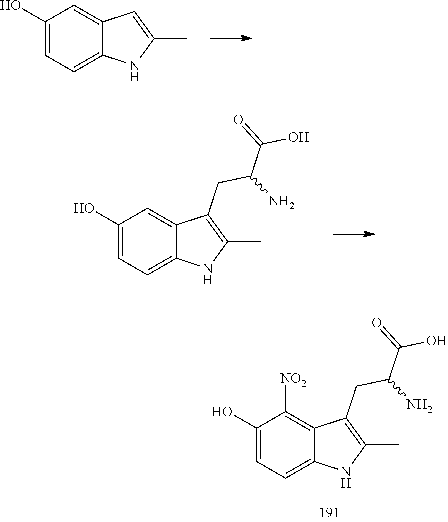 [SMILES: Cc1cc2cc(O)ccc2[nH]1 ; Cc1[nH]c2ccc(O)cc2c1CC(N)C(=O)O ; Cc1[nH]c2ccc(O)c([N+](=O)[O-])c2c1CC(N)C(=O)O]

Example 191 can be prepared from 5-hydroxy-2-methyl-indole as shown above.

### Example 192: Preparation of 2-amino-3-(6-hydroxy-2-methyl-4-nitro-1H-indol-3-yl)propanoic acid (192)

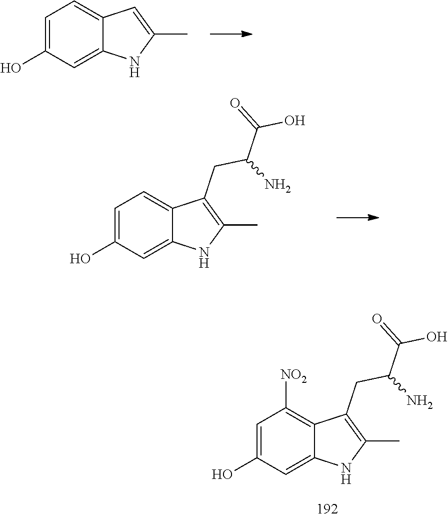 [SMILES: Cc1cc2ccc(O)cc2[nH]1 ; Cc1[nH]c2cc(O)ccc2c1CC(N)C(=O)O ; Cc1[nH]c2cc(O)cc([N+](=O)[O-])c2c1CC(N)C(=O)O]

Example 192 can be prepared from 6-hydroxy-2-methyl-indole as shown above.

### Example 193: Preparation of 2-amino-3-(7-hydroxy-2-methyl-4-nitro-1H-indol-3-yl)propanoic acid (193)

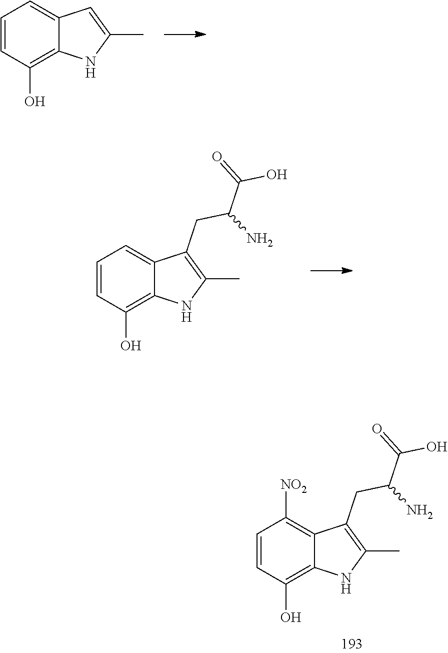 [SMILES: Cc1cc2cccc(O)c2[nH]1 ; Cc1[nH]c2c(O)cccc2c1CC(N)C(=O)O ; Cc1[nH]c2c(O)ccc([N+](=O)[O-])c2c1CC(N)C(=O)O]

Example 193 can be prepared from 7-hydroxy-2-methyl-indole as shown above.

### Example 194: Preparation of 2-amino-3-(4-hydroxy-2-methyl-7-nitro-1H-indol-3-yl)propanoic acid (194)

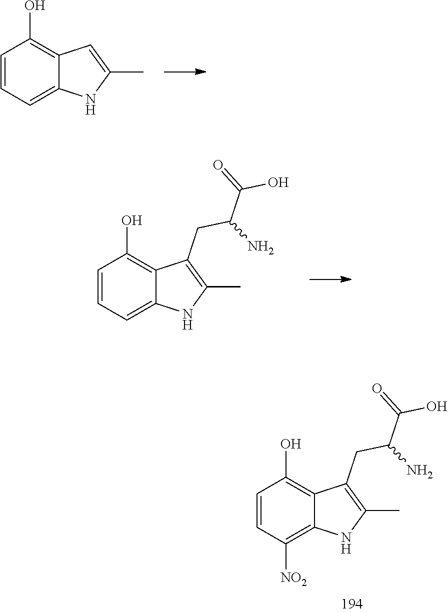 [SMILES: Cc1cc2c(O)cccc2[nH]1 ; Cc1[nH]c2cccc(O)c2c1CC(N)C(=O)O ; Cc1[nH]c2c([N+](=O)[O-])ccc(O)c2c1CC(N)C(=O)O]

Example 194 can be prepared from 4-hydroxy-2-methyl-indole as shown above.

### Example 195: Preparation of 2-amino-3-(2-methyl-4-nitro-5-phenyl-1H-indol-3-yl)propanoic acid (195)

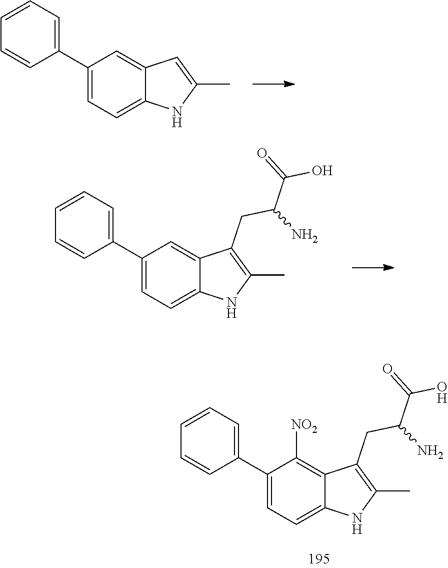 [SMILES: Cc1cc2cc(-c3ccccc3)ccc2[nH]1 ; Cc1[nH]c2ccc(-c3ccccc3)cc2c1CC(N)C(=O)O ; Cc1[nH]c2ccc(-c3ccccc3)c([N+](=O)[O-])c2c1CC(N)C(=O)O]

Example 195 can be prepared from 5-phenyl-2-methyl-indole as shown above.

### Example 196: Preparation of 2-amino-3-(2-methyl-4-nitro-6-phenyl-1H-indol-3-yl)propanoic acid (196)

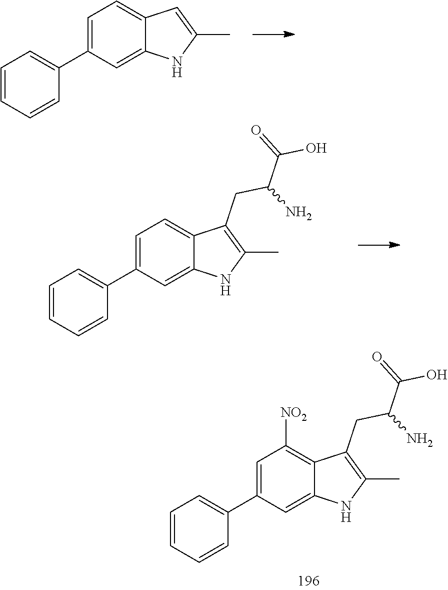 [SMILES: Cc1cc2ccc(-c3ccccc3)cc2[nH]1 ; Cc1[nH]c2cc(-c3ccccc3)ccc2c1CC(N)C(=O)O ; Cc1[nH]c2cc(-c3ccccc3)cc([N+](=O)[O-])c2c1CC(N)C(=O)O]

Example 196 can be prepared from 6-phenyl-2-methyl-indole as shown above.

### Example 197: Preparation of 2-amino-3-(2-methyl-4-nitro-7-phenyl-1H-indol-3-yl)propanoic acid (197)

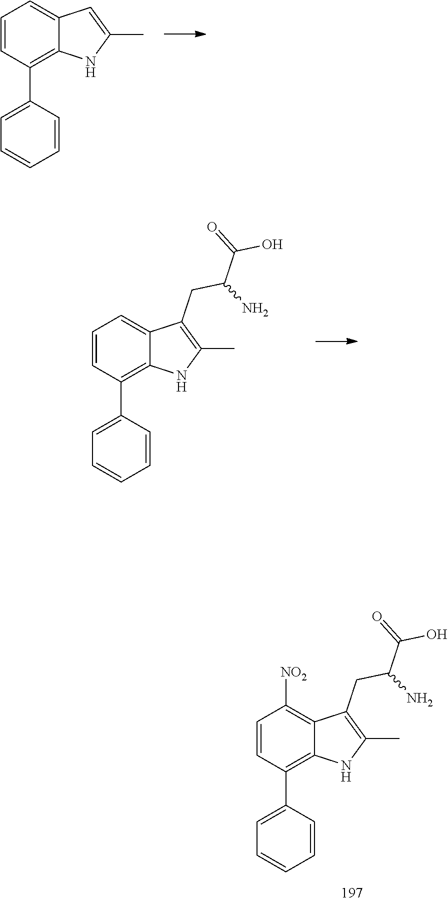 [SMILES: Cc1cc2cccc(-c3ccccc3)c2[nH]1 ; Cc1[nH]c2c(-c3ccccc3)cccc2c1CC(N)C(=O)O ; Cc1[nH]c2c(-c3ccccc3)ccc([N+](=O)[O-])c2c1CC(N)C(=O)O]

Example 197 can be prepared from 7-phenyl-2-methyl-indole as shown above.

### Example 198: Preparation of 2-amino-3-(2-methyl-7-nitro-4-phenyl-1H-indol-3-yl)propanoic acid (19)

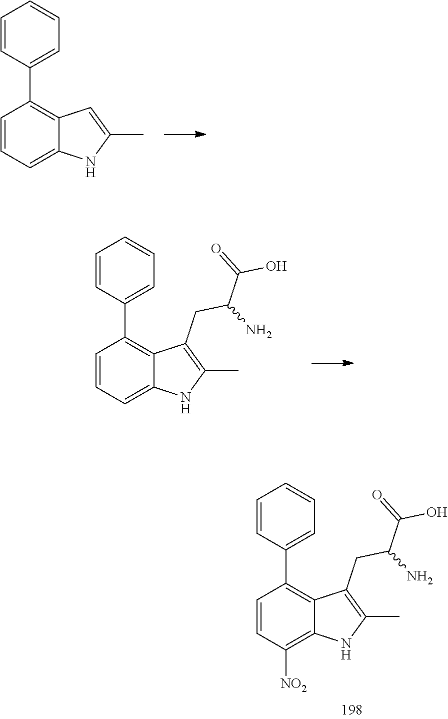 [SMILES: Cc1cc2c(-c3ccccc3)cccc2[nH]1 ; Cc1[nH]c2cccc(-c3ccccc3)c2c1CC(N)C(=O)O ; Cc1[nH]c2c([N+](=O)[O-])ccc(-c3ccccc3)c2c1CC(N)C(=O)O]

Example 198 can be prepared from 4-phenyl-2-methyl-indole as shown above.

### Example 199: Preparation of 2-amino-3-(5-cyclopropyl-2-methyl-4-nitro-1H-indol-3-yl)propanoic acid (199)

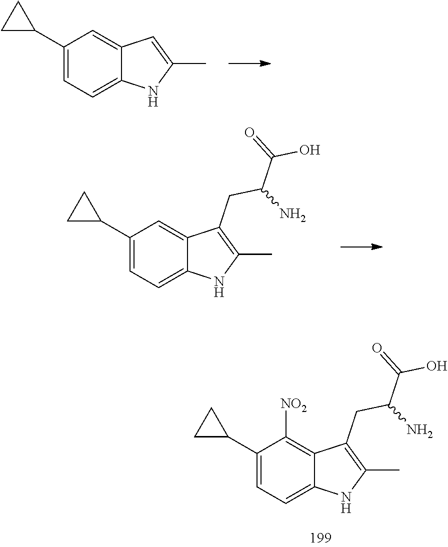 [SMILES: Cc1cc2cc(C3CC3)ccc2[nH]1 ; Cc1[nH]c2ccc(C3CC3)cc2c1CC(N)C(=O)O ; Cc1[nH]c2ccc(C3CC3)c([N+](=O)[O-])c2c1CC(N)C(=O)O]

Example 199 can be prepared from 5-cyclopropyl-2-methyl-indole as shown above.

### Example 200: Preparation of 2-amino-3-(6-cyclopropyl-2-methyl-4-nitro-1H-indol-3-yl)propanoic acid (200)

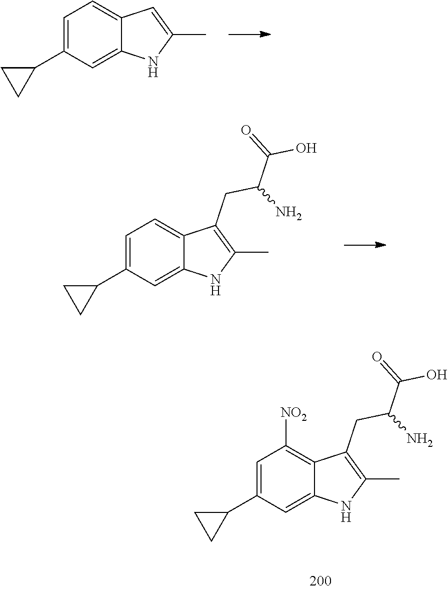 [SMILES: Cc1cc2ccc(C3CC3)cc2[nH]1 ; Cc1[nH]c2cc(C3CC3)ccc2c1CC(N)C(=O)O ; Cc1[nH]c2cc(C3CC3)cc([N+](=O)[O-])c2c1CC(N)C(=O)O]

Example 200 can be prepared from 6-cyclopropyl-2-methyl-indole as shown above.

### Example 201: Preparation of 2-amino-3-(7-cyclopropyl-2-methyl-4-nitro-1H-indol-3-yl)propanoic acid (201)

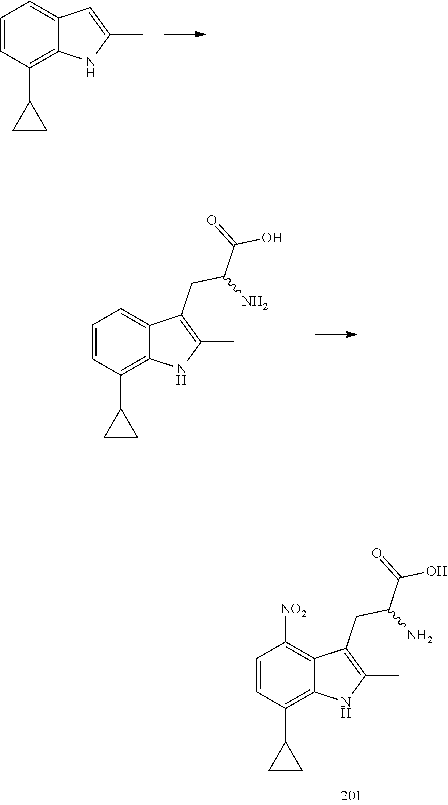 [SMILES: Cc1cc2cccc(C3CC3)c2[nH]1 ; Cc1[nH]c2c(C3CC3)cccc2c1CC(N)C(=O)O ; Cc1[nH]c2c(C3CC3)ccc([N+](=O)[O-])c2c1CC(N)C(=O)O]

Example 201 can be prepared from 7-cyclopropyl-2-methyl-indole as shown above.

### Example 202: Preparation of 2-amino-3-(4-cyclopropyl-2-methyl-7-nitro-IN-indol-3-yl)propanoic acid (202)

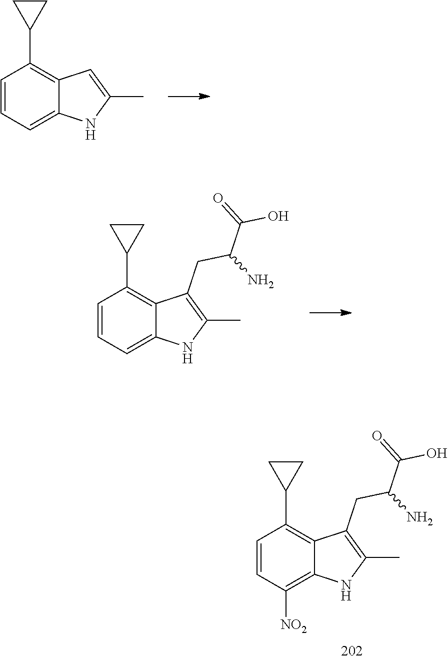 [SMILES: Cc1cc2c(C3CC3)cccc2[nH]1 ; Cc1[nH]c2cccc(C3CC3)c2c1CC(N)C(=O)O ; Cc1[nH]c2c([N+](=O)[O-])ccc(C3CC3)c2c1CC(N)C(=O)O]

Example 202 can be prepared from 4-cyclopropyl-2-methyl-indole as shown above.

### Example 203: Preparation of 2-amino-3-(2-methyl-4-nitro-5-vinyl-1HR-indol-3-yl)propanoic acid (203)

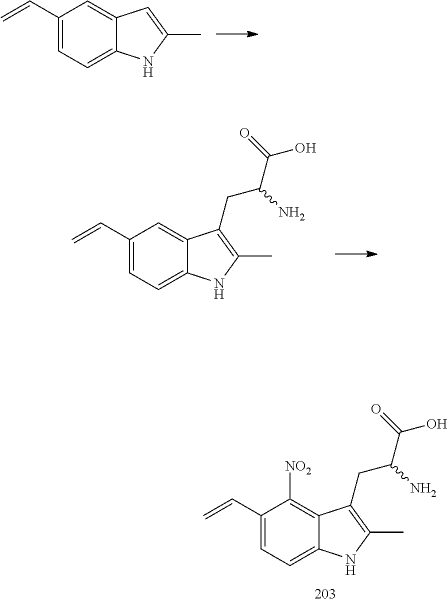 [SMILES: C=Cc1ccc2[nH]c(C)cc2c1 ; C=Cc1ccc2[nH]c(C)c(CC(N)C(=O)O)c2c1 ; C=Cc1ccc2[nH]c(C)c(CC(N)C(=O)O)c2c1[N+](=O)[O-]]

Example 203 can be prepared from 5-vinyl-2-methyl-indole as shown above.

### Example 204: Preparation of 2-amino-3-(2-methyl-4-nitro-6-vinyl-1H-indol-3-yl)propanoic acid (204)

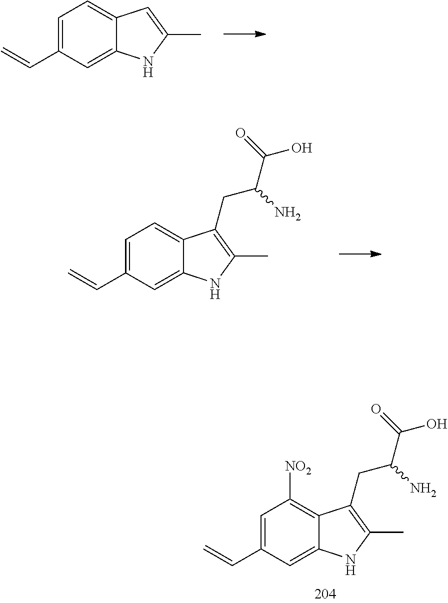 [SMILES: C=Cc1ccc2cc(C)[nH]c2c1 ; C=Cc1ccc2c(CC(N)C(=O)O)c(C)[nH]c2c1 ; C=Cc1cc([N+](=O)[O-])c2c(CC(N)C(=O)O)c(C)[nH]c2c1]

Example 204 can be prepared from 6-vinyl-2-methyl-indole as shown above.

### Example 205: Preparation of 2-amino-3-(2-methyl-4-nitro-7-vinyl-1H-indol-3-yl)propanoic acid (205)

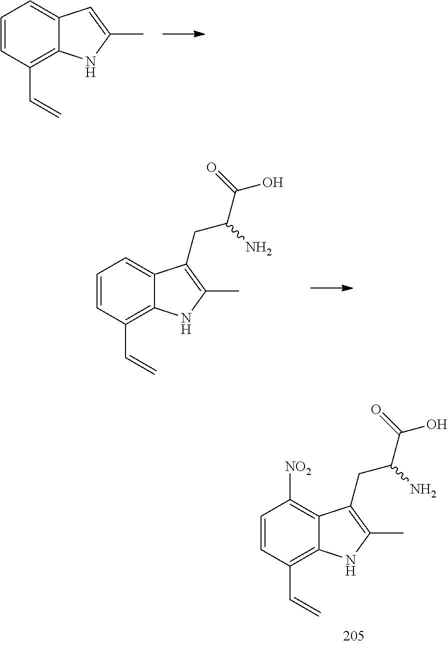 [SMILES: C=Cc1cccc2cc(C)[nH]c12 ; C=Cc1cccc2c(CC(N)C(=O)O)c(C)[nH]c12 ; C=Cc1ccc([N+](=O)[O-])c2c(CC(N)C(=O)O)c(C)[nH]c12]

Example 205 can be prepared from 7-vinyl-2-methyl-indole as shown above.

### Example 206: Preparation of 2-amino-3-(2-methyl-7-nitro-4-vinyl-1H-indol-3-yl)propanoic acid (206)

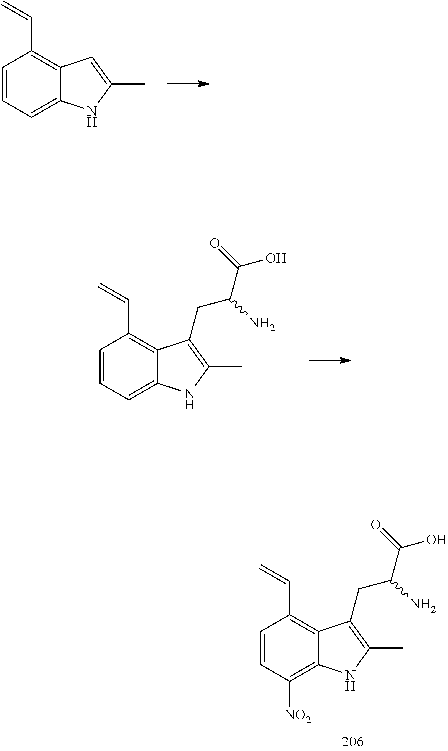 [SMILES: C=Cc1cccc2[nH]c(C)cc12 ; C=Cc1cccc2[nH]c(C)c(CC(N)C(=O)O)c12 ; C=Cc1ccc([N+](=O)[O-])c2[nH]c(C)c(CC(N)C(=O)O)c12]

Example 206 can be prepared from 4-vinyl-2-methyl-indole as shown above.

### Example 207: Preparation of 2-amino-3-(5-ethynyl-2-methyl-4-nitro-1H-indol-3-yl)propanoic acid (207)

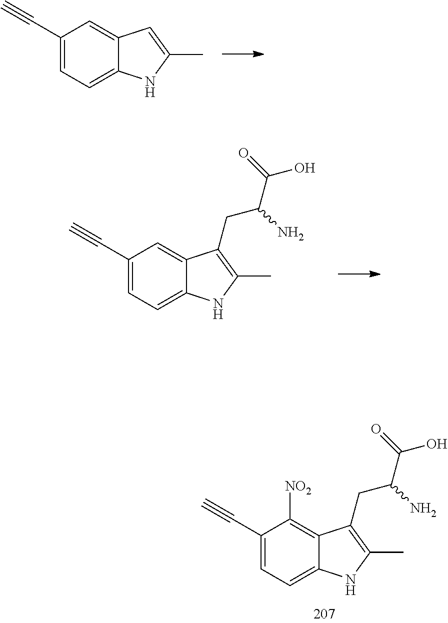 [SMILES: C#Cc1ccc2[nH]c(C)cc2c1 ; C#Cc1ccc2[nH]c(C)c(CC(N)C(=O)O)c2c1 ; C#Cc1ccc2[nH]c(C)c(CC(N)C(=O)O)c2c1[N+](=O)[O-]]

Example 207 can be prepared from 5-ethynyl-2-methyl-indole as shown above.

### Example 208: Preparation of 2-amino-3-(6-ethynyl-2-methyl-4-nitro-1H-indol-3-yl)propanoic acid (208)

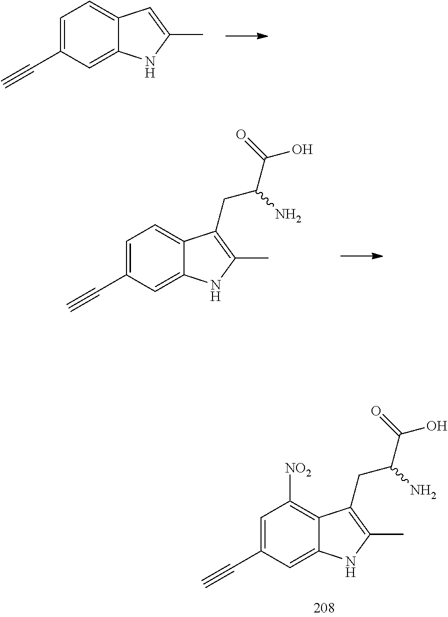 [SMILES: C#Cc1ccc2cc(C)[nH]c2c1 ; C#Cc1ccc2c(CC(N)C(=O)O)c(C)[nH]c2c1 ; C#Cc1cc([N+](=O)[O-])c2c(CC(N)C(=O)O)c(C)[nH]c2c1]

Example 208 can be prepared from 6-ethynyl-2-methyl-indole as shown above.

### Example 209: Preparation of 2-amino-3-(7-ethynyl-2-methyl-4-nitro-1H-indol-3-yl)propanoic acid (209)

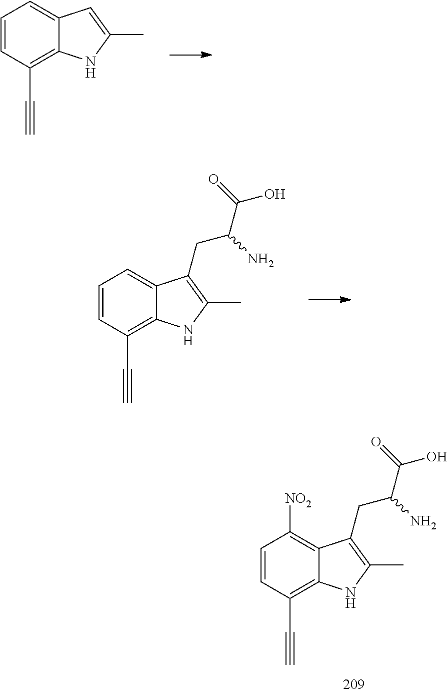 [SMILES: C#Cc1cccc2cc(C)[nH]c12 ; C#Cc1cccc2c(CC(N)C(=O)O)c(C)[nH]c12 ; C#Cc1ccc([N+](=O)[O-])c2c(CC(N)C(=O)O)c(C)[nH]c12]

Example 209 can be prepared from 7-ethynyl-2-methyl-indole as shown above.

### Example 210: Preparation of 2-amino-3-(4-ethynyl-2-methyl-7-nitro-1H-indol-3-yl)propanoic acid (210)

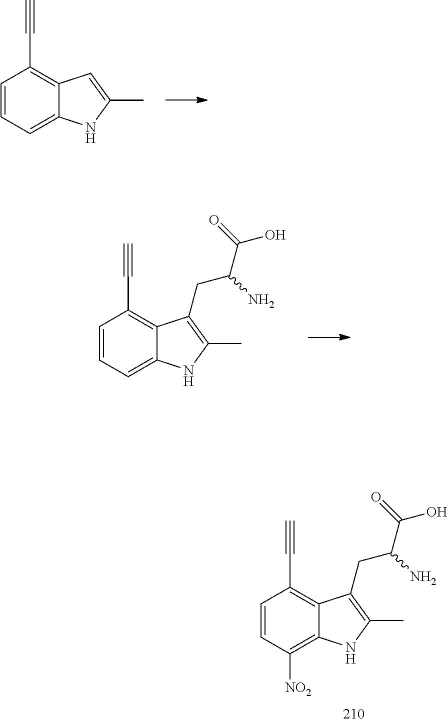 [SMILES: C#Cc1cccc2[nH]c(C)cc12 ; C#Cc1cccc2[nH]c(C)c(CC(N)C(=O)O)c12 ; C#Cc1ccc([N+](=O)[O-])c2[nH]c(C)c(CC(N)C(=O)O)c12]

Example 210 can be prepared from 4-ethynyl-2-methyl-indole as shown above.

### Example 211: Preparation of 2-amino-3-(2-methyl-5-morpholino-4-nitro-1H-indol-3-yl)propanoic acid (211)

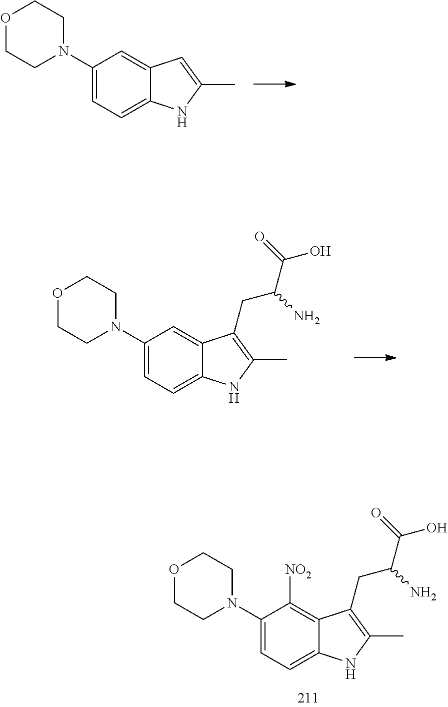 [SMILES: Cc1cc2cc(N3CCOCC3)ccc2[nH]1 ; Cc1[nH]c2ccc(N3CCOCC3)cc2c1CC(N)C(=O)O ; Cc1[nH]c2ccc(N3CCOCC3)c([N+](=O)[O-])c2c1CC(N)C(=O)O]

Example 211 can be prepared from 5-morpholino-2-methyl-indole as shown above.

### Example 212: Preparation of 2-amino-3-(2-methyl-6-morpholino-4-nitro-1H-indol-3-yl)propanoic acid (212)

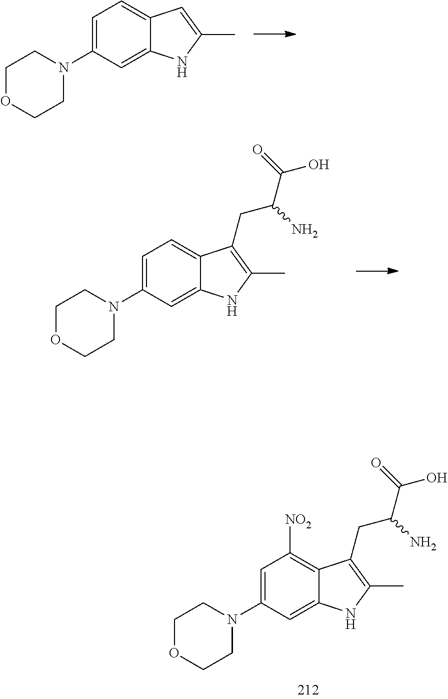 [SMILES: Cc1cc2ccc(N3CCOCC3)cc2[nH]1 ; Cc1[nH]c2cc(N3CCOCC3)ccc2c1CC(N)C(=O)O ; Cc1[nH]c2cc(N3CCOCC3)cc([N+](=O)[O-])c2c1CC(N)C(=O)O]

Example 212 can be prepared from 6-morpholino-2-methyl-indole as shown above.

### Example 213: Preparation of 2-amino-3-(2-methyl-7-morpholino-4-nitro-1H-indol-3-yl)propanoic acid (213)

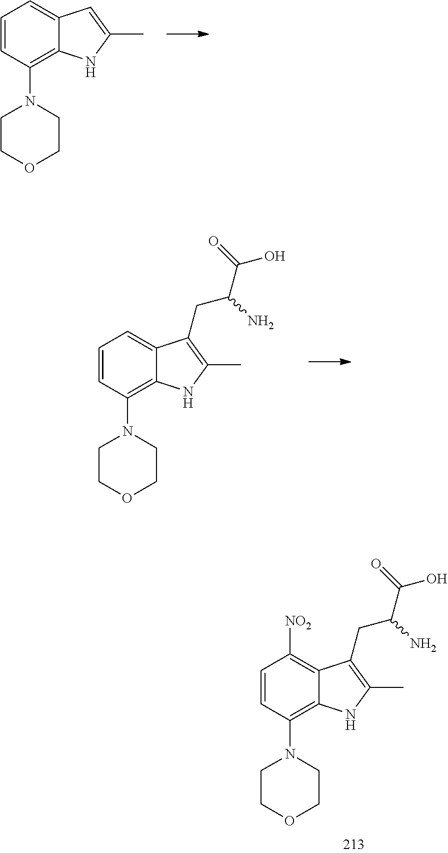 [SMILES: Cc1cc2cccc(N3CCOCC3)c2[nH]1 ; Cc1[nH]c2c(N3CCOCC3)cccc2c1CC(N)C(=O)O ; Cc1[nH]c2c(N3CCOCC3)ccc([N+](=O)[O-])c2c1CC(N)C(=O)O]

Example 213 can be prepared from 7-morpholino-2-methyl-indole as shown above.

### Example 214: Preparation of 2-amino-3-(2-methyl-4-morpholino-7-nitro-1H-indol-3-yl)propanoic acid (214)

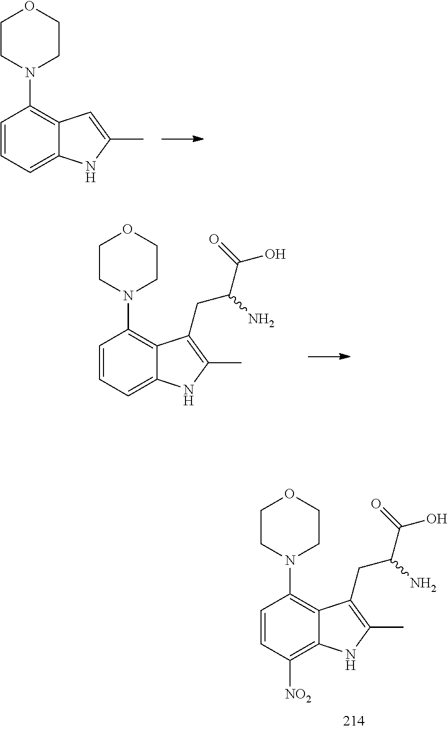 [SMILES: Cc1cc2c(N3CCOCC3)cccc2[nH]1 ; Cc1[nH]c2cccc(N3CCOCC3)c2c1CC(N)C(=O)O ; Cc1[nH]c2c([N+](=O)[O-])ccc(N3CCOCC3)c2c1CC(N)C(=O)O]

Example 214 can be prepared from 4-morpholino-2-methyl-indole as shown above.

### Example 215: Preparation of 2-amino-3-(2-methyl-5-(methylthio)-4-nitro-1H-indol-3-yl)propanoic acid (215)

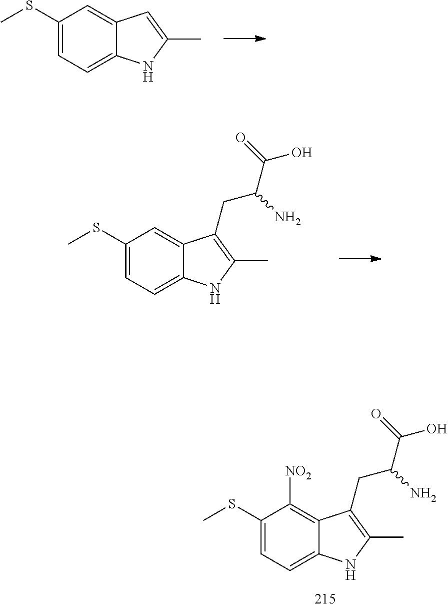

Example 215 can be prepared from 5-(methylthio)-2-methyl-indole as shown above.

### Example 216: Preparation of 2-amino-3-(2-methyl-6-(methylthio)-4-nitro-1H-4-indol-3-yl)propanoic acid (216)

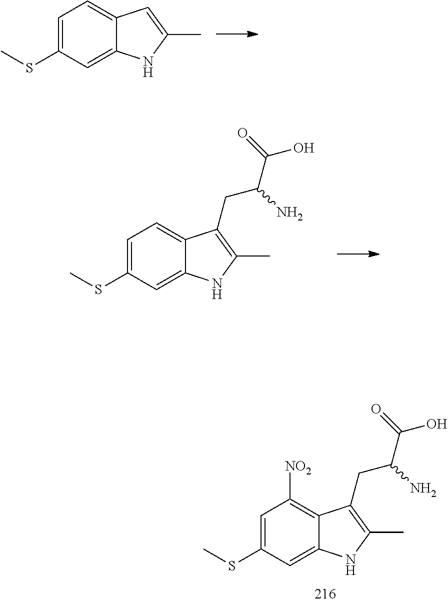

Example 216 can be prepared from 6-(methylthio)-2-methyl-indole as shown above.

### Example 217: Preparation of 2-amino-3-(2-methyl-7-(methylthio)-4-nitro-1H-indol-3-yl)propanoic acid (217)

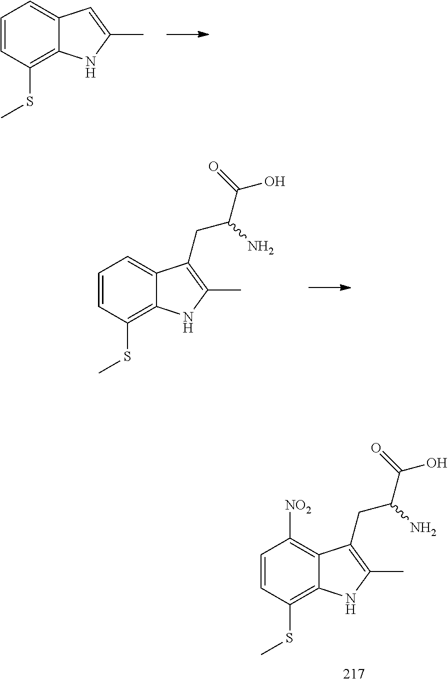 [SMILES: CSc1cccc2cc(C)[nH]c12 ; CSc1cccc2c(CC(N)C(=O)O)c(C)[nH]c12 ; CSc1ccc([N+](=O)[O-])c2c(CC(N)C(=O)O)c(C)[nH]c12]

Example 217 can be prepared from 7-(methylthio)-2-methyl-indole as shown above.

### Example 218: Preparation of 2-amino-3-(2-methyl-4-(methylthio)-7-nitro-1H-indol-3-yl)propanoic acid (218)

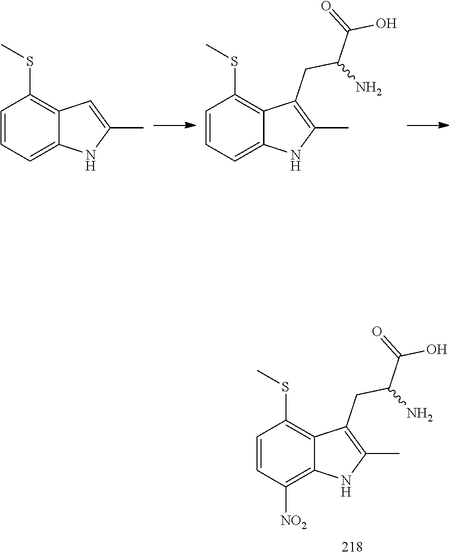 [SMILES: CSc1cccc2[nH]c(C)cc12 ; CSc1cccc2[nH]c(C)c(CC(N)C(=O)O)c12 ; CSc1ccc([N+](=O)[O-])c2[nH]c(C)c(CC(N)C(=O)O)c12]

Example 218 can be prepared from 4-(methylthio)-2-methyl-indole as shown above.

### Example 219: Preparation of 2-amino-3-(2-ethyl-4-nitro-5-(pyridin-4-yl)-1H-indol-3-yl)propanoic acid (219)

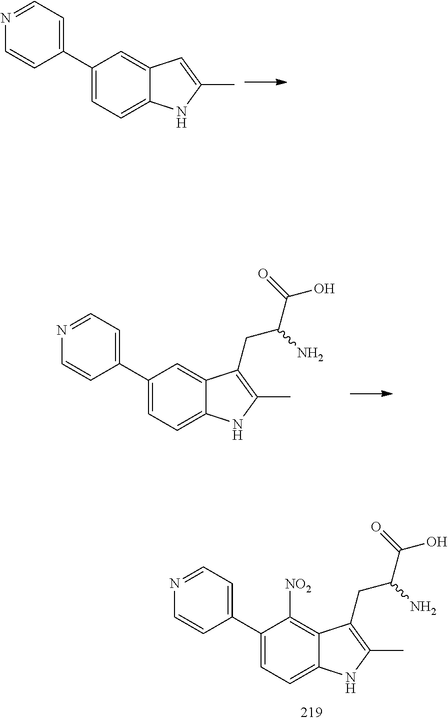 [SMILES: Cc1cc2cc(-c3ccncc3)ccc2[nH]1 ; Cc1[nH]c2ccc(-c3ccncc3)cc2c1CC(N)C(=O)O ; Cc1[nH]c2ccc(-c3ccncc3)c([N+](=O)[O-])c2c1CC(N)C(=O)O]

Example 219 can be prepared from 5-(pyridin-4-yl)-2-methyl-indole as shown above.

### Example 220: Preparation of 2-amino-3-(2-methyl-4-nitro-6-(pyridin-4-yl)-1H-indol-3-yl)propanoic acid (220)

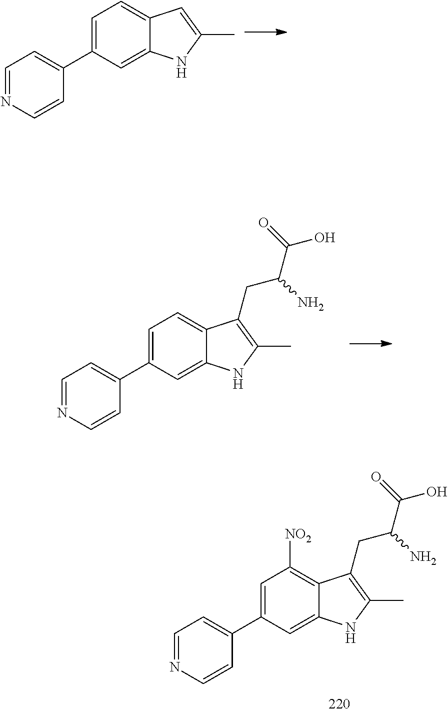 [SMILES: Cc1cc2ccc(-c3ccncc3)cc2[nH]1 ; Cc1[nH]c2cc(-c3ccncc3)ccc2c1CC(N)C(=O)O ; Cc1[nH]c2cc(-c3ccncc3)cc([N+](=O)[O-])c2c1CC(N)C(=O)O]

Example 220 can be prepared from 6-(pyridin-4-yl)-2-methyl-indole as shown above.

### Example 221: Preparation of 2-amino-3-(2-methyl-4-nitro-7-(pyridin-4-yl)-IX-indol-3-yl)propanoic acid (221)

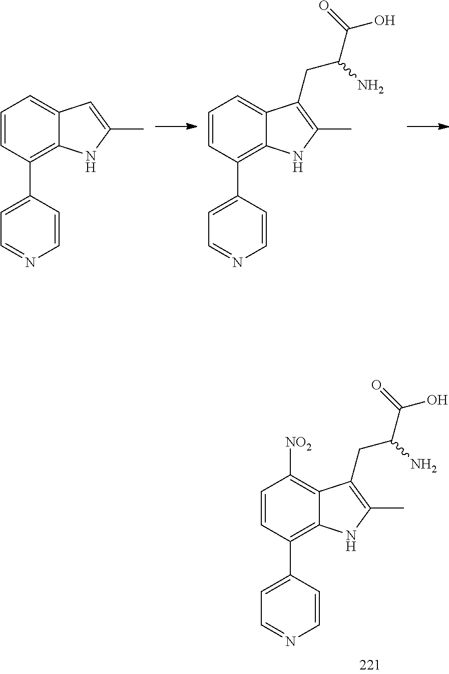 [SMILES: Cc1cc2cccc(-c3ccncc3)c2[nH]1 ; Cc1[nH]c2c(-c3ccncc3)cccc2c1CC(N)C(=O)O ; Cc1[nH]c2c(-c3ccncc3)ccc([N+](=O)[O-])c2c1CC(N)C(=O)O]

Example 221 can be prepared from 7-(pyridin-4-yl)-2-methyl-indole as shown above.

### Example 222: Preparation of 2-amino-3-(2-methyl-7-nitro-4-(pyridin-4-yl)-1H-indol-3-yl)propanoic acid (222)

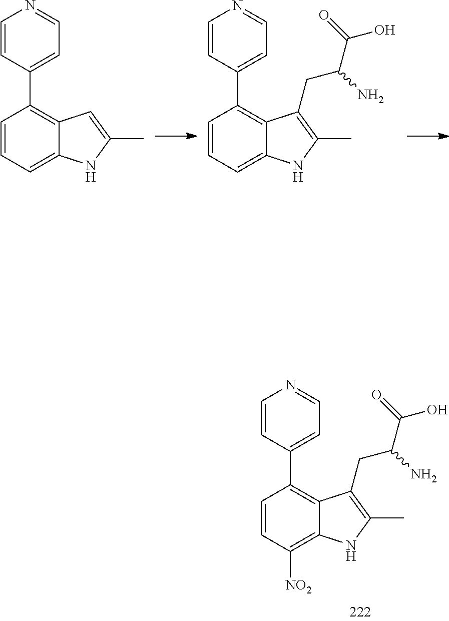 [SMILES: Cc1cc2c(-c3ccncc3)cccc2[nH]1 ; Cc1[nH]c2cccc(-c3ccncc3)c2c1CC(N)C(=O)O ; Cc1[nH]c2c([N+](=O)[O-])ccc(-c3ccncc3)c2c1CC(N)C(=O)O]

Example 222 can be prepared from 4-(pyridin-4-yl-2-methyl-indole as shown above.

### Example 223: Preparation of 2-amino-3-(1,2,5-trimethyl-4-nitro-1H-indol-3-yl)propanoic acid (223)

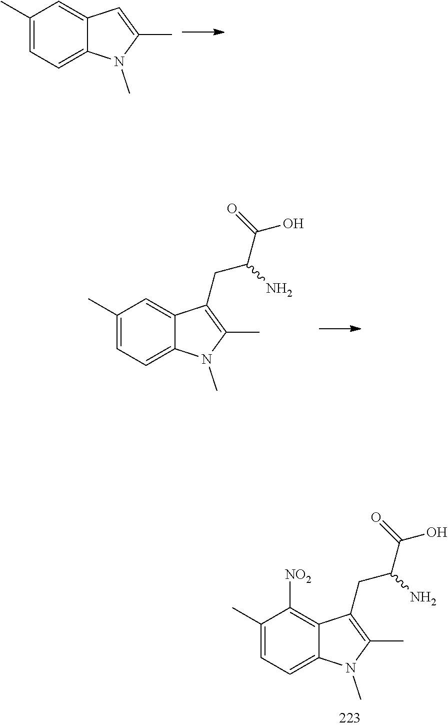

Example 223 can be prepared from 1,2,5-trimethyl-1H-indole as shown above.

### Example 224: Preparation of 2-amino-3-(1,2,6-trimethyl-4-nitro-1H-indol-3-yl)propanoic acid (224)

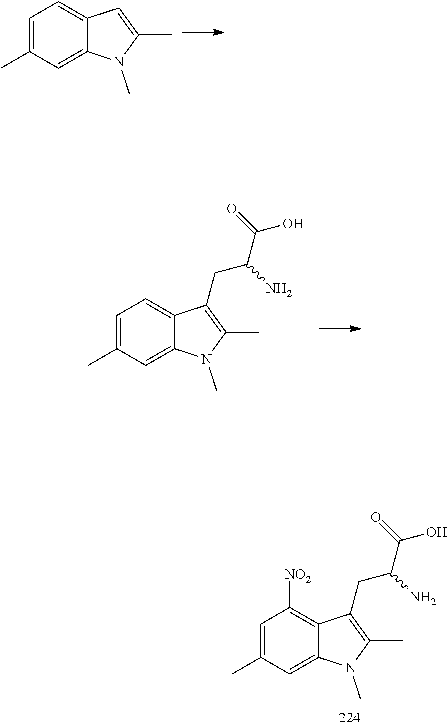

Example 224 can be prepared from 1,2,6-trimethyl-1H-indole as shown above.

### Example 225: Preparation of 2-amino-3-(1,2,7-trimethyl-4-nitro-1H-indol-3-yl)propanoic acid (225)

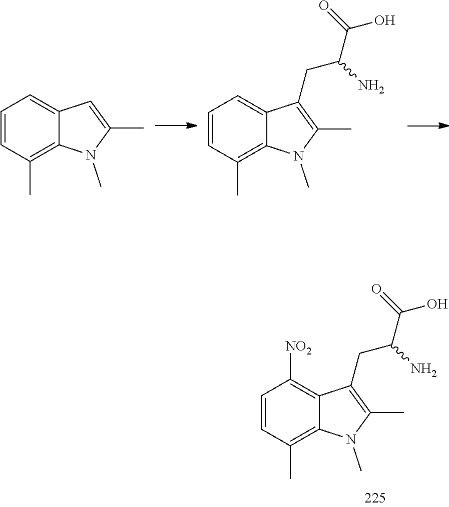 [SMILES: Cc1cccc2cc(C)n(C)c12 ; Cc1cccc2c(CC(N)C(=O)O)c(C)n(C)c12 ; Cc1ccc([N+](=O)[O-])c2c(CC(N)C(=O)O)c(C)n(C)c12]

Example 225 can be prepared from 1,2,7-trimethyl-1H-indole as shown above.

### Example 226: Preparation of 2-amino-3-(1,2,4-trimethyl-7-nitro-1H-indol-3-yl)propanoic acid (226)

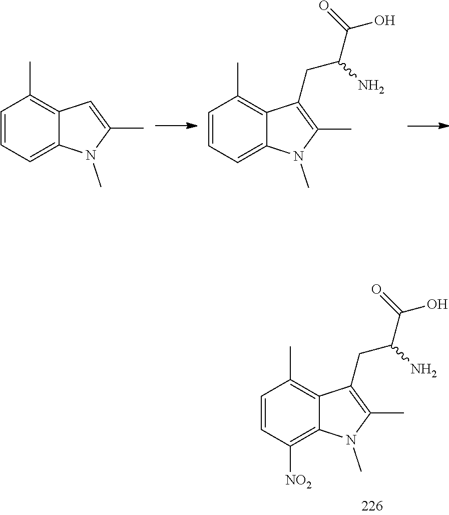 [SMILES: Cc1cccc2c1cc(C)n2C ; Cc1cccc2c1c(CC(N)C(=O)O)c(C)n2C ; Cc1ccc([N+](=O)[O-])c2c1c(CC(N)C(=O)O)c(C)n2C]

Example 226 can be prepared from 1,2,4-trimethyl-1H-indole as shown above.

### Example 227: Preparation of 2-amino-3-(6-fluoro-1,2-dimethyl-4-nitro-1H-indol-3-yl)propanoic acid (227)

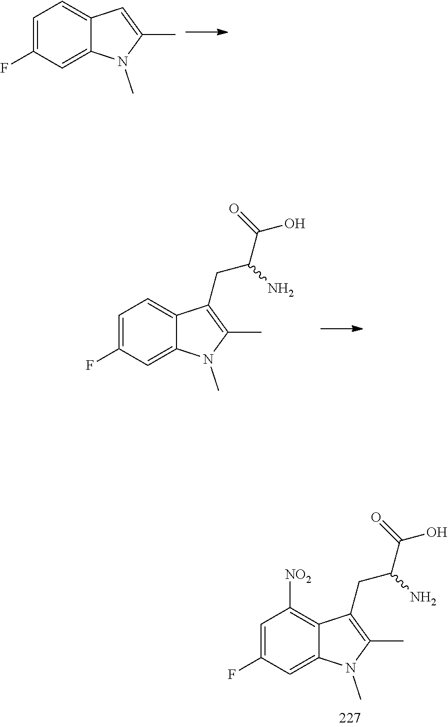 [SMILES: Cc1cc2ccc(F)cc2n1C ; Cc1c(CC(N)C(=O)O)c2ccc(F)cc2n1C ; Cc1c(CC(N)C(=O)O)c2c([N+](=O)[O-])cc(F)cc2n1C]

Example 227 can be prepared from 6-fluoro-1,2-dimethyl-1H-indole as shown above.

### Example 228: Preparation of 2-amino-3-(7-fluoro-1,2-dimethyl-4-nitro-1H-indol-3-yl)propanoic acid (228)

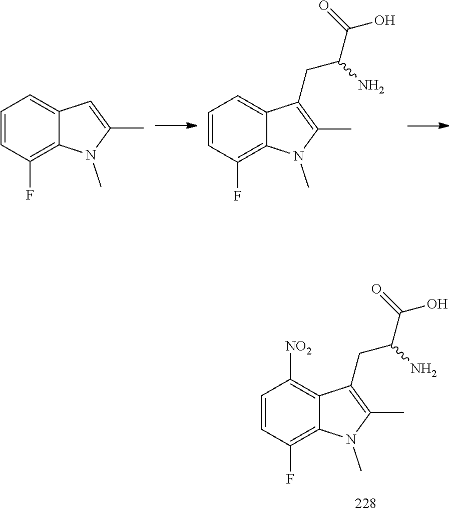 [SMILES: Cc1cc2cccc(F)c2n1C ; Cc1c(CC(N)C(=O)O)c2cccc(F)c2n1C ; Cc1c(CC(N)C(=O)O)c2c([N+](=O)[O-])ccc(F)c2n1C]

Example 228 can be prepared from 7-fluoro-1,2-dimethyl-indole as shown above.

### Example 229: Preparation of 2-amino-3-(4-fluoro-1,2-dimethyl-7-nitro-1H-indol-3-yl)propanoic acid (229)

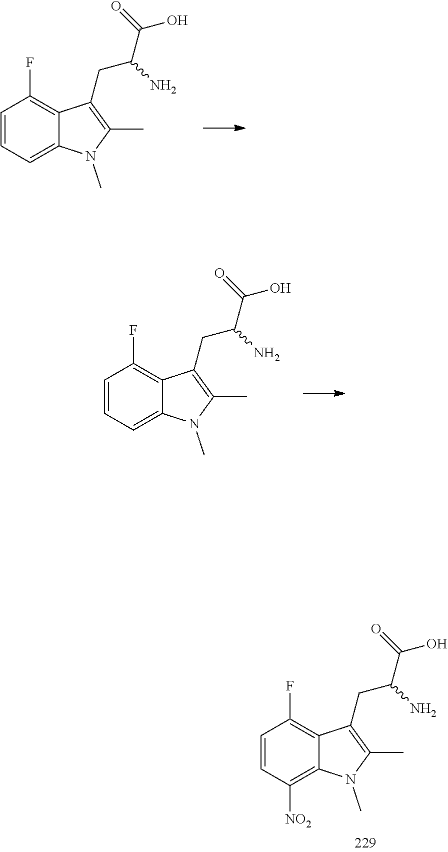 [SMILES: Cc1c(CC(N)C(=O)O)c2c(F)cccc2n1C ; Cc1c(CC(N)C(=O)O)c2c(F)cccc2n1C ; Cc1c(CC(N)C(=O)O)c2c(F)ccc([N+](=O)[O-])c2n1C]

Example 229 can be prepared from 4-fluoro-1,2-dimethyl-indole as shown above.

### Example 230: Preparation of 2-amino-3-(5-chloro-1,2-dimethyl-4-nitro-1H-indol-3-yl)propanoic acid (230)

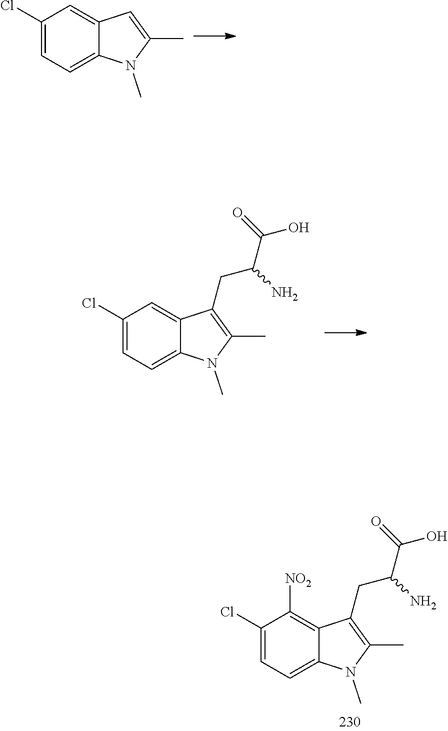 [SMILES: Cc1cc2cc(Cl)ccc2n1C ; Cc1c(CC(N)C(=O)O)c2cc(Cl)ccc2n1C ; Cc1c(CC(N)C(=O)O)c2c([N+](=O)[O-])c(Cl)ccc2n1C]

Example 230 can be prepared from 5-chloro-1,2-dimethyl-indole as shown above.

### Example 231: Preparation of 2-amino-3-(6-chloro-1,2-dimethyl-4-nitro-1H-indol-3-yl)propanoic acid (231)

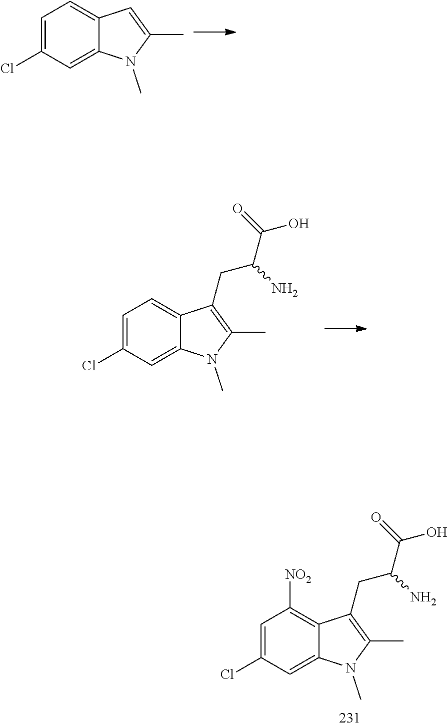 [SMILES: Cc1cc2ccc(Cl)cc2n1C ; Cc1c(CC(N)C(=O)O)c2ccc(Cl)cc2n1C ; Cc1c(CC(N)C(=O)O)c2c([N+](=O)[O-])cc(Cl)cc2n1C]

Example 231 can be prepared from 6-chloro-1,2-dimethyl-indole as shown above.

### Example 232: Preparation of 2-amino-3-(7-chloro-1,2-dimethyl-4-nitro-1H-indol-3-yl)propanoic acid (232)

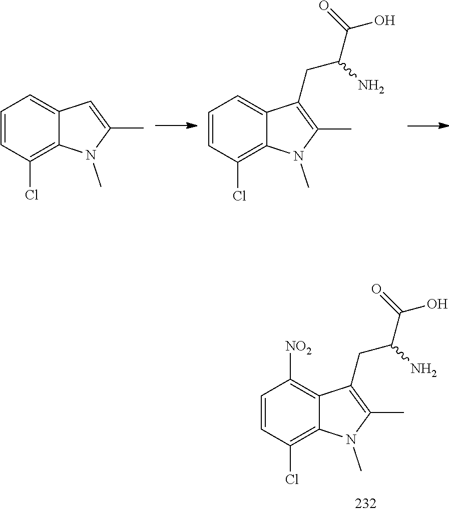 [SMILES: Cc1cc2cccc(Cl)c2n1C ; Cc1c(CC(N)C(=O)O)c2cccc(Cl)c2n1C ; Cc1c(CC(N)C(=O)O)c2c([N+](=O)[O-])ccc(Cl)c2n1C]

Example 232 can be prepared from 7-chloro-1,2-dimethyl-indole as shown above.

### Example 233: Preparation of 2-amino-3-(4-chloro-1,2-dimethyl-7-nitro-1H-indol-3-yl)propanoic acid (233)

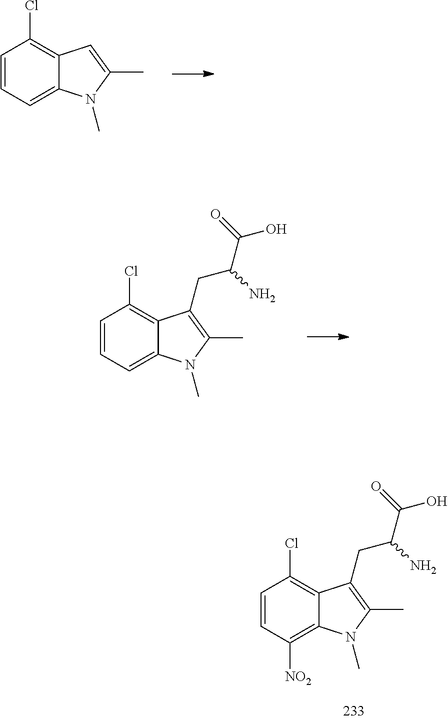 [SMILES: Cc1cc2c(Cl)cccc2n1C ; Cc1c(CC(N)C(=O)O)c2c(Cl)cccc2n1C ; Cc1c(CC(N)C(=O)O)c2c(Cl)ccc([N+](=O)[O-])c2n1C]

Example 233 can be prepared from 4-chloro-1,2-dimethyl-indole as shown above.

### Example 234: Preparation of 2-amino-3-(5-bromo-1,2-dimethyl-4-nitro-indol-3-yl)propanoic acid (234)

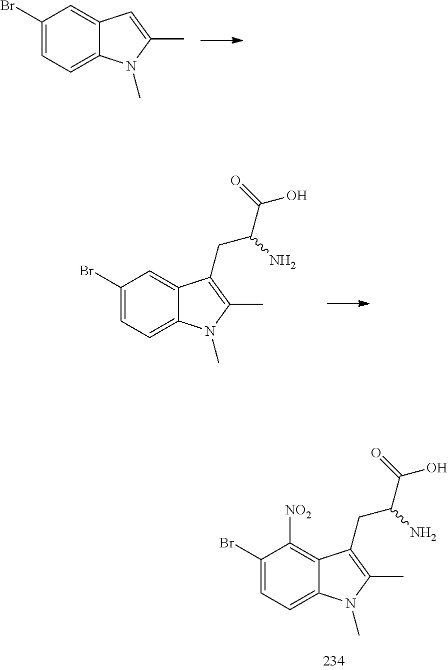 [SMILES: Cc1cc2cc(Br)ccc2n1C ; Cc1c(CC(N)C(=O)O)c2cc(Br)ccc2n1C ; Cc1c(CC(N)C(=O)O)c2c([N+](=O)[O-])c(Br)ccc2n1C]

Example 234 can be prepared from 5-bromo-1,2-dimethyl-indole as shown above.

### Example 235: Preparation of 2-amino-3-(6-bromo-1,2-dimethyl-4-nitro-1H-indol-3-yl)propanoic acid (235)

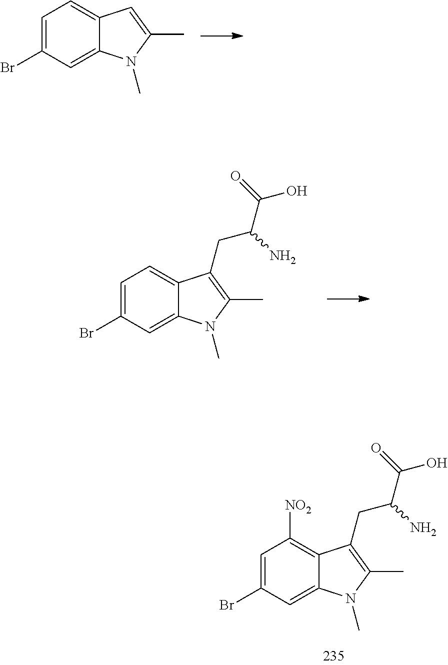 [SMILES: Cc1cc2ccc(Br)cc2n1C ; Cc1c(CC(N)C(=O)O)c2ccc(Br)cc2n1C ; Cc1c(CC(N)C(=O)O)c2c([N+](=O)[O-])cc(Br)cc2n1C]

Example 235 can be prepared from 6-bromo-1,2-dimethyl-indole as shown above.

### Example 236: Preparation of 2-amino-(7-bromo-1,2-dimethyl-4-nitro-1H-indol-3-yl)propanoic acid (236)

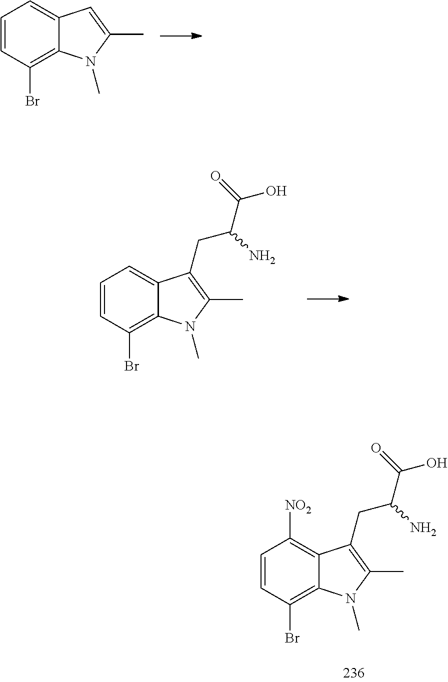 [SMILES: Cc1cc2cccc(Br)c2n1C ; Cc1c(CC(N)C(=O)O)c2cccc(Br)c2n1C ; Cc1c(CC(N)C(=O)O)c2c([N+](=O)[O-])ccc(Br)c2n1C]

Example 236 can be prepared from 7-bromo-1,2-dimethyl-indole as shown above.

### Example 237: Preparation of 2-amino-3-(4-bromo-1,2-dimethyl-7-nitro-1H-indol-3-yl)propanoic acid (237)

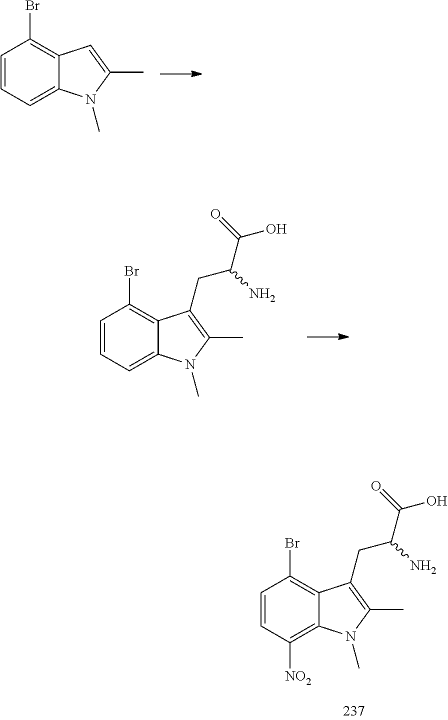 [SMILES: Cc1cc2c(Br)cccc2n1C ; Cc1c(CC(N)C(=O)O)c2c(Br)cccc2n1C ; Cc1c(CC(N)C(=O)O)c2c(Br)ccc([N+](=O)[O-])c2n1C]

Example 237 can be prepared from 4-bromo-1,2-dimethyl-indole as shown above.

### Example 238: Preparation of 2-amino-3-(5-methoxy-1,2-dimethyl-4-nitro-1H-indol-3-yl)propanoic acid (238)

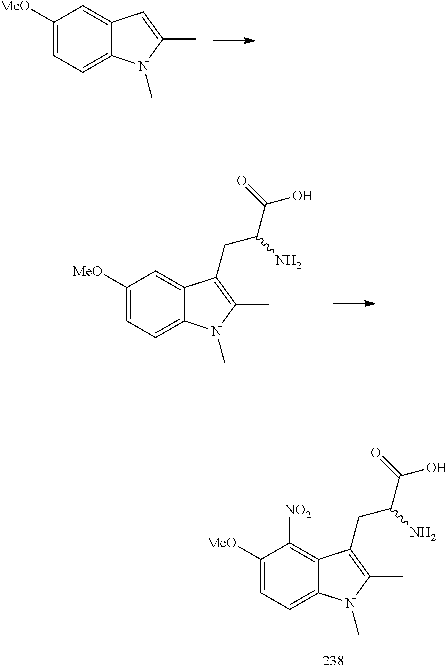 [SMILES: COc1ccc2c(c1)cc(C)n2C ; COc1ccc2c(c1)c(CC(N)C(=O)O)c(C)n2C ; COc1ccc2c(c(CC(N)C(=O)O)c(C)n2C)c1[N+](=O)[O-]]

Example 238 can be prepared from 5-methoxy-1,2-dimethyl-indole as shown above.

### Example 239: Preparation of 2-amino-3-(6-methoxy-1,2-dimethyl-4-nitro-1H-indol-3-yl)propanoic acid (239)

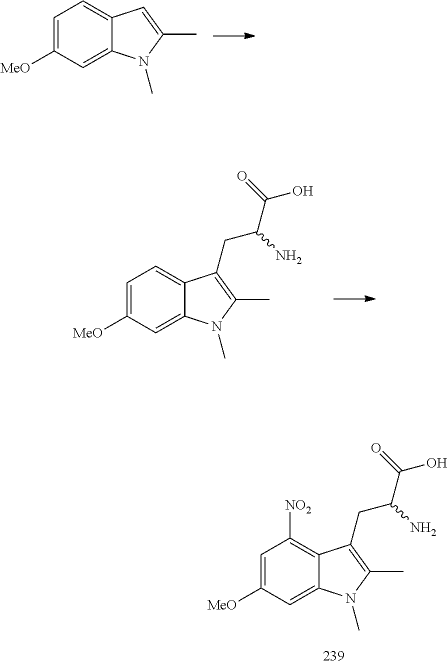 [SMILES: COc1ccc2cc(C)n(C)c2c1 ; COc1ccc2c(CC(N)C(=O)O)c(C)n(C)c2c1 ; COc1cc([N+](=O)[O-])c2c(CC(N)C(=O)O)c(C)n(C)c2c1]

Example 239 can be prepared from 6-methoxy-1,2-dimethyl-indole as shown above.

### Example 240: Preparation of 2-amin-3-(7-methoxy-1,2-dimethyl-4-nitro-1H-indol-3-yl)propanoic acid (240)

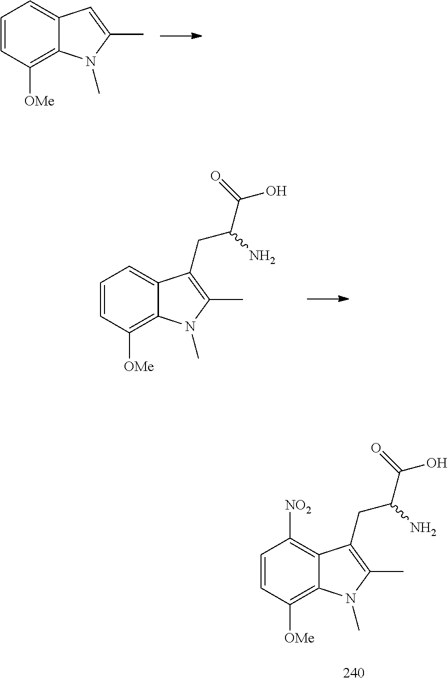 [SMILES: COc1cccc2cc(C)n(C)c12 ; COc1cccc2c(CC(N)C(=O)O)c(C)n(C)c12 ; COc1ccc([N+](=O)[O-])c2c(CC(N)C(=O)O)c(C)n(C)c12]

Example 240 can be prepared from 7-methoxy-1,2-dimethyl-indole as shown above.

### Example 241: Preparation of 2-amino-3-(4-methoxy-1,2-dimethyl-7-nitro-1H-indol-3-yl)propanoic acid (241)

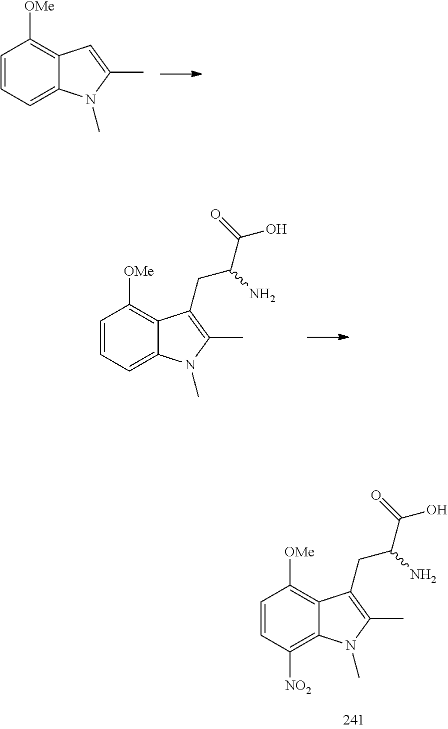 [SMILES: COc1cccc2c1cc(C)n2C ; COc1cccc2c1c(CC(N)C(=O)O)c(C)n2C ; COc1ccc([N+](=O)[O-])c2c1c(CC(N)C(=O)O)c(C)n2C]

Example 241 can be prepared from 4-methoxy-1,2-dimethyl-indole as shown above.

### Example 242: Preparation of 2-amino-3-(5-amino-1,2-dimethyl-4-nitro-1H-indol-3-yl)propanoic acid (242)

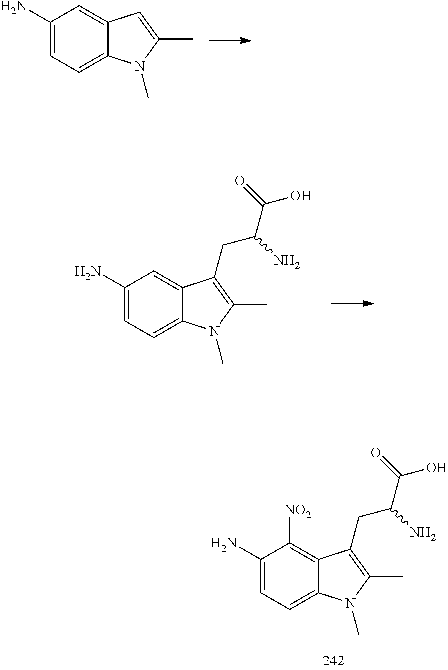 [SMILES: Cc1cc2cc(N)ccc2n1C ; Cc1c(CC(N)C(=O)O)c2cc(N)ccc2n1C ; Cc1c(CC(N)C(=O)O)c2c([N+](=O)[O-])c(N)ccc2n1C]

Example 242 can be prepared from 5-amino-1,2-dimethyl-indole as shown above.

### Example 243: Preparation of 2-amino-3-(6-amino-1,2-dimethyl-4-nitro-1H-indol-3-yl)propanoic acid (243)

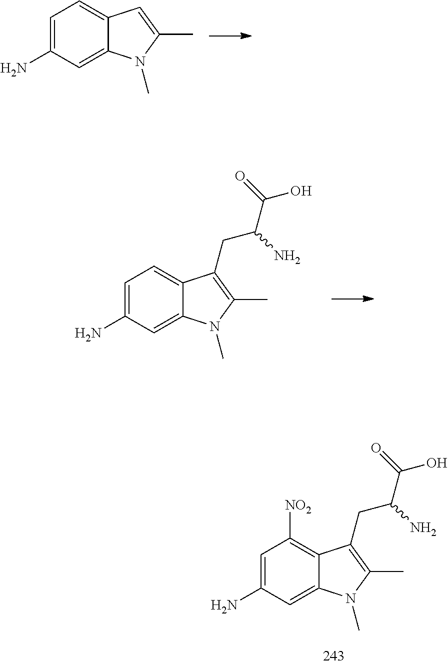 [SMILES: Cc1cc2ccc(N)cc2n1C ; Cc1c(CC(N)C(=O)O)c2ccc(N)cc2n1C ; Cc1c(CC(N)C(=O)O)c2c([N+](=O)[O-])cc(N)cc2n1C]

Example 243 can be prepared from 6-amino-1,2-dimethyl-indole as shown above.

### Example 244: Preparation of 2-amino-3-(7-amino-1,2-dimethyl-4-nitro-1H-indol-3-yl)propanoic acid (244)

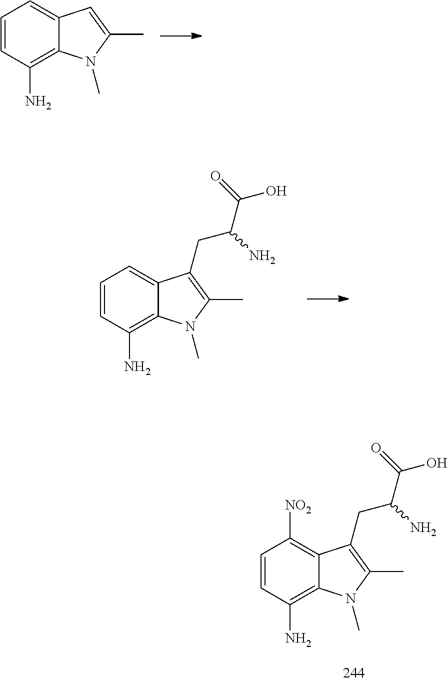 [SMILES: Cc1cc2cccc(N)c2n1C ; Cc1c(CC(N)C(=O)O)c2cccc(N)c2n1C ; Cc1c(CC(N)C(=O)O)c2c([N+](=O)[O-])ccc(N)c2n1C]

Example 244 can be prepared from 7-amino-1,2-dimethyl-indole as shown above.

### Example 245: Preparation of 2-amino-3-(4-amino-1,2-dimethyl-7-nitro-1H-indol-3-yl)propanoic acid (245)

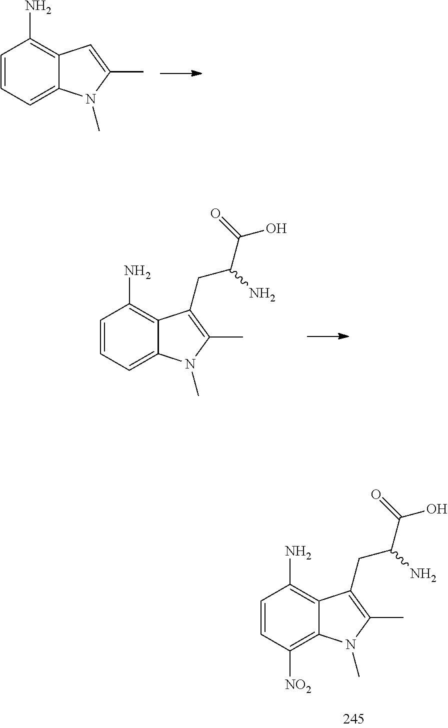 [SMILES: Cc1cc2c(N)cccc2n1C ; Cc1c(CC(N)C(=O)O)c2c(N)cccc2n1C ; Cc1c(CC(N)C(=O)O)c2c(N)ccc([N+](=O)[O-])c2n1C]

Example 245 can be prepared from 4-amino-1,2-dimethyl-indole as shown above.

### Example 246: Preparation of 2-amino-3-(5-hydroxy-1,2-dimethyl-4-nitro-1H-indol-3-yl)propanoic acid (246)

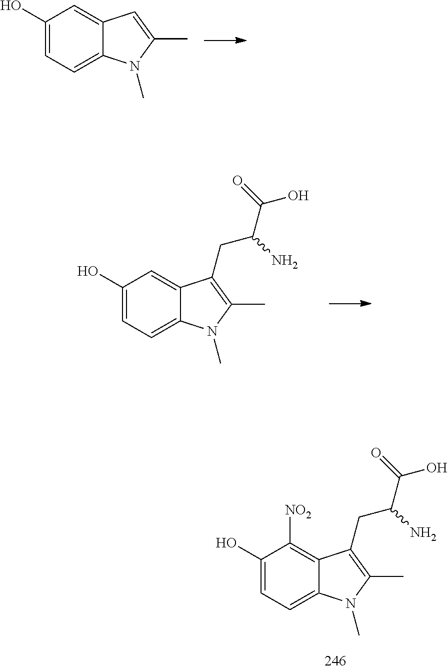 [SMILES: Cc1cc2cc(O)ccc2n1C ; Cc1c(CC(N)C(=O)O)c2cc(O)ccc2n1C ; Cc1c(CC(N)C(=O)O)c2c([N+](=O)[O-])c(O)ccc2n1C]

Example 246 can be prepared from 5-hydroxy-1,2-dimethyl-indole as shown above.

### Example 247: Preparation of 2-amino-3-(6-hydroxy-1,2-dimethyl-4-nitro-1H-indol-3-yl)propanoic acid (247)

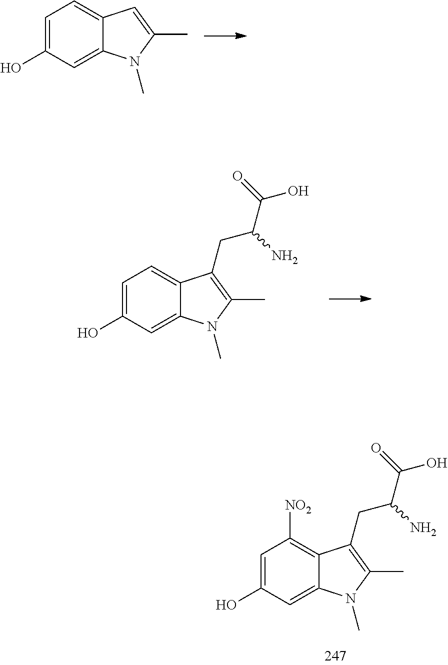 [SMILES: Cc1cc2ccc(O)cc2n1C ; Cc1c(CC(N)C(=O)O)c2ccc(O)cc2n1C ; Cc1c(CC(N)C(=O)O)c2c([N+](=O)[O-])cc(O)cc2n1C]

Example 247 can be prepared from 6-hydroxy-1,2-dimethyl-indole as shown above.

### Example 248: Preparation of 2-amino-3-(7-hydroxy-1,2-dimethyl-4-nitro-1H-indol-3-yl)propanoic acid (248)

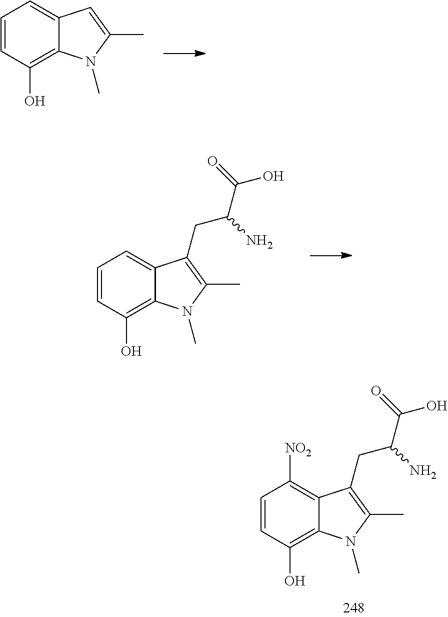 [SMILES: Cc1cc2cccc(O)c2n1C ; Cc1c(CC(N)C(=O)O)c2cccc(O)c2n1C ; Cc1c(CC(N)C(=O)O)c2c([N+](=O)[O-])ccc(O)c2n1C]

Example 248 can be prepared from 7-hydroxy-1,2-dimethyl-indole as shown above.

### Example 249: Preparation of 2-amino-3-(4-hydroxy-1,2-dimethyl-7-nitro-1H-indol-3-yl)propanoic acid (249)

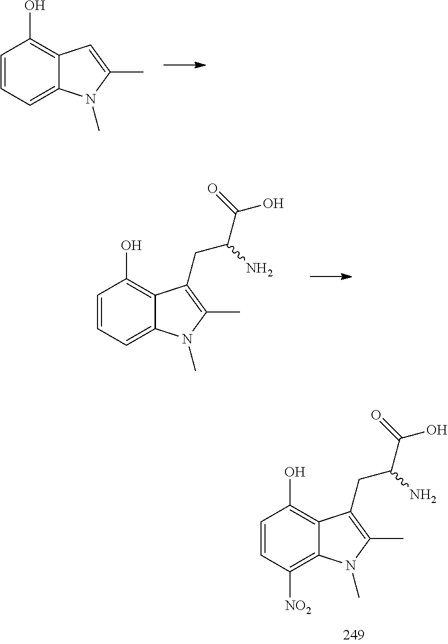 [SMILES: Cc1cc2c(O)cccc2n1C ; Cc1c(CC(N)C(=O)O)c2c(O)cccc2n1C ; Cc1c(CC(N)C(=O)O)c2c(O)ccc([N+](=O)[O-])c2n1C]

Example 249 can be prepared from 4-hydroxy-1,2-dimethyl-indole as shown above.

### Example 250: Preparation of 2-amino-3-(1,2-dimethyl-4-nitro-5-phenyl-1H-indol-3-yl)propanoic acid (250)

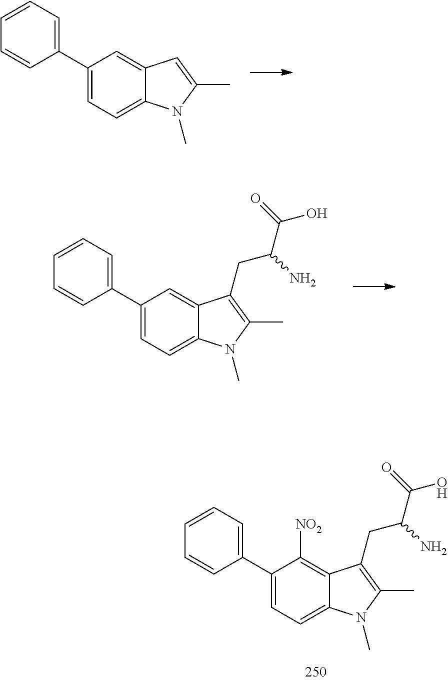 [SMILES: Cc1cc2cc(-c3ccccc3)ccc2n1C ; Cc1c(CC(N)C(=O)O)c2cc(-c3ccccc3)ccc2n1C ; Cc1c(CC(N)C(=O)O)c2c([N+](=O)[O-])c(-c3ccccc3)ccc2n1C]

Example 250 can be prepared from 5-phenyl-1,2-dimethyl-indole as shown above.

### Example 251: Preparation of 2-amino-3-(1,2-dimethyl-4-nitro-6-phenyl-1H-indol-3-yl)propanoic acid (251)

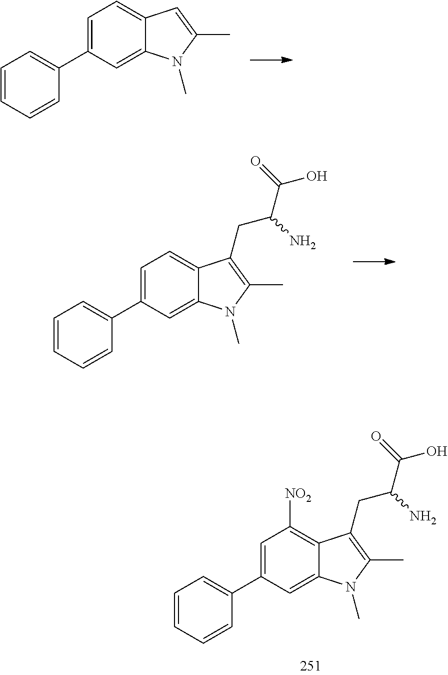 [SMILES: Cc1cc2ccc(-c3ccccc3)cc2n1C ; Cc1c(CC(N)C(=O)O)c2ccc(-c3ccccc3)cc2n1C ; Cc1c(CC(N)C(=O)O)c2c([N+](=O)[O-])cc(-c3ccccc3)cc2n1C]

Example 251 can be prepared from 6-phenyl-1,2-dimethyl-indole as shown above.

### Example 252: Preparation of 2-amino-3-(1,2-dimethyl-4-nitro-7-phenyl-1H-indol-3-yl)propanoic acid (250)

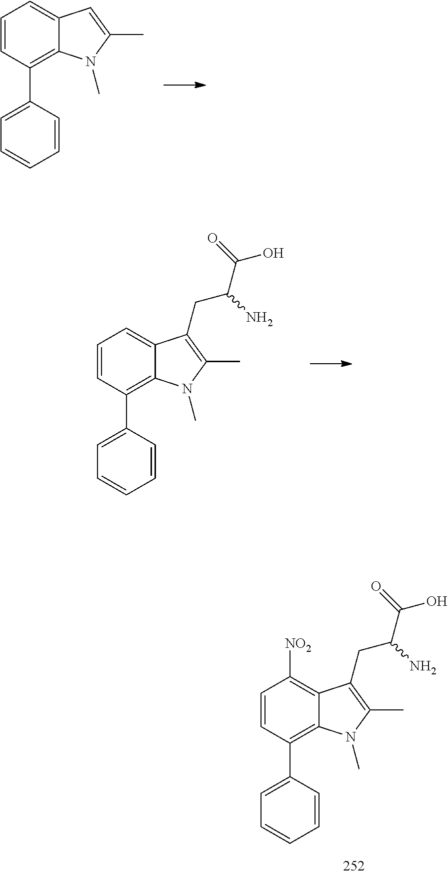 [SMILES: Cc1cc2cccc(-c3ccccc3)c2n1C ; Cc1c(CC(N)C(=O)O)c2cccc(-c3ccccc3)c2n1C ; Cc1c(CC(N)C(=O)O)c2c([N+](=O)[O-])ccc(-c3ccccc3)c2n1C]

Example 252 can be prepared from 7-phenyl-1,2-dimethyl-indole as shown above.

### Example 253: Preparation of 2-amino-3-(1,2-dimethyl-7-nitro-4-phenyl-1H-indol-3-yl)propanoic acid (253)

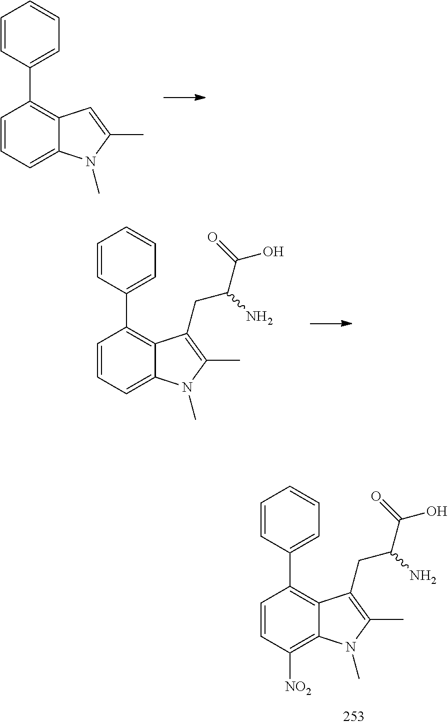

Example 253 can be prepared from 4-phenyl-1,2-dimethyl-indole as shown above.

### Example 254: Preparation of 2-amino-3-(5-cyclopropyl-1,2-dimethyl-4-nitro-1H-indol-3-yl)propanoic acid (254)

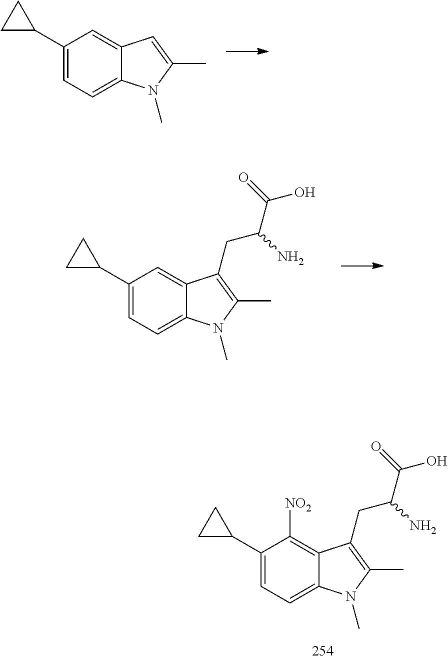 [SMILES: Cc1cc2cc(C3CC3)ccc2n1C ; Cc1c(CC(N)C(=O)O)c2cc(C3CC3)ccc2n1C ; Cc1c(CC(N)C(=O)O)c2c([N+](=O)[O-])c(C3CC3)ccc2n1C]

Example 254 can be prepared from 5-cyclopropyl-1,2-dimethyl-indole as shown above.

### Example 255: Preparation of 2-amino-3-(6-cyclopropyl-1,2-dimethyl-4-nitro-1H-indol-3-yl)propanoic acid (255)

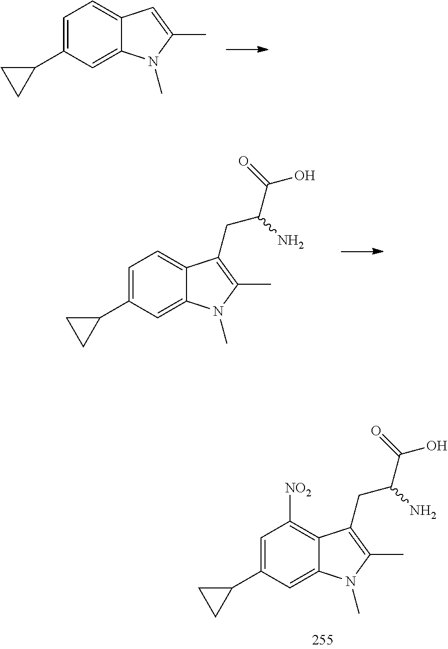 [SMILES: Cc1cc2ccc(C3CC3)cc2n1C ; Cc1c(CC(N)C(=O)O)c2ccc(C3CC3)cc2n1C ; Cc1c(CC(N)C(=O)O)c2c([N+](=O)[O-])cc(C3CC3)cc2n1C]

Example 255 can be prepared from 6-cyclopropyl-1,2-dimethyl-indole as shown above.

### Example 256: Preparation of 2-amino-3-(7-cyclopropyl-1,2-dimethyl-4-nitro-1H-indol-3-yl)propanoic acid (256)

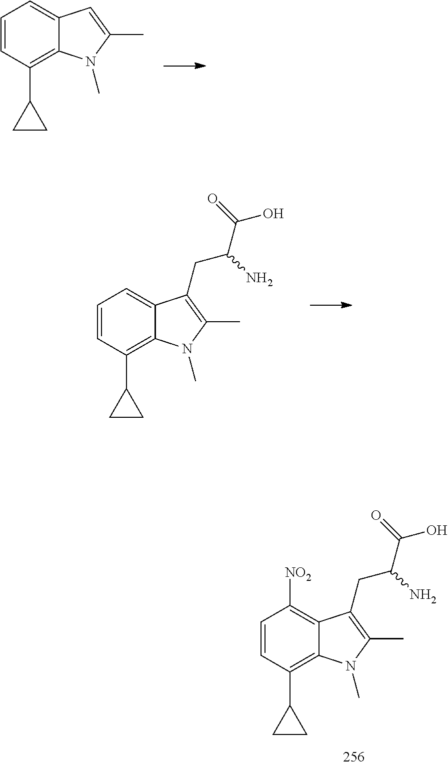 [SMILES: Cc1cc2cccc(C3CC3)c2n1C ; Cc1c(CC(N)C(=O)O)c2cccc(C3CC3)c2n1C ; Cc1c(CC(N)C(=O)O)c2c([N+](=O)[O-])ccc(C3CC3)c2n1C]

Example 256 can be prepared from 7-cyclopropyl-1,2-dimethyl-indole as shown above.

### Example 257: Preparation of 2-amino-3-(4-cyclopropyl-1,2-dimethyl-7-nitro-1H-indol-3-yl)propanoic acid (257)

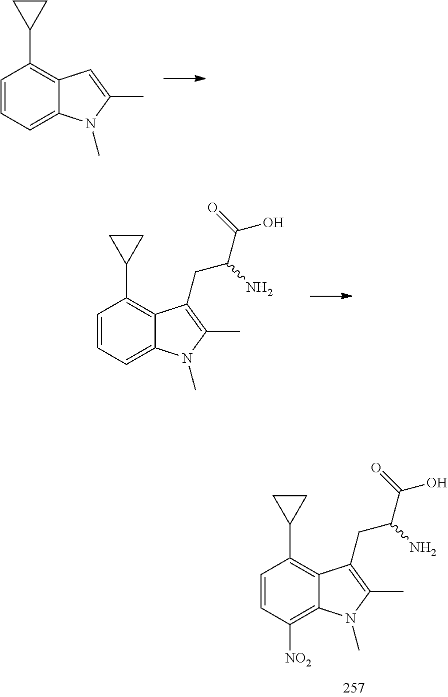 [SMILES: Cc1cc2c(C3CC3)cccc2n1C ; Cc1c(CC(N)C(=O)O)c2c(C3CC3)cccc2n1C ; Cc1c(CC(N)C(=O)O)c2c(C3CC3)ccc([N+](=O)[O-])c2n1C]

Example 257 can be prepared from 4-cyclopropyl-1,2-dimethyl-indole as shown above.

### Example 258: Preparation of 2-amino-3-(1,2-dimethyl-4-nitro-5-vinyl-1H-indol-3-yl)propanoic acid (258)

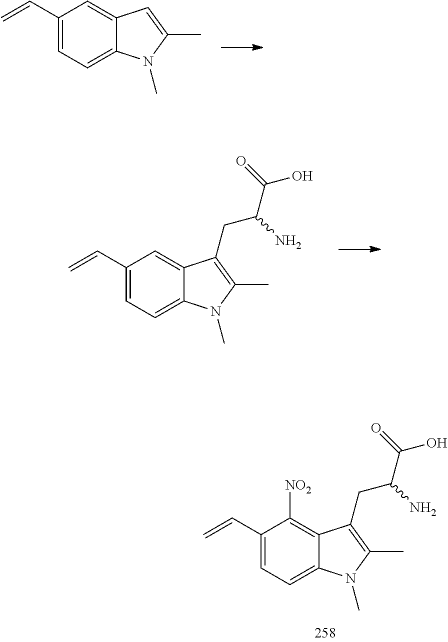 [SMILES: C=Cc1ccc2c(c1)cc(C)n2C ; C=Cc1ccc2c(c1)c(CC(N)C(=O)O)c(C)n2C ; C=Cc1ccc2c(c(CC(N)C(=O)O)c(C)n2C)c1[N+](=O)[O-]]

Example 258 can be prepared from 5-vinyl-1,2-dimethyl-indole as shown above.

### Example 259: Preparation of 2-amino-3-(1,2-didemethyl-4-nitro-6-vinyl-1H-indol-3-yl)propanoic acid (259)

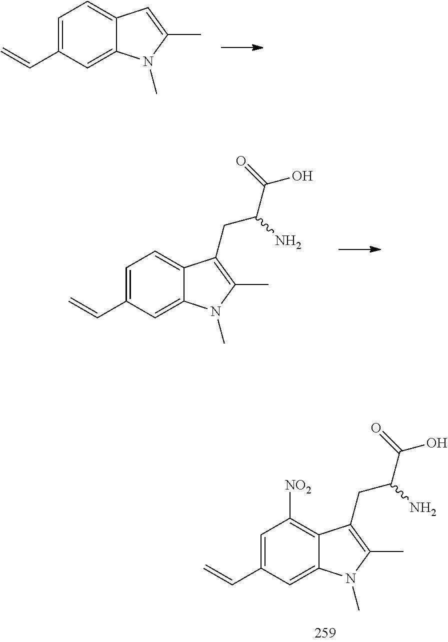 [SMILES: C=Cc1ccc2cc(C)n(C)c2c1 ; C=Cc1ccc2c(CC(N)C(=O)O)c(C)n(C)c2c1 ; C=Cc1cc([N+](=O)[O-])c2c(CC(N)C(=O)O)c(C)n(C)c2c1]

Example 259 can be prepared from 6-vinyl-1,2-dimethyl-indole as shown above.

### Example 260: Preparation of 2-amino-3-(1,2-dimethyl-4-nitro-7-vinyl-1H-indol-3-yl)propanoic acid (260)

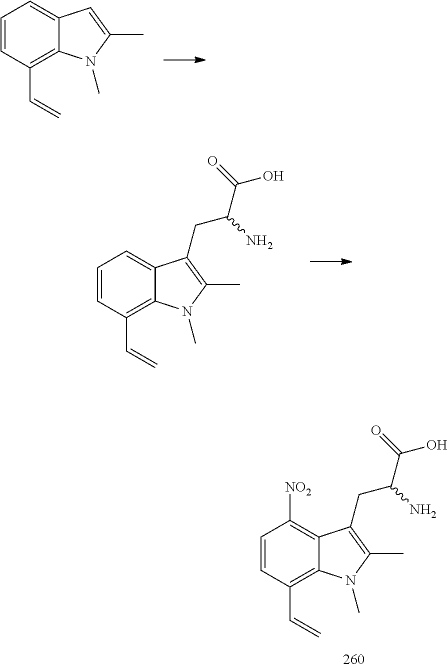 [SMILES: C=Cc1cccc2cc(C)n(C)c12 ; C=Cc1cccc2c(CC(N)C(=O)O)c(C)n(C)c12 ; C=Cc1ccc([N+](=O)[O-])c2c(CC(N)C(=O)O)c(C)n(C)c12]

Example 260 can be prepared from 7-vinyl-1,2-dimethyl-indole as shown above.

### Example 261: Preparation of 2-amino-3-(1,2-dimethyl-7-nitro-4-vinyl-1H-indol-3-yl)propanoic acid (261)

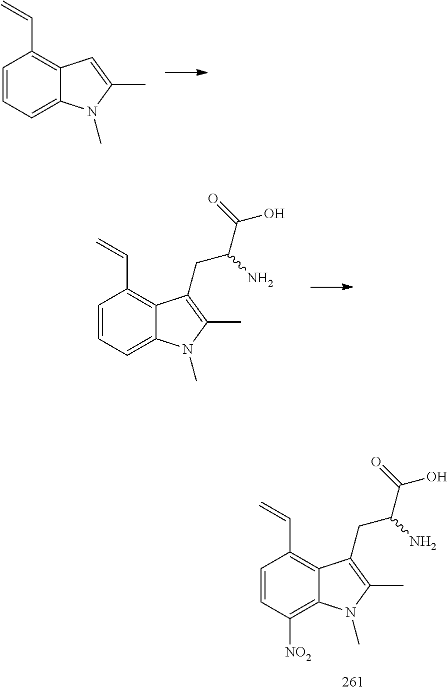 [SMILES: C=Cc1cccc2c1cc(C)n2C ; C=Cc1cccc2c1c(CC(N)C(=O)O)c(C)n2C ; C=Cc1ccc([N+](=O)[O-])c2c1c(CC(N)C(=O)O)c(C)n2C]

Example 261 can be prepared from 4-vinyl-1,2-dimethyl-indole as shown above.

### Example 262: Preparation of 2-amino-3-(5-ethynyl-1,2-dimethyl-4-nitro-1H-indol-3-yl)propanoic acid (262)

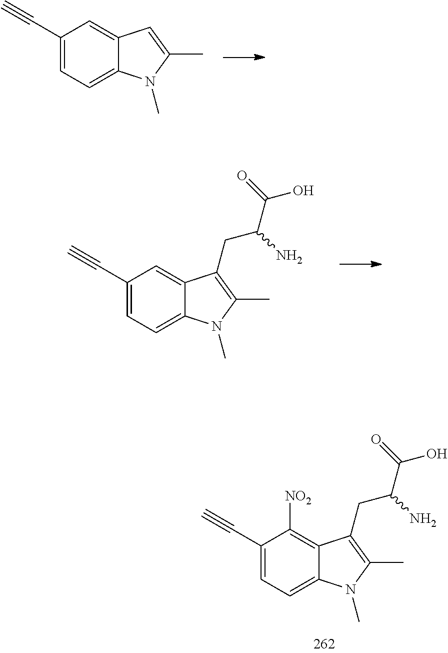 [SMILES: C#Cc1ccc2c(c1)cc(C)n2C ; C#Cc1ccc2c(c1)c(CC(N)C(=O)O)c(C)n2C ; C#Cc1ccc2c(c(CC(N)C(=O)O)c(C)n2C)c1[N+](=O)[O-]]

Example 262 can be prepared from 5-ethynyl-1,2-dimethyl-indole as shown above.

### Example 263: Preparation of 2-amino-3-(6-ethynyl-1,2-dimethyl-4-nitro-1H-indol-3-yl)propanoic acid (263)

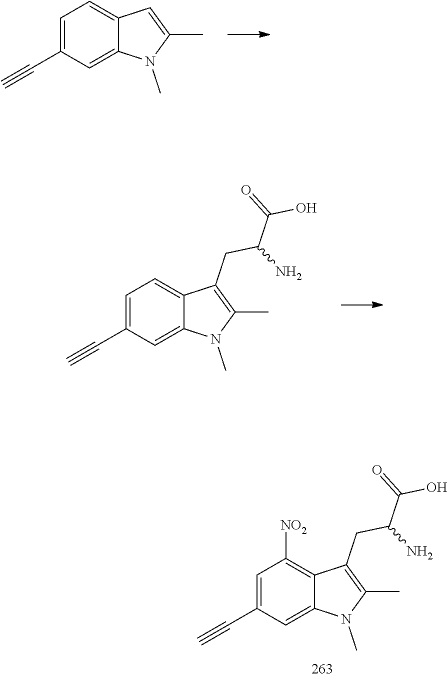 [SMILES: C#Cc1ccc2cc(C)n(C)c2c1 ; C#Cc1ccc2c(CC(N)C(=O)O)c(C)n(C)c2c1 ; C#Cc1cc([N+](=O)[O-])c2c(CC(N)C(=O)O)c(C)n(C)c2c1]

Example 263 can be prepared from 6-ethynyl-1,2-dimethyl-indole as shown above.

### Example 264: Preparation of 2-amino-3-(7-ethynyl-1,2-dimethyl-4-nitro-1H-indol-3-yl)propanoic acid (264)

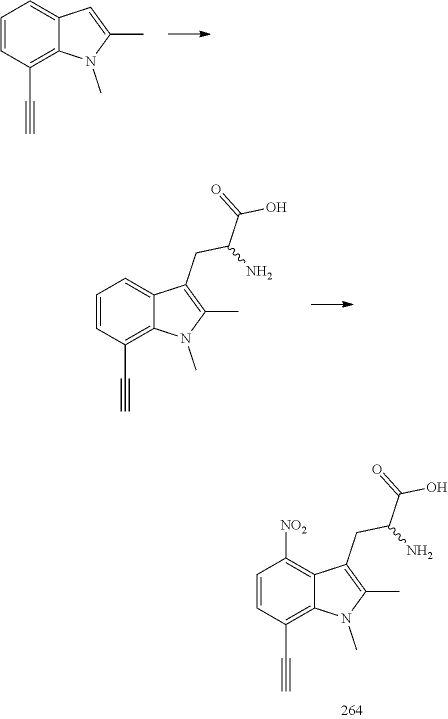 [SMILES: C#Cc1cccc2cc(C)n(C)c12 ; C#Cc1cccc2c(CC(N)C(=O)O)c(C)n(C)c12 ; C#Cc1ccc([N+](=O)[O-])c2c(CC(N)C(=O)O)c(C)n(C)c12]

Example 264 can be prepared from 7-ethynyl-1,2-dimethyl-indole as shown above.

### Example 265: Preparation of 2-amino-3-(4-ethynyl-1,2-dimethyl-7-nitro-1H-indol-3-yl)propanoic acid (265)

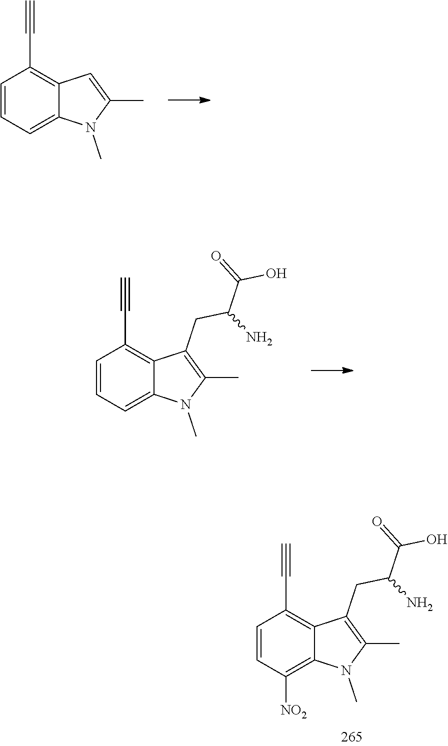 [SMILES: C#Cc1cccc2c1cc(C)n2C ; C#Cc1cccc2c1c(CC(N)C(=O)O)c(C)n2C ; C#Cc1ccc([N+](=O)[O-])c2c1c(CC(N)C(=O)O)c(C)n2C]

Example 265 can be prepared from 4-ethynyl-1,2-dimethyl-indole as shown above.

### Example 266: Preparation of 2-amino-3-(1,2-dimethyl-5-morpholino-4-nitro-1H-indol-3-yl)propanoic acid (266)

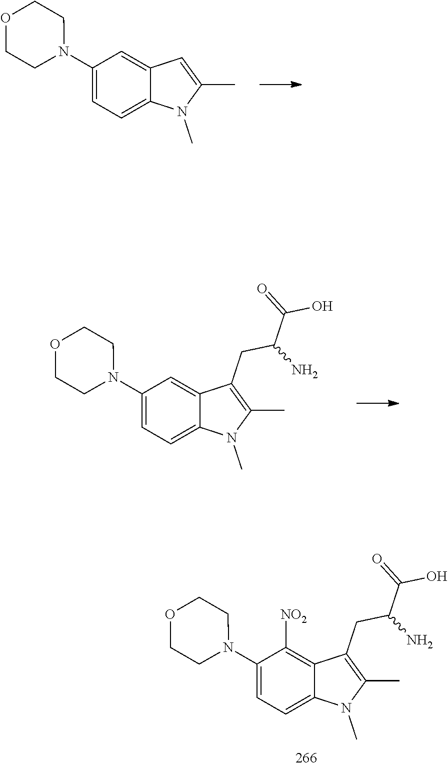 [SMILES: Cc1cc2cc(N3CCOCC3)ccc2n1C ; Cc1c(CC(N)C(=O)O)c2cc(N3CCOCC3)ccc2n1C ; Cc1c(CC(N)C(=O)O)c2c([N+](=O)[O-])c(N3CCOCC3)ccc2n1C]

Example 266 can be prepared from 5-morpholino-1,2-dimethyl-indole as shown above.

### Example 267: Preparation of 2-amin-3-(1,2-dimethyl-6-morpholino-4-nitro-1H-indol-3-yl)propanoic acid (267)

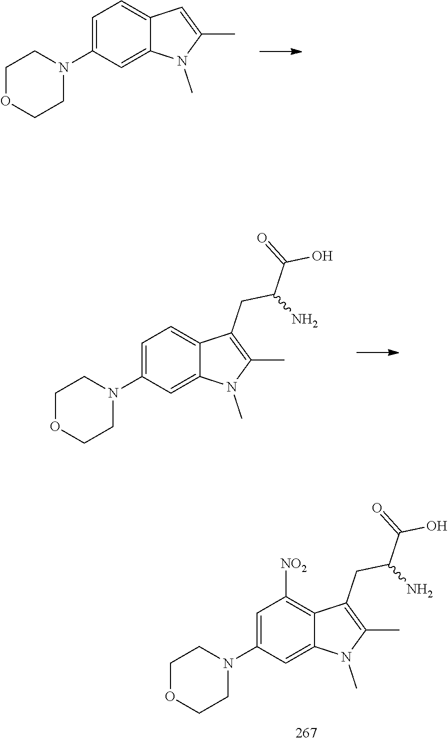 [SMILES: Cc1cc2ccc(N3CCOCC3)cc2n1C ; Cc1c(CC(N)C(=O)O)c2ccc(N3CCOCC3)cc2n1C ; Cc1c(CC(N)C(=O)O)c2c([N+](=O)[O-])cc(N3CCOCC3)cc2n1C]

Example 267 can be prepared from 6-morpholino-1,2-dimethyl-indole as shown above.

### Example 268: Preparation of 2-amino-3-(1,2-dimethyl-7-morpholino-4-nitro-1H-indol-3-yl)propanoic acid (268)

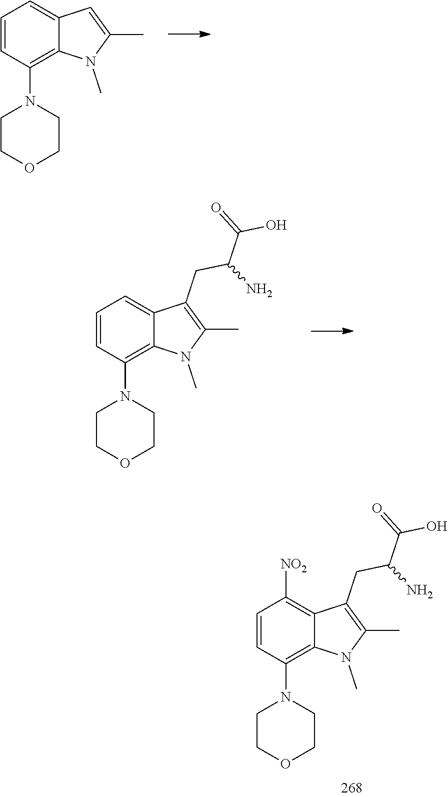 [SMILES: Cc1cc2cccc(N3CCOCC3)c2n1C ; Cc1c(CC(N)C(=O)O)c2cccc(N3CCOCC3)c2n1C ; Cc1c(CC(N)C(=O)O)c2c([N+](=O)[O-])ccc(N3CCOCC3)c2n1C]

Example 268 can be prepared from 7-morpholino-1,2-dimethyl-indole as shown above.

### Example 269: Preparation of 2-amino-3-(1,2-dimethyl-4-morpholino-7-nitro-1H-indol-3-yl)propanoic acid (269)

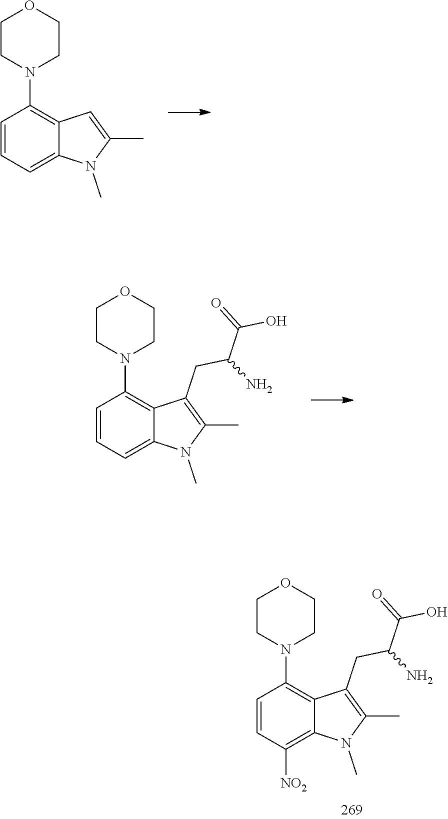 [SMILES: Cc1cc2c(N3CCOCC3)cccc2n1C ; Cc1c(CC(N)C(=O)O)c2c(N3CCOCC3)cccc2n1C ; Cc1c(CC(N)C(=O)O)c2c(N3CCOCC3)ccc([N+](=O)[O-])c2n1C]

Example 269 can be prepared from 4-morpholino-1,2-dimethyl-indole as shown above.

### Example 270: Preparation of 2-amino-3-(1,2-dimethyl-4-methylthio)-4-nitro-1H-indol-3-yl)propanoic acid (270)

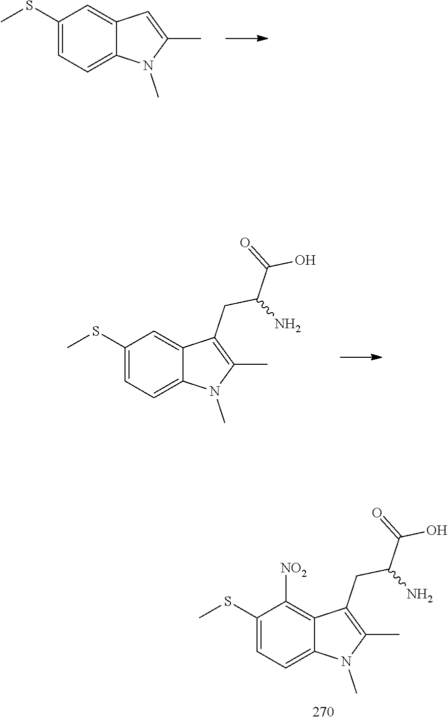

Example 270 can be prepared from 5-(methylthio)-1,2-dimethyl-indole as shown above.

### Example 271: Preparation of 2-amino-3-(1,2-dimethyl-6-(methylthio)-4-nitro-1H-indol-3-yl)propanoic acid (271)

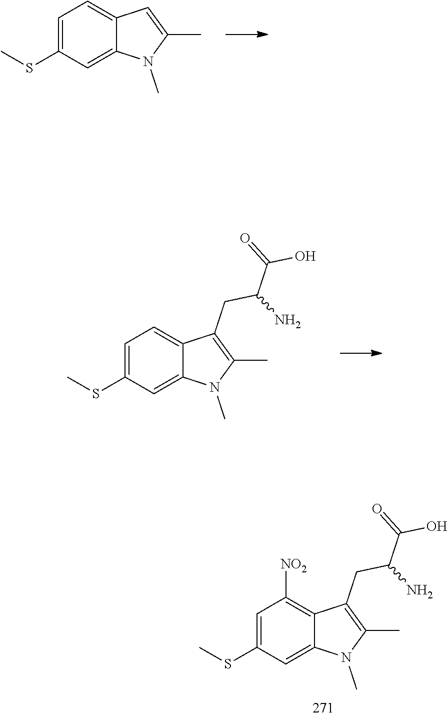

Example 271 can be prepared from 6-(methylthio)-1,2-dimethyl-indole as shown above.

### Example 272: Preparation of 2-amino-3-(1,2-dimethyl-7-(methylthio)-4-nitro-1H-indol-3-yl)propanoic acid (272)

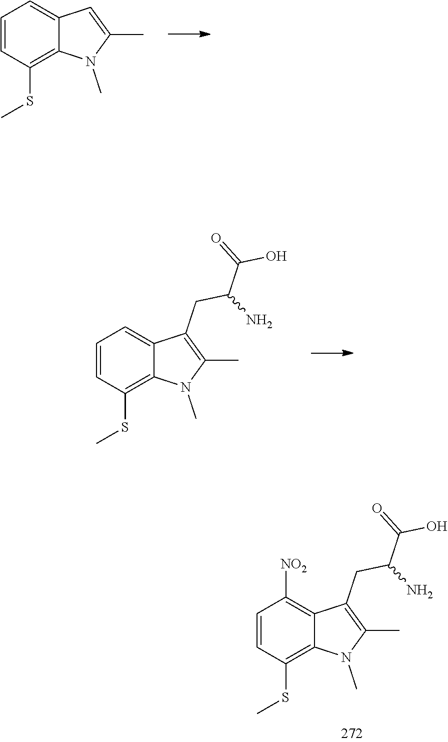 [SMILES: CSc1cccc2cc(C)n(C)c12 ; CSc1cccc2c(CC(N)C(=O)O)c(C)n(C)c12 ; CSc1ccc([N+](=O)[O-])c2c(CC(N)C(=O)O)c(C)n(C)c12]

Example 272 can be prepared from 7-(methylthio)-1,2-dimethyl-indole as shown above.

### Example 273: Preparation of 2-amino-3-(1,2-dimethyl-4-(methylthio)-7-nitro-1H-indol-3-yl)propanoic acid (273)

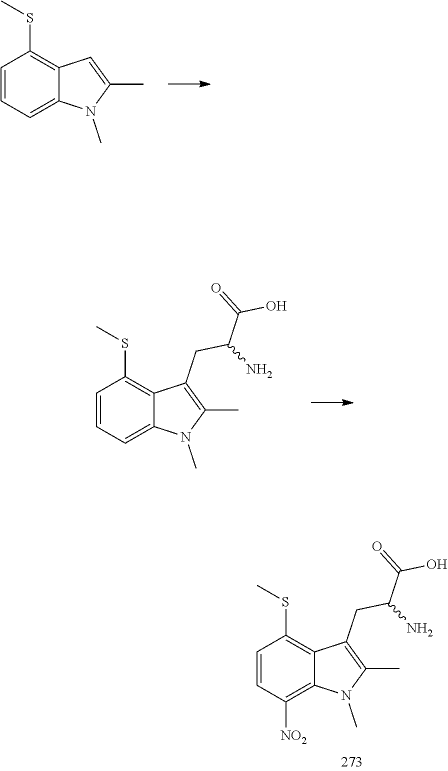 [SMILES: CSc1cccc2c1cc(C)n2C ; CSc1cccc2c1c(CC(N)C(=O)O)c(C)n2C ; CSc1ccc([N+](=O)[O-])c2c1c(CC(N)C(=O)O)c(C)n2C]

Example 273 can be prepared from 4-(methylthio)-1,2-dimethyl-indole as shown above.

### Example 274: Preparation of 2-amino-3-(1,2-dimethyl-4-nitro-5-(pyridin-4-yl)-1H-indol-3-yl)propanoic acid (274)

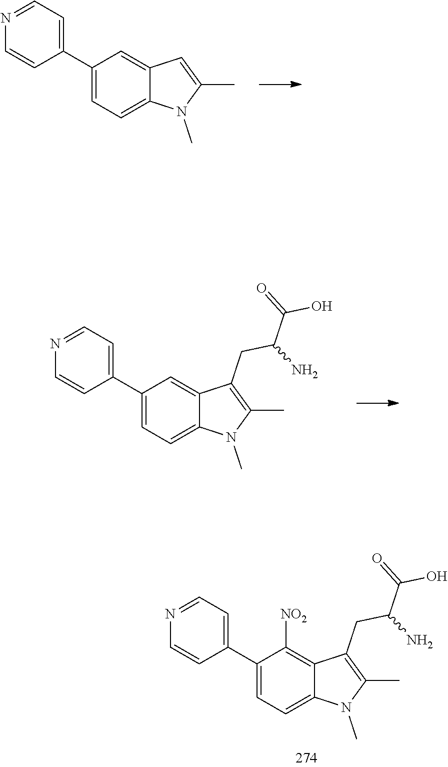 [SMILES: Cc1cc2cc(-c3ccncc3)ccc2n1C ; Cc1c(CC(N)C(=O)O)c2cc(-c3ccncc3)ccc2n1C ; Cc1c(CC(N)C(=O)O)c2c([N+](=O)[O-])c(-c3ccncc3)ccc2n1C]

Example 274 can be prepared from 5-(pyridin-4-yl)-1,2-dimethyl-indole as shown above.

### Example 275: Preparation of 2-amino-3-(1,2-dimethyl-4-nitro-6-(pyridin-4-yl)-1H-indol-3-yl)propanoic acid (275)

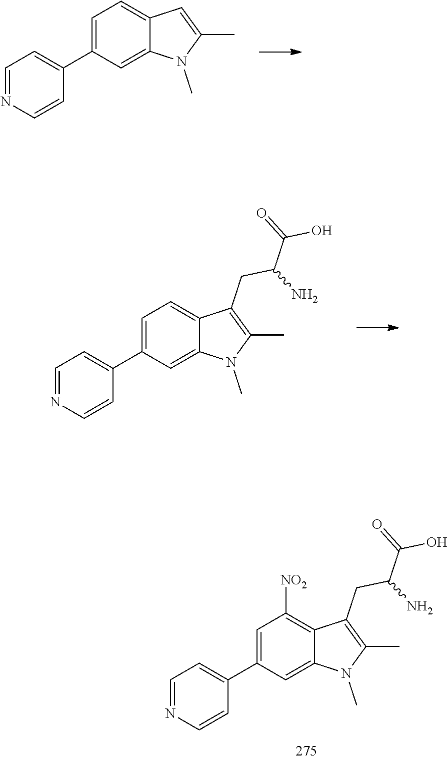 [SMILES: Cc1cc2ccc(-c3ccncc3)cc2n1C ; Cc1c(CC(N)C(=O)O)c2ccc(-c3ccncc3)cc2n1C ; Cc1c(CC(N)C(=O)O)c2c([N+](=O)[O-])cc(-c3ccncc3)cc2n1C]

Example 275 can be prepared from 6-(pyridin-4-yl)-1,2-dimethyl-indole as shown above.

### Example 276: Preparation of 2-amino-3-(1,2-dimethyl-4-nitro-7-(pyridin-4-yl)-1H-indol-3-yl)propanoic acid (276)

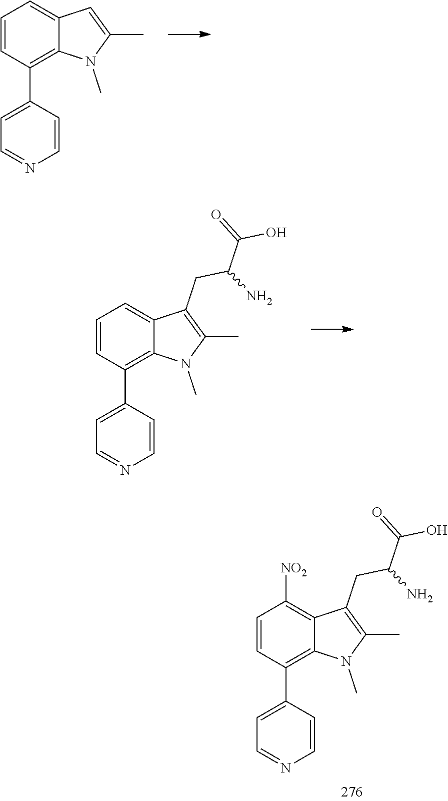 [SMILES: Cc1cc2cccc(-c3ccncc3)c2n1C ; Cc1c(CC(N)C(=O)O)c2cccc(-c3ccncc3)c2n1C ; Cc1c(CC(N)C(=O)O)c2c([N+](=O)[O-])ccc(-c3ccncc3)c2n1C]

Example 276 can be prepared from 7-(pyridin-4-yl)-1,2-dimethyl-indole as shown above.

### Example 277: Preparation of 2-amino-3-(1,2-dimethyl-7-nitro-4-(pyridin-4-yl)-1H-indol-3-yl)propanoic acid (277)

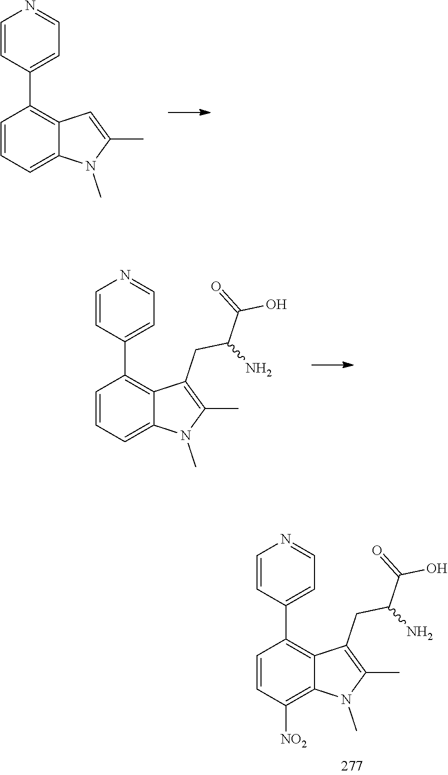 [SMILES: Cc1cc2c(-c3ccncc3)cccc2n1C ; Cc1c(CC(N)C(=O)O)c2c(-c3ccncc3)cccc2n1C ; Cc1c(CC(N)C(=O)O)c2c(-c3ccncc3)ccc([N+](=O)[O-])c2n1C]

Example 277 can be prepared from 4-(pyridin-4-yl)-1,2-dimethyl-indole as shown above.

### Example 278: Genetic Engineering of E. coli for Further Improving the Productivity

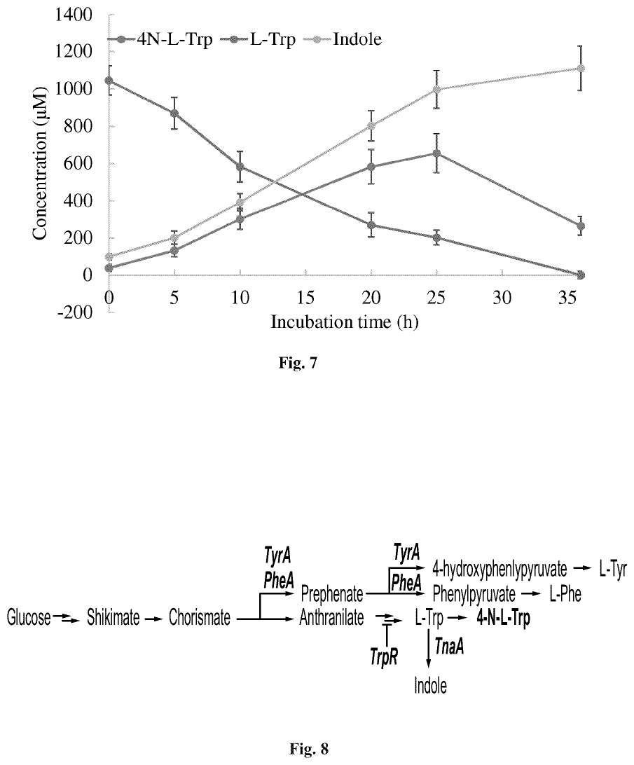

In E. coli, L-Trp has been observed to be converted into indole, pyruvate and NH₃ by tryptophanase TnaA. L-Trp consumption and the formation of 4-NO₂-L-Trp and indole was monitored during the whole cell transformation process (FIG. 7). The concentration of L-Trp in the medium was constantly decreased. Concomitantly, the concentration of indole reached as high as 1100 μM after 36 h, while the concentration of 4-NO₂-L-Trp was topped at 600 μM at 25 h and then decreased. This result indicated that L-Trp degradation is a strong competitive pathway of 4-NO₂-L-Trp synthesis. Therefore, inhibiting the L-Trp degradation pathway, in some embodiments, improves the productivity of 4-NO₂-L-Trp in whole cell systems. Accordingly, the tryptophanase encoding gene tnaA was knocked down in the engineered E. coli strain by the markerless Red recombination method,

During the biological synthesis of 1-Trp in E. coli, TrpR has been observed to repress the transcription of genes involved in L-Trp synthesis and transport when high concentration of L-Trp are present. In addition, in the biosynthesis pathways of aromatic amino acids, carbon flux from chorismite has been observed to flow to the synthesis of L-Phe, L-Tyr and L-Trp (FIG. 8). In some embodiments, to improve the cellular availability of L-Trp, the negative regulator trpR of L-Trp biosynthesis and tyrA and pheA which catalyze the first two steps of the L-Tyr and L-Phe branch pathways, respectively, are knocked out. A triple knockout E. coli strain (ΔtrpRΔtyrAΔpheA) was produced, and L-Trp concentration in serial fermentation cultures of the mutant is evaluated. The mutant is also transformed with a plasmid combination of pETDUET-GDH-BsNOS and pET28b-TB14 to evaluate the productivity of 4-NO₂-L-Trp. Examples of primers used to generate gene knockout of target metabolic genes are shown in Table 2.

TABLE 2
Primers used for the gene knockout.

| | |
|---|---|
| tnaA_fw | ACATCCTTATAGCCACTCTGTAGTATTAATTAAACTTCTTTAAGTT |
| | TTGCATTCCGGGGATCCGTCGACC (SEQ ID NO: 27) |
| tnaA_rv | AATATTCACAGGGATCACTGTAATTAAAATAAATGAAGGATTAT |
| | GTAATGTGTAGGCTGGAGCTGCTTCG (SEQ ID NO: 28) |
| trpR-FRT_fw | TACAACCGGGGGAGGCATTTTGCTTCCCCCGCTAACAATGGCGA |
| | CATATTGTGTAGGCTGGAGCTGCTTC (SEQ ID NO: 29) |
| trpR-FRT_rv | ATTCGGTGCACGATGCCTGATGCGCCACGTCTTATCAGGCCTACA |
| | AAACATATGAATATCCTCCTTA (SEQ ID NO: 30) |
| pheA-FRT_fw | GGCCTCCCAAATCGGGGGGCCTTTTTTATTGATAACAAAAAGGC |
| | AACACTGTGTAGGCTGGAGCTGCTTC (SEQ ID NO: 31) |
| pheA-FRT_rv | GCCAGTAATAATCCAGTGCCGGATGATTCACATCATCCGGCACCT |
| | TTTCACATATGAATATCCTCCTTA (SEQ ID NO: 32) |
| tyrA-FRT_fw | TCAGGATCTGAACGGGCAGCTGACGGCTCGCGTGGCTTAAGAGG |
| | TTTATTGTGTAGGCTGGAGCTGCTTC (SEQ ID NO: 33) |
| tyrA-FRT_rv | CAACCTGATGAAAAGGTGCCGGATGATGTGAATCATCCGGCACT |
| | GGATTACATATGAATATCCTCCTTA (SEQ ID NO: 34) |

### Example 279: Direct Aromatic Nitration System for Synthesis of Nitrotryptophans in Escherichia coli

This example describes design of a biosynthetic pathway for nitrotrp production in E. coli. The production of Nitrotrp and its derivatives primarily uses complicated, heavily polluting synthetic methods, while biocatalytic nitration processes typically require the use of costly, unstable nitric oxide donors. Native thaxtomin-producing plant pathogenic Streptomyces species produce trace amounts of Nitrotrp along with N—CH₃-Nitrotrp and the txtB-inactivated mutant accumulates only up to 6 mg/L of Nitrotrp after 5-day fermentation. TxtB is a nonribosomal peptide synthase that utilizes Nitrotrp as substrate to synthesize thaxtomin D. Production of up to 0.22 g/L of thaxtomins within 6 days by heterologously expressing the thaxtomin gene cluster from S. scabiei 87.22 in S. albus 11074 (S. albus-thx2) has been observed. The S. albus-thx2 and its mutant carrying only the txtE and txtD genes have been observed to produce up to 80 mg/L of Nitrotrp derivatives, mainly N-acetyl-Nitrotrp, but not Nitrotrp.

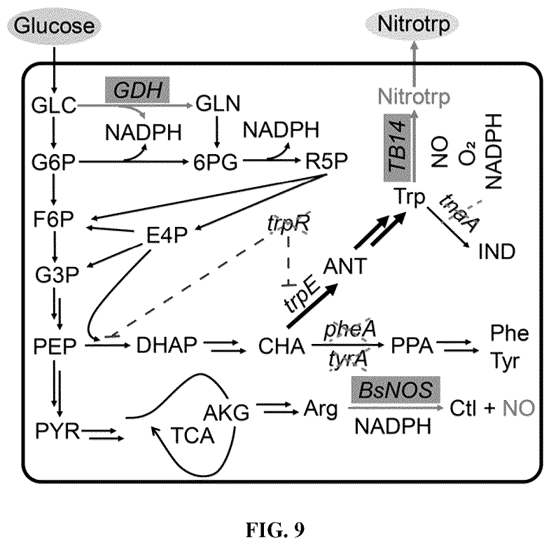

A biosynthetic route to Nitrotrp, in some embodiments, comprises TxtE for 1-Trp nitration and one bacterial NOS for the generation of NO from 1-Arg (FIG. 9). Both TxtE and NOS require reducing agent NADPH for their reactions. In E. coli, NADPH is primarily produced in the pentose phosphate pathway (FIG. 9), but the predominant reducing equivalent is NADH, which potentially limits the production of Nitrotrp. Instead, a glucose dehydrogenase (GDH), specifically Bacillus subtilis GDH, was included in an engineered biosynthetic route to regenerate NADPH from NADP⁺ via converting glucose into glucono-1,5-lactone (FIG. 9). which is then entered the pentose phosphate pathway. A self-sufficient TxtE variant. TB14, was used to construct this pathway. For the bacterial NOS, TxtD, which is naturally coexpressed with TxtE to produce Nitrotrp, was initially selected for use in several Streptomyces strains. However, both wild type and codon-optimized txtD genes from multiple thaxtomin-producing Streptomyces strains were either not expressed or formed inclusion body in E. coli. Codon-optimized NOS from Bacillus subtilis (BsNOS) in E. coli was included in the engineered pathway (FIG. 9). Of note, although E. coli encodes no NOS, its unspecific redox partners support the reaction of BsNOS.

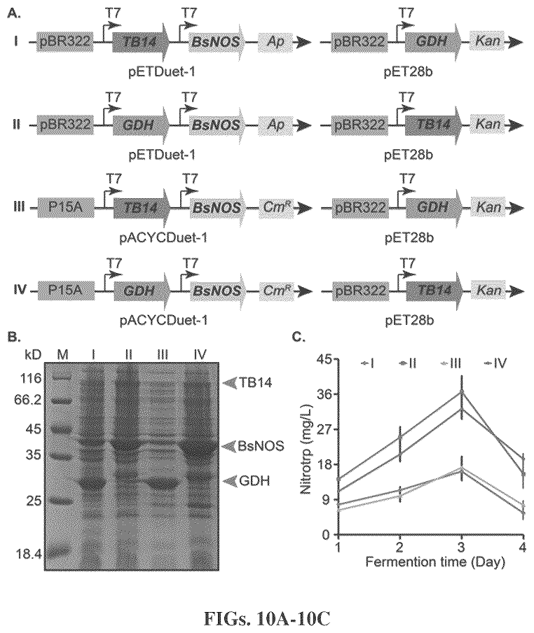

TB14 and BsNOS were cloned into the first and second multiple cloning sites (MCS) of pETDuet-1, respectively (FIG. 10A). Each gene is preceded by a T7 promoter/lac operator. The pETDuet-1 vector contains an ampicillin resistant marker (Ap). The GDH gene was expressed under the control of the T7 promoter in pET28b carrying a kanamycin resistant marker (Kan) (FIG. 10A). Both pET vectors have a medium copy number (15 to 60) in E. coli BL21 strain. The above two constructs were co-transformed into E. coli BL21-GOLD (DE3) (Table 3).

TABLE 3
Bacterial strains

| Name | Function |
|---|---|
| E. coli DH5α | Routine molecular biology studies |
| E. coli BL21-GOLD | Protein expression and production |
| (DE3) | host |
| E. coli ΔtnaA | E. coli BL21-GOLD (DE3) carrying |
| | inactivated tnaA |
| E. coli ΔtrpRtyrApheA | E. coli BL21-GOLD (DE3) carrying |
| | inactivated trpR, tyrA, and pheA |
| E. coli-I | E. coli BL21-GOLD (DE3) carrying the |
| | pathway I |
| E. coli-II | E. coli BL21-GOLD (DE3) carrying the |
| | pathway II |
| E. coli-III | E. coli BL21-GOLD (DE3) carrying the |
| | pathway III |
| E. coli-IV | E. coli BL21-GOLD (DE3) carrying the |
| | pathway IV |
| E. coli-II-TB14 | E. coli BL21-GOLD (DE3) carrying the |
| | pathway II without TB14 |
| E. coli-II-BsNOS | E. coli BL21-GOLD (DE3) carrying the |
| | pathway II without BsNOS |
| E. coli-II-GDH | E. coli BL21-GOLD (DE3) carrying the |
| | pathway II without GDH |
| E. coli ΔtrpRtyrApheA-II | E. coli ΔtrpRtyrApheA carrying the |
| | pathway II |
| E. coli-TB14 | E. coli BL21-GOLD (DE3) carrying |
| | only TB14 |

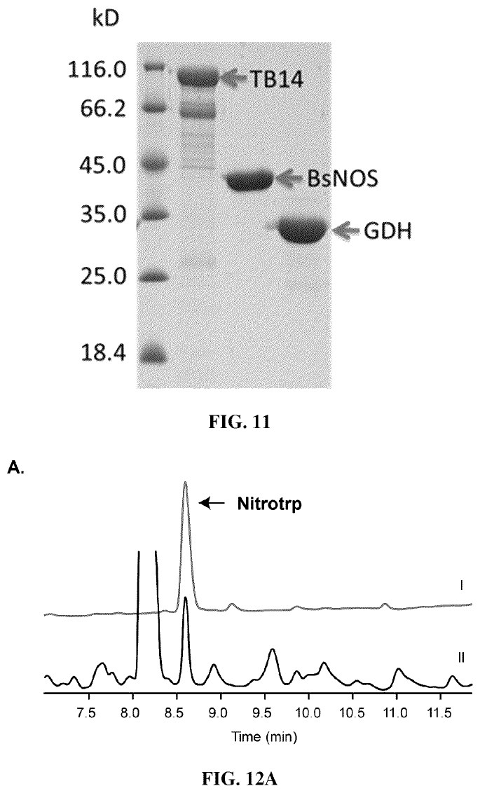

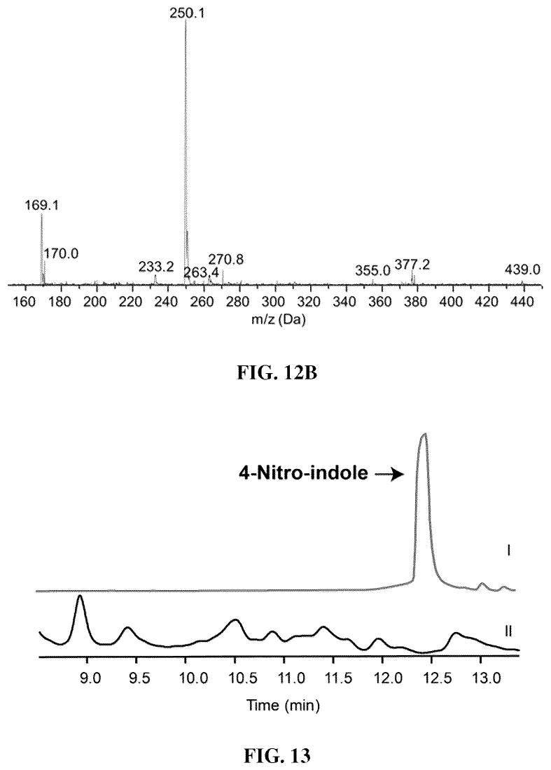

After the selection with both ampicillin and kanamycin, one positive colony (E. coli-I) was picked up to express proteins in TB medium induced with 0.5 mM IPTG for 20 h. The SDS-PAGE analysis of the soluble crude extract revealed the successful overexpression of BsNOS (42 kD) and GDH (28 kD) (Lane I, FIG. 10B and FIG. 11). By contrast, TB14 (110 kD) was expressed to a low level, different from the high solubility of TB14 when expressed alone in the same host. This result indicated that the co-expression with BsNOS and/or GDH negatively influenced the expression level of TB14 in E. coli. Nevertheless, E. coli-I was used for the synthesis of Nitrotrp from cellular 1-Trp and 1-Arg in the M9 minimal medium that has been most widely used for the whole cell transformations. After 24 h, HPLC analysis revealed 7.8±0.7 mg/L of Nitrotrp in the fermentation medium (FIG. 10C, FIG. 12A), which was further confirmed in LC-MS analysis (FIG. 12B). The titer of Nitrotrp increased until day 3, reaching 16.2±2.3 mg/L. and then quickly dropped to 5.5±1.5 mg/L on day 4 (FIG. 10C). TnaA is one tryptophanase in E. coli that is known to catalyze the β-elimination of 1-Trp to produce indole (FIG. 9). HPLC analysis of the fermentation medium failed to identify 4-nitro-indole, the potential product of Nitrotrp degradation by TnaA (FIG. 13). The tnaA gene from E. coli was cloned and recombinant enzyme was prepared (FIG. 14A). Recombinant TnaA produced indole from 1-Trp: degraded Nitrotrp was not observed in an in vitro assay (FIG. 14B). On the other hand, E. coli encodes two oxygen-insensitive nitroreductases NsfA and NsfB that are known to reduce nitroaromatics: one or both of these enzymes are involved in the rapid degradation of Nitrotrp in E. coli-I (FIG. 10C). Data described herein indicate a novel cell-based biocatalytic route to Nitrotrp.

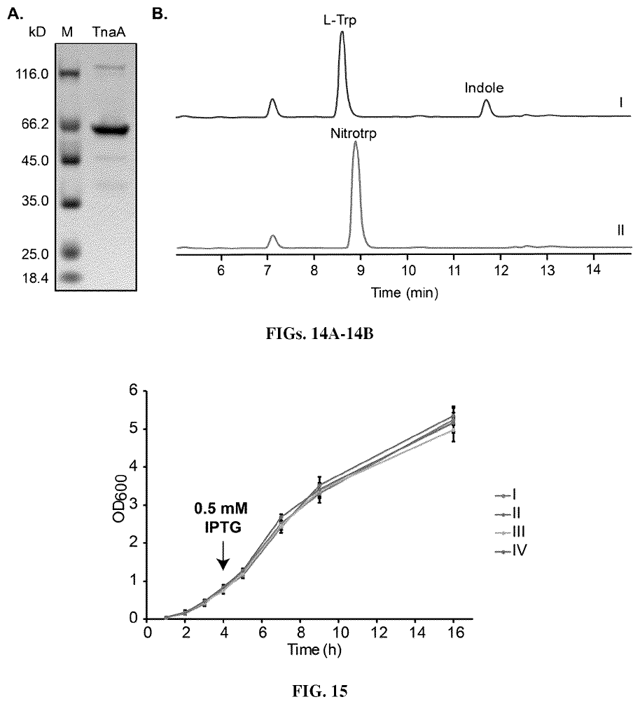

Despite the successful production of Nitrotrp by E. coli-I (I, FIG. 10A), low expression level of TB14 may constrain the nitration process. To address this potential issue, three additional pathways were constructed by varying copy numbers and replicons of plasmid backbones and coexpression of these genes (FIG. 10A). As high expression level is obtained when TB14 is expressed alone, the second design shuffled the TB14 and GDH genes between pETDuet-1 and pET28b (II, FIG. 10A). Furthermore. coexpression of the two genes was investigated in the pACYCDuet-1 backbone that has a low copy number (˜10), contains two T7 promoters in two MCSs, carries the P15A replicon, and includes a chloramphenicol resistant marker (CmR) (III-IV, FIG. 10A). These features allowed the assessment of the effects of improved plasmid stability with two different replicons and varied gene dosages on the production of Nitrotrp. The three new Nitrotrp pathways were transformed into E. coli BL21-GOLD (DE3) to generate E. coli-II, -II and -IV as described above (Table 3). All four strains showed similar growth rates when cultured in TB medium and induced with 0.5 mM IPTG (FIG. 15). SDS-PAGE analysis revealed increased levels of TB14 but the decreased levels of GDH in E. coli-II and -IV in comparison to E. coli-I and —III (FIG. 108). Another finding of the SDS-PAGE analysis was that the co-expression of TB14 and BsNOS on the plasmids of both low and medium copy numbers yielded the same low levels of both enzymes. The three E. coli strains were further fermented along with E. coli-I in the M9 medium for 4 days. HPLC analysis revealed that E. coli-III and -IV, which both carried the separately expressed TB14, produced more Nitrotrp than E. coli-I and —III (FIG. 10C). This data indicates that the low level of GDH in E. coli-II and -IV is sufficient to support the reactions of TB14 and BsNOS (FIG. 10B). The titer of Nitrotrp by all four strains increased until day 3 and then dropped at day 4. The highest titer of Nitrotrp was observed at 36.5 t 4.0 mg/L with E. coli-II on day 3, the 2.3-fold improvement compared with E. coli-I at day 3 (FIG. 10C). E. coli-II was therefore selected for subsequent studies.

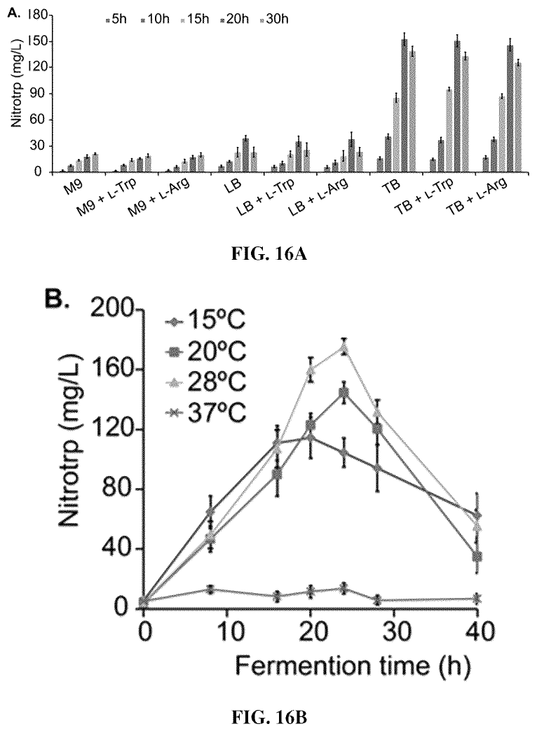

The fermentation processes in two commonly used, nutritionally rich media, LB and TB were investigated. As cellular 1-Trp and 1-Arg are consumed to produce Nitrotrp (FIG. 9). the effects of supplemented amino acids (5 mM) in M9. LB and TB media on the production of Nitrotrp by E. coli-II at 20° C. were also examined. HPLC analysis measured the concentration of Nitrotrp in the cell-free media at 5 h, 10 h, 15 h, 20 h, and 30 h (FIG. 16A). E. coli-II reached the highest titer of Nitrotrp in the M9 medium at day 3 (FIG. 10C). but the highest amount appeared after 20 h in both LB and TB, which was then decreased at 30 h. The titer of Nitrotrp was increased from 18.0±2.0 mg/L. in M9 to 39.0±3.3 mg/L in LB and 152.8±10.5 mg/L in TB after 20 h, indicating the faster and increased production of Nitrotrp in nutritionally rich media (FIG. 16A). Supplementation of 1-Trp or 1-Arg in all three media resulted in no increase in the production of Nitrotrp.

Temperature effects on the whole-cell nitration process were also examined. Fermentation experiments were performed at 20° C., 15° C., 28° C., and 37° C. Crude extracts of fermentation media of E. coli-II cultured at the four temperatures were prepared at 0 h, 8 h, 16 h, 20 h, 24 h, 28 h, and 40 h. HPLC analysis indicated a similar level of Nitrotrp at 8 h and 16 h when E. coli-II was fermented at 15° C. 20° C. and 28° C. (FIG. 16B). At 15° C., the titer of Nitrotrp remained largely unchanged (about 110 mg/L) from 16 h to 24 h and then decreased to about 63 mg/L at 40 h. By contrast, E col-IL produced the highest amount of Nitrotrp after 24 h at 20° C. and 28° C., and fermentation at 28° C. resulted in production of 175.5±5.3 mg/L Nitrotryp, E. coli-II produced less than 14 mg/L of Nitrotrp at any time point when fermented at 37° C. Data indicate increased production of Nitrotrp in E. coli from 36.5 mg/L for 3 days to 175.5 mg/L for 1 day based upon changes to fermentation media and temperature conditions.

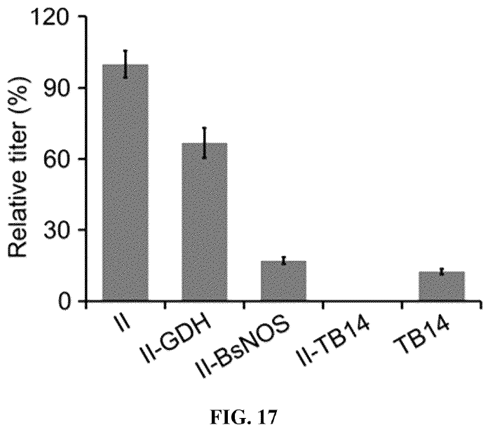

Nitrotrp pathway II comprises TB14, BsNOS, and GDH (FIG. 10A) that together lead to the production of 175 mg/L of Nitrotrp by E. coli-II (FIG. 16B). Removal of each enzyme was investigated. TB14. BsNOS, and GDH genes were removed individually from the pathway II and the three resultants were transformed into E. coli BL21-GOLD (DE3) to generate E. coli-II-TB14, -BsNOS, and -GDH. As the control, pBT28b-TB14 was used to create E. coli-TB14 following the same procedure (Table 3). These four new strains along with E. coli-II were cultured in TB at 28° C. for 24 h. HPLC analysis then quantitated the titers of Nitrotrp by all five strains (FIG. 17). E. coli-II-TB14 completely lost the ability to produce Nitrotrp. E. coli-II-BsNOS retained 17.2±1.5% of the titer of E. coli-II, indicating the presence of additional sources of NO for the TB14 reaction (FIG. 17). For example in some embodiments, an indoor atmosphere can supply more than 2 μM of NO for the biotransformation. Similarly, E. coli-TB14 also produced a relatively low level of Nitrtrp (12.5±1.2% of the titer of E. coli-II). Among the three enzymes in the pathway II, GDH was observed to provide a supporting role to the reactions of TB14 and BsNOS as E. coli-II-GDH showed 66.8±6.4% of the titer of E. coli-II (FIGS. 9 and 17).

The production of Nitrotrp consumes cellular 1-Trp and 1-Arg of E. coli (FIG. 9). The concentrations of 1-Trp in TB were measured at varying time points when fermenting E. coli-II; a quick decrease from 260.8±12.0 mg/L at 0 h to 25.3±9.8 mg/L after 24 h was observed (FIG. 18). However, 1-Trp supplementation to TB was not observed to have an effect on improving the production of Nitrotrp (FIG. 16A), indicating the increased cellular availability of 1-Trp may be more important to increase Nitrotrp production. Tuning of 1-Trp metabolic pathways in E. coli (FIG. 9) was investigated. Of note, 1-Arg biosynthesis was not selected as the primary engineering target as the increased 1-Arg may lead, in some embodiments, to the higher production of NO that can be detrimental to E. coli. The tnaA gene in E. coli BL21-GOLD(DE3) was knocked out by the λ red recombination approach. to prevent the conversion of cellular 1-Trp into indole (FIGS. 9 and 19). Pathway II was then transferred into the E. coli ΔtnaA mutant for the production of Nitrotrp (Table 3). Neither recombinant TB14 and BsNOS nor GDH was detectable from soluble protein fraction of E. coli AtnaA-II by SDS-PAGE analysis (FIG. 20). leading to no observed production of Nitrotrp.

Increasing the metabolic flux to 1-Trp biosynthesis was investigated (FIG. 9). The biosynthetic pathways of 1-Trp, 1-Phe, and 1-Tyr require the same cellular metabolite chorismate that is produced from the shikimate biosynthetic pathway. TyrA and PheA convert chorismate into prephenate for the production of 1-Tyr and 1-Phe, while TrpE produces anthranilate from chorismate to synthesize 1-Trp. The inactivation of both tyrA and pheA genes in E. coli BL21-GOLD(DE3), in some embodiments, eliminates the competitive consumption of chorismate for the increased production of 1-Trp in the fermentation stage. TrpR has also been observed to provide a negative feedback regulation on the 1-Trp biosynthesis by acting on 1-Trp biosynthetic gene and the shikimate biosynthetic gene (FIG. 9). and the inactivation of the trpR gene has been observed to overproduce 1-Trp in E. coli. These three genes were inactivated in E. coli BL21-GOLD (DE3) to create E. coli ΔtrpRtyrApheA using the λ red recombination approach (Table 3. FIG. 19). Both wild type E. coli and the ΔtrpRtyrApheA mutant were fermented in TB medium under the same conditions for 20 h. HPLC analysis revealed 273±33 mg/L of free 1-Trp in the fermentation medium of the mutant and 142±25 mg/L for the wild type, indicating an increased intracellular supply of 1-Trp in the mutant. E. coli ΔtrpRtyrApheA-II was then generated by transforming the designed Nitrotrp pathway II into the mutant. HPLC analysis revealed the concentration of 1-Trp in the culture of E. coli ΔtrpRtyrApheA-I was decreasing in the fermentation process but remained higher than E. coli-l at the majority of time points (FIG. 18), indicating that the mutant production strain may have a slower rate to consume medium 1-Trp than E. coli-II as it synthesizes more cellular 1-Trp, E. coli ΔtrpRtyrApheA-III produced a higher level of Nitrotrp than E. coli-II at 10 h and 20 h (FIG. 18), and reached the highest titer, 191.8±10.3 mg/L, at 20 h, which was about 10% increased compared with E. coli-II at 24 h (FIG. 16B).

Production of Nitrotrp analogs was examined by feeding eight unnatural racemic 1-Trp analogs (except for 5-F-1-Trp) (5 mM) to the fermentation medium of E. coli-II (FIGS. 21 and 22A). HPLC analysis revealed that α-Me-Trp, 4-F-Trp, 4-Me-Trp, 5-MeO-Trp, 5-Me-Trp, 5-F-1-Trp, 6-F-Trp and 7-Me-Trp all were nitrated along with 1-Trp to varying extents by E. coli-II (FIG. 22B). The 1-amino acid substrate was observed to be nitrated by TB14 in in vitro studies. The strain demonstrated the highest nitration activity toward 5-Me-Trp, followed by 5-F-Trp and 4-Me-Trp, while only about 2.5 mg/L of the nitro product was produced from fed 4-F-Trp (FIG. 21). This data agreed with the observed in vitro catalytic performance of TB14 toward these substrates. The titer of nitro-5-Me-Trp reached 61.5 t 5.5 mg/L, along with 80.3±10.4 mg/L of Nitrotrp after culturing E. coli-1H for 24 h (FIG. 21).

### Materials and Methods

### General Chemicals, DNA Sub-Cloning, and Bacterial Strains

Molecular biology reagents and enzymes were purchased from Fisher Scientific. Primers were ordered from Sigma-Aldrich, Racemic 4-Me-Trp was from MP Biomedical (Santa Ana, CA). Other chemicals and solvents were purchased from Sigma-Aldrich or Fisher Scientific. Escherichia coli DH5α (Life Technologies) was used for molecular biology work, while E. coli BL21-GOLD (DE3) (Agilent) was used for protein overexpression and the development of the whole cell nitration systems (Table 3). E. coli strains were grown in M9, LB or TB. DNA sequencing was performed at Eurofins. A Shimadzu Prominence UHPLC system (Kyoto, Japan) fitted with an Agilent Poroshell 120 EC-C18 column (2.7 μm, 3.0×50 mm), coupled with a PDA detector was used for HPLC analysis.

### Creation of Nitrotrp Biosynthetic Pathways

BsNOS, TB14 and GDH genes were amplified from pET15b-BsNOS. pET28b-TB14, and pET21b-GDH, respectively using primers listed in Table 4. PCR amplicons were analyzed by agarose gel and extracted with GeneJET Gel Extraction Kit (Thermo). Purified PCR products, pACYCDuet-1, pETDuet-1, and pET28b were digested with corresponding restriction enzymes, purified and then ligated to create expression constructs. All inserts in the constructs were sequenced to exclude potential errors introduced during PCR amplification and gene manipulation.

### Whole-cell Biotransformation

E. coli BL21-GOLD (DE3) competent cells were transformed with the designed pathway I-IV individually (Table 3). Positive colonies of E. coli-I, to -IV were selected on LB agar supplemented with 0.1 mg/mL ampicillin and 0.05 mg/mL kanamycin or 0.05 mg/mL chloramphenicol and 0.05 mg/mL kanamycin. One colony of each strain was then grown in LB with proper antibiotics at 37° C., 250 rpm overnight. The seed cultures were used to inoculate 100 mL of TB with proper antibiotics and 1× trace metal solution (1000× stock solution: 50 mM FeCl₃, 20 mM CaCl₂, 10 mM MnSO₄, 10 mM ZnSO₄, 2 mM CoSO₄, 2 mM CuCl₂, 2 mM NiCl₂, 2 mM Na₂MoO₄, and 2 mM H₃BO₃) for culturing at 37° C., 250 rpm until OD₆₀₀ reached 0.6-0.8. We then induced protein expression by 0.5 mM IPTG at 18° C., 250 rpm for 20 h. For the evaluation of protein expression, cell pellets were then collected after centrifugation (5.000 g, 10 min, and 4° C.). and resuspended in the suitable volume of lysis buffer (cell biomass:volume=1:4) [25 mM Tris-HCl, pH 8.0, 100 mM NaCl, 20 mM imidazole, 3 mM 0-mercaptoethanol (BME) and 10% glycerol]. Soluble proteins were released by sonication and collected after centrifugation at 35,000×g at 4° C. for 30 min. Clear supernatants (20 μL) was mixed with dye and subject to SDS-PAGE analysis. For the whole cell biotransformation, bacterial cells in TB were harvested after centrifugation (2,000 g at 4° C. for 10 min) and resuspended to OD₆₀₀=30 in fresh test media (M9, LB, or TB with or without 5 mM 1-Trp or 1-Arg). The fermentation was then performed at different temperatures, 250 rpm and aliquots (0.1 mL) of the fermentation culture were taken at various time points. The whole-cell biotransformation in aliquots was quenched by mixing with 0.2 mL of methanol. After centrifugation at 14.000 rpm for 30 minutes. the supernatant was subject to HPLC analysis. All experiments were independently repeated at least twice.

Inactivation of Genes in E. coli

Inactivation of tnaA, trpR, tyrA, and pheA in E. coli BL21-GOLD (DE3) was performed following the λ red recombination protocol (FIG. 19). Specific primers used were included in Table 4.

### TnaA Assay

The tnaA gene was amplified from E. coli genomic DNA using primers listed in Table 4. PCR amplicons were analyzed by agarose gel and extracted with GeneJET Gel Extraction Kit (Thermo). Purified PCR products and pET28b were digested with corresponding restriction enzymes, purified and then ligated to create expression constructs. Insert in the construct was sequenced to exclude potential errors introduced during PCR amplification and gene manipulation. Recombinant TnaA was prepared in E. coli BL21-GOLD (DE3). The enzyme assay (0.1 mL) contained 100 mM potassium phosphate buffer (pH 8.3), 0.2 mM pyridoxal 5-phosphate and 0.1 μM purified tnaA. The reaction mixtures were pre-warmed at 37° C. for 5 minutes. and initiated by adding 0.5 mM 1-Trp or Nitrotrp as substrate. After 10 minutes, the reactions were quenched by mixing well with 0.2 mL of methanol. After centrifugation at 14,000 rpm for 30 minutes. the supernatant was subject to HPLC analysis. All experiments were independently repeated at least twice.

### HPLC and LC-MS Methods

For HPLC analysis, the C18 column was kept at 30° C. and ran first with 5% solvent B (acetonitrile, 0.1% formic acid) for 2 min and then a linear gradient of 5-15% solvent B in 5 min, followed by another linear gradient of 15-95% solvent B in 10 min. The column was further cleaned with 95% solvent B for 3 min and then re-equilibrated with 5% solvent B for 2 min. Solvent A was water with 0.1% formic acid. The flow rate was set as 0.5 mL/min, and the products were detected at 211 nm with a PDA detector. The concentrations of Nitrotrp and/or 1-Trp in the samples were determined on the basis of standard curves of two authentic compounds after HPLC analysis (FIGS. 22A-22B). LC-MS analysis was performed by established protocols.

TABLE 4

| Name | Sequence (5′→3′) | Function |
|---|---|---|
| TB14FN | ATACCATGGTGACCGTCCCCTCGCCG (SEQ ID NO: 35) | TB14 cloning |
| TB14RH | ATCAAGCTTCCCAGCCCACACGTCTTTTGC (SEQ ID NO: 36) | TB14 cloning |
| GDHFB | CAGGATCCGATGTATAAAGATCTGGAAGGTAAAGTGGTG | GDH cloning |
| | (SEQ ID NO: 37) | |
| GDHRH | CAAAGCTTTTAGCCACGACCTGCCTGAAAG (SEQ ID NO: 38) | GDH cloning |
| BsNOSFN | ACTCATATGATGGAAGAAAAAGAAATC (SEQ ID NO: 39) | BsNOS cloning |
| BsNOSRH | ACTAAGCTTCTATTCATACGGTTTGTC (SEQ ID NO: 40) | BsNOS cloning |
| tnaAFB | ACTGGATCCGATGGAAAACTTTAAACATCTCC (SEQ ID NO: 41) | tnaA cloning |
| tnaARE | ACTGAATTCGAAACTTCTTTAAGTTTTGCGGTG (SEQ ID NO: 42) | tnaA cloning |
| trpR-F | TACAACCGGGGGAGGCATTTTGCTTCCCCCGCTAACAATGGCGAC | trpR knock out |
| | ATATTGTGTAGGCTGGAGCTGCTTC (SEQ ID NO: 43) | |
| trpR-R | ATTCGGTGCACGATGCCTGATGCGCCACGTCTTATCAGGCCTACA | trpR knock out |
| | AAACATATGAATATCCTCCTTA (SEQ ID NO: 44) | |
| pheA-F | GGCCTCCCAAATCGGGGGGCCTTTTTTATTGATAACAAAAAGGCA | pheA knock out |
| | ACACTGTGTAGGCTGGAGCTGCTTC (SEQ ID NO: 45) | |
| pheA-R | GCCAGTAATAATCCAGTGCCGGATGATTCACATCATCCGGCACCTT | pheA knock out |
| | TTCACATATGAATATCCTCCTTA (SEQ ID NO: 46) | |
| tyrA-F | TCAGGATCTGAACGGGCAGCTGACGGCTCGCGTGGCTTAAGAGG | tyrA knock out |
| | TTTATTGTGTAGGCTGGAGCTGCTTC (SEQ ID NO: 47) | |
| tyrA-R | CAACCTGATGAAAAGGTGCCGGATGATGTGAATCATCCGGCACTG | tyrA knock out |
| | GATTACATATGAATATCCTCCTTA (SEQ ID NO: 48) | |
| tnaA-F | GGATCACTGTAATTAAAATAAATGAAGGATTATGTAATGGTGTAGGC | tnaA knock out |
| | TGGAGCTGCTTC (SEQ ID NO: 49) | |
| tnaA-R | GTGGCTAACATCCTTATAGCCACTCTGTAGTATTAATTACATATGAAT | tnaA knock out |
| | ATCCTCCTTA (SEQ ID NO: 50) | |

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an.” as used herein in the specification and in the claims. unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment. to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements. and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as 11 indicating exclusive alternatives (i.e. “one or the other but not both”) when preceded by terms of exclusivity, such as “either,” “one of,” “only one of.” or “exactly one of.” “Consisting essentially of,” when used in the claims. shall have its ordinary meaning as used in the field of patent law.

As used herein in the specification and in the claims, the phrase “at least one.” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus. as a non-limiting example, “at least one of A and B” (or, equivalently. “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B. with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like am to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1-39. (canceled)

40. A method for producing a compound of Formula (I) or Formula (IV): 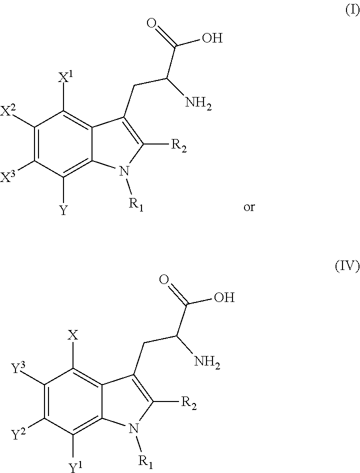
wherein:
X¹ is halogen or C₁₋₆ alkyl;
each X² and X³ is, independently, hydrogen, halogen, or C₁₋₆ alkyl;
each Y¹, Y², and Y³ is, independently, hydrogen, halogen or C₁₋₆ alkyl;
Y is NO₂;
X is NO₂;
R₁ is hydrogen or C₁₋₆ alkyl; and
R₂ is hydrogen or C₁₋₆ alkyl;
the method comprising:
(i) introducing into a bacterial cell culture comprising a recombinant bacterial cell comprising one or more isolated nucleic acids engineered to express:
a fusion protein comprising a TxtE enzyme linked to a catalytic domain of a CYP102A1 (P450BM3) reductase enzyme via an amino acid linker sequence that is between 14 and 27 amino acids in length, wherein the fusion protein is a TB14 fusion protein having the sequence set forth in SEQ ID NO: 1;
a nitric oxide synthase (NOS) enzyme; and, optionally
a glucose dehydrogenase (GDH) enzyme; and
one or more L-Trp molecules and/or one or more L-Trp analogue molecules;
(ii) growing the bacterial cell culture under conditions under which the fusion protein catalyzes a nitration reaction which produces one or more compounds of Formula (I) or Formula (IV); and, optionally,
(iii) isolating the compounds of Formula (I) or Formula (IV).

41. The method of claim 40, wherein:
Y is NO₂, and each R₁ and R₂ is hydrogen;
Y is NO₂, R₁ is methyl, and R₂ is hydrogen;
Y is NO₂, R₁ is hydrogen, and R₂ is methyl; or
Y is NO₂, and each R₁ and R₂ is methyl.

42. The method of claim 40, wherein X¹ is a halogen selected from bromo, chloro, or fluoro.

43. The method of claim 40, wherein each X² and X³ is hydrogen.

44. The method of claim 40, wherein:
X is NO₂, and each R₁ and R₂ is hydrogen;
X is NO₂, R₁ is methyl, and R₂ is hydrogen;
X is NO₂, R₁ is hydrogen, and R₂ is methyl; or
X is NO₂, and each R₁ and R₂ is methyl.

45. The method of claim 44, wherein Y¹ is a halogen selected from bromo, chloro, or fluoro, and each Y² and Y³ is hydrogen.

46. The method of claim 44, wherein Y² is a halogen selected from bromo, chloro, or fluoro, and each Y¹ and Y² is hydrogen.

47. The method of claim 44, wherein Y³ is a halogen selected from bromo, chloro, or fluoro, and each Y¹ and Y² is hydrogen.

48. The method of claim 40, wherein the compound of Formula (I) is:
(S)-2-amino-3-(4-fluoro-7-nitro-1H-indol-3-yl)propanoic acid;
(S)-2-amino-3-(4-chloro-7-nitro-1H-indol-3-yl)propanoic acid;
(S)-2-amino-3-(4-bromo-7-nitro-1H-indol-3-yl)propanoic acid;
2-amino-3-(4-fluoro-7-nitro-1H-indol-3-yl)propanoic acid;
2-amino-3-(4-chloro-7-nitro-1H-indol-3-yl)propanoic acid;
2-amino-3-(4-bromo-7-nitro-1H-indol-3-yl)propanoic acid;
2-amino-3-(4-fluoro-1-methyl-7-nitro-1H-indol-3-yl)propanoic acid;
2-amino-3-(4-chloro-1-methyl-7-nitro-1H-indol-3-yl)propanoic acid;
2-amino-3-(4-bromo-1-methyl-7-nitro-1H-indol-3-yl)propanoic acid;
2-amino-3-(4-fluoro-2-methyl-7-nitro-1H-indol-3-yl)propanoic acid;
2-amino-3-(4-chloro-2-methyl-7-nitro-1H-indol-3-yl)propanoic acid;
2-amino-3-(4-bromo-2-methyl-7-nitro-1H-indol-3-yl)propanoic acid;
2-amino-3-(4-fluoro-1,2-dimethyl-7-nitro-1H-indol-3-yl)propanoic acid;
2-amino-3-(4-chloro-1,2-dimethyl-7-nitro-1H-indol-3-yl)propanoic acid; or
2-amino-3-(4-bromo-1,2-dimethyl-7-nitro-1H-indol-3-yl)propanoic acid.

49. The method of claim 40, wherein the compound of Formula (IV) is:
(S)-2-amino-3-(5-fluoro-4-nitro-1H-indol-3-yl)propanoic acid;
(S)-2-amino-3-(6-fluoro-4-nitro-1H-indol-3-yl)propanoic acid;
(S)-2-amino-3-(7-fluoro-4-nitro-1H-indol-3-yl)propanoic acid;
(S)-2-amino-3-(5-chloro-4-nitro-1H-indol-3-yl)propanoic acid;
(S)-2-amino-3-(6-chloro-4-nitro-1H-indol-3-yl)propanoic acid;
(S)-2-amino-3-(7-chloro-4-nitro-1H-indol-3-yl)propanoic acid;
(S)-2-amino-3-(5-bromo-4-nitro-1H-indol-3-yl)propanoic acid;
(S)-2-amino-3-(6-bromo-4-nitro-1H-indol-3-yl)propanoic acid;
(S)-2-amino-3-(7-bromo-4-nitro-1H-indol-3-yl)propanoic acid;
2-amino-3-(6-fluoro-4-nitro-1H-indol-3-yl)propanoic acid;
2-amino-3-(7-fluoro-4-nitro-1H-indol-3-yl)propanoic acid;
2-amino-3-(5-chloro-4-nitro-1H-indol-3-yl)propanoic acid;
2-amino-3-(6-chloro-4-nitro-1H-indol-3-yl)propanoic acid;
2-amino-3-(7-chloro-4-nitro-1H-indol-3-yl)propanoic acid;
2-amino-3-(5-bromo-4-nitro-1H-indol-3-yl)propanoic acid;
2-amino-3-(6-bromo-4-nitro-1H-indol-3-yl)propanoic acid;
2-amino-3-(7-bromo-4-nitro-1H-indol-3-yl)propanoic acid;
2-amino-3-(6-fluoro-1-methyl-4-nitro-1H-indol-3-yl)propanoic acid;
2-amino-3-(7-fluoro-1-methyl-4-nitro-1H-indol-3-yl)propanoic acid;
2-amino-3-(5-chloro-1-methyl-4-nitro-1H-indol-3-yl)propanoic acid;
2-amino-3-(6-chloro-1-methyl-4-nitro-1H-indol-3-yl)propanoic acid;
2-amino-3-(7-chloro-1-methyl-4-nitro-1H-indol-3-yl)propanoic acid;
2-amino-3-(5-bromo-1-methyl-4-nitro-1H-indol-3-yl)propanoic acid;
2-amino-3-(6-bromo-1-methyl-4-nitro-1H-indol-3-yl)propanoic acid;
2-amino-3-(7-bromo-1-methyl-4-nitro-1H-indol-3-yl)propanoic acid;
2-amino-3-(6-fluoro-2-methyl-4-nitro-1H-indol-3-yl)propanoic acid;
2-amino-3-(7-fluoro-2-methyl-4-nitro-1H-indol-3-yl)propanoic acid;
2-amino-3-(5-chloro-2-methyl-4-nitro-1H-indol-3-yl)propanoic acid;
2-amino-3-(6-chloro-2-methyl-4-nitro-1H-indol-3-yl)propanoic acid;
2-amino-3-(7-chloro-2-methyl-4-nitro-1H-indol-3-yl)propanoic acid;
2-amino-3-(5-bromo-2-methyl-4-nitro-1H-indol-3-yl)propanoic acid;
2-amino-3-(6-bromo-2-methyl-4-nitro-1H-indol-3-yl)propanoic acid;
2-amino-3-(7-bromo-2-methyl-4-nitro-1H-indol-3-yl)propanoic acid;
2-amino-3-(6-fluoro-1,2-dimethyl-4-nitro-1H-indol-3-yl)propanoic acid;
2-amino-3-(7-fluoro-1,2-dimethyl-4-nitro-1H-indol-3-yl)propanoic acid;
2-amino-3-(5-chloro-1,2-dimethyl-4-nitro-1H-indol-3-yl)propanoic acid;
2-amino-3-(6-chloro-1,2-dimethyl-4-nitro-1H-indol-3-yl)propanoic acid;
2-amino-3-(7-chloro-1,2-dimethyl-4-nitro-1H-indol-3-yl)propanoic acid;
2-amino-3-(5-bromo-1,2-dimethyl-4-nitro-1H-indol-3-yl)propanoic acid;
2-amino-3-(6-bromo-1,2-dimethyl-4-nitro-1H-indol-3-yl)propanoic acid;
2-amino-3-(7-bromo-1,2-dimethyl-4-nitro-1H-indol-3-yl)propanoic acid; or
2-amino-3-(5-methoxy-1,2-dimethyl-4-nitro-1H-indol-3-yl)propanoic acid.

50. The method of claim 40, wherein the step of growing the bacterial cell culture comprises introducing one or more antibiotic into the bacterial cell culture, optionally wherein the one or more antibiotic is selected from ampicillin and kanamycin.

51. The method of claim 40, wherein the step of growing the bacterial cell culture comprises introducing one or more inducer into the bacterial cell culture, optionally wherein the one or more inducer is Isopropyl β-D-1-thiogalactopyranoside (IPTG).

52. The method of claim 40, wherein the step of growing the bacterial cell is performed at a temperature below 37° C.

53. The method of claim 40, wherein the bacterial cell culture is grown for up to 25 hours.

54. The method of claim 40, wherein the step of isolating the one or more compounds of Formula (I) or Formula (IV) comprises lysing the recombinant bacterial cell.

55. The method of claim 54, wherein the step of isolating further comprises performing high-pressure liquid chromatography (HPLC) on the bacterial cell lysate, or performing a liquid extraction method on the bacterial lysate.

56. The method of claim 40, wherein:
(a) the NOS enzyme is a bacterial NOS enzyme, optionally wherein the NOS enzyme is a Bacillus subtilis enzyme or wherein the NOS enzyme is encoded by the sequence set forth in SEQ ID NO: 3; and/or
(b) the GDH enzyme is a bacterial GDH enzyme, optionally wherein the bacterial GDH enzyme is a Bacillus megaterium GDH enzyme.

57. The method of claim 40, wherein an isolated nucleic acid engineered to express the NOS enzyme and an isolated nucleic acid engineered to express the GDH enzyme are located on the same plasmid.

58. The method of claim 57, wherein the isolated nucleic acid engineered to express the fusion protein is located on a plasmid that does not contain an isolated nucleic acid engineered to express the NOS enzyme or an isolated nucleic acid engineered to express the GDH enzyme.

59. The method of claim 40, wherein the bacterial cell is a Gram-negative bacterial cell, optionally wherein the bacterial cell is an E. coli cell.
